(19) United States
(12) Patent Application Publication
(10) Pub. No.: US 2022/0275041 A1
(43) Pub. Date: Sep. 1, 2022

(54) MODIFIED IFNL3 POLYPEPTIDES COMPRISING A PHARMACOKINETIC ENHANCING MOIETY AND THEIR USES

(71) Applicant: Exalt Therapeutics, LLC, Las Vegas, NV (US)

(72) Inventors: John W. WALLEN, III, Las Vegas, NV (US); Matthew CALDEMEYER, Las Vegas, NV (US)

(21) Appl. No.: 17/269,363

(22) PCT Filed: Aug. 22, 2019
(86) PCT No.: PCT/US2019/047783

§ 371 Date: Feb. 18, 2021

Related U.S. Application Data
(60) Provisional application No. 62/765,399, filed on Aug. 23, 2018.

(51) Int. Cl. C07K 14/555

(52) U.S. Cl. CPC C07K 14/555; C07K 2319/30; C07K 2319/31; A61K 38/00

(57) ABSTRACT

Non-human IFNL3 polypeptides and their uses thereof are provided. Exemplary embodiments provide IFNL3 polypeptides which include one or more amino acid substitutions, additions, or deletions with natural or non-naturally encoded amino acids, and/or linkage or fusion to other biologically active molecules including other IFNL3 polypeptides, as well as PK enhancing moieties (PKEMs). Additionally, use of said IFNL3 polypeptides for innate immune system stimulation, as a vaccine adjuvant, as well as treatment or prevention of diseases such as viral and bacterial infections, and inflammation, is also provided.

### CROSS-REFERENCE

This application is a national phase entry of International Application No. PCT/US2019/047783 filed on Aug. 22, 2019, which claims the benefit of U.S. Provisional Application No. 62/765,399, filed Aug. 23, 2018, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 22, 2021, is named 55980-701_831_SL.txt and is 23,705 bytes in size.

### BACKGROUND OF THE INVENTION

The recently classified type III interferon (IFN) group consists of three IFN-λ (lambda) molecules called IFN-λ1, IFN-λ2 and IFN-λ3 (also called IL29, IL28A and IL28B respectively). These IFNs signal through a receptor complex consisting of IL10R2 (also called CRF2-4) and IL28RA (also called IFNLR1, CRF2-12). Recently, a new protein with a similar function related to IFN-λ3 was found around the same genomic locus and was designated IFN-λ4. Its intracellular signaling was through IFNLR1 and therefore thought to be a type III interferon. In contrast to type I IFNs, whose receptor is ubiquitously expressed, type III IFNs signal and function largely at barrier epithelial surfaces, such as the respiratory and gastrointestinal tracts, as well as the blood-brain barrier.

The present disclosure relates to non-human Interferon Lambda type 3 (IFNL3) polypeptides and uses thereof in veterinary medicine and animal health. In exemplary embodiments, the IFNL3 polypeptides, such as bovine IFNL3, ovine IFNL3, avian IFNL3, equine IFNL3, canine IFNL3, feline IFNL3, and porcine IFNL3 polypeptides, may include one or more amino acid modifications and/or post-translational modifications that enhance or modulate the biological activity of IFNL3, or pharmacokinetic, pharmacodynamic, or time-action properties of the IFNL3 polypeptide, including linkage to other biologically active molecules such as a half-life extending or pharmacokinetic enhancing moiety. The disclosure further provides pharmaceutical compositions and medical use of such IFNL3 polypeptides.

Viral diseases, and the frequent bacterial sequalae, remain a pervasive problem affecting global animal health, food safety and productivity. Similarly companion animals also suffer from the same problems. These diseases cause significant damage to livestock herds, as well as major annual economic losses, on a global scale. Current vaccine-based regimens for treatment of viral infections are not adequately effective. Vaccine efficacy is negatively impacted by viral pathogen variance, viral antigen mutations, and poor immunogenicity. Inadequate dosing regimens or timing of vaccine administration often fail to induce sufficient immune response. Vaccines have a delayed onset of action and are ineffective in animals with a suppressed immune system. No emergency-use therapeutic exists to attenuate and control acute viral outbreaks which can lead to the destruction of a large portion, or all, of the affected herd. Undertreated viral infections may cause morbidity, mortality and often morph into complex secondary bacterial infections only treatable with antibiotics. There exists an unmet need for a veterinary drug to stimulate the innate immune system of livestock with a natural interferon to prevent and treat various infections using protein based technology in a species-specific manner applicable to a broad range of agricultural animals.

Stimulation of the innate immune system in livestock and companion animals has been the focus of major R&D efforts, and the commercialization of products that mimic infectious agents attempting to accomplish this have been made. Stimulating the innate immune system can lead to unwanted, and even harmful systemic effects, such as inflammation. An approach that initiates stimulation of the innate immune system (IIS) but avoids or significantly lessens the undesirable effects including systemic inflammation, would be advantageous. Such a veterinary drug represents a disruptive new platform for treating viral infections in livestock, complementing and possibly replacing the current standard treatments of antibiotics, vaccines and synthetic stimulants.

Many life-threatening viral infections take effect in the initial postpartum period when offspring are immunocompromised. Enteric viruses (for example, rotavirus, coronavirus, pestivirus) impact porcine, poultry, bovine, avian and ovine livestock globally. Agricultural animals are subjected to a high level of stress, often in inclement and crowded conditions, during transportation, feedlots and production environments. Within such conditions, viral shedding from infected animals creates conditions for the rapid spreading of viral infections throughout the entire herd. Many respiratory viruses (e.g. BRSV, PI-3, BVDV, FMDV, PRRSV, bronchitis, influenza) can have a devastating impact on animal morbidity and mortality, and are known to be precursors to bacterial diseases that require the use of antibiotics. Foot and mouth disease virus (FMDV) is a threat to global food supply and security because no technology exists to stop disease outbreaks except for mass animal culling practices. Porcine reproductive and respiratory system virus (PRRSV) causes respiratory illness and breeding difficulty in large quantities of pigs globally, engendering significant losses in global production yield. Preventive treatment using a natural cytokine that stimulates innate immunity would protect newborns during the first few weeks of life, especially prior to maternal colostrum development, or prevent and mitigate viral outbreaks.

Thus, there is an urgent need to develop a means to safely and effectively stimulate the innate immune system in livestock or companion animal subjects, in particular subjects at high risk of exposure or susceptibility to contracting infectious diseases. The ability to stimulate the IIS by administering IFNL3 or a biologically active form thereof may reduce virus, bacterial, and other infectious agent-mediated morbidity and mortality, and may have an adjuvant effect when used in connection with vaccination.

### SUMMARY

The present invention relates to the use of the interferon lambda type 3 (IFNL3) or a biologically active fragment or modified form of the IFNL3, where the IFNL3 or fragment or modified form thereof is capable of stimulating the innate immune system (IIS) and modulate infectious diseases. cDNA encoding IFNL3 protein has been made and used to express and produce recombinant biologically active IFNL3 protein from multiple non-human species. DNA encoding bovine Interferon lambda 3 is found in GenBank accession number NM_001281901, avian Interferon lambda 3 is found in GenBank accession number NM_001128496, ovine Interferon lambda 3 is found in GenBank accession number NC_019471.2, porcine Interferon lambda 3 is found in GenBank accession number NM_001166490, horse interferon lambda 3 variant 1 is found in GenBank accession number XM_005596230.2, horse interferon lambda 3 variant 2 is found in GenBank accession number XM_023651251.1, canine interferon lambda 3 is found at GenBank Accession number KC754970.1, and feline is found in GenBank accession number XM_006941349.1. Any of the known nucleotide sequences encoding the various species-specific IFNL3 proteins of the present invention are suitable for use herein. The IFNL3 proteins of the present invention may be expressed in recombinant host cells in wild-type form, or in a form that has deleted some or all of the amino acids of the secretion signal peptide since IFNL3 is a secreted protein. Amino acid substitutions, deletions, or additions may also be made using techniques widely known in the art.

As disclosed herein, the present invention includes a pegylated non-human interferon lambda 3 (IFNL3) polypeptide or fragment thereof comprising a sequence having at least 80% identity to SEQ ID NO: 1; SEQ ID NO:2; SEQ ID NO:3; SEQ ID NO 4; SEQ ID NO 5; SEQ ID NO: 6; SEQ ID NO: 7; SEQ ID NO: 8; or SEQ ID NO: 9, or fragment thereof, wherein said polypeptide is covalently linked to at least one polyethylene glycol.

The present invention is drawn to therapeutic proteins for prevention and treatment and/or prevention of viral, bacterial infections, as well as other infectious agents, and is based upon a recombinantly produced engineered protein that stimulates the innate immune system of the animal. IFNL3 is an ideal protein choice for stimulation of innate immunity, and will effectively modulate infectious diseases. IFNL3 is a naturally occurring protein found in a wide variety of non-human species that is produced following natural stimulation of the innate immune system by infectious agents, and exerts its effects directly on cells of the animal. Its primary role in humans is to combat infectious diseases by stimulating cells to activate and express a specific subset of genes that directly impact virus replication. IFNL3 has also been shown to protect cells from bacterial infection and other invasive microorganisms. IFNL3 stimulation of innate immunity in livestock will enable an entirely new approach to preventing and treating infectious disease in agricultural animals in a species-specific manner.

The present invention provides IFNL3 polypeptides and modified IFNL3s, as well as compositions and therapeutic uses thereof. In exemplary embodiments, the modified IFNL3s exhibit enhanced or modulated biological activity, pharmacokinetic, pharmacodynamics, or time-action properties, including an increased or enhanced in vivo half-life relative to wild-type IFNL3, such as an in vivo half-life of at least 1, 2, 3, 4, 5, 6, 9, 10, 12, 15, 20, 25 hours, multiple days, one or more weeks, or longer.

In some embodiments, the IFNL3 polypeptide comprises wild-type or a modified non-human IFNL3 polypeptide. Said IFNL3 or modified IFNL3 protein has at least 80% identity to the IFNL3 polypeptide of SEQ ID NO: 1, SEQ ID NO: 2, SEQ ID NO: 3, SEQ ID NO: 4, SEQ ID NO 5; SEQ ID NO: 6; SEQ ID NO: 7; SEQ ID NO: 8; or SEQ ID NO: 9.

In exemplary embodiments, the IFNL3 or modified IFNL3 retains one or more properties of wild-type IFNL3 that are indicative of clinical efficacy, including inhibiting virus replication, in vitro or in vivo, as a vaccine adjuvant, and efficacy for treatment and/or prevention of viral, bacterial, parasitic, and other infectious diseases, or in a surrogate model thereof. In exemplary embodiments the IFNL3 or modified IFNL3 is linked to at least one pharmacokinetic enhancing moiety (PKEM). Exemplary PKEM include but are not limited to acyl groups, alkyl groups, lipids, serum albumin, XTEN molecules, Fc molecules, adnectins, polymers, and albumin binding moieties. For example, the acyl group may comprise a C8-C30 acyl, such as a C12 acyl, C14 acyl, C16 acyl, C18 acyl, or C20 acyl. In some embodiments the PKEM molecule is linked to the N-terminus or C-terminus of the IFNL3 or modified IFNL3 polypeptide, or at another site. The attachment of a PKEM to an IFNL3 polypeptide of the present invention is referred to herein as “PKEMylation”.

The PKEM may be linked to any amino acid residue of the IFNL3 or modified IFNL3 amino acid sequence, such as before position 1 (i.e. at the N-terminus), 1, 2, 3, 4, 5, 6, 7, 8, 9, 10, 11, 12, 13, 14, 15, 16, 17, 18, 19, 20, 21, 22, 23, 24, 25, and each amino acid position through the final C-terminal amino acid, and after the last amino acid in the sequence (i.e., at the C-terminus of the protein), or the corresponding amino acids in SEQ ID NO: 1, SEQ ID NO: 2, SEQ ID NO: 3; SEQ ID NO 4; SEQ ID NO 5; SEQ ID NO: 6, SEQ ID NO: 7; SEQ ID NO: 8; or SEQ ID NO: 9.

In further exemplary embodiments the PKEM may be linked to any single position in the IFNL3 amino acid sequence, or a combination of more than one of these sites, e.g., 2, 3, 4, or more sites, or at least one of these sites in combination with other sites. Positions in said polypeptide chain may be substituted with another amino acid, such as cysteine (Cys, C) or a non-naturally occurring amino acid (e.g. para-acetyl phenylalanine, para-azido phenylalanine), e.g., in conjunction with linkage of the PKEM to the IFNL3 polypeptide.

In an exemplary embodiment, the IFNL3 or modified IFNL3 may include at least one non-naturally encoded amino acid incorporated at any desired position in the amino acid sequence of the protein. Said non-naturally encoded amino acid may be linked to the PKEM, a linker, a biologically active molecule, or another IFNL3 polypeptide. For example, the IFNL3 or modified IFNL3 may include a PKEM linked to a non-naturally encoded amino acid at any position of the IFNL3 polypeptide by, for example, oxime bond formation, or through a click chemistry reaction.

In another aspect, the disclosure provides an IFNL3 or modified IFNL3 polypeptide comprising a substitution of a naturally encoded or non-naturally encoded amino acid substituted in the amino acid sequence, wherein: (a) the IFNL3 polypeptide comprises an IFNL3 polypeptide that has a sequence at least 80% identical to SEQ ID NO: 1, or at least 80% identical to SEQ ID NO: 2 or SEQ ID NO: 3; SEQ ID NO 4; SEQ ID NO 5; SEQ ID NO: 6; SEQ ID NO: 7; SEQ ID NO: 8; or SEQ ID NO: 9; and (b) the substituted naturally encoded or non-naturally encoded amino acid is linked to a PKEM.

In another aspect, the disclosure provides an IFNL3 or modified IFNL3 polypeptide comprising up to one, two, three, or four amino acid substitutions selected from substitution with naturally encoded or non-naturally encoded amino acids. In another aspect, the disclosure provides an IFNL3 or modified IFNL3 polypeptide comprising at least one natural amino acid substitution and/or at least one non-naturally encoded amino acid substitution and substituted amino acid is linked to a linker, polymer, or biologically active molecule.

Said IFNL3 polypeptide may include a non-naturally encoded amino acid having the structure:

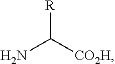

wherein the R group is any substituent other than the side chain found in alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, pyrrolysine, or selenocysteine.

As stated above, in exemplary embodiments the IFNL3 or modified IFNL3 is linked to at least one PKEM comprising an XTEN molecule. XTEN molecules are also referred to as unstructured recombinant polymers, unstructured recombinant polypeptides, or URPs, and are generally described in Schellenberger et al., Nat Biotechnol., 2009 December; 27(12):1186-90, U.S. Pub. No. 2012/0220011, U.S. Pat. No. 7,846,445, and WO/2012/162542, each of which is hereby incorporated by reference in its entirety. As disclosed therein, the half-life of the IFNL3 or modified IFNL3 polypeptide may be varied by varying the constitution of the XTEN molecule, e.g., by varying its size. For example, an XTEN molecule may be selected in order to achieve a desired half-life, such as in the range of 1 to 50 hours, such as at least 1, 2, 5, 10, 12, 15, 20, or 25 hours, or longer.

Exemplary XTEN molecules include a URP comprising at least 40 contiguous amino acids, wherein: (a) the URP comprises at least three different types of amino acids selected from the group consisting of glycine (G), aspartate (D), alanine (A), serine (S), threonine (T), glutamate (E) and proline (P) residues, wherein the sum of said group of amino acids contained in the URP constitutes more than about 80% of the total amino acids of the URP, and wherein said URP comprises more than one proline residue, and wherein said URP possesses reduced sensitivity to proteolytic degradation relative to a corresponding URP lacking said more than one proline residue; (b) at least 50% of the amino acids of said URP are devoid of secondary structure as determined by Chou-Fasman algorithm; and (c) the Tepitope score of said URP is less than −5. Additional exemplary XTEN molecules comprise an unstructured recombinant polymer (URP) comprising at least about 40 contiguous amino acids, and wherein (a) the sum of glycine (G), aspartate (D), alanine (A), serine (S), threonine (T), glutamate (E) and proline (P) residues contained in the URP, constitutes at least 80% of the total amino acids of the URP, and the remainder, when present, consists of arginine or lysine, and the remainder does not contain methionine, cysteine, asparagine, and glutamine, wherein said URP comprises at least three different types of amino acids selected from glycine (G), aspartate (D), alanine (A), serine (S), threonine (T), glutamate (E) and proline (P); (b) at least 50% of the at least 40 contiguous amino acids in said URP are devoid of secondary structure as determined by Chou-Fasman algorithm; and (c) wherein the URP has a Tepitope score less than −4.

Multiple IFNL3 polypeptide molecules may be linked to an XTEN molecule, e.g., up to 1, 2, 3, 4, 5, or more IFNL3 polypeptide molecules per XTEN molecule. For example the IFNL3 polypeptide molecules may be linked to sites on differing portions of the XTEN molecule, e.g., near the N-terminus, near the C-terminus, or near the middle (mid-way between the N- and C-termini) thereof. In this context, the term “near” generally means linked to a site within a region of about 20%, about 15%, about 10%, or about 5% of the residues at the respective terminus or centered at the middle of the XTEN molecule.

Additional exemplary XTEN molecules include a hydrophobic residue (e.g., F, I, L, M, V, W or Y), a side chain amide-containing residue (e.g., N or Q) or a positively charged side chain residue (e.g., H, K or R). In some embodiments, the duration enhancing moiety includes A, E, G, P, S or T. In some embodiments, the XTEN includes glycine at 10-20%, 20-30%, 30-40%, 40-50%, 50-60%, 60-70%, 70-80%, 80-90%, 90-99%, or even glycine at 100%. Said XTEN molecules may be further linked to a polyethylene glycol polymer (PEG). Said IFNL3 or modified IFNL3 or modified IFNL3 polypeptide or polypeptides may be linked to said XTEN molecules through a dibenzylcyclooctyne (DBCO).

In exemplary embodiments the IFNL3 or modified IFNL3 is linked to at least one PKEM comprising an adnectin. Exemplary adnectins are disclosed in U.S. 2011/0305663, which is hereby incorporated by reference in its entirety. Said adnectin may be based on a tenth fibronectin type III domain and may bind to serum albumin. Said adnectin may comprise one or more of a BC loop, a DE loop, and an FG loop, or comprises a polypeptide selected from SEQ ID NO: 5, 6, 7, 8, 12, 16, 20, and 24-44 of U.S. Pub. No. 2011/0305663.

Also as stated above, in exemplary embodiments the IFNL3 or modified IFNL3 is linked to at least one PKEM comprising serum albumin, such as human or non-human serum albumin. For example said IFNL3 or modified IFNL3 polypeptide may be linked to the Cys 34 residue of said human or non-human serum albumin. In one embodiment the albumin binding moiety comprises a carboxylic acid group, such as HOOC(CH₂)ₛCO—, wherein s is an integer from 12 to 22, such as 10, 12, 16 or 18.

Also as stated above, in exemplary embodiments the IFNL3 or modified IFNL3 is linked to at least one PKEM comprising an acyl group. An exemplary acyl group has from 6 to 40 carbon atoms, from 8 to 26 carbon atoms or from 14 to 22 carbon atoms, such as 16, 17, 18, 19, 20 carbon atoms, which may be branched or unbranched. In another embodiment the PKEM comprises an acyl group selected from CH₃(CH₂)ᵣCO—, wherein r is an integer from 4 to 38, such as an integer from 4 to 24, an integer from 6 to 20, or 10 or 12, preferably selected from the group comprising CH₃(CH₂)₆CO—, CH₃(CH₂)₈CO—, CH₃(CH₂)₁₀CO—, CH₃(CH₂)_{1,2}CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—. In one embodiment the acyl group may comprise a group which can be negatively charged at pH 7.4.

In exemplary embodiments the acyl group may comprise a terminal acidic group and may comprise at least two acidic groups wherein one acidic group is attached terminally. For example, the acyl group may comprise a linear or branched lipophilic moiety containing 4-40 carbon atoms having a distal acidic group. Additional exemplary acyl groups are disclosed in U.S. 2012/0295847, which is hereby incorporated by reference in its entirety.

In exemplary embodiments, the PKEM is linked to the IFNL3 or modified IFNL3 polypeptide through a linker. For example, the linker can comprise one or two amino acids which at one end bind to the PKEM—such as an albumin binding moiety—and at the other end bind to any available position on the polypeptide backbone. Additional exemplary linkers include a hydrophilic linker such as a chemical moiety which comprises at least 5 non-hydrogen atoms where 30-50% of these are either N or O. Additional exemplary linkers which may link said PKEM to said IFNL3 or modified IFNL3 are disclosed in U.S. 2012/0295847 and WO/2012/168430, each of which is hereby incorporated by reference in its entirety.

In further exemplary embodiments, in addition to said PKEM said IFNL3 or modified IFNL3 polypeptide also includes a polyethylene glycol, which may be of a molecular weight within a range of about 5 kDa to 100 kDa, or another size. Said polyethylene glycol may be linked to the IFNL3 at any suitable position in the amino acid sequence of the polypeptide, including but not limited to the N-terminus or the C-terminus.

In exemplary embodiments the IFNL3 or modified IFNL3 may be a fusion protein comprising IFNL3 and a proteinaceous PKEM (e.g., albumin, an Fc chain, certain XTEN molecules, or a PKE adnectin), which may be fused to any suitable amino acid of the IFNL3 polypeptide and may be fused to the N- or C-terminus thereof.

The PKEM may be covalently linked to said IFNL3 or modified IFNL3, e.g., covalently linked to a naturally encoded or a non-naturally encoded amino acid. For example, the IFNL3 or modified IFNL3 may comprise a PKEM linked to a cysteine via a thiol linkage, or linked to N-terminal amines using aldehyde derivatives via reductive alkylation.

Optionally, multiple IFNL3 or modified IFNL3 molecules may be joined by a linker polypeptide, wherein said linker polypeptide optionally is 1, 1-2, 1-3, 1-4, 1-5, 1-6, 1-7, 1-8, 1-9, 1-10, 1-11, 1-12 amino acids in length, and longer in length, wherein optionally the N-terminus of one IFNL3 polypeptide is fused to the C-terminus of the linker polypeptide and the N-terminus of the linker polypeptide is fused to the N-terminus of another IFNL3 polypeptide. Further exemplary linker polypeptides which may be utilized are disclosed in WO/2013/004607, which is hereby incorporated by reference in its entirety.

In another embodiment two IFNL3 polypeptides are linked to form a homodimer of IFNL3, or a homodimer of a modified IFNL3, or a heterodimer of IFNL3 and a modified IFNL3, or a heterodimer of different modified IFNL3 polypeptides, or any combination of IFNL3 polypeptides. The IFNL3 dimer may be formed by chemical linking of their respective N-termini. It is understood that linking two IFNL3 polypeptides each having a molecular weight of about 20 kDa, in a head-to-head fashion each at their N-terminus, may result in a biologically active molecule that would have an overall molecular weight of about 40 kDa. A polypeptide of this size may have significantly reduced kidney clearance from the bloodstream, which may in turn result in a significantly increased circulating half-life after administration to a subject. Therefore, a dimer of IFNL3 polypeptides may have the same half-life extending effect compared to monomeric IFNL3, as observed using a single IFNL3 polypeptide chemically linked to a non-IFNL3 PKEM.

In an exemplary embodiment, a PKEM is linked to a naturally encoded amino acid or a non-naturally encoded amino acid, which may comprise a functional group which reacts and thereby forms a covalent bond with functional group on the PKEM.

In another exemplary embodiment, the disclosure provides one or more polynucleotides encoding the IFNL3. Said IFNL3-coding polynucleotides may be contained in different molecules or in the same molecules, e.g., joined in any order and optionally joined via a nucleotide sequence that encodes a connecting peptide. Said polynucleotide may be isolated. Said polynucleotide may be contained in one or more vectors, plasmids, etc., such as an expression vector. Additional exemplary embodiments provide a composition for translation of said polynucleotides, such as a cell or in vitro translation system comprising said polynucleotides. Further exemplary embodiments provide a host cells, such as a prokaryotic cell (e.g., E. coli) or eukaryotic cell (e.g., a yeast or mammalian cell) comprising said polynucleotide and optionally further comprising an orthogonal RS or tRNA. Additional exemplary embodiments provide a method of producing an IFNL3 or modified IFNL3, comprising causing a cell or in vitro translation system to translate said polynucleotide or an mRNA transcribed therefrom. Optionally, the IFNL3 or modified IFNL3 may include one or more natural variant sequences of IFNL3, such as the IFNL3 isoform 2. Additional natural variant sequences which may be present include sequences in the nucleic acid that encode the secretion signal sequence.

In exemplary embodiments, the IFNL3 or modified IFNL3 has one or more biological activities of IFNL3, such as the inhibition of virus replication. Said anti-virus replication activity may be determined using any of the methods described herein, e.g., in the examples, or any other method known in the art.

Another embodiment provides a pharmaceutical composition or medicament comprising an IFNL3 or modified IFNL3. A further embodiment provides the use of said IFNL3 or modified IFNL3 in the prevention and/or treatment of a variety of diseases including but not limited to diseases caused by viruses, bacteria, parasites, or any other infectious agent, or as an adjuvant for vaccines. Exemplary embodiments of the present disclosure provide use of an IFNL3 or modified IFNL3 as a medicament for the prophylaxis and/or treatment of viral diseases. In addition, an IFNL3 or modified IFNL3 of the disclosure can be used as a medicament for the prophylaxis and/or treatment of infectious diseases or as an adjuvant for vaccines in newborn or unborn animals.

In addition, the present disclosure provides the use of an IFNL3 or modified IFNL3 of the disclosure as a medicament for the prophylaxis and/or treatment of bacterial infections. In addition, the present disclosure provides the use of an IFNL3 or modified IFNL3 of the disclosure as a medicament for the prophylaxis and/or treatment of parasitic infections.

The present disclosure furthermore provides the use of IFNL3 or modified IFNL3 for preparing a medicament for the treatment and/or prevention of disorders, in particular inflammation or the disorders mentioned above. The present disclosure furthermore provides a method for the treatment and/or prevention of disorders, in particular the disorders mentioned above, using an effective amount of at least one IFNL3 or modified IFNL3 of the disclosure. The present disclosure furthermore provides an IFNL3 or modified IFNL3 for use in a method for the treatment and/or prophylaxis of disease with reduced levels of inflammation.

The present disclosure furthermore provides an IFNL3 or modified IFNL3 for use as an adjuvant in connection with a vaccine for the treatment and/or prophylaxis of diseases of animals, including livestock and companion animals.

The present disclosure further provides combinations of at least one modified IFNL3 with at least one additional drug such as antibiotics, vaccines, another interferon including but not limited to interferon alpha, interferon beta, or interferon gamma, interferon lambda 1, interferon lambda 2, interferon lambda 4, antiviral drug or antiparasitic drug, or immune stimulant. In another embodiment, an IFNL3 or modified IFNL3 is administered to stimulate the innate immune system of the animal. In another embodiment, the IFNL3 or modified IFNL3 is administered as an anti-inflammatory agent. In another embodiment, the IFNL3 or modified IFNL3 is administered in combination with another cytokine or interferon.

Other embodiments provide combinations comprising at least one of the IFNL3 or modified IFNL3 and also one or more further active ingredients selected from the group consisting of cytokines, immune stimulants, vaccines, antibiotics, antiviral agents, antiparasitic drugs, and also their use for the treatment and/or prevention of the disorders mentioned above.

The present disclosure furthermore provides medicaments comprising at least one modified IFNL3, usually together with one or more inert nontoxic pharmaceutically suitable auxiliaries, and also their use for the purposes mentioned above.

In another aspect, the disclosure provides an IFNL3 or modified IFNL3 polypeptide comprising: at least 80% identity to SEQ ID NO: 1, 2, 3, 4, 5, 6, 7, 8, or 9 and at least one PKEM linked to the IFNL3 polypeptide, which PKEM is optionally linked to at least one amino acid contained in said IFNL3 or modified IFNL3, wherein said IFNL3 polypeptide is biologically active, and wherein said PKEM optionally comprises at least one acyl group, lipid, alkyl group, carbohydrate, polypeptide, polynucleotide, polysaccharide, antibody or antibody fragment, sialic acid(s), a prodrug, serum albumin, XTEN molecule, Fc molecule, adnectin, fibronectin, a biologically active molecule, or a combination thereof.

The IFNL3 or modified IFNL3 polypeptide sequence may be at least 90% identical to SEQ ID NO: 1, 2, 3, 4, 5, 6, 7, 8, or 9. Said IFNL3 or modified IFNL3 may comprise zero, one, two, three, or four amino acid substitutions, insertions, or deletions (from either the N-terminus or C-terminus or internally), wherein said substitutions are with natural or non-naturally encoded amino acids. Said PKEM may be linked to said naturally encoded or non-naturally encoded amino acid that is substituted in the IFNL3 amino acid sequence.

In another aspect, the disclosure provides an isolated cell, vector, plasmid, prokaryotic cell, eukaryotic cell, virus, insect cell, mammalian cell, yeast, bacterium, or cell-free translation system comprising one or more polynucleotides that encode the IFNL3 or modified IFNL3 of the present invention to express the IFNL3 or modified IFNL3 polypeptide. The method of expression may produce any IFNL3 or modified IFNL3 polypeptide as herein described.

In another aspect, the disclosure provides a method of producing any IFNL3 or modified IFNL3 polypeptide as herein described, comprising chemically synthesizing said IFNL3 or modified IFNL3 polypeptide.

The method may further comprise purifying said IFNL3 or modified IFNL3 polypeptide to provide a substantially pure IFNL3 polypeptide composition.

In further embodiments, the IFNL3 may be administered daily or at any other desirable and effective frequency, in an injectable formulation, an orally-available formulation, as a sustained release formulation, as a prodrug formulation, or as a continuous infusion.

The invention further provides a method of inhibiting infectious agents, including but not limited to inhibiting virus, bacteria, parasitic organism replication.

The invention further provides a method of enhancing the immune response to vaccines. The IFNL3 proteins of the present invention may be used as adjuvants in conjunction with vaccination, for example.

In some embodiments, the IFNL3 polypeptide comprises one or more post-translational modifications, including but not limited to glycosylation. In some embodiments, the IFNL3 polypeptide is linked to a linker, polymer, or biologically active molecule. In some embodiments, the IFNL3 polypeptide is linked to a bifunctional or multi-functional polymer, bifunctional or multi-functional linker, or at least one additional IFNL3 polypeptide or other interferon or cytokine.

In some embodiments, the IFNL3 or modified IFNL3 is linked to a PKEM. In some embodiments, the IFNL3 is linked to the PKEM with a linker or is bonded to the PKEM. In some embodiments, the PKEM is a bifunctional or multi-functional molecule. In some embodiments, the bifunctional or multi-functional molecule is linked to one or more additional polypeptides. In some embodiments, the additional polypeptide is an IFNL3 or another interferon or cytokine polypeptide. In some embodiments, the IFNL3 polypeptide comprises at least two amino acids linked to a PKEM. In some embodiments, at least one amino acid is a non-naturally encoded amino acid.

In some embodiments, one or more naturally encoded or non-naturally encoded amino acids are incorporated in one or more of the following positions in any of the IFNL3 polypeptide, or proIFNL3 polypeptides, IFNL3 analogs, proIFNL3, or modified IFNL3 polypeptide amino acid sequence before position 1 (i.e. at the N-terminus), 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and at each individual amino acid position up to and including after the last amino acid at the C terminus (i.e., at the carboxyl terminus of the protein of SEQ ID NO: 1, the corresponding position in SEQ ID NO: 2, or SEQ ID NO:3; SEQ ID NO 4; SEQ ID NO 5; SEQ ID NO: 6; SEQ ID NO: 7; SEQ ID NO: 8; or SEQ ID NO: 9

The sites selected for incorporation, deletion, addition, or substitution of a naturally encoded or of a non-naturally encoded amino acid that enhance or modulate pharmacokinetic, pharmacodynamic, or time-action properties of the IFNL3 polypeptide, and/or for linkage to a PKEM or other biologically active molecule may be selected based upon a variety of factors which may be predicted to influence the activity and half-life of the resulting modified IFNL3 polypeptide. One factor to consider is the evolutionary conservation of a residue, which may indicate the ability of that site to tolerate a sequence change. Another factor to consider is the residue's participation in forming multimers including homodimerization, binding of IFNL3 modulators, and receptor binding, or proximity to residues involved in the above activities, wherein modification of that residue can enhance or modulate pharmacokinetic, pharmacodynamic, or time-action properties of the IFNL3 polypeptide, and/or for linkage to a PKEM or other biologically active molecule might interfere with activity. Yet another factor to consider is proximity to residues which may interact with the PKEM. For example, where the PKEM is a hydrophobic molecule that binds to serum albumin, proximity to surface-exposed hydrophobic residues may cause the PKEM to bind to the IFNL3 or modified IFNL3 polypeptide and potentially interfere with the ability of the PKEM to bind to serum albumin effectively increase half-life. Likewise, a hydrophilic PKEM could interact with proximate hydrophilic residues and therefore decrease the ability of the PKEM to improve half-life. Still another factor to consider is the effect on activity or half-life resulting from any previously produced modified polypeptide. Still another factor to consider is whether a particular site is known to be chemically or enzymatically unstable, as modification of an unstable residue could potentially improve any adverse effects on half-life resulting from this instability. These factors are considered illustrative rather than exhaustive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the SDS PAGE, Western Blot and HPLC results for IFNL3 protein EXLT-01: Panel A, and Panel B and PanelC show the SDS PAGE, Western Blot and HPLC results, respectively, for IFNL3 protein EXLT-01.

FIG. 2 illustrates the SDS PAGE, Western Blot and HPLC results for IFNL3 protein EXLT-02: Panel A, and Panel B and Panel C show the SDS PAGE, Western Blot and HPLC results, respectively, for IFNL3 protein EXLT-02.

FIG. 3 illustrates the SDS PAGE, Western Blot and HPLC results for IFNL3 protein EXLT-03: Panel A, and Panel B and Panel C show the SDS PAGE, Western Blot and HPLC results, respectively, for IFNL3 protein EXLT-03.

FIG. 4 illustrates the SDS PAGE, Western Blot and HPLC results for IFNL3 protein EXLT-04: Panel A, and Panel B show the SDS PAGE, and Western Blot results, respectively, for IFNL3 protein EXLT-04.

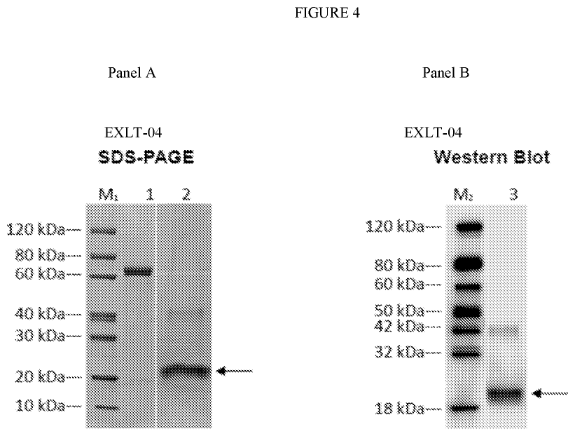

FIG. 5 illustrates the SDS PAGE, Western Blot and HPLC results for IFNL3 protein EXLT-05: Panel A, and Panel B show the SDS PAGE, and Western Blot results, respectively, for IFNL3 protein EXLT-05.

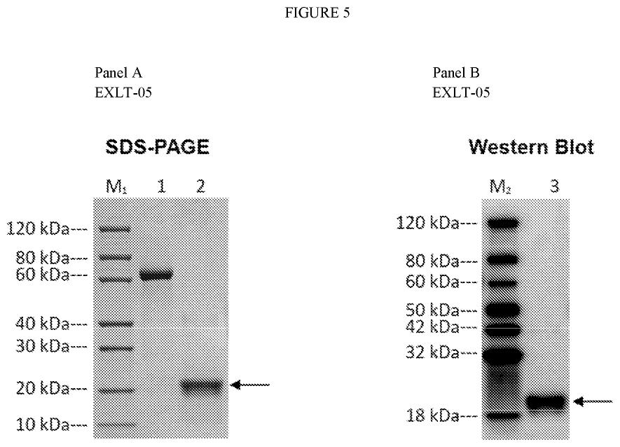

FIG. 6 illustrates the SDS PAGE, Western Blot and HPLC results for IFNL3 protein EXLT-06: Panel A, and Panel B show the SDS PAGE, and Western Blot results, respectively, for IFNL3 protein EXLT-06.

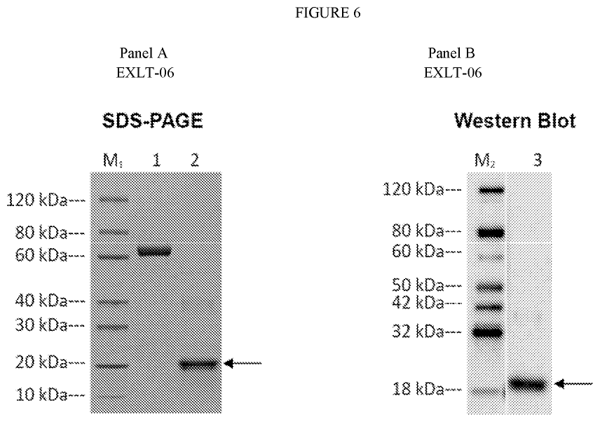

FIG. 7 illustrates the SDS PAGE, Western Blot and HPLC results for IFNL3 protein EXLT-07: Panel A, and Panel B show the SDS PAGE, and Western Blot results, respectively, for IFNL3 protein EXLT-07.

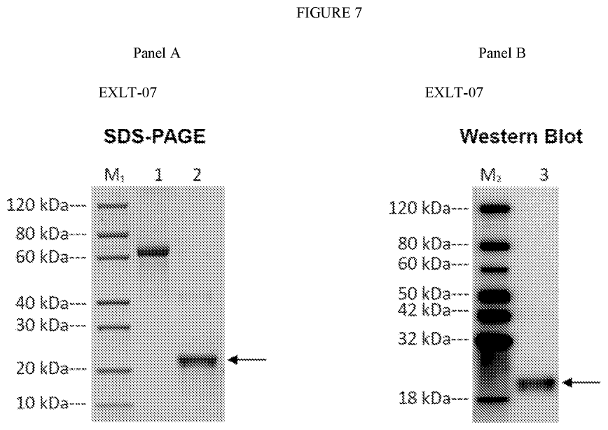

FIG. 8 illustrates the SDS PAGE, Western Blot and HPLC results for IFNL3 protein EXLT-08: Panel A, and Panel B show the SDS PAGE, and Western Blot results, respectively, for IFNL3 protein EXLT-08.

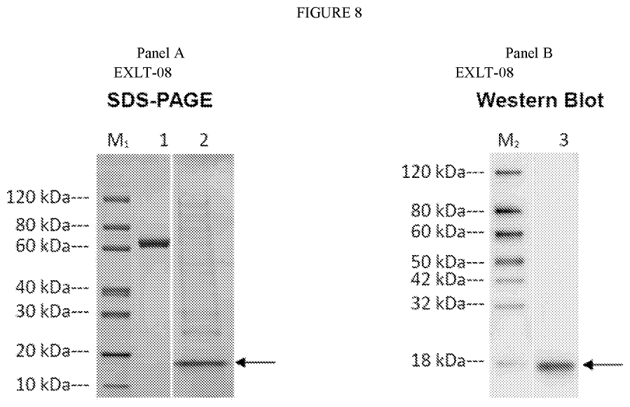

FIG. 9 illustrates the SDS PAGE, Western Blot and HPLC results for IFNL3 protein EXLT-09: Panel A, and Panel B show the SDS PAGE, and Western Blot results, respectively, for IFNL3 protein EXLT-09.

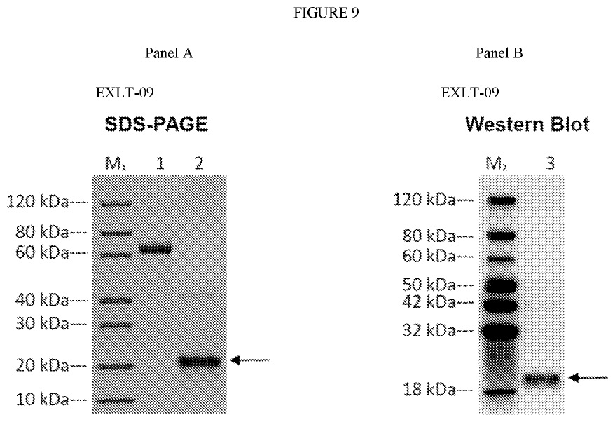

FIG. 10 shows the antivirus activity in a plaque reduction assay for EXLT-01, EXLT-02 and EXLT-03 on MDBK cells infected with BVDV.

FIG. 11 shows virus plaque inhibition assay results for Non-PEGylated EXLT-01 and PEGylated EXLT-01 on MDBK cells infected with BVDV: Panel A and Panel B show virus plaque inhibition assay results for Non-PEGylated EXLT-01 and PEGylated EXLT-01 respectively.

### DETAILED DESCRIPTION

It is to be understood that this invention is not limited to the particular methodology, protocols, cell lines, constructs, and reagents described herein and as such may vary. It is also to be understood that the terminology used herein is for the purpose of describing particular embodiments only, and is not intended to limit the scope of the present invention, which will be limited only by the appended claims.

As used herein and in the appended claims, the singular forms “a,” “an,” and “the” include plural reference unless the context clearly indicates otherwise. Thus, for example, reference to an “IFNL3” or “IFNL3 polypeptide” and various hyphenated and unhyphenated forms is a reference to one or more such proteins and includes equivalents thereof known to those of ordinary skill in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

The term “IFNL3” as used herein, refers to non-human IFNL3, unless the context indicates otherwise, whose amino acid sequence and spatial structure are well-known, including but not limited to the amino acid sequences set forth in SEQ ID NO: 1; SEQ ID NO:2; SEQ ID NO:3; SEQ ID NO 4; SEQ ID NO 5; SEQ ID NO: 6; SEQ ID NO: 7; SEQ ID NO: 8; or SEQ ID NO: 9. IFNL3 is comprised of about 175-185 amino acid long polypeptide chain that contains intra-chain disulfide bonds.

The terms “wild-type IFNL3,” “WT IFNL3,” and “wt IFNL3” refer to an IFNL3 polypeptide having the amino acid sequence of the naturally occurring form of the protein, including but not limited to SEQ ID NO: 1 or SEQ ID NO: 2, or SEQ ID NO:3; SEQ ID NO 4; SEQ ID NO 5; SEQ ID NO: 6; SEQ ID NO: 7; SEQ ID NO: 8; or SEQ ID NO: 9. The terms “wild-type IFNL3,” “WT IFNL3,” and “wt rIFNL3” also refer to a non-human IFNL3 polypeptide having the amino acid sequence SEQ ID NO: 1 or SEQ ID NO: 2, or SEQ ID NO:3; SEQ ID NO 4; SEQ ID NO 5; SEQ ID NO: 6; SEQ ID NO: 7; SEQ ID NO: 8; or SEQ ID NO: 9. WT IFNL3 mature polypeptides in monomeric form each have a predicted molecular weight of about 20 kDa.

The term “IFNL3 analog” as used herein is a protein exhibiting one or more of the biological activities of IFNL3, which may include the activity of stimulating the IIS. For example, the term “IFNL3 analog” includes a protein that differs from the wild-type IFNL3 by having one or more amino acid deletions, one or more amino acid replacements, and/or one or more amino acid additions, and/or post-translational modifications that do not destroy the IFNL3 activity of the IFNL3 analog. An IFNL3 analog having an isoelectric point that is different from the isoelectric point of wild-type IFNL3 is one non-limiting example of an IFNL3 analog. Other examples of IFNL3 analogs include but are not limited to amino acid differences that enhance or modulate biological activity, pharmacokinetic, pharmacodynamic, or time-action properties of the IFNL3 polypeptide, and/or are useful for linkage to a PKEM or other biologically active molecule.

In the present specification, whenever the term IFNL3 is used in a plural or a generic sense it is intended to encompass both naturally occurring IFNL3s and IFNL3 analogues and derivatives thereof.

By “IFNL3 polypeptide” as used herein is meant a compound having a molecular structure similar to that of non-human IFNL3, and which has at least one IFNL3 biologic activity. As used herein, “IFNL3” shall include those polypeptides and proteins that have at least one biological activity of an IFNL3, as well as IFNL3 analogs, IFNL3 isoforms, IFNL3 mimetics, IFNL3 fragments, hybrid IFNL3 proteins, fusion proteins oligomers and multimers, homologues, glycosylation pattern variants, and muteins, thereof, regardless of the biological activity of same, and further regardless of the method of synthesis or manufacture thereof including, but not limited to, recombinant (whether produced from cDNA, genomic DNA, synthetic DNA or other form of nucleic acid), synthetic, transgenic, and gene activated methods. The amino acid sequence and polynucleotide sequence for IFNL3 are shown in Tables 4 and 5 herein. The term “IFNL3 polypeptide” also includes the pharmaceutically acceptable salts and prodrugs, and prodrugs of the salts, polymorphs, hydrates, solvates, biologically-active fragments, biologically active variants and stereoisomers of the naturally-occurring IFNL3 as well as variants of the naturally-occurring IFNL3 and polypeptide fusions thereof. Fusions comprising additional amino acids at the amino terminus, carboxyl terminus, or both, are encompassed by the term “IFNL3 polypeptide.” Exemplary fusions include, but are not limited to, e.g., methionyl IFNL3 in which a methionine is linked to the N-terminus of IFNL3 resulting from the recombinant expression of the mature form of IFNL3 lacking the leader or signal peptide or portion thereof (a methionine is linked to the N-terminus of IFNL3 resulting from the recombinant expression), fusions for the purpose of purification (including, but not limited to, to poly-histidine or affinity epitopes), fusions with serum albumin binding peptides and fusions with serum proteins such as serum albumin. Chimeric molecules comprising IFNL3 and one or more other molecules are also included. The chimeric molecule can contain specific regions or fragments of one or both of the IFNL3 and the other molecule(s). Any such fragments can be prepared from the proteins by standard biochemical methods, or by expressing a polynucleotide encoding the fragment. IFNL3, or a fragment thereof, can be produced as a fusion protein comprising human serum albumin (HSA), Fc, or a portion thereof. Such fusion constructs are suitable for enhancing expression of the IFNL3, or fragment thereof, in an eukaryotic host cell. Exemplary HSA portions include the N-terminal polypeptide (amino acids 1-369, 1-419, and intermediate lengths starting with amino acid 1), as disclosed in U.S. Pat. No. 5,766,883, and publication WO 97/24445, which are incorporated by reference herein. Other chimeric polypeptides can include a HSA protein with IFNL3, or fragments thereof, attached to each of the C-terminal and N-terminal ends of the HSA. Such HSA constructs are disclosed in U.S. Pat. No. 5,876,969, which is incorporated by reference herein. Other fusions may be created by fusion of IFNL3 with a) the Fc portion of an immunoglobulin; b) an analog of the Fc portion of an immunoglobulin; and c) fragments of the Fc portion of an immunoglobulin. The Fc portion of the immunoglobulin may be from the same species as the IFNL3, such as bovine Fc, porcine Fc, canine Fc, feline Fc, ovine Fc, avian Fc, or equine Fc.

Various references disclose modification of polypeptides by polymer conjugation or glycosylation. The term “IFNL3 polypeptide” includes polypeptides conjugated to a PKEM and may be comprised of one or more additional derivatizations of cysteine, lysine, or other residues. In addition, the IFNL3 polypeptide may comprise a linker or polymer, wherein the amino acid to which the linker or polymer is conjugated may be a non-natural amino acid according to the present invention, or may be conjugated to a naturally encoded amino acid utilizing techniques known in the art such as coupling to lysine or cysteine.

The term “IFNL3 polypeptide” also includes glycosylated IFNL3, such as but not limited to, polypeptides glycosylated at any amino acid position, N-linked, or O-linked glycosylated forms of the polypeptide. Variants containing single nucleotide changes are also considered as biologically active variants of IFNL3 polypeptide. In addition, splice variants are also included. The term “IFNL3 polypeptide” also includes IFNL3 polypeptide heterodimers, homodimers, heteromultimers, or homomultimers of any one or more IFNL3 polypeptides or any other polypeptide, protein, carbohydrate, polymer, small molecule, linker, ligand, or other biologically active molecule of any type, linked by chemical means or expressed as a fusion protein, as well as polypeptide analogues containing, for example, specific deletions or other modifications yet maintain biological activity.

The term “IFNL3 polypeptide” or “IFNL3” encompasses IFNL3 polypeptides comprising one or more amino acid substitutions, additions or deletions. IFNL3 polypeptides of the present invention may be comprised of modifications with one or more natural amino acids in conjunction with one or more non-natural amino acid modification. Exemplary substitutions in a wide variety of amino acid positions in naturally-occurring IFNL3 polypeptides, including but not limited to substitutions that modulate pharmaceutical stability, that modulate one or more of the biological activities of the IFNL3 polypeptide, such as but not limited to, increase or decrease enzymatic activity, increase or decrease solubility of the IFNL3 polypeptide, increase or decrease protease susceptibility, increase or decrease homodimerization, increase or decrease zinc binding, increase or decrease stability of the IFNL3 polypeptide, etc. and are encompassed by the term “IFNL3 polypeptide.” In some embodiments, the IFNL3 polypeptide is linked to a PKEM or other biologically active molecule, present in an IFNL3 polypeptide binding region of the IFNL3 molecule.

In some embodiments, the IFNL3 polypeptides further comprise an addition, substitution or deletion that modulates biological activity of the IFNL3 polypeptide. For example, the additions, substitutions or deletions may modulate one or more properties or activities of IFNL3. For example, the additions, substitutions or deletions may modulate affinity for the IFNL3 receptor, modulate circulating half-life, modulate therapeutic half-life, modulate stability of the polypeptide, modulate cleavage by proteases, modulate dimerization of IFNL3, modulate dose, modulate release or bio-availability, facilitate purification, or improve or alter a particular route of administration. Similarly, IFNL3 polypeptides may comprise protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification or other traits of the polypeptide.

The term “IFNL3 polypeptide” also encompasses homodimers, heterodimers, homomultimers, and heteromultimers that are linked, including but not limited to those linked directly via the N-termini, the C-termini, a naturally encoded or non-naturally encoded amino acid side chains, either to the same or different naturally encoded or non-naturally encoded amino acid side chains, to naturally-encoded amino acid side chains, or indirectly via a linker. Exemplary linkers including but are not limited to, small organic compounds, or a PKEM.

The term “pharmacokinetic enhancing moiety” (also referred to herein as “PKEM”) refers to a pharmaceutically acceptable moiety, domain, or “vehicle” covalently linked (“conjugated”) to the IFNL3 polypeptide directly or via a linker, that prevents or mitigates in vivo clearance or proteolytic degradation or other activity-diminishing chemical modification of the IFNL3 polypeptide, increases half-life or other pharmacokinetic properties such as but not limited to increasing the rate of absorption, reduces toxicity, improves solubility, increases biological activity, catalytic efficiency and/or target selectivity of the IFNL3 polypeptide, increases manufacturability, and/or reduces immunogenicity of the IFNL3 polypeptide, compared to an unconjugated form of the IFNL3 polypeptide. The term “pharmacokinetic enhancing moiety” includes non-proteinaceous PKEM, such as polyethylene glycol (PEG) or hydroxyethyl starch (HES), and proteinaceous PKEM, such as serum albumin, transferrin, adnectins (e.g., PKE adnectins), XTEN's, or Fc domain.

The term “albumin binding moiety” as used herein refers to any chemical group capable of binding to albumin, i.e. has albumin binding affinity. In one embodiment the albumin binding moiety is an acyl group.

“Albumin binding affinity” may be determined by several methods known within the art. In one method the compound to be measured is radiolabeled with e.g. 1251 or 3H and incubated with immobilized albumin (Kurtzhals et. al., Biochem. J., 312, 725-731 (1995)). The binding of the compound relative to a standard is calculated. In another method a related compound is radiolabeled and its binding to albumin immobilized on e.g. SPA beads (scintillation proximity assay beads, PerkinElmer cat no. RPNQ0001) is competed by a dilution series of the compound to be measured. The EC50 value for the competition is a measure of the affinity of the compound. In a third method, the substrate affinity or potency of a compound is measured at different concentrations of albumin, and the shift in relative affinity or potency of the compound as a function of albumin concentration reflects its affinity for albumin.

A “non-naturally encoded amino acid” refers to an amino acid that is not one of the 20 common amino acids or pyrrolysine or selenocysteine. Other terms that may be used synonymously with the term “non-naturally encoded amino acid” are “non-natural amino acid,” “unnatural amino acid,” “non-naturally-occurring amino acid,” and variously hyphenated and non-hyphenated versions thereof. The term “non-naturally encoded amino acid” also includes, but is not limited to, amino acids that occur by modification (e.g. post-translational modifications) of a naturally encoded amino acid (including but not limited to, the 20 common amino acids or pyrrolysine and selenocysteine) but are not themselves naturally incorporated into a growing polypeptide chain by the translation complex. Examples of such non-naturally-occurring amino acids include, but are not limited to, para-acetylphenylalanine, N-acetylglucosaminyl-L-serine, N-acetylglucosaminyl-L-threonine, and O-phosphotyrosine.

The term “substantially purified” refers to an IFNL3 polypeptide that may be substantially or essentially free of components that normally accompany or interact with the protein as found in its naturally occurring environment, i.e. a native cell, or host cell in the case of recombinantly produced IFNL3 polypeptides. IFNL3 polypeptide that may be substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the IFNL3 polypeptide or variant thereof is recombinantly produced by the host cells, the protein may be present at about 30%, about 25%, about 20%, about 15%, about 10%, about 5%, about 4%, about 3%, about 2%, or about 1% or less of the dry weight of the cells. When the IFNL3 polypeptide or variant thereof is recombinantly produced by the host cells, the protein may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. Thus, “substantially purified” IFNL3 polypeptide as produced by the methods of the present invention may have a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

The term “isolated,” when applied to a nucleic acid or protein, denotes that the nucleic acid or protein is free of at least some of the cellular components with which it is associated in the natural state, or that the nucleic acid or protein has been concentrated to a level greater than the concentration of its in vivo or in vitro production. It can be in a homogeneous state. Isolated substances can be in either a dry or semi-dry state, or in solution, including but not limited to, an aqueous solution. It can be a component of a pharmaceutical composition that comprises additional pharmaceutically acceptable carriers and/or excipients. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to substantially one band in an electrophoretic gel. Particularly, it may mean that the nucleic acid or protein is at least 85% pure, at least 90% pure, at least 95% pure, at least 99% or greater pure.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “medium” or “media” includes any culture medium, solution, solid, semi-solid, or rigid support that may support or contain any host cell, including bacterial host cells, yeast host cells, insect host cells, plant host cells, eukaryotic host cells, mammalian host cells, CHO cells, prokaryotic host cells, E. coli, or Pseudomonas host cells, and cell contents. Thus, the term may encompass a medium in which the host cell has been grown, e.g., a medium into which the IFNL3 polypeptide has been secreted, including a medium either before or after a proliferation step. The term also may encompass buffers or reagents that contain host cell lysates, such as in the case where the IFNL3 polypeptide is produced intracellularly and the host cells are lysed or disrupted to release the IFNL3 polypeptide.

“Reducing agent,” as used herein with respect to protein refolding, is defined as any compound or material which maintains sulfhydryl groups in the reduced state and reduces intra- or intermolecular disulfide bonds. Suitable reducing agents include, but are not limited to, dithiothreitol (DTT), 2-mercaptoethanol, dithioerythritol, cysteine, cystamine (2-aminoethanethiol), and reduced glutathione. It is readily apparent to those of ordinary skill in the art that a wide variety of reducing agents are suitable for use in the methods and compositions of the present invention.

“Oxidizing agent,” as used herein with respect to protein refolding, is defined as any compound or material which is capable of removing an electron from a compound being oxidized. Suitable oxidizing agents include, but are not limited to, oxidized glutathione, cystine, cystamine, oxidized dithiothreitol, oxidized erythritol, and oxygen. It is readily apparent to those of ordinary skill in the art that a wide variety of oxidizing agents are suitable for use in the methods of the present invention.

“Denaturing agent” or “denaturant,” as used herein, is defined as any compound or material which will cause a reversible unfolding of a protein. The strength of a denaturing agent or denaturant will be determined both by the properties and the concentration of the particular denaturing agent or denaturant. Suitable denaturing agents or denaturants may be chaotropes, detergents, organic solvents, water miscible solvents, phospholipids, or a combination of two or more such agents. Suitable chaotropes include, but are not limited to, urea, guanidine, and sodium thiocyanate. Useful detergents may include, but are not limited to, strong detergents such as sodium dodecyl sulfate, or polyoxyethylene ethers (e.g. Tween or Triton detergents), Sarkosyl, mild non-ionic detergents (e.g., digitonin), mild cationic detergents such as N->2,3-(Dioleyloxy)-propyl-N,N,N-trimethylammonium, mild ionic detergents (e.g. sodium cholate or sodium deoxycholate) or zwitterionic detergents including, but not limited to, sulfobetaines (Zwittergent), 3-(3-chlolamidopropyl)dimethylammonio-1-propane sulfate (CHAPS), and 3-(3-chlolamidopropyl)dimethylammonio-2-hydroxy-1-propane sulfonate (CHAPSO). Organic, water miscible solvents such as acetonitrile, lower alkanols (especially C2-C4 alkanols such as ethanol or isopropanol), or lower alkanediols (especially C2-C4 alkanediols such as ethylene-glycol) may be used as denaturants. Phospholipids useful in the present invention may be naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

“Refolding,” as used herein describes any process, reaction or method which transforms disulfide bond containing polypeptides from an improperly folded or unfolded state to a native or properly folded conformation with respect to disulfide bonds.

“Cofolding,” as used herein, refers specifically to refolding processes, reactions, or methods which employ at least two polypeptides which interact with each other and result in the transformation of unfolded or improperly folded polypeptides to native, properly folded polypeptides.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as homoserine, norleucine, methionine sulfoxide, methionine methyl sulfonium. Such analogs have modified R groups (such as norleucine) or modified peptide backbones, but retain the same basic chemical structure as a naturally occurring amino acid. Reference to an amino acid includes, for example, naturally occurring protogenic L-amino acids; D-amino acids, chemically modified amino acids such as amino acid variants and derivatives; naturally occurring non-protogenic amino acids such as β-alanine, ornithine, etc.; and chemically synthesized compounds having properties known in the art to be characteristic of amino acids. Examples of non-naturally occurring amino acids include, but are not limited to, para-acetylphenylalanine, α-methyl amino acids (e.g., α-methyl alanine), D-amino acids, histidine-like amino acids (e.g., 2-amino-histidine, β-hydroxy-histidine, homohistidine, α-fluoromethyl-histidine and α-methyl-histidine), amino acids having an extra methylene in the side chain (“homo” amino acids), and amino acids in which a carboxylic acid functional group in the side chain is replaced with a sulfonic acid group (e.g., cystic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles of the invention.

Conservative substitution tables providing functionally similar amino acids are known to those of ordinary skill in the art. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins: Structures and Molecular Properties (W H Freeman & Co.; 2nd edition (December 1993).

An “amino terminus modification group” refers to any molecule that can be attached to the amino terminus of a polypeptide. Similarly, a “carboxy terminus modification group” refers to any molecule that can be attached to the carboxy terminus of a polypeptide. Terminus modification groups include, but are not limited to, various PKEM, an amino acid such as glycine, linkers, or other molecules that provide a reactive chemical functional group.

The terms “functional group”, “active moiety”, “activating group”, “leaving group”, “reactive site”, “chemically reactive group” and “chemically reactive moiety” are used in the art and herein to refer to distinct, definable portions or units of a molecule. The terms are somewhat synonymous in the chemical arts and are used herein to indicate the portions of molecules that perform some function or activity and are reactive with other molecules.

The term “linkage” or “linker” is used herein to refer to groups or bonds that normally are formed as the result of a chemical reaction and typically are covalent linkages. Hydrolytically stable linkages means that the linkages are substantially stable in water and do not react with water at useful pH values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage can be degraded by one or more enzymes. Hydrolytically degradable linkages include, but are not limited to, carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, and a carboxyl group of a peptide; and oligonucleotide linkages formed by a phosphoramidite group, including but not limited to, at the end of a polymer, and a 5′ hydroxyl group of an oligonucleotide. Linkers include but are not limited to short linear, branched, multi-armed, or dendrimeric molecules such as polymers.

The term “biologically active molecule”, “biologically active moiety” or “biologically active agent” when used herein means any substance which can affect any physical or biochemical properties of a biological system, pathway, molecule, or interaction relating to an organism, including but not limited to, viruses, bacteria, bacteriophage, transposon, prion, insects, fungi, plants, animals, and humans. In particular, as used herein, biologically active molecules include, but are not limited to, any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, polymers, enzymes, small molecule drugs, vaccines, immunogens, hard drugs, soft drugs, carbohydrates, inorganic atoms or molecules, dyes, lipids, nucleosides, radionuclides, oligonucleotides, toxoids, toxins, prokaryotic and eukaryotic cells, viruses, polysaccharides, nucleic acids and portions thereof obtained or derived from viruses, bacteria, insects, animals or any other cell or cell type, liposomes, microparticles and micelles. The IFNL3 polypeptides may be added in a micellular formulation; see U.S. Pat. No. 5,833,948, which is incorporated by reference herein in its entirety. Classes of biologically active agents that are suitable for use with the invention include, but are not limited to, drugs, prodrugs, radionuclides, imaging agents, polymers, antibiotics, fungicides, anti-viral agents, anti-inflammatory agents, anti-tumor agents, cardiovascular agents, anti-anxiety agents, hormones, growth factors, steroidal agents, microbially derived toxins, and the like.

For example, a “biologically active” modified IFNL3 polypeptide may exhibit one or more of the activities of wild-type IFNL3, including without limitation inhibition of infectious agents, inhibition of virus replication, inhibition of bacterial replication, adjuvant activity for vaccines, and other biological activities as disclosed herein or as are known or become known for IFNL3.

The term “epoxide”, “epoxy group” or “oxirane” depicts a chemical functional group consisting of a three-membered ring arrangement of two carbon atoms and one oxygen atom. The two carbon atoms in the three-membered ring may be independently substituted. The term “epoxide” may also depict a molecule or compound that comprises at least one epoxy group.

The term “epoxide-containing compound” means any compound that is an epoxide or a compound which contains an epoxide moiety. Exemplary epoxide containing compounds are alkylene oxides and in particular lower alkylene oxides such as ethylene oxide, propylene oxide, butylene oxide, alcohol epoxides such as glycidol, and epihalohydrins such as epichlorohydrin, epibromohydrin, epiiodohydrin, 1,2-epoxy-4-chlorobutane, 1,2-epoxy-4-bromobutane, 1,2-epoxy-4-iodobutane, 2,3-epoxy-4-chlorobutane, 2,3-epoxy-4-bromobutane, 2,3-epoxy-4-iodobutane, 2,3-epoxy-5-chloropentane, 2,3-epoxy-5-bromopentane, 1,2-epoxy-5-chloropentane, etc., epoxy compounds such as 2,2-bis(p-1,2-epoxypropoxyphenyl)-propane1,4-bis(1,2-epoxypropoxy)benzene-, N,N′-bis(2,3-epoxypropyl)piperazine, etc.

The terms “electrophilic group”, “electrophile” and the like as used herein refers to an atom or group of atoms that can accept an electron pair to form a covalent bond. The “electrophilic group” used herein includes but is not limited to halide, carbonyl and epoxide containing compounds. Common electrophiles may be halides such as thiophosgene, glycerin dichlorohydrin, phthaloyl chloride, succinyl chloride, chloroacetyl chloride, chlorosucciriyl chloride, etc.; ketones such as chloroacetone, bromoacetone, etc.; aldehydes such as glyoxal, etc.; isocyanates such as hexamethylene diisocyanate, tolylene diisocyanate, meta-xylylene diisocyanate, cyclohexylmethane-4,4-diisocyanate, etc and derivatives of these compounds.

The terms “nucleophilic group”, “nucleophile” and the like as used herein refers to an atom or group of atoms that have an electron pair capable of forming a covalent bond. Groups of this type may be ionizable groups that react as anionic groups. The “nucleophilic group” used herein includes but is not limited to hydroxyl, primary amines, secondary amines, tertiary amines and thiols.

Table 1 provides various starting electrophiles and nucleophiles which may be combined to create a desired functional group. The information provided is meant to be illustrative and not limiting to the synthetic techniques described herein.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | Carbodiimides | carboxylic acids |
| Esters | Diazoalkanes | carboxylic acids |
| Thioethers | Epoxides | Thiols |
| Thioethers | Haloacetamides | Thiols |
| Ammotriazines | Halotriazines | amines/anilines |
| Triazinyl ethers | Halotriazines | alcohols/phenols |
| Amidines | imido esters | amines/anilines |
| Ureas | Isocyanates | amines/anilines |
| Urethanes | Isocyanates | alcohols/phenols |
| Thioureas | Isothiocyanates | amines/anilines |
| Thioethers | Maleimides | Thiols |
| Phosphite esters | Phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Non-limiting examples of carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other non-limiting examples of carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-limiting examples of non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a heteroatom, including, but not limited to, oxygen, sulfur, or nitrogen.

The term “ether” or “ether containing” refers to a class of organic compounds of general formula R—O—R, wherein R is carbon. The term “ether” or “ether containing” as used herein is intended to exclude those compounds where R is not carbon for example sialyl ethers, Si—O—Si.

The term “polyamine” refers to an organic compound having at least two positively amino groups selected from the group comprising primary amino groups secondary amino groups and tertiary amino groups. Accordingly, a polyamine covers diamines, triamines and higher amines.

The terms “chemically coupled” and “chemically couple” and grammatical variations thereof refer to the covalent and noncovalent bonding of molecules and include specifically, but not exclusively, covalent bonding, electrostatic bonding, hydrogen bonding and van der Waals' bonding. The terms encompass both indirect and direct bonding of molecules. Thus, if a first compound is chemically coupled to a second compound, that connection may be through a direct chemical bond, or through an indirect chemical bond via other compounds, linkers or connectors.

A “bifunctional polymer” refers to a polymer comprising two discrete functional groups that are capable of reacting specifically with other moieties (including but not limited to, amino acid side groups) to form covalent or non-covalent linkages. A bifunctional linker having one functional group reactive with a group on a particular biologically active component, and another group reactive with a group on a second biological component, may be used to form a conjugate that includes the first biologically active component, the bifunctional linker and the second biologically active component. Many procedures and linker molecules for attachment of various compounds to peptides are known. See, e.g., European Patent Application No. 188,256; U.S. Pat. Nos. 4,671,958, 4,659,839, 4,414,148, 4,699,784; 4,680,338; and 4,569,789 which are incorporated by reference herein. A “multi-functional polymer” refers to a polymer comprising two or more discrete functional groups that are capable of reacting specifically with other moieties (including but not limited to, amino acid side groups) to form covalent or non-covalent linkages. A bi-functional polymer or multi-functional polymer may be any desired length or molecular weight, and may be selected to provide a particular desired spacing or conformation between one or more molecules linked to the IFNL3.

Where substituent groups are specified by their conventional chemical formulas, written from left to right, they equally encompass the chemically identical substituents that would result from writing the structure from right to left, for example, the structure CH2O is equivalent to the structure —OCH2.

The term “substituents” includes but is not limited to “non-interfering substituents”. “Non-interfering substituents” are those groups that yield stable compounds. Suitable non-interfering substituents or radicals include, but are not limited to, halo, C1-C10 alkyl, C2-C10 alkenyl, C2-C10 alkynyl, C1-C10 alkoxy, C1-C12 aralkyl, C1-C12 alkaryl, C3-C12 cycloalkyl, C3-C12 cycloalkenyl, phenyl, substituted phenyl, toluoyl, xylenyl, biphenyl, C2-C12 alkoxyalkyl, C2-C12 alkoxyaryl, C7-C12 aryloxyalkyl, C7-C12 oxyaryl, C1-C6 alkylsulfinyl, C1-C10 alkylsulfonyl, —(CH2)m-O—(C1-C10 alkyl) wherein m is from 1 to 8, aryl, substituted aryl, substituted alkoxy, fluoroalkyl, heterocyclic radical, substituted heterocyclic radical, nitroalkyl, —NO2, —CN, —NRC(O)—(C1-C10 alkyl), —C(O)—(C1-C10 alkyl), C2-C10 alkyl thioalkyl, —C(O)O—(C1-C10 alkyl), —OH, —SO2, ═S, —COOH, —NR2, carbonyl, —C(O)—(C1-C10 alkyl)-CF3, —C(O)—CF3, —C(O)NR2, —(C1-C10 aryl)-S—(C6-C10 aryl), —C(O)—(C1-C10 aryl), —(CH2)m-O—(—(CH2)m-O—(C1-C10 alkyl) wherein each m is from 1 to 8, —C(O)NR2, —C(S)NR2, —SO2NR2, —NRC(O) NR2, —NRC(S) NR2, salts thereof, and the like. Each R as used herein is H, alkyl or substituted alkyl, aryl or substituted aryl, aralkyl, or alkaryl.

The term “halogen” includes fluorine, chlorine, iodine, and bromine.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C1-C10 means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “alkylene” by itself or as part of another substituent means a divalent radical derived from an alkane, as exemplified, but not limited, by the structures —CH2CH2- and —CH2CH2CH2CH2-, and further includes those groups described below as “heteroalkylene.” Typically, an alkyl (or alkylene) group will have from 1 to 24 carbon atoms, with those groups having 10 or fewer carbon atoms being a particular embodiment of the methods and compositions described herein. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH2-CH2-O—CH3, —CH2-CH2-NH—CH3, —CH2-CH2-N(CH3)-CH3, —CH2-S—CH2-CH3, —CH2-CH2, —S(O)—CH3, —CH2-CH2-S(O)₂—CH3, —CH═CH—O—CH3, —Si(CH3)₃, —CH2-CH═N—OCH3, and —CH═CH—N(CH3)-CH3. Up to two heteroatoms may be consecutive, such as, for example, —CH2-NH—OCH3 and —CH2-O—Si(CH3)3. Similarly, the term “heteroalkylene” by itself or as part of another substituent means a divalent radical derived from heteroalkyl, as exemplified, but not limited by, —CH2-CH2-S—CH2 CH2- and —CH2-S—CH2-CH2-NH—CH2-. For heteroalkylene groups, the same or different heteroatoms can also occupy either or both of the chain termini (including but not limited to, alkyleneoxy, alkylenedioxy, alkyleneamino, alkylenediamino, aminooxyalkylene, and the like). Still further, for alkylene and heteroalkylene linking groups, no orientation of the linking group is implied by the direction in which the formula of the linking group is written. For example, the formula —C(O)₂R′ represents both —C(O)₂R′ and —R′C(O)₂.

The terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated, partially unsaturated and fully unsaturated ring linkages. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. Additionally, the term encompasses bicyclic and tricyclic ring structures. Similarly, the term “heterocycloalkylene” by itself or as part of another substituent means a divalent radical derived from heterocycloalkyl, and the term “cycloalkylene” by itself or as part of another substituent means a divalent radical derived from cycloalkyl.

As used herein, the term “water soluble polymer” refers to any polymer that is soluble in aqueous solvents. Linkage of water soluble polymers to IFNL3 polypeptides can result in changes including, but not limited to, increased or modulated serum half-life, or increased or modulated therapeutic half-life relative to the unmodified form, modulated immunogenicity, modulated physical association characteristics such as aggregation and multimer formation, altered receptor, activity modulator, or other IFNL3 polypeptide binding, altered binding to one or more binding partners, and altered IFNL3 dimerization or multimerization. The water soluble polymer may or may not have its own biological activity, and may be utilized as a linker for attaching IFNL3 to other substances, including but not limited to one or more IFNL3 polypeptides, or one or more biologically active molecules. Suitable polymers include, but are not limited to, polyethylene glycol, polyethylene glycol propionaldehyde, mono C1-C10 alkoxy or aryloxy derivatives thereof (described in U.S. Pat. No. 5,252,714 which is incorporated by reference herein), monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, and the like, or mixtures thereof. Examples of such water soluble polymers include, but are not limited to, polyethylene glycol and serum albumin.

As used herein, the term “polyalkylene glycol” or “poly(alkene glycol)” refers to polyethylene glycol (poly(ethylene glycol)), polypropylene glycol, polybutylene glycol, and derivatives thereof. The term “polyalkylene glycol” encompasses both linear and branched polymers and average molecular weights of between 0.1 kDa and 100 kDa. Other exemplary embodiments are listed, for example, in commercial supplier catalogs, such as Shearwater Corporation's catalog “Polyethylene Glycol and Derivatives for Biomedical Applications” (2001).

The term “aryl” means, unless otherwise stated, a polyunsaturated, aromatic, hydrocarbon substituent which can be a single ring or multiple rings (including but not limited to, from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (including but not limited to, aryloxy, arylthioxy, arylalkyl) includes both aryl and heteroaryl rings as defined above. Thus, the term “arylalkyl” is meant to include those radicals in which an aryl group is attached to an alkyl group (including but not limited to, benzyl, phenethyl, pyridylmethyl and the like) including those alkyl groups in which a carbon atom (including but not limited to, a methylene group) has been replaced by, for example, an oxygen atom (including but not limited to, phenoxymethyl, 2-pyridyloxymethyl, 3-(1-naphthyloxy)propyl, and the like).

Each of the above terms (including but not limited to, “alkyl,” “heteroalkyl,” “aryl” and “heteroaryl”) are meant to include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, OC(O)R′, —C(O)R′, —CO2R′, —CONR′R″, —OC(O)NR′R″, —NR″C(O)R′, NR′ C(O)NR″R′″, —NR″C(O)2R′, —NR—C(NR′R″R′″)═NR″″, NR C(NR′R″)═NR′″, —S(O)R′, —S(O)2R′, —S(O)2NR′R″, NRSO2R′, —CN and —NO2 in a number ranging from zero to (2 m′+1), where m′ is the total number of carbon atoms in such a radical. R′, R″, R′″ and R″″ each independently refer to hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (including but not limited to, —CF3 and —CH2CF3) and acyl (including but not limited to, —C(O)CH3, —C(O)CF3, —C(O)CH2OCH3, and the like).

Similar to the substituents described for the alkyl radical, substituents for the aryl and heteroaryl groups are varied and are selected from, but are not limited to: halogen, OR′, ═O, ═NR′, ═N—OR′, —NR′R″, —SR′, -halogen, —SiR′R″R′″, OC(O)R′, —C(O)R′, CO2R′, —CONR′R″, —OC(O)NR′R″, —NR″C(O)R′, NR′ C(O)NR″R′″, —NR″C(O)₂R′, NR—C(NR′R″R′″)═NR″″, NR C(NR′R″)═NR′″, —S(O)R′, —S(O)₂R′, —S(O)₂NR′R″, NRSO2R′, —CN and —NO2, —R′, —N3, —CH(Ph)2, fluoro(C1-C4)alkoxy, and fluoro(C1-C4)alkyl, in a number ranging from zero to the total number of open valences on the aromatic ring system; and where R′, R″, R′″ and R″″ are independently selected from hydrogen, alkyl, heteroalkyl, aryl and heteroaryl. When a compound of the invention includes more than one R group, for example, each of the R groups is independently selected as are each R′, R″, R′″ and R″″ groups when more than one of these groups is present.

As used herein, the term “modulated serum half-life” means the positive or negative change in circulating half-life of an IFNL3 or modified IFNL3 relative to its non-modified form. Serum half-life is measured by taking blood samples at various time points after administration of IFNL3, and determining the concentration of that molecule in each sample. Correlation of the serum concentration with time allows calculation of the serum half-life. Increased serum half-life desirably has at least about two-fold, but a smaller increase may be useful, for example where it enables a satisfactory dosing regimen or avoids a toxic effect. In some embodiments, the increase is at least about three-fold, at least about five-fold, or at least about ten-fold or more. Modulated serum half life can be accomplished using a PKEM.

The term “modulated therapeutic half-life” as used herein means the positive or negative change in the half-life of the therapeutically effective amount of IFNL3, relative to its non-modified form. Therapeutic half-life is measured by measuring pharmacokinetic and/or pharmacodynamic properties of the molecule at various time points after administration. Increased therapeutic half-life desirably enables a particular beneficial dosing regimen, a particular beneficial total dose, or avoids an undesired effect. In some embodiments, the increased therapeutic half-life results from increased potency, increased enzymatic activity, increased therapeutic efficacy, increased or decreased binding of the modified molecule to its target, increased or decreased breakdown of the molecule by enzymes such as proteases, or an increase or decrease in another parameter or mechanism of action of the non-modified molecule or an increase or decrease in clearance of the molecule.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present invention, including homologs from species other than human, may be obtained by a process comprising the steps of screening a library under stringent hybridization conditions with a labeled probe having a polynucleotide sequence of the invention or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information available at the World Wide Web at ncbi.nlm.nih.gov. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, PNA, or other nucleic acid mimics, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, typically about 0.01 to 1.0 M sodium ion concentration (or other salts) at pH 7.0 to 8.3 and the temperature is at least about 30° C. for short probes (including but not limited to, 10 to 50 nucleotides) and at least about 60° C. for long probes (including but not limited to, greater than 50 nucleotides). Stringent conditions may also be achieved with the addition of destabilizing agents such as formamide. For selective or specific hybridization, a positive signal may be at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C. Such washes can be performed for 5, 15, 30, 60, 120, or more minutes.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “non-eukaryote” refers to non-eukaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

The term “subject” as used herein, refers to an animal, in some embodiments a mammal, and in other embodiments a human, who is the object of treatment, observation or experiment. An animal may be a companion animal (e.g., dogs, cats, and the like), farm animal (e.g., cows, sheep, pigs, horses, and the like) or a laboratory animal (e.g., rats, mice, guinea pigs, and the like).

The term “effective amount” as used herein refers to that amount of the IFNL3 polypeptide being administered which will prevent or relieve to some extent one or more of the symptoms of the disease, condition or disorder being treated. Compositions containing the IFNL3 polypeptides described herein can be administered for prophylactic, enhancing, and/or therapeutic treatments.

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system. When used in a subject, amounts effective for this use will depend on the severity and course of the disease, disorder or condition, previous therapy, the subject's health status and response to the drugs, and the judgment of the treating physician.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

The term “protected” refers to the presence of a “protecting group” or moiety that prevents reaction of the chemically reactive functional group under certain reaction conditions. The protecting group will vary depending on the type of chemically reactive group being protected. For example, if the chemically reactive group is an amine or a hydrazide, the protecting group can be selected from the group of tert-butyloxycarbonyl (t-Boc) and 9-fluorenylmethoxycarbonyl (Fmoc). If the chemically reactive group is a thiol, the protecting group can be orthopyridyldisulfide. If the chemically reactive group is a carboxylic acid, such as butanoic or propionic acid, or a hydroxyl group, the protecting group can be benzyl or an alkyl group such as methyl, ethyl, or tert-butyl. Other protecting groups known in the art may also be used in or with the methods and compositions described herein, including photolabile groups such as Nvoc and MeNvoc. Other protecting groups known in the art may also be used in or with the methods and compositions described herein.

By way of example only, blocking/protecting groups may be selected from:

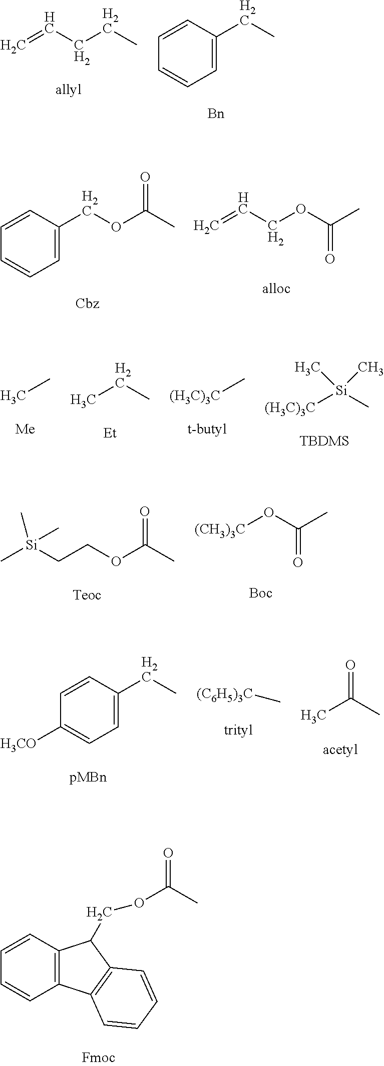

Other protecting groups are described in Greene and Wuts, Protective Groups in Organic Synthesis, 3rd Ed., John Wiley & Sons, New York, N.Y., 1999, which is incorporated herein by reference in its entirety.

The term “treating” is used to refer to either prophylactic and/or therapeutic treatments.

The term “therapeutically effective amount” refers to an amount which gives the desired benefit to a subject. The amount will vary from one individual to another and will depend upon a number of factors, including the overall physical condition of the subject and the underlying cause of the condition to be treated. The amount of modified IFNL3 polypeptide used for therapy gives an acceptable rate of change and maintains desired response at a beneficial level.

IFNL3 polypeptides presented herein may include isotopically-labelled compounds with one or more atoms replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as 2H, 3H, 13C, 14C, 15N, 18O, 17O, 35S, 18F, 36Cl, respectively. Certain isotopically-labelled compounds described herein, for example those into which radioactive isotopes such as 3H and 14C are incorporated, may be useful in drug and/or substrate tissue distribution assays. Further, substitution with isotopes such as deuterium, i.e., 2H, can afford certain therapeutic advantages resulting from greater metabolic stability, for example increased in vivo half-life or reduced dosage requirements.

All isomers including but not limited to diastereomers, enantiomers, and mixtures thereof are considered as part of the compositions described herein. In additional or further embodiments, the amino acid polypeptides are metabolized upon administration to an organism in need to produce a metabolite that is then used to produce a desired effect, including a desired therapeutic effect. In further or additional embodiments are active metabolites of polypeptides.

In some situations, non-naturally encoded amino acid polypeptides may exist as tautomers. In addition, the non-naturally encoded amino acid polypeptides described herein can exist in unsolvated as well as solvated forms with pharmaceutically acceptable solvents such as water, ethanol, and the like. The solvated forms are also considered to be disclosed herein. Those of ordinary skill in the art will recognize that some of the compounds herein can exist in several tautomeric forms. All such tautomeric forms are considered as part of the compositions described herein.

Unless otherwise indicated, conventional methods of mass spectroscopy, NMR, HPLC, protein chemistry, biochemistry, recombinant DNA techniques and pharmacology, within the skill of the art are employed.

The IFNL3 polypeptide disclosed herein can be a pegylated interferon lambda 3 (IFNL3) polypeptide or fragment thereof. More particularly, the pegylated IFNL3 polypeptide can include a sequence having at least 80% identity to SEQ ID NO: 1; SEQ ID NO:2; SEQ ID NO:3; SEQ ID NO 4; SEQ ID NO 5; SEQ ID NO: 6; SEQ ID NO: 7; SEQ ID NO: 8; or SEQ ID NO: 9, or fragment thereof, wherein said polypeptide is covalently linked to at least one polyethylene glycol.

IFNL3 polypeptides including IFNL3 polypeptides comprising at least one amino acid substitution, addition, deletion or insertion are provided in the invention. In certain embodiments of the invention, the IFNL3 polypeptide includes at least one post-translational modification. In one embodiment, the at least one post-translational modification comprises attachment of a molecule including but not limited to, a PKEM, a label, a dye, a polymer, a water-soluble polymer, a derivative of polyethylene glycol, a photocrosslinker, a radionuclide, a cytotoxic compound, a drug, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, a neutron-capture agent, or any combination of the above or any other desirable compound or substance, comprising a second reactive group to at least one amino acid comprising a first reactive group utilizing chemistry methodology that is known to one of ordinary skill in the art to be suitable for the particular reactive groups. In certain embodiments, the post-translational modification is made in vivo in a eukaryotic cell or in a non-eukaryotic cell. A linker, polymer, PKEM, or other molecule may attach the molecule to the polypeptide. The molecule may be linked directly to the polypeptide.

In certain embodiments, the IFNL3 protein includes at least one post-translational modification that is made in vivo by one host cell, where the post-translational modification is not normally made by another host cell type. In certain embodiments, the IFNL3 protein includes at least one post-translational modification that is made in vivo by a eukaryotic cell, where the post-translational modification is not normally made by a non-eukaryotic cell. Examples of post-translational modifications include, but are not limited to, glycosylation, acetylation, acylation, lipid-modification, palmitoylation, palmitate addition, phosphorylation, glycolipid-linkage modification, and the like.

In some embodiments, the IFNL3 polypeptide comprises one or more post-translational modification including but not limited to glycosylation, acetylation, acylation, lipid-modification, palmitoylation, palmitate addition, phosphorylation, or glycolipid-linkage modification of the polypeptide. In one embodiment, the post-translational modification comprises attachment of an oligosaccharide to an asparagine by a GlcNAc-asparagine linkage (including but not limited to, where the oligosaccharide comprises (GlcNAc-Man)₂-Man-GlcNAc-GlcNAc, and the like). In another embodiment, the post-translational modification comprises attachment of an oligosaccharide (including but not limited to, Gal-GalNAc, Gal-GlcNAc, etc.) to a serine or threonine by a GalNAc-serine, a GalNAc-threonine, a GlcNAc-serine, or a GlcNAc-threonine linkage. In certain embodiments, a protein or polypeptide of the invention can comprise a secretion or localization sequence, an epitope tag, a FLAG tag, a histidine tag comprising one or more histidine residues, a GST fusion, and/or the like. Examples of secretion signal sequences include, but are not limited to, a prokaryotic secretion signal sequence, a eukaryotic secretion signal sequence, a eukaryotic secretion signal sequence 5′-optimized for bacterial expression, a novel secretion signal sequence, pectate lyase secretion signal sequence, Omp A secretion signal sequence, and a phage secretion signal sequence. Examples of secretion signal sequences, include, but are not limited to, STII (prokaryotic), Fd GIII and M13 (phage), Bgl2 (yeast), and the signal sequence bla derived from a transposon. Any such sequence may be modified to provide a desired result with the polypeptide, including but not limited to, substituting one signal sequence with a different signal sequence, substituting a leader sequence with a different leader sequence, etc.

The amino acid side chains can then be modified by utilizing chemistry methodologies known to those of ordinary skill in the art to be suitable for the particular functional groups or substituents. Known chemistry methodologies of a wide variety are suitable for use in the present invention to incorporate a PKEM into the protein. Such methodologies include but are not limited to a Huisgen [3+2] cycloaddition reaction (see, e.g., Padwa, A. in Comprehensive Organic Synthesis, Vol. 4, (1991) Ed. Trost, B. M., Pergamon, Oxford, p. 1069-1109; and, Huisgen, R. in 1,3-Dipolar Cycloaddition Chemistry, (1984) Ed. Padwa, A., Wiley, New York, p. 1-176) with, including but not limited to, acetylene or azide derivatives, respectively.

The present invention provides conjugates of substances having a wide variety of functional groups, substituents or moieties, with other substances including but not limited to a PKEM; a label; a dye; a polymer; a water-soluble polymer; a derivative of polyethylene glycol; a photocrosslinker; a radionuclide; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance. The present invention also includes conjugates of substances having azide or acetylene moieties with PKEM derivatives having the corresponding acetylene or azide moieties. For example, a PKEM containing an azide moiety can be coupled to a biologically active molecule at a position in the protein that contains a non-genetically encoded amino acid bearing an acetylene functionality.

As described herein, the present disclosures provide IFNL3 polypeptides coupled to another molecule having the formula IFNL3-L-M, wherein L is a linking group or a chemical bond, and M is any other molecule. In some embodiments, L is stable in vivo. In some embodiments, L is hydrolyzable in vivo. In some embodiments, L is metastable in vivo.

IFNL3 and M can be linked together through L using standard linking agents and procedures known to those skilled in the art. In some aspects, IFNL3 and M are fused directly and L is a bond. In other aspects, IFNL3 and M are fused through a linking group L. For example, in some embodiments, IFNL3 and M are linked together via a peptide bond, optionally through a peptide or amino acid spacer. In some embodiments, IFNL3 and M are linked together through chemical conjugation, optionally through a linking group (L). In some embodiments, L is directly conjugated to each of IFNL3 and M.

Chemical conjugation can occur by reacting a nucleophilic reactive group of one compound to an electrophilic reactive group of another compound. In some embodiments when L is a bond, IFNL3 is conjugated to M either by reacting a nucleophilic reactive moiety on IFNL3 with an electrophilic reactive moiety on Y, or by reacting an electrophilic reactive moiety on IFNL3 with a nucleophilic reactive moiety on M. In embodiments when L is a group that links IFNL3 and M together, IFNL3 and/or M can be conjugated to L either by reacting a nucleophilic reactive moiety on IFNL3 and/or M with an electrophilic reactive moiety on L, or by reacting an electrophilic reactive moiety on IFNL3 and/or M with a nucleophilic reactive moiety on L. Nonlimiting examples of nucleophilic reactive groups include amino, thiol, and hydroxyl. Nonlimiting examples of electrophilic reactive groups include carboxyl, acyl chloride, anhydride, ester, succinimide ester, alkyl halide, sulfonate ester, maleimido, haloacetyl, and isocyanate. In embodiments where IFNL3 and M are conjugated together by reacting a carboxylic acid with an amine, an activating agent can be used to form an activated ester of the carboxylic acid.

The activated ester of the carboxylic acid can be, for example, N-hydroxysuccinimide (NHS), tosylate (Tos), mesylate, triflate, a carbodiimide, or a hexafluorophosphate. In some embodiments, the carbodiimide is 1,3-dicyclohexylcarbodiimide (DCC), 1,1′-carbonyldiimidazole (CDI), 1-ethyl-3-(3-dimethylaminopropyl)carbodiimide hydrochloride (EDC), or 1,3-diisopropylcarbodiimide (DICD). In some embodiments, the hexafluorophosphate is selected from a group consisting of hexafluorophosphate benzotriazol-1-yl-oxy-tris(dimethylamino)phosphonium hexafluorophosphate (BOP), benzotriazol-1-yl-oxytripyrrolidinophosphonium hexafluorophosphate (PyBOP), 2-(1H-7-azabenzotriazol-1-yl)-1,1,3,3-tetramethyl uronium hexafluorophosphate (HATU), and o-benzotriazole-N,N,N′,N′-tetramethyl-uronium-hexafluoro-phosphate (HBTU).

In some embodiments, IFNL3 comprises a nucleophilic reactive group (e.g. the amino group, thiol group, or hydroxyl group of the side chain of lysine, cysteine or serine) that is capable of conjugating to an electrophilic reactive group on M or L. In some embodiments, IFNL3 comprises an electrophilic reactive group (e.g. the carboxylate group of the side chain of Asp or Glu) that is capable of conjugating to a nucleophilic reactive group on M or L. In some embodiments, IFNL3 is chemically modified to comprise a reactive group that is capable of conjugating directly to M or to L. In some embodiments, IFNL3 is modified at the N-terminus or C-terminus to comprise a natural or nonnatural amino acid with a nucleophilic side chain. In exemplary embodiments, the N-terminus or C-terminus amino acid of IFNL3 is selected from the group consisting of lysine, ornithine, serine, cysteine, and homocysteine. For example, the N-terminus or C-terminus amino acid of IFNL3 can be modified to comprise a lysine residue. In some embodiments, IFNL3 is modified at the N-terminus or C-terminus amino acid to comprise a natural or nonnatural amino acid with an electrophilic side chain such as, for example, Asp and Glu. In some embodiments, an internal amino acid of IFNL3 is substituted with a natural or nonnatural amino acid having a nucleophilic side chain, as previously described herein. In exemplary embodiments, the internal amino acid of IFNL3 that is substituted is selected from the group consisting of lysine, ornithine, serine, cysteine, and homocysteine. For example, an internal amino acid of IFNL3 can be substituted with a lysine residue. In some embodiments, an internal amino acid of IFNL3 is substituted with a natural or nonnatural amino acid with an electrophilic side chain, such as, for example, Asp and Glu.

In some embodiments, M comprises a reactive group that is capable of conjugating directly to IFNL3 or to L. In some embodiments, M comprises a nucleophilic reactive group (e.g. amine, thiol, hydroxyl) that is capable of conjugating to an electrophilic reactive group on IFNL3 or L. In some embodiments, M comprises electrophilic reactive group (e.g. carboxyl group, activated form of a carboxyl group, compound with a leaving group) that is capable of conjugating to a nucleophilic reactive group on IFNL3 or L. In some embodiments, M is chemically modified to comprise either a nucleophilic reactive group that is capable of conjugating to an electrophilic reactive group on IFNL3 or L. In some embodiments, M is chemically modified to comprise an electrophilic reactive group that is capable of conjugating to a nucleophilic reactive group on IFNL3 or L.

In some embodiments, conjugation can be carried out through organosilanes, for example, aminosilane treated with glutaraldehyde; carbonyldiimidazole (CDI) activation of silanol groups; or utilization of dendrimers. A variety of dendrimers are known in the art and include poly (amidoamine) (PAMAM) dendrimers, which are synthesized by the divergent method starting from ammonia or ethylenediamine initiator core reagents; a sub-class of PAMAM dendrimers based on a tris-aminoethylene-imine core; radially layered poly(amidoamine-organosilicon) dendrimers (PAMAMOS), which are inverted unimolecular micelles that consist of hydrophilic, nucleophilic polyamidoamine (PAMAM) interiors and hydrophobic organosilicon (OS) exteriors; Poly (Propylene Imine) (PPI) dendrimers, which are generally poly-alkyl amines having primary amines as end groups, while the dendrimer interior consists of numerous of tertiary tris-propylene amines; Poly (Propylene Amine) (POPAM) dendrimers; Diaminobutane (DAB) dendrimers; amphiphilic dendrimers; micellar dendrimers which are unimolecular micelles of water soluble hyper branched polyphenylenes; polylysine dendrimers; and dendrimers based on poly-benzyl ether hyper branched skeleton.

In some embodiments, conjugation can be carried out through olefin metathesis. In some embodiments, M and IFNL3, M and L, or IFNL3 and L both comprise an alkene or alkyne moiety that is capable of undergoing metathesis. In some embodiments a suitable catalyst (e.g. copper, ruthenium) is used to accelerate the metathesis reaction. Suitable methods of performing olefin metathesis reactions are described in the art. See, for example, Schafmeister et al., J. Am. Chem. Soc. 122: 5891-5892 (2000), Walensky et al., Science 305: 1466-1470 (2004), and Blackwell et al., Angew, Chem., Int. Ed. 37: 3281-3284 (1998).

In some embodiments, conjugation can be carried out using click chemistry. A “click reaction” is wide in scope and easy to perform, uses only readily available reagents, and is insensitive to oxygen and water. In some embodiments, the click reaction is a cycloaddition reaction between an alkynyl group and an azido group to form a triazolyl group. In some embodiments, the click reaction uses a copper or ruthenium catalyst. Suitable methods of performing click reactions are described in the art. See, for example, Kolb et al., Drug Discovery Today 8: 1128 (2003); Kolb et al., Angew. Chem. Int. Ed. 40:2004 (2001); Rostovtsev et al., Angew. Chem. Int. Ed. 41:2596 (2002); Tomoe et al., J. Org. Chem. 67:3057 (2002); Manetsch et al., J. Am. Chem. Soc. 126: 12809 (2004); Lewis et al., Angew. Chem. Int. Ed. 41: 1053 (2002); Speers, J. Am. Chem. Soc. 125:4686 (2003); Chan et al. Org. Lett. 6:2853 (2004); Zhang et al., J. Am. Chem. Soc. 127: 15998 (2005); and Waser et al., J. Am. Chem. Soc. 127:8294 (2005).

Indirect conjugation via high affinity specific binding partners, e.g. streptavidin/biotin or avidin/biotin or lectin/carbohydrate is also contemplated.

In some embodiments, IFNL3 and/or M are functionalized to comprise a nucleophilic reactive group or an electrophilic reactive group with an organic derivatizing agent. This derivatizing agent is capable of reacting with selected side chains or the N- or C-terminal residues of targeted amino acids on IFNL3 and functional groups on M. Reactive groups on IFNL3 and/or M include, e.g., aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, IFNL3 and/or M can be linked to each other indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Cysteinyl residues most commonly are reacted with a-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidazole and tetranitromethane are used to form 0-acetyl tyrosyl species and 3-nitro derivatives, respectively.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group.

Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981).

In some embodiments, L is a bond. In these embodiments, IFNL3 and M are conjugated together by reacting a nucleophilic reactive moiety on IFNL3 with and electrophilic reactive moiety on M. In alternative embodiments, IFNL3 and M are conjugated together by reacting an electrophilic reactive moiety on IFNL3 with a nucleophilic moiety on M. In exemplary embodiments, L is an amide bond that forms upon reaction of an amine on IFNL3 (e.g. an F-amine of a lysine residue) with a carboxyl group on M. In alternative embodiments, IFNL3 and or M are derivatized with a derivatizing agent before conjugation.

In some embodiments, L is a linking group. In some embodiments, L is a bifunctional linker and comprises only two reactive groups before conjugation to IFNL3 and M. In embodiments where both IFNL3 and M have electrophilic reactive groups, L comprises two of the same or two different nucleophilic groups (e.g. amine, hydroxyl, thiol) before conjugation to IFNL3 and M. In embodiments where both IFNL3 and M have nucleophilic reactive groups, L comprises two of the same or two different electrophic groups (e.g. carboxyl group, activated form of a carboxyl group, compound with a leaving group) before conjugation to IFNL3 and M. In embodiments where one of IFNL3 or M has a nucleophilic reactive group and the other of IFNL3 or M has an electrophic reactive group, L comprises one nucleophilic reactive group and one electrophic group before conjugation to IFNL3 and M.

L can be any molecule with at least two reactive groups (before conjugation to IFNL3 and M) capable of reacting with each of IFNL3 and M. In some embodiments L has only two reactive groups and is bifunctional. L (before conjugation to the peptides) can be represented by Formula VI:

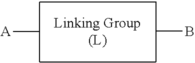

wherein A and B are independently nucleophilic or electrophic reactive groups. In some embodiments A and B are either both nucleophilic groups or both electrophic groups. In some embodiments one of A or B is a nucleophilic group and the other of A or B is an electrophic group. Nonlimiting combinations of A and B are shown below.

TABLE 2

| Both Nucleophilic | | Both Electrophilic | | Nucleophilic/Electrophilic | |
|---|---|---|---|---|---|
| A | B | A | B | A | B |
| Amino | Amino | Carboxyl | Carboxyl | Amino | Carboxyl |
| Amino | Thiol | Carboxyl | Acyl | Amino | Acyl chloride |
| | | | chloride | | |
| Amino | Hydroxyl | Carboxyl | Anhydride | Amino | Anhydride |
| Thiol | Amino | Carboxyl | Ester | Amino | Ester |
| Thiol | Thiol | Carboxyl | NHS | Amino | NHS |
| Thiol | Hydroxyl | Carboxyl | Halogen | Amino | Halogen |
| Hydroxyl | Amino | Carboxyl | Sulfonate | Amino | Sulfonate ester |
| | | | ester | | |
| Hydroxyl | Thiol | Carboxyl | Maleimido | Amino | Maleimido |
| Hydroxyl | Hydroxyl | Carboxyl | Haloacetyl | Amino | Haloacetyl |
| | | Carboxyl | Isocyanate | Amino | Isocyanate |
| | | Acyl | Carboxyl | Thiol | Carboxyl |
| | | chloride | | | |
| | | Acyl chloride | Acyl chloride | Thiol | Acyl chloride |
| | | Acyl chloride | Anhydride | Thiol | Anhydride |
| | | Acyl chloride | Ester | Thiol | Ester |
| | | Acyl chloride | NHS | Thiol | NHS |
| | | Acyl chloride | Halogen | Thiol | Halogen |
| | | Acyl chloride | Sulfonate ester | Thiol | Sulfonate ester |
| | | Acyl chloride | Maleimido | Thiol | Maleimido |
| | | Acyl chloride | Haloacetyl | Thiol | Haloacetyl |
| | | Acyl chloride | Isocyanate | Thiol | Isocyanate |
| | | Anhydride | Carboxyl | Hydroxyl | Carboxyl |
| | | Anhydride | Acyl chloride | Hydroxyl | Acyl chloride |
| | | Anhydride | Anhydride | Hydroxyl | Anhydride |
| | | Anhydride | Ester | Hydroxyl | Ester |
| | | Anhydride | NHS | Hydroxyl | NHS |
| | | Anhydride | Halogen | Hydroxyl | Halogen |
| | | Anhydride | Sulfonate ester | Hydroxyl | Sulfonate ester |
| | | Anhydride | Maleimido | Hydroxyl | Maleimido |
| | | Anhydride | Haloacetyl | Hydroxyl | Haloacetyl |
| | | Anhydride | Isocyanate | Hydroxyl | Isocyanate |

| | |
|---|---|
| Ester | Carboxyl |
| Ester | Acyl chloride |
| Ester | Anhydride |
| Ester | Ester |
| Ester | NHS |
| Ester | Halogen |
| Ester | Sulfonate ester |
| Ester | Maleimido |
| Ester | Haloacetyl |
| Ester | Isocyanate |
| NHS | Carboxyl |
| NHS | Acyl chloride |
| NHS | Anhydride |
| NHS | Ester |
| NHS | NHS |
| NHS | Halogen |
| NHS | Sulfonate ester |
| NHS | Maleimido |
| NHS | Haloacetyl |
| NHS | Isocyanate |
| Halogen | Carboxyl |
| Halogen | Acyl chloride |
| Halogen | Anhydride |
| Halogen | Ester |
| Halogen | NHS |
| Halogen | Halogen |
| Halogen | Sulfonate ester |
| Halogen | Maleimido |
| Halogen | Haloacetyl |
| Halogen | Isocyanate |
| Sulfonate ester | Carboxyl |
| Sulfonate ester | Acyl chloride |
| Sulfonate ester | Anhydride |
| Sulfonate ester | Ester |
| Sulfonate ester | NHS |
| Sulfonate ester | Halogen |
| Sulfonate ester | Sulfonate ester |
| Sulfonate ester | Maleimido |
| Sulfonate ester | Haloacetyl |
| Sulfonate ester | Isocyanate |
| Maleimido | Carboxyl |
| Maleimido | Acyl chloride |
| Maleimido | Anhydride |
| Maleimido | Ester |
| Maleimido | NHS |
| Maleimido | Halogen |
| Maleimido | Sulfonate ester |
| Maleimido | Maleimido |
| Maleimido | Haloacetyl |
| Maleimido | Isocyanate |
| Haloacetyl | Carboxyl |
| Haloacetyl | Acyl chloride |
| Haloacetyl | Anhydride |
| Haloacetyl | Ester |
| Haloacetyl | NHS |
| Haloacetyl | Halogen |
| Haloacetyl | Sulfonate ester |
| Haloacetyl | Maleimido |
| Haloacetyl | Haloacetyl |
| Haloacetyl | Isocyanate |
| Isocyanate | Carboxyl |
| Isocyanate | Acyl chloride |
| Isocyanate | Anhydride |
| Isocyanate | Ester |
| Isocyanate | NHS |
| Isocyanate | Halogen |
| Isocyanate | Sulfonate ester |
| Isocyanate | Maleimido |
| Isocyanate | Haloacetyl |
| Isocyanate | Isocyanate |

In some embodiments, A and B may include alkene and/or alkyne functional groups that are suitable for olefin metathesis reactions. In some embodiments, A and B include moieties that are suitable for click chemistry (e.g. alkene, alkynes, nitriles, azides). Other nonlimiting examples of reactive groups (A and B) include pyridyldithiol, aryl azide, diazirine, carbodiimide, and hydrazide.

In some embodiments, L is hydrophobic. Hydrophobic linkers are known in the art. See, e.g., Bioconjugate Techniques, G. T. Hermanson (Academic Press, San Diego, Calif., 1996), which is incorporated by reference in its entirety. Suitable hydrophobic linking groups known in the art include, for example, 8-hydroxy octanoic acid and 8-mercaptooctanoic acid. Before conjugation to the peptides of the composition, the hydrophobic linking group comprises at least two reactive groups (A and B), as described herein and as shown below:

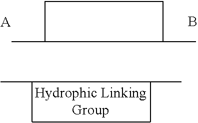

In some embodiments, the hydrophobic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on IFNL3 or M and the carboxylic acid or activated carboxylic acid can be coupled to an amine on IFNL3 or M with or without the use of a coupling reagent. Any coupling agent known to one skilled in the art can be used to couple the carboxylic acid with the free amine such as, for example, DCC, DIC, HATU, HBTU, TBTU, and other activating agents described herein. In specific embodiments, the hydrophilic linking group comprises an aliphatic chain of 2 to 100 methylene groups wherein A and B are carboxyl groups or derivatives thereof (e.g. succinic acid). In other specific embodiments the L is iodoacetic acid.

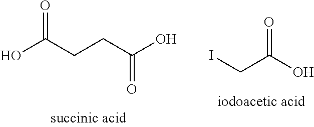 [SMILES: O=C(O)CCC(=O)O ; O=C(O)CI]

In some embodiments, the linking group is hydrophilic such as, for example, polyalkylene glycol. Before conjugation to the peptides of the composition, the hydrophilic linking group comprises at least two reactive groups (A and B), as described herein and as shown below:

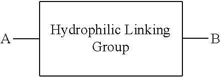

In specific embodiments, the linking group is polyethylene glycol (PEG). The PEG in certain embodiments has a molecular weight of about 100 Daltons to about 10,000 Daltons, e.g. about 500 Daltons to about 5000 Daltons. The PEG in some embodiments has a molecular weight of about 10,000 Daltons to about 40,000 Daltons.

In some embodiments, the hydrophilic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on IFNL3 or M and the carboxylic acid or activated carboxylic acid can be coupled to an amine on IFNL3 or M with or without the use of a coupling reagent. Any appropriate coupling agent known to one skilled in the art can be used to couple the carboxylic acid with the amine such as, for example, DCC, DIC, HATU, HBTU, TBTU, and other activating agents described herein. In some embodiments, the linking group is maleimido-PKEM(20-40 kDa)-COOH, iodoacetyl-PKEM(20-40 kDa)-COOH, maleimido-PKEM(20-40 kDa)—NHS, or iodoacetyl-PKEM(20-40 kDa)—NHS.

In some embodiments, the linking group is comprised of an amino acid, a dipeptide, a tripeptide, or a polypeptide, wherein the amino acid, dipeptide, tripeptide, or polypeptide comprises at least two activating groups, as described herein. In some embodiments, the linking group (L) comprises a moiety selected from the group consisting of: amino, ether, thioether, maleimido, disulfide, amide, ester, thioester, alkene, cycloalkene, alkyne, trizoyl, carbamate, carbonate, cathepsin B-cleavable, and hydrazone.

In some embodiments, L comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

In some embodiments, L is stable in biological fluids such as blood or blood fractions. In some embodiments, L is stable in blood serum for at least 5 minutes, e.g. less than 25%, 20%, 15%, 10% or 5% of the conjugate is cleaved when incubated in serum for a period of 5 minutes. In other embodiments, L is stable in blood serum for at least 10, or 20, or 25, or 30, or 60, or 90, or 120 minutes, or 3, 4, 5, 6, 7, 8, 9, 10, 12, 15, 18 or 24 hours. In these embodiments, L does not comprise a functional group that is capable of undergoing hydrolysis in vivo. In some exemplary embodiments, L is stable in blood serum for at least about 72 hours. Nonlimiting examples of functional groups that are not capable of undergoing significant hydrolysis in vivo include amides, ethers, and thioethers. For example, the following compound does not undergoing significant hydrolysis in vivo:

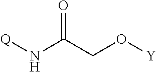

In some embodiments, L is hydrolyzable in vivo. In these embodiments, L comprises a functional group that is capable of undergoing hydrolysis in vivo. Nonlimiting examples of functional groups that are capable of undergoing hydrolysis in vivo include esters, anhydrides, and thioesters. For example the following compound is capable of undergoing hydrolysis in vivo because it comprises an ester group:

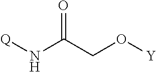

In some exemplary embodiments L is labile and undergoes substantial hydrolysis within 3 hours in blood plasma at 37° C., with complete hydrolysis within 6 hours. In some exemplary embodiments, L is not labile.

In some embodiments, L is metastable in vivo. In these embodiments, L comprises a functional group that is capable of being chemically or enzymatically cleaved in vivo (e.g., an acid-labile, reduction-labile, or enzyme-labile functional group), optionally over a period of time. In these embodiments, L can comprise, for example, a hydrazone moiety, a disulfide moiety, or a cathepsin-cleavable moiety. When L is metastable, and without intending to be bound by any particular theory, the IFNL3-L-M conjugate is stable in an extracellular environment, e.g., stable in blood serum for the time periods described above, but labile in the intracellular environment or conditions that mimic the intracellular environment, so that it cleaves upon entry into a cell. In some embodiments when L is metastable, L is stable in blood serum for at least about 24, 25, 26, 27, 28, 29, 30, 31, 32, 33, 34, 35, 36, 42, or 48 hours, for example, at least about 48, 54, 60, 66, or 72 hours, or about 24-48, 48-72, 24-60, 36-48, 36-72, or 48-72 hours.

### General Recombinant Nucleic Acid Methods

In numerous embodiments of the present invention, nucleic acids encoding an IFNL3 polypeptide of interest will be isolated, cloned and often altered using recombinant methods. Such embodiments are used, including but not limited to, for protein expression or during the generation of variants, derivatives, expression cassettes, or other sequences derived from an IFNL3 polypeptide. In some embodiments, the sequences encoding the polypeptides of the invention are operably linked to a heterologous promoter.

A nucleotide sequence encoding an IFNL3 polypeptide may be synthesized on the basis of the amino acid sequence of the parent polypeptide, including but not limited to, having the amino acid sequence shown in SEQ ID NO: 1 or SEQ ID NO: 2, or SEQ ID NO:3; SEQ ID NO 4; SEQ ID NO 5; SEQ ID NO: 6; SEQ ID NO: 7; SEQ ID NO: 8; or SEQ ID NO: 9 and then changing the nucleotide sequence so as to effect introduction (i.e., incorporation or substitution) or removal (i.e., deletion or substitution) of the relevant amino acid residue(s). The nucleotide sequence may be conveniently modified by site-directed mutagenesis in accordance with conventional methods. Alternatively, the nucleotide sequence may be prepared by chemical synthesis, including but not limited to, by using an oligonucleotide synthesizer, wherein oligonucleotides are designed based on the amino acid sequence of the desired polypeptide, and preferably selecting those codons that are favored in the host cell in which the recombinant polypeptide will be produced.

This invention utilizes routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

General texts which describe molecular biological techniques include Berger and Kimmel, Guide to Molecular Cloning Techniques, Methods in Enzymology volume 152 Academic Press, Inc., San Diego, Calif. (Berger); Sambrook et al., Molecular Cloning A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). These texts describe mutagenesis, the use of vectors, promoters and many other relevant topics related to, including but not limited to, the generation of genes or polynucleotides that include selector codons for production of proteins that include unnatural amino acids, orthogonal tRNAs, orthogonal synthetases, and pairs thereof.

Various types of mutagenesis are used in the invention for a variety of purposes, including but not limited to, to produce novel IFNL3 polypeptides of interest. They include but are not limited to site-directed, random point mutagenesis, homologous recombination, DNA shuffling or other recursive mutagenesis methods, chimeric construction, mutagenesis using uracil containing templates, oligonucleotide-directed mutagenesis, phosphorothioate-modified DNA mutagenesis, mutagenesis using gapped duplex DNA or the like, PCT-mediated mutagenesis, or any combination thereof. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, including but not limited to, involving chimeric constructs, are also included in the present invention. In one embodiment, mutagenesis can be guided by known information of the naturally occurring molecule or altered or mutated naturally occurring molecule, including but not limited to, sequence, sequence comparisons, physical properties, secondary, tertiary, or quaternary structure, crystal structure or the like.

The texts and examples found herein describe these procedures. Additional information is found in the following publications and references cited within: Ling et al., Approaches to DNA mutagenesis: an overview, Anal Biochem. 254(2): 157-178 (1997); Dale et al., Oligonucleotide-directed random mutagenesis using the phosphorothioate method, Methods Mol. Biol. 57:369-374 (1996); Smith, In vitro mutagenesis, Ann. Rev. Genet. 19:423-462 (1985); Botstein & Shortle, Strategies and applications of in vitro mutagenesis, Science 229:1193-1201 (1985); Carter, Site-directed mutagenesis, Biochem. J. 237:1-7 (1986); Kunkel, The efficiency of oligonucleotide directed mutagenesis, in Nucleic Acids & Molecular Biology (Eckstein, F. and Lilley, D. M. J. eds., Springer Verlag, Berlin) (1987); Kunkel, Rapid and efficient site-specific mutagenesis without phenotypic selection, Proc. Natl. Acad. Sci. USA 82:488-492 (1985); Kunkel et al., Rapid and efficient site-specific mutagenesis without phenotypic selection, Methods in Enzymol. 154, 367-382 (1987); Bass et al., Mutant Trp repressors with new DNA-binding specificities, Science 242:240-245 (1988); Zoller & Smith, Oligonucleotide-directed mutagenesis using M13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); Zoller & Smith, Oligonucleotide-directed mutagenesis: a simple method using two oligonucleotide primers and a single-stranded DNA template, Methods in Enzymol. 154:329-350 (1987); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8785 (1985); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Sayers et al., 5′-3′ Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Kramer et al., The gapped duplex DNA approach to oligonucleotide-directed mutation construction, Nucl. Acids Res. 12: 9441-9456 (1984); Kramer & Fritz Oligonucleotide-directed construction of mutations via gapped duplex DNA, Methods in Enzymol. 154:350-367 (1987); Kramer et al., Improved enzymatic in vitro reactions in the gapped duplex DNA approach to oligonucleotide-directed construction of mutations, Nucl. Acids Res. 16: 7207 (1988); Fritz et al., Oligonucleotide-directed construction of mutations: a gapped duplex DNA procedure without enzymatic reactions in vitro, Nucl. Acids Res. 16: 6987-6999 (1988); Kramer et al., Different base/base mismatches are corrected with different efficiencies by the methyl-directed DNA mismatch-repair system of E. coli, Cell 38:879-887 (1984); Carter et al., Improved oligonucleotide site-directed mutagenesis using M13 vectors, Nucl. Acids Res. 13: 4431-4443 (1985); Carter, Improved oligonucleotide-directed mutagenesis using M13 vectors, Methods in Enzymol. 154: 382-403 (1987); Eghtedarzadeh & Henikoff, Use of oligonucleotides to generate large deletions, Nucl. Acids Res. 14: 5115 (1986); Wells et al., Importance of hydrogen-bond formation in stabilizing the transition state of subtilisin, Phil. Trans. R. Soc. Lond. A 317: 415-423 (1986); Nambiar et al., Total synthesis and cloning of a gene coding for the ribonuclease S protein, Science 223: 1299-1301 (1984); Sakmar and Khorana, Total synthesis and expression of a gene for the alpha-subunit of bovine rod outer segment guanine nucleotide-binding protein (transducin), Nucl. Acids Res. 14: 6361-6372 (1988); Wells et al., Cassette mutagenesis: an efficient method for generation of multiple mutations at defined sites, Gene 34:315-323 (1985); Grundström et al., Oligonucleotide-directed mutagenesis by microscale ‘shot-gun’ gene synthesis, Nucl. Acids Res. 13: 3305-3316 (1985); Mandecki, Oligonucleotide-directed double-strand break repair in plasmids of Escherichia coli: a method for site-specific mutagenesis, Proc. Natl. Acad. Sci. USA, 83:7177-7181 (1986); Arnold, Protein engineering for unusual environments, Current Opinion in Biotechnology 4:450-455 (1993); Sieber, et al., Nature Biotechnology, 19:456-460 (2001); W. P. C. Stemmer, Nature 370, 389-91 (1994); and, I. A. Lorimer, I. Pastan, Nucleic Acids Res. 23, 3067-8 (1995). Additional details on many of the above methods can be found in Methods in Enzymology Volume 154, which also describes useful controls for trouble-shooting problems with various mutagenesis methods.

The invention also relates to eukaryotic host cells, non-eukaryotic host cells, and organisms for the in vivo incorporation of an unnatural amino acid via orthogonal tRNA/RS pairs. Host cells are genetically engineered (including but not limited to, transformed, transduced or transfected) with the polynucleotides of the invention or constructs which include a polynucleotide of the invention, including but not limited to, a vector of the invention, which can be, for example, a cloning vector or an expression vector. For example, the coding regions for the orthogonal tRNA, the orthogonal tRNA synthetase, and the protein to be derivatized are operably linked to gene expression control elements that are functional in the desired host cell. The vector can be, for example, in the form of a plasmid, a cosmid, a phage, a bacterium, a virus, a naked polynucleotide, or a conjugated polynucleotide. The vectors are introduced into cells and/or microorganisms by standard methods including electroporation (Fromm et al., Proc. Natl. Acad. Sci. USA 82, 5824 (1985)), infection by viral vectors, high velocity ballistic penetration by small particles with the nucleic acid either within the matrix of small beads or particles, or on the surface (Klein et al., Nature 327, 70-73 (1987)), and/or the like. Techniques suitable for the transfer of nucleic acid into cells in vitro include the use of liposomes, microinjection, cell fusion, DEAE-dextran, the calcium phosphate precipitation method, etc. In vivo gene transfer techniques include, but are not limited to, transfection with viral (typically retroviral) vectors and viral coat protein-liposome mediated transfection [Dzau et al., Trends in Biotechnology 11:205-210 (1993)]. In some situations it may be desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life.

The engineered host cells can be cultured in conventional nutrient media modified as appropriate for such activities as, for example, screening steps, activating promoters or selecting transformants. These cells can optionally be cultured into transgenic organisms. Other useful references, including but not limited to for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg N.Y.) and Atlas and Parks (eds.) The Handbook of Microbiological Media (1993) CRC Press, Boca Raton, Fla.

Several well-known methods of introducing target nucleic acids into cells are available, any of which can be used in the invention. These include: fusion of the recipient cells with bacterial protoplasts containing the DNA, electroporation, projectile bombardment, and infection with viral vectors (discussed further, below), etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, kits are commercially available for the purification of plasmids from bacteria, (see, e.g., EasyPrep™, FlexiPrep™, both from Pharmacia Biotech; StrataClean™ from Stratagene; and, QIAprep™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (including but not limited to, shuttle vectors) and selection markers for both prokaryotic and eukaryotic systems. Vectors are suitable for replication and integration in prokaryotes, eukaryotes, or both. See, Gillam & Smith, Gene 8:81 (1979); Roberts, et al., Nature, 328:731 (1987); Schneider, E., et al., Protein Expr. Purif 6(1):10-14 (1995); Ausubel, Sambrook, Berger (all supra). A catalogue of bacteria and bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books, NY. In addition, essentially any nucleic acid (and virtually any labeled nucleic acid, whether standard or non-standard) can be custom or standard ordered from any of a variety of commercial sources, such as the Midland Certified Reagent Company (Midland, Tex. available on the World Wide Web at mcrc.com), The Great American Gene Company (Ramona, Calif. available on the World Wide Web at genco.com), ExpressGen Inc. (Chicago, Ill. available on the World Wide Web at expressgen.com), Operon Technologies Inc. (Alameda, Calif.) and many others.

In yet another aspect, the post-translation modification includes proteolytic processing of precursors (including but not limited to, proIFNL3 or a variant or analog thereof), assembly into a multisubunit protein or macromolecular assembly, translation to another site in the cell (including but not limited to, to organelles, such as the endoplasmic reticulum, the Golgi apparatus, the nucleus, lysosomes, peroxisomes, mitochondria, chloroplasts, vacuoles, etc., or through the secretory pathway). In certain embodiments, the protein comprises a secretion or localization sequence, an epitope tag, a FLAG tag, a polyhistidine tag, a GST fusion, or the like.

### Location of Modifications in IFNL3 Polypeptides

The present invention contemplates the substitution, addition, deletion, or incorporation of one or more naturally encoded or non-naturally-occurring amino acids into IFNL3 polypeptides. One or more of these modifications may be incorporated at a particular position which does not disrupt activity of the polypeptide. This can be achieved by making “conservative” substitutions, including but not limited to, substituting hydrophobic amino acids with hydrophobic amino acids, bulky amino acids for bulky amino acids, hydrophilic amino acids for hydrophilic amino acids and/or inserting the non-naturally-occurring amino acid in a location that is not required for activity. The three-dimensional crystal structure of mammalian IFNL3 polypeptides has been determined. IFNL3 is known to comprise several disulfide bonds. Cysteine residues, and in particular unpaired cysteine residues, may be involved in post-translational chemical reactions due to the SH functional group, and as such the cysteine residues are a target for replacement in order to test for the ability to modulate the stability of the IFNL3 polypeptide, its receptor binding activity, or other biological property of the polypeptide. Therefore the remaining cysteine residues may individually, or in combination, or entirely, be substituted with another amino acid, such as but not limited to serine or alanine, in order to remove them from the IFNL3 polypeptide and test the biological properties of the resulting IFNL3 polypeptide.

A variety of biochemical and structural approaches can be employed to select the desired sites for substitution with a naturally encoded or non-naturally encoded amino acid within the IFNL3 polypeptide. It is readily apparent to those of ordinary skill in the art that any position of the polypeptide chain is suitable for selection to incorporate a naturally encoded or non-naturally encoded amino acid, and selection may be based on rational design or by random selection for any or no particular desired purpose. Selection of desired sites may be for producing an IFNL3 molecule having any desired property or activity, including but not limited to, agonists, super-agonists, inverse agonists, antagonists, receptor binding modulators, receptor activity modulators, dimer or multimer formation, no change to activity or property compared to the native molecule, or manipulating any physical or chemical property of the polypeptide such as solubility, aggregation, or stability. For example, locations in the polypeptide required for biological activity of IFNL3 polypeptides can be identified using point mutation analysis, alanine scanning, saturation mutagenesis and screening for biological activity, or homolog scanning methods known in the art. Other methods can be used to identify residues for modification of IFNL3 polypeptides include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994)), and rational design using Protein Design Automation® technology. (See U.S. Pat. Nos. 6,188,965; 6,269,312; 6,403,312; WO98/47089, which are incorporated by reference). Residues that are critical for IFNL3 bioactivity, residues that are involved with pharmaceutical stability, antibody epitopes, or receptor, activity modulator, or other IFNL3 polypeptide binding residues may be mutated. U.S. Pat. Nos. 5,580,723; 5,834,250; 6,013,478; 6,428,954; and 6,451,561, which are incorporated by reference herein, describe methods for the systematic analysis of the structure and function of polypeptides, such as IFNL3 by identifying active domains which influence the activity of the polypeptide with a target substance. Residues other than those identified as critical to biological activity by alanine or homolog scanning mutagenesis may be good candidates for substitution, deletion, or insertion depending on the desired activity sought for the polypeptide. Alternatively, the sites identified as critical to biological activity may also be good candidates for substitution, insertion or deletion, again depending on the desired activity sought for the polypeptide. Another alternative would be to simply make serial substitutions in each position on the polypeptide chain with a non-naturally encoded amino acid and observe the effect on the activities of the polypeptide. It is readily apparent to those of ordinary skill in the art that any means, technique, or method for selecting a position for substitution with a non-natural amino acid into any polypeptide is suitable for use in the present invention.

The structure and activity of mutants of IFNL3 polypeptides can also be examined to determine regions of the protein that are likely to be tolerant of addition or deletion of amino acids, or of substitution with a naturally encoded or non-naturally encoded amino acid. In a similar manner, protease digestion and monoclonal antibodies can be used to identify regions of IFNL3 that are responsible for binding the IFNL3 to its receptor, modulators of activity, or dimerization. Once residues that are likely to be intolerant to addition or deletion of amino acids, or of substitution with naturally encoded or non-naturally encoded amino acids have been eliminated, the impact of proposed substitutions or other changes at each of the remaining positions can be examined. Models may be generated from the three-dimensional crystal structures of other IFNL3 family members as well. Protein Data Bank (PDB, available on the World Wide Web at rcsb.org) is a centralized database containing structural data of large molecules such as IFNL3 and other proteins and nucleic acids. Models may be made investigating the secondary and tertiary structure of polypeptides, if three-dimensional structural data is not available. Thus, those of ordinary skill in the art can readily identify amino acid positions that can be candidates for addition or deletion of amino acids, or of substitution with naturally encoded or non-naturally encoded amino acids.

In some embodiments, the IFNL3 polypeptides of the invention comprise one or more addition or deletion of amino acids, or of substitution of naturally encoded or non-naturally encoded amino acids positioned in a region of the protein that does not disrupt the structure of the polypeptide.

Exemplary residues of incorporation of a naturally encoded or a non-naturally encoded amino acid may be those that are excluded from potential receptor, modulator or dimerization binding regions, may be fully or partially solvent exposed, have minimal or no hydrogen-bonding interactions with nearby residues, may be minimally exposed to nearby reactive residues, may be on one or more of the exposed faces, may be a site or sites that are juxtaposed to a second IFNL3, or other molecule or fragment thereof, may be in regions that are highly flexible, or structurally rigid, as predicted by the three-dimensional, secondary, tertiary, or quaternary structure of IFNL3, or coupled or not coupled to another biologically active molecule, or may modulate the conformation of the IFNL3 itself or a dimer or multimer comprising one or more IFNL3, by altering the flexibility or rigidity of the complete structure as desired.

One of ordinary skill in the art recognizes that such analysis of IFNL3 enables the determination of which amino acid residues are surface exposed compared to amino acid residues that are buried within the tertiary structure of the protein. Therefore, it is an embodiment of the present invention to substitute, insert or delete one or more amino acid for an amino acid that is a surface exposed residue.

An examination of the crystal structure of IFNL3 and its interaction with the IFNL3 receptor, modulator, or another IFNL3 molecule can indicate which certain amino acid residues have side chains that are fully or partially accessible to solvent. The side chain of an amino acid at these positions may point away from the protein surface and out into the solvent.

A wide variety of non-naturally encoded amino acids can be substituted for, or incorporated into, a given position in an IFNL3 polypeptide. In general, a particular non-naturally encoded amino acid is selected for incorporation based on an examination of the three dimensional crystal structure of an IFNL3 polypeptide or other IFNL3 family member or IFNL3 analog, a preference for conservative substitutions (i.e., aryl-based non-naturally encoded amino acids, such as p-acetylphenylalanine or O-propargyltyrosine substituting for Phe, Tyr or Trp), and the specific conjugation chemistry that one desires to introduce into the IFNL3 polypeptide (e.g., the introduction of 4-azidophenylalanine if one wants to effect a Huisgen [3+2] cycloaddition with a PKEM bearing an alkyne moiety or a amide bond formation with a PKEM that bears an aryl ester that, in turn, incorporates a phosphine moiety)

In one embodiment, the method further includes incorporating into the protein the unnatural amino acid, where the unnatural amino acid comprises a first reactive group; and contacting the protein with a molecule (including but not limited to, a PKEM, a label, a dye, a polymer, a water-soluble polymer, a derivative of polyethylene glycol, a photocrosslinker, a radionuclide, a cytotoxic compound, a drug, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, a neutron-capture agent, or any combination of the above, or any other desirable compound or substance) that comprises a second reactive group. The first reactive group reacts with the second reactive group to attach the molecule to the unnatural amino acid through a [3+2] cycloaddition. In one embodiment, the first reactive group is an alkynyl or azido moiety and the second reactive group is an azido or alkynyl moiety. For example, the first reactive group is the alkynyl moiety (including but not limited to, in unnatural amino acid p-propargyloxyphenylalanine) and the second reactive group is the azido moiety. In another example, the first reactive group is the azido moiety (including but not limited to, in the unnatural amino acid p-azido-L-phenylalanine) and the second reactive group is the alkynyl moiety.

In some cases, the naturally encoded or non-naturally encoded amino acid substitution(s) will be combined with other additions, substitutions or deletions within the IFNL3 polypeptide to affect other biological traits of the IFNL3 polypeptide. In some cases, the other additions, substitutions or deletions may increase the stability (including but not limited to, resistance to proteolytic degradation) of the IFNL3 polypeptide or increase affinity of the IFNL3 polypeptide for its receptor, activity modulator, or other IFNL3 polypeptide. In some cases, the other additions, substitutions or deletions may increase the pharmaceutical stability of the IFNL3 polypeptide. In some cases, the other additions, substitutions or deletions may enhance the activity/efficacy of the IFNL3 polypeptide. In some cases, the other additions, substitutions or deletions may increase the solubility (including but not limited to, when expressed in E. coli or other host cells) of the IFNL3 polypeptide or increase the expression and production levels of the protein in the host cells. In some embodiments additions, substitutions or deletions may increase the IFNL3 polypeptide solubility following expression in E. coli or other recombinant host cells. In some embodiments sites are selected for substitution with a naturally encoded or non-natural amino acid in addition to another site for incorporation of a non-natural amino acid that results in increasing the polypeptide solubility following expression in E. coli or other recombinant host cells. In some embodiments, the IFNL3 polypeptides comprise another addition, substitution or deletion that modulates affinity for the IFNL3 polypeptide receptor, modulator such as zinc, binding proteins, or associated ligand, modulates IFNL3 activity, modulates circulating half-life, modulates release or bio-availability, facilitates purification, or improves or alters a particular route of administration. In some embodiments, the IFNL3 polypeptides comprise an addition, substitution or deletion that increases the affinity of the IFNL3 variant for its receptor, modulator, or other IFNL3 polypeptides. Similarly, IFNL3 polypeptides can comprise chemical or enzyme cleavage sequences, protease cleavage sequences, reactive groups, antibody-binding domains (including but not limited to, FLAG or poly-His) or other affinity based sequences (including, but not limited to, FLAG, poly-His, GST, etc.) or linked molecules (including, but not limited to, biotin) that improve detection (including, but not limited to, GFP), purification, transport through tissues or cell membranes, prodrug release or activation, IFNL3 size reduction, or other traits of the polypeptide.

In some embodiments, the substitution of a naturally encoded or non-naturally encoded amino acid generates an IFNL3 polypeptide that has decreased activity but has greater stability when compared to unmodified IFNL3. Increasing stability may actually result in an IFNL3 polypeptide that has, for example, an increased circulation time after administration to a subject even though the IFNL3 polypeptide has a decreased inflammatory activity, which may in certain cases be more desirable than the wild type IFNL3. In some embodiments, a naturally encoded or non-naturally encoded amino acid is substituted or added in a region involved with receptor, modulator, or IFNL3 activity. In some embodiments, the modified IFNL3 polypeptide comprises at least one substitution that causes the IFNL3 to act as an antagonist of IFNL3 which may be useful to modulate the activity of an IFNL3 polypeptide that has been administered to a subject.

In some cases, 1, 2, 3, 4, 5, 6, 7, 8, 9, 10, or more amino acids are substituted with one or more naturally encoded or non-naturally-encoded amino acids. In some cases, the IFNL3 polypeptide further includes 1, 2, 3, 4, 5, 6, 7, 8, 9, 10, or more substitutions of one or more non-naturally encoded amino acids for naturally occurring amino acids. For example, in some embodiments, one or more residues in IFNL3 are substituted with one or more non-naturally encoded amino acids. In some cases, the one or more non-naturally encoded residues are linked to one or more lower molecular weight PKEM, thereby enhancing binding affinity and comparable serum half-life relative to the species attached to a single, higher molecular weight PKEM.

In some embodiments, up to two of the following residues of IFNL3 are substituted with one or more naturally encoded or non-naturally encoded amino acids.

### Expression in Non-Eukaryotes and Eukaryotes

To obtain high level expression of a cloned IFNL3 polynucleotide, one typically subclones polynucleotides encoding an IFNL3 polypeptide of the invention into an expression vector that contains a strong promoter to direct transcription, a transcription/translation terminator, and if for a nucleic acid encoding a protein, a ribosome binding site for translational initiation. Suitable bacterial promoters are known to those of ordinary skill in the art and described, e.g., in Sambrook et al. and Ausubel et al.

Bacterial expression systems for expressing IFNL3 polypeptides of the invention are available in, including but not limited to, E. coli, Bacillus sp., Pseudomonas fluorescens, Pseudomonas aeruginosa, Pseudomonas putida, and Salmonella (Palva et al., Gene 22:229-235 (1983); Mosbach et al., Nature 302:543-545 (1983)). Kits for such expression systems are commercially available. Eukaryotic expression systems for mammalian cells, yeast, and insect cells are known to those of ordinary skill in the art and are also commercially available. In cases where orthogonal tRNAs and aminoacyl tRNA synthetases (described above) are used to express the IFNL3 polypeptides of the invention, host cells for expression are selected based on their ability to use the orthogonal components. Exemplary host cells include Gram-positive bacteria (including but not limited to B. brevis, B. subtilis, or Streptomyces) and Gram-negative bacteria (E. coli, Pseudomonas fluorescens, Pseudomonas aeruginosa, Pseudomonas putida), as well as yeast and other eukaryotic cells. Cells comprising O-tRNA/O—RS pairs can be used as described herein.

A eukaryotic host cell or non-eukaryotic host cell of the present invention provides the ability to synthesize proteins that comprise unnatural amino acids in large useful quantities. In one aspect, the composition optionally includes, including but not limited to, at least 10 micrograms, at least 50 micrograms, at least 75 micrograms, at least 100 micrograms, at least 200 micrograms, at least 250 micrograms, at least 500 micrograms, at least 1 milligram, at least 10 milligrams, at least 100 milligrams, at least one gram, or more of the protein that comprises an unnatural amino acid, or an amount that can be achieved with in vivo protein production methods (details on recombinant protein production and purification are provided herein). In another aspect, the protein is optionally present in the composition at a concentration of, including but not limited to, at least 10 micrograms of protein per liter, at least 50 micrograms of protein per liter, at least 75 micrograms of protein per liter, at least 100 micrograms of protein per liter, at least 200 micrograms of protein per liter, at least 250 micrograms of protein per liter, at least 500 micrograms of protein per liter, at least 1 milligram of protein per liter, or at least 10 milligrams of protein per liter or more, in, including but not limited to, a cell lysate, a buffer, a pharmaceutical buffer, or other liquid suspension (including but not limited to, in a volume of, including but not limited to, anywhere from about 1 nl to about 100 L or more). The production of large quantities (including but not limited to, greater that that typically possible with other methods, including but not limited to, in vitro translation) of a protein in a eukaryotic cell including at least one unnatural amino acid is a feature of the invention.

A eukaryotic host cell or non-eukaryotic host cell of the present invention provides the ability to biosynthesize proteins that comprise unnatural amino acids in large useful quantities. For example, proteins comprising an unnatural amino acid can be produced at a concentration of, including but not limited to, at least 10 μg/liter, at least 50 μg/liter, at least 75 μg/liter, at least 100 μg/liter, at least 200 μg/liter, at least 250 μg/liter, or at least 500 μg/liter, at least 1 mg/liter, at least 2 mg/liter, at least 3 mg/liter, at least 4 mg/liter, at least 5 mg/liter, at least 6 mg/liter, at least 7 mg/liter, at least 8 mg/liter, at least 9 mg/liter, at least 10 mg/liter, at least 20, 30, 40, 50, 60, 70, 80, 90, 100, 200, 300, 400, 500, 600, 700, 800, 900 mg/liter, 1 g/liter, 5 g/liter, 10 g/liter or more of protein in a cell extract, cell lysate, culture medium, a buffer, and/or the like.

A number of vectors suitable for expression of IFNL3 are commercially available. Useful expression vectors for eukaryotic hosts, include but are not limited to, vectors comprising expression control sequences from SV40, bovine papilloma virus, adenovirus and cytomegalovirus. Such vectors include pCDNA3.1(+)\Hyg (Invitrogen, Carlsbad, Calif., USA) and pCI-neo (Stratagene, La Jolla, Calif., USA). Bacterial plasmids, such as plasmids from E. coli, including pBR322, pET3a and pET12a, wider host range plasmids, such as RP4, phage DNAs, e.g., the numerous derivatives of phage lambda, e.g., NM989, and other DNA phages, such as M13 and filamentous single stranded DNA phages may be used. The 2μ plasmid and derivatives thereof, the POT1 vector (U.S. Pat. No. 4,931,373 which is incorporated by reference), the pJSO37 vector described in (Okkels, Ann. N.Y. Aced. Sci. 782, 202 207, 1996) and pPICZ A, B or C (Invitrogen) may be used with yeast host cells. For insect cells, the vectors include but are not limited to, pVL941, pBG311 (Cate et al., “Isolation of the Bovine and Human Genes for Mullerian Inhibiting Substance and Expression of the Human Gene In Animal Cells”, Cell, 45, pp. 685 98 (1986), pBluebac 4.5 and pMelbac (Invitrogen, Carlsbad, Calif.).

The nucleotide sequence encoding an IFNL3 polypeptide may or may not also include sequence that encodes a signal peptide. The signal peptide is present when the polypeptide is to be secreted from the cells in which it is expressed. Such signal peptide may be any sequence. The signal peptide may be prokaryotic or eukaryotic. Coloma, M (1992) J. Imm. Methods 152:89 104) describe a signal peptide for use in mammalian cells (murine Ig kappa light chain signal peptide). Other signal peptides include but are not limited to, the α-factor signal peptide from S. cerevisiae (U.S. Pat. No. 4,870,008 which is incorporated by reference herein), the signal peptide of mouse salivary amylase (O. Hagenbuchle et al., Nature 289, 1981, pp. 643-646), a modified carboxypeptidase signal peptide (L. A. Valls et al., Cell 48, 1987, pp. 887-897), the yeast BARI signal peptide (WO 87/02670, which is incorporated by reference herein), and the yeast aspartic protease 3 (YAP3) signal peptide (cf M. Egel-Mitani et al., Yeast 6, 1990, pp. 127-137). A IFNL3 polypeptide sequence of the disclosure or encoding polynucleotide may include a natural IFNL3 signal peptide or coding sequence, such as MPRLFFFHLLGVCLLLNQFSRAVA (SEQ ID NO: 19) (SwissProt accession no P04090) or any natural sequence variant thereof e.g., as described above.

Examples of suitable mammalian host cells are known to those of ordinary skill in the art. Such host cells may be Chinese hamster ovary (CHO) cells, (e.g. CHO-K1; ATCC CCL-61), Green Monkey cells (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture or on whole plants. These cell lines and others are available from public depositories such as the American Type Culture Collection, Rockville, Md. In order to provide improved glycosylation of the IFNL3 polypeptide, a mammalian host cell may be modified to express sialyltransferase, e.g. 1,6-sialyltransferase, e.g. as described in U.S. Pat. No. 5,047,335, which is incorporated by reference herein.

Methods for the introduction of exogenous DNA into mammalian host cells include but are not limited to, calcium phosphare-mediated transfection, electroporation, DEAE-dextran mediated transfection, liposome-mediated transfection, viral vectors and the transfection methods described by Life Technologies Ltd, Paisley, UK using Lipofectamin 2000 and Roche Diagnostics Corporation, Indianapolis, USA using FuGENE 6. These methods are well known in the art and are described by Ausbel et al. (eds.), 1996, Current Protocols in Molecular Biology, John Wiley & Sons, New York, USA. The cultivation of mammalian cells may be performed according to established methods, e.g. as disclosed in (Animal Cell Biotechnology, Methods and Protocols, Edited by Nigel Jenkins, 1999, Human Press Inc. Totowa, N.J., USA and Harrison Mass. and Rae I F, General Techniques of Cell Culture, Cambridge University Press 1997).

### Expression Systems, Culture, and Isolation

IFNL3 polypeptides may be expressed in any number of suitable expression systems including, for example, yeast, insect cells, mammalian cells, and bacteria. A description of exemplary expression systems is provided below.

Yeast As used herein, the term “yeast” includes any of the various yeasts capable of expressing a gene encoding an IFNL3 polypeptide. Such yeasts include, but are not limited to, ascosporogenous yeasts (Endomycetales), basidiosporogenous yeasts and yeasts belonging to the Fungi imperfecti (Blastomycetes) group. The ascosporogenous yeasts are divided into two families, Spermophthoraceae and Saccharomycetaceae. The latter is comprised of four subfamilies, Schizosaccharomycoideae (e.g., genus Schizosaccharomyces), Nadsonioideae, Lipomycoideae and Saccharomycoideae (e.g., genera Pichia, Kluyveromyces and Saccharomyces). The basidiosporogenous yeasts include the genera Leucosporidium, Rhodosporidium, Sporidiobolus, Filobasidium, and Filobasidiella. Yeasts belonging to the Fungi Imperfecti (Blastomycetes) group are divided into two families, Sporobolomycetaceae (e.g., genera Sporobolomyces and Bullera) and Cryptococcaceae (e.g., genus Candida).

Of particular interest for use with the present invention are species within the genera Pichia, Kluyveromyces, Saccharomyces, Schizosaccharomyces, Hansenula, Torulopsis, and Candida, including, but not limited to, P. pastoris, P. guillerimondii, S. cerevisiae, S. carlsbergensis, S. diastaticus, S. douglasii, S. kluyveri, S. norbensis, S. oviformis, K. lactis, K. fragilis, C. albicans, C. maltosa, and H. polymorpha.

The selection of suitable yeast for expression of IFNL3 polypeptides is within the skill of one of ordinary skill in the art. In selecting yeast hosts for expression, suitable hosts may include those shown to have, for example, good secretion capacity, low proteolytic activity, good secretion capacity, good soluble protein production, and overall robustness. Yeast is generally available from a variety of sources including, but not limited to, the Yeast Genetic Stock Center, Department of Biophysics and Medical Physics, University of California (Berkeley, Calif.), and the American Type Culture Collection (“ATCC”) (Manassas, Va.).

The term “yeast host” or “yeast host cell” includes yeast that can be, or has been, used as a recipient for recombinant vectors or other transfer DNA. The term includes the progeny of the original yeast host cell that has received the recombinant vectors or other transfer DNA. It is understood that the progeny of a single parental cell may not necessarily be completely identical in morphology or in genomic or total DNA complement to the original parent, due to accidental or deliberate mutation. Progeny of the parental cell that are sufficiently similar to the parent to be characterized by the relevant property, such as the presence of a nucleotide sequence encoding an IFNL3 polypeptide, are included in the progeny intended by this definition.

Expression and transformation vectors, including extrachromosomal replicons or integrating vectors, have been developed for transformation into many yeast hosts. For example, expression vectors have been developed for S. cerevisiae (Sikorski et al., GENETICS (1989) 122:19; Ito et al., J. BACTERIOL. (1983) 153:163; Hinnen et al., PROC. NATL. ACAD. SCI. USA (1978) 75:1929); C. albicans (Kurtz et al., MOL. CELL. BIOL. (1986) 6:142); C. maltosa (Kunze et al., J. BASIC MICROBIOL. (1985) 25:141); H. polymorpha (Gleeson et al., J. GEN. MICROBIOL. (1986) 132:3459; Roggenkamp et al., MOL. GENETICS AND GENOMICS (1986) 202:302); K. fragilis (Das et al., J. BACTERIOL. (1984) 158:1165); K. lactis (De Louvencourt et al., J. BACTERIOL. (1983) 154:737; Van den Berg et al., BIOTECHNOLOGY (NY) (1990) 8:135); P. guillerimondii (Kunze et al., J. BASIC MICROBIOL. (1985) 25:141); P. pastoris (U.S. Pat. Nos. 5,324,639; 4,929,555; and U.S. Pat. No. 4,837,148; Cregg et al., MOL. CELL. BIOL. (1985) 5:3376); Schizosaccharomyces pombe (Beach et al., NATURE (1982) 300:706); and Y. lipolytica; A. nidulans (Ballance et al., BIOCHEM. BIOPHYS. RES. COMMUN. (1983) 112:284-89; Tilbum et al., GENE (1983) 26:205-221; and Yelton et al., PROC. NATL. ACAD. SCI. USA (1984) 81:1470-74); A. niger (Kelly and Hynes, EMBO J. (1985) 4:475-479); T. reesei (EP 0 244 234); and filamentous fungi such as, e.g., Neurospora, Penicillium, Tolypocladium (WO 91/00357), each incorporated by reference herein.

Control sequences for yeast vectors are known to those of ordinary skill in the art and include, but are not limited to, promoter regions from genes such as alcohol dehydrogenase (ADH) (EP 0 284 044); enolase; glucokinase; glucose-6-phosphate isomerase; glyceraldehyde-3-phosphate-dehydrogenase (GAP or GAPDH); hexokinase; phosphofructokinase; 3-phosphoglycerate mutase; and pyruvate kinase (PyK) (EP 0 329 203). The yeast PHO5 gene, encoding acid phosphatase, also may provide useful promoter sequences (Miyanohara et al., PROC. NATL. ACAD. SCI. USA (1983) 80:1). Other suitable promoter sequences for use with yeast hosts may include the promoters for 3-phosphoglycerate kinase (Hitzeman et al., J. BIOL. CHEM. (1980) 255:12073); and other glycolytic enzymes, such as pyruvate decarboxylase, triosephosphate isomerase, and phosphoglucose isomerase (Holland et al., BIOCHEMISTRY (1978) 17:4900; Hess et al., J. ADV. ENZYME REG. (1969) 7:149). Inducible yeast promoters having the additional advantage of transcription controlled by growth conditions may include the promoter regions for alcohol dehydrogenase 2; isocytochrome C; acid phosphatase; metallothionein; glyceraldehyde-3-phosphate dehydrogenase; degradative enzymes associated with nitrogen metabolism; and enzymes responsible for maltose and galactose utilization. Suitable vectors and promoters for use in yeast expression are further described in EP 0 073 657.

Yeast enhancers also may be used with yeast promoters. In addition, synthetic promoters may also function as yeast promoters. For example, the upstream activating sequences (UAS) of a yeast promoter may be joined with the transcription activation region of another yeast promoter, creating a synthetic hybrid promoter. Examples of such hybrid promoters include the ADH regulatory sequence linked to the GAP transcription activation region. See U.S. Pat. Nos. 4,880,734 and 4,876,197, which are incorporated by reference herein. Other examples of hybrid promoters include promoters that consist of the regulatory sequences of the ADH2, GAL4, GAL10, or PHO5 genes, combined with the transcriptional activation region of a glycolytic enzyme gene such as GAP or PyK. See EP 0 164 556. Furthermore, a yeast promoter may include naturally occurring promoters of non-yeast origin that have the ability to bind yeast RNA polymerase and initiate transcription.

Other control elements that may comprise part of the yeast expression vectors include terminators, for example, from GAPDH or the enolase genes (Holland et al., J. BIOL. CHEM. (1981) 256:1385). In addition, the origin of replication from the 2μ plasmid origin is suitable for yeast. A suitable selection gene for use in yeast is the trp1 gene present in the yeast plasmid. See Tschumper et al., GENE (1980) 10:157; Kingsman et al., GENE (1979) 7:141. The trp1 gene provides a selection marker for a mutant strain of yeast lacking the ability to grow in tryptophan. Similarly, Leu2-deficient yeast strains (ATCC 20,622 or 38,626) are complemented by known plasmids bearing the Leu2 gene.

Methods of introducing exogenous DNA into yeast hosts are known to those of ordinary skill in the art, and typically include, but are not limited to, either the transformation of spheroplasts or of intact yeast host cells treated with alkali cations. For example, transformation of yeast can be carried out according to the method described in Hsiao et al., PROC. NATL. ACAD. SCI. USA (1979) 76:3829 and Van Solingen et al., J. BACT. (1977) 130:946. However, other methods for introducing DNA into cells such as by nuclear injection, electroporation, or protoplast fusion may also be used as described generally in SAMBROOK ET AL., MOLECULAR CLONING: A LAB. MANUAL (2001). Yeast host cells may then be cultured using standard techniques known to those of ordinary skill in the art.

Other methods for expressing heterologous proteins in yeast host cells are known to those of ordinary skill in the art. See generally U.S. Patent Publication No. 20020055169, U.S. Pat. Nos. 6,361,969; 6,312,923; 6,183,985; 6,083,723; 6,017,731; 5,674,706; 5,629,203; 5,602,034; and 5,089,398; U.S. Reexamined Pat. Nos. RE37,343 and RE35,749; PCT Published Patent Applications WO 99/07862; WO 98/37208; and WO 98/26080; European Patent Applications EP 0 946 736; EP 0 732 403; EP 0 480 480; WO 90/10277; EP 0 340 986; EP 0 329 203; EP 0 324 274; and EP 0 164 556. See also Gellissen et al., ANTONIE VAN LEEUWENHOEK (1992) 62 (1-2):79-93; Romanos et al., YEAST (1992) 8(6):423-488; Goeddel, METHODS IN ENZYMOLOGY (1990) 185:3-7, each incorporated by reference herein.

The yeast host strains may be grown in fermentors during the amplification stage using standard feed batch fermentation methods known to those of ordinary skill in the art. The fermentation methods may be adapted to account for differences in a particular yeast host's carbon utilization pathway or mode of expression control. For example, fermentation of a Saccharomyces yeast host may require a single glucose feed, complex nitrogen source (e.g., casein hydrolysates), and multiple vitamin supplementation. In contrast, the methylotrophic yeast P. pastoris may require glycerol, methanol, and trace mineral feeds, but only simple ammonium (nitrogen) salts for optimal growth and expression. See, e.g., U.S. Pat. No. 5,324,639; Elliott et al., J. PROTEIN CHEM. (1990) 9:95; and Fieschko et al., BIOTECH. BIOENG. (1987) 29:1113, incorporated by reference herein.

Such fermentation methods, however, may have certain common features independent of the yeast host strain employed. For example, a growth limiting nutrient, typically carbon, may be added to the fermentor during the amplification phase to allow maximal growth. In addition, fermentation methods generally employ a fermentation medium designed to contain adequate amounts of carbon, nitrogen, basal salts, phosphorus, and other minor nutrients (vitamins, trace minerals and salts, etc.). Examples of fermentation media suitable for use with Pichia are described in U.S. Pat. Nos. 5,324,639 and 5,231,178, which are incorporated by reference herein.

Baculovirus-Infected Insect Cells The term “insect host” or “insect host cell” refers to a insect that can be, or has been, used as a recipient for recombinant vectors or other transfer DNA. The term includes the progeny of the original insect host cell that has been transfected. It is understood that the progeny of a single parental cell may not necessarily be completely identical in morphology or in genomic or total DNA complement to the original parent, due to accidental or deliberate mutation. Progeny of the parental cell that are sufficiently similar to the parent to be characterized by the relevant property, such as the presence of a nucleotide sequence encoding an IFNL3 polypeptide, are included in the progeny intended by this definition. Baculovirus expression of IFNL3 polypeptides is useful in the present invention and the use of rDNA technology, polypeptides or precursors thereof because IFNL3 may be biosynthesized in any number of host cells including bacteria, mammalian cells, insect cells, yeast or fungi. An embodiment of the present invention includes biosynthesis of IFNL3, modified IFNL3, IFNL3 polypeptides, or IFNL3 analogs in bacteria, yeast or mammalian cells. Another embodiment of the present invention involves biosynthesis done in E. coli or a yeast. Examples of biosynthesis in mammalian cells and transgenic animals are described in Hakola, K. [Molecular and Cellular Endocrinology, 127:59-69, (1997)].

The selection of suitable insect cells for expression of IFNL3 polypeptides is known to those of ordinary skill in the art. Several insect species are well described in the art and are commercially available including Aedes aegypti, Bombyx mori, Drosophila melanogaster, Spodoptera frugiperda, and Trichoplusia ni. In selecting insect hosts for expression, suitable hosts may include those shown to have, inter alia, good secretion capacity, low proteolytic activity, and overall robustness. Insect are generally available from a variety of sources including, but not limited to, the Insect Genetic Stock Center, Department of Biophysics and Medical Physics, University of California (Berkeley, Calif.); and the American Type Culture Collection (“ATCC”) (Manassas, Va.).

Generally, the components of a baculovirus-infected insect expression system include a transfer vector, usually a bacterial plasmid, which contains both a fragment of the baculovirus genome, and a convenient restriction site for insertion of the heterologous gene to be expressed; a wild type baculovirus with sequences homologous to the baculovirus-specific fragment in the transfer vector (this allows for the homologous recombination of the heterologous gene in to the baculovirus genome); and appropriate insect host cells and growth media. The materials, methods and techniques used in constructing vectors, transfecting cells, picking plaques, growing cells in culture, and the like are known in the art and manuals are available describing these techniques.

After inserting the heterologous gene into the transfer vector, the vector and the wild type viral genome are transfected into an insect host cell where the vector and viral genome recombine. The packaged recombinant virus is expressed and recombinant plaques are identified and purified. Materials and methods for baculovirus/insect cell expression systems are commercially available in kit form from, for example, Invitrogen Corp. (Carlsbad, Calif.). These techniques are generally known to those of ordinary skill in the art and fully described in SUMMERS AND SMITH, TEXAS AGRICULTURAL EXPERIMENT STATION BULLETIN NO. 1555 (1987), herein incorporated by reference. See also, RICHARDSON, 39 METHODS IN MOLECULAR BIOLOGY: BACULOVIRUS EXPRESSION PROTOCOLS (1995); AUSUBEL ET AL., CURRENT PROTOCOLS IN MOLECULAR BIOLOGY 16.9-16.11 (1994); KING AND POSSEE, THE BACULOVIRUS SYSTEM: A LABORATORY GUIDE (1992); and O'REILLY ET AL., BACULOVIRUS EXPRESSION VECTORS: A LABORATORY MANUAL (1992).

Indeed, the production of various heterologous proteins using baculovirus/insect cell expression systems is known to those of ordinary skill in the art. See, e.g., U.S. Pat. Nos. 6,368,825; 6,342,216; 6,338,846; 6,261,805; 6,245,528, 6,225,060; 6,183,987; 6,168,932; 6,126,944; 6,096,304; 6,013,433; 5,965,393; 5,939,285; 5,891,676; 5,871,986; 5,861,279; 5,858,368; 5,843,733; 5,762,939; 5,753,220; 5,605,827; 5,583,023; 5,571,709; 5,516,657; 5,290,686; WO 02/06305; WO 01/90390; WO 01/27301; WO 01/05956; WO 00/55345; WO 00/20032; WO 99/51721; WO 99/45130; WO 99/31257; WO 99/10515; WO 99/09193; WO 97/26332; WO 96/29400; WO 96/25496; WO 96/06161; WO 95/20672; WO 93/03173; WO 92/16619; WO 92/02628; WO 92/01801; WO 90/14428; WO 90/10078; WO 90/02566; WO 90/02186; WO 90/01556; WO 89/01038; WO 89/01037; WO 88/07082, which are incorporated by reference herein.

Vectors that are useful in baculovirus/insect cell expression systems are known in the art and include, for example, insect expression and transfer vectors derived from the baculovirus Autographa californica nuclear polyhedrosis virus (AcNPV), which is a helper-independent, viral expression vector. Viral expression vectors derived from this system usually use the strong viral polyhedrin gene promoter to drive expression of heterologous genes. See generally, O'Reilly E T AL., BACULOVIRUS EXPRESSION VECTORS: A LABORATORY MANUAL (1992).

Prior to inserting the foreign gene into the baculovirus genome, the above-described components, comprising a promoter, leader (if desired), coding sequence of interest, and transcription termination sequence, are typically assembled into an intermediate transplacement construct (transfer vector). Intermediate transplacement constructs are often maintained in a replicon, such as an extra chromosomal element (e.g., plasmids) capable of stable maintenance in a host, such as bacteria. The replicon will have a replication system, thus allowing it to be maintained in a suitable host for cloning and amplification. More specifically, the plasmid may contain the polyhedrin poly adenylation signal (Miller, ANN. REV. MICROBIOL. (1988) 42:177) and a prokaryotic ampicillin-resistance (amp) gene and origin of replication for selection and propagation in E. coli.

One commonly used transfer vector for introducing foreign genes into AcNPV is pAc373. Many other vectors, known to those of skill in the art, have also been designed including, for example, pVL985, which alters the polyhedrin start codon from ATG to ATT, and which introduces a BamHI cloning site 32 base pairs downstream from the ATT. See Luckow and Summers, VIROLOGY 170:31 (1989). Other commercially available vectors include, for example, PBlueBac4.5/V5-His; pBlueBacHis2; pMelBac; pBlueBac4.5 (Invitrogen Corp., Carlsbad, Calif.).

After insertion of the heterologous gene, the transfer vector and wild type baculoviral genome are co-transfected into an insect cell host. Methods for introducing heterologous DNA into the desired site in the baculovirus virus are known in the art. See SUMMERS AND SMITH, TEXAS AGRICULTURAL EXPERIMENT STATION BULLETIN NO. 1555 (1987); Smith et al., MOL. CELL. BIOL. (1983) 3:2156; Luckow and Summers, VIROLOGY (1989) 170:31. For example, the insertion can be into a gene such as the polyhedrin gene, by homologous double crossover recombination; insertion can also be into a restriction enzyme site engineered into the desired baculovirus gene. See Miller et al., BIOESSAYS (1989) 11(4):91.

Transfection may be accomplished by electroporation. See TROTTER AND WOOD, 39 METHODS IN MOLECULAR BIOLOGY (1995); Mann and King, J. GEN. VIROL. (1989) 70:3501. Alternatively, liposomes may be used to transfect the insect cells with the recombinant expression vector and the baculovirus. See, e.g., Liebman et al., BIOTECHNIQUES (1999) 26(1):36; Graves et al., BIOCHEMISTRY (1998) 37:6050; Nomura et al., J. BIOL. CHEM. (1998) 273(22):13570; Schmidt et al., PROTEIN EXPRESSION AND PURIFICATION (1998) 12:323; Siffert et al., NATURE GENETICS (1998) 18:45; TILKINS ET AL., CELL BIOLOGY: A LABORATORY HANDBOOK 145-154 (1998); Cai et al., PROTEIN EXPRESSION AND PURIFICATION (1997) 10:263; Dolphin et al., NATURE GENETICS (1997) 17:491; Kost et al., GENE (1997) 190:139; Jakobsson et al., J. BIOL. CHEM. (1996) 271:22203; Rowles et al., J. BIOL. CHEM. (1996) 271(37):22376; Reverey et al., J. BIOL. CHEM. (1996) 271(39):23607-10; Stanley et al., J. BIOL. CHEM. (1995) 270:4121; Sisk et al., J. VIROL. (1994) 68(2):766; and Peng et al., BIOTECHNIQUES (1993) 14(2):274. Commercially available liposomes include, for example, Cellfectin® and Lipofectin® (Invitrogen, Corp., Carlsbad, Calif.). In addition, calcium phosphate transfection may be used. See TROTTER AND WOOD, 39 METHODS IN MOLECULAR BIOLOGY (1995); Kitts, NAR (1990) 18(19):5667; and Mann and King, J. GEN. VIROL. (1989) 70:3501.

Baculovirus expression vectors usually contain a baculovirus promoter. A baculovirus promoter is any DNA sequence capable of binding a baculovirus RNA polymerase and initiating the downstream (3′) transcription of a coding sequence (e.g., structural gene) into mRNA. A promoter will have a transcription initiation region which is usually placed proximal to the 5′ end of the coding sequence. This transcription initiation region typically includes an RNA polymerase binding site and a transcription initiation site. A baculovirus promoter may also have a second domain called an enhancer, which, if present, is usually distal to the structural gene. Moreover, expression may be either regulated or constitutive.

Structural genes, abundantly transcribed at late times in the infection cycle, provide particularly useful promoter sequences. Examples include sequences derived from the gene encoding the viral polyhedron protein (FRIESEN ET AL., The Regulation of Baculovirus Gene Expression in THE MOLECULAR BIOLOGY OF BACULOVIRUSES (1986); EP 0 127 839 and 0 155 476) and the gene encoding the p10 protein (Vlak et al., J. GEN. VIROL. (1988) 69:765).

The newly formed baculovirus expression vector is packaged into an infectious recombinant baculovirus and subsequently grown plaques may be purified by techniques known to those of ordinary skill in the art. See Miller et al., BIOESSAYS (1989) 11(4):91; SUMMERS AND SMITH, TEXAS AGRICULTURAL EXPERIMENT STATION BULLETIN NO. 1555 (1987).

Recombinant baculovirus expression vectors have been developed for infection into several insect cells. For example, recombinant baculoviruses have been developed for, inter alia, Aedes aegypti (ATCC No. CCL-125), Bombyx mori (ATCC No. CRL-8910), Drosophila melanogaster (ATCC No. 1963), Spodoptera frugiperda, and Trichoplusia ni. See Wright, NATURE (1986) 321:718; Carbonell et al., J. VIROL. (1985) 56:153; Smith et al., MOL. CELL. BIOL. (1983) 3:2156. See generally, Fraser et al., IN VITRO CELL. DEV. BIOL. (1989) 25:225. More specifically, the cell lines used for baculovirus expression vector systems commonly include, but are not limited to, Sf9 (Spodoptera frugiperda) (ATCC No. CRL-1711), Sf21 (Spodoptera frugiperda) (Invitrogen Corp., Cat. No. 11497-013 (Carlsbad, Calif.)), Tri-368 (Trichoplusia ni), and High-Five™ BTI-TN-5B1-4 (Trichoplusia ni).

Cells and culture media are commercially available for both direct and fusion expression of heterologous polypeptides in a baculovirus/expression, and cell culture technology is generally known to those of ordinary skill in the art.

E. coli, Pseudomonas species, and other Prokaryotes Bacterial expression techniques are known to those of ordinary skill in the art. A wide variety of vectors are available for use in bacterial hosts. The vectors may be single copy or low or high multicopy vectors. Vectors may serve for cloning and/or expression. In view of the ample literature concerning vectors, commercial availability of many vectors, and even manuals describing vectors and their restriction maps and characteristics, no extensive discussion is required here. As is well-known, the vectors normally involve markers allowing for selection, which markers may provide for cytotoxic agent resistance, prototrophy or immunity. Frequently, a plurality of markers is present, which provide for different characteristics.

A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region typically includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al., ANNU. REV. GENET. (1984) 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al., NATURE (1977) 198:1056], and maltose. Additional examples include promoter sequences derived from biosynthetic enzymes such as tryptophan (trp) [Goeddel et al., NUC. ACIDS RES. (1980) 8:4057; Yelverton et al., NUCL. ACIDS RES. (1981) 9:731; U.S. Pat. No. 4,738,921; EP Pub. Nos. 036 776 and 121 775, which are incorporated by reference herein]. The β-galactosidase (bla) promoter system [Weissmann (1981) “The cloning of interferon and other mistakes.” In Interferon 3 (Ed. I. Gresser)], bacteriophage lambda PL [Shimatake et al., NATURE (1981) 292:128] and T5 [U.S. Pat. No. 4,689,406, which are incorporated by reference herein] promoter systems also provide useful promoter sequences. Preferred methods of the present invention utilize strong promoters, such as the T7 promoter to induce IFNL3 polypeptides at high levels. Examples of such vectors are known to those of ordinary skill in the art and include the pET29 series from Novagen, and the pPOP vectors described in WO99/05297, which is incorporated by reference herein. Such expression systems produce high levels of IFNL3 polypeptides in the host without compromising host cell viability or growth parameters. pET19 (Novagen) is another vector known in the art.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433, which is incorporated by reference herein]. For example, the tac promoter is a hybrid trp-lac promoter comprised of both trp promoter and lac operon sequences that is regulated by the lac repressor [Amann et al., GENE (1983) 25:167; de Boer et al., PROC. NATL. ACAD. SCI. (1983) 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al., J. MOL. BIOL. (1986) 189:113; Tabor et al., Proc Natl. Acad. Sci. (1985) 82:1074]. In addition, a hybrid promoter can also be comprised of a bacteriophage promoter and an E. coli operator region (EP Pub. No. 267 851).

In addition to a functioning promoter sequence, an efficient ribosome binding site is also useful for the expression of foreign genes in prokaryotes. In E. coli, the ribosome binding site is called the Shine-Delgarno (SD) sequence and includes an initiation codon (ATG) and a sequence 3-9 nucleotides in length located 3-11 nucleotides upstream of the initiation codon [Shine et al., NATURE (1975) 254:34]. The SD sequence is thought to promote binding of mRNA to the ribosome by the pairing of bases between the SD sequence and the 3′ and of E. coli 16S rRNA [Steitz et al. “Genetic signals and nucleotide sequences in messenger RNA”, In Biological Regulation and Development: Gene Expression (Ed. R. F. Goldberger, 1979)]. To express eukaryotic genes and prokaryotic genes with weak ribosome-binding site [Sambrook et al. “Expression of cloned genes in Escherichia coli”, Molecular Cloning: A Laboratory Manual, 1989].

The term “bacterial host” or “bacterial host cell” refers to a bacterial that can be, or has been, used as a recipient for recombinant vectors or other transfer DNA. The term includes the progeny of the original bacterial host cell that has been transfected. It is understood that the progeny of a single parental cell may not necessarily be completely identical in morphology or in genomic or total DNA complement to the original parent, due to accidental or deliberate mutation. Progeny of the parental cell that are sufficiently similar to the parent to be characterized by the relevant property, such as the presence of a nucleotide sequence encoding an IFNL3 polypeptide, are included in the progeny intended by this definition.

The selection of suitable host bacteria for expression of IFNL3 polypeptides is known to those of ordinary skill in the art. In selecting bacterial hosts for expression, suitable hosts may include those shown to have, inter alia, good inclusion body formation capacity, low proteolytic activity, and overall robustness. Bacterial hosts are generally available from a variety of sources including, but not limited to, the Bacterial Genetic Stock Center, Department of Biophysics and Medical Physics, University of California (Berkeley, Calif.); and the American Type Culture Collection (“ATCC”) (Manassas, Va.). Industrial/pharmaceutical fermentation generally use bacterial derived from K strains (e.g. W3110) or from bacteria derived from B strains (e.g. BL21). These strains are particularly useful because their growth parameters are extremely well known and robust. In addition, these strains are non-pathogenic, which is commercially important for safety and environmental reasons. Other examples of suitable E. coli hosts include, but are not limited to, strains of BL21, DH10B, or derivatives thereof. In another embodiment of the methods of the present invention, the E. coli host is a protease minus strain including, but not limited to, OMP- and LON-. The host cell strain may be a species of Pseudomonas, including but not limited to, Pseudomonas fluorescens, Pseudomonas aeruginosa, and Pseudomonas putida. Pseudomonas fluorescens biovar 1, designated strain MB101, is known to be useful for recombinant production and is available for therapeutic protein production processes. Examples of a Pseudomonas expression system include the system available from The Dow Chemical Company as a host strain (Midland, Mich. available on the World Wide Web at dow.com).

Once a recombinant host cell strain has been established (i.e., the expression construct has been introduced into the host cell and host cells with the proper expression construct are isolated), the recombinant host cell strain is cultured under conditions appropriate for production of IFNL3 polypeptides. As will be apparent to one of skill in the art, the method of culture of the recombinant host cell strain will be dependent on the nature of the expression construct utilized and the identity of the host cell. Recombinant host strains are normally cultured using methods that are known to those of ordinary skill in the art. Recombinant host cells are typically cultured in liquid medium containing assimilable sources of carbon, nitrogen, and inorganic salts and, optionally, containing vitamins, amino acids, growth factors, and other proteinaceous culture supplements known to those of ordinary skill in the art. Liquid media for culture of host cells may optionally contain antibiotics or anti-fungals to prevent the growth of undesirable microorganisms and/or compounds including, but not limited to, antibiotics to select for host cells containing the expression vector.

Recombinant host cells may be cultured in batch or continuous formats, with either cell harvesting (in the case where the IFNL3 polypeptide accumulates intracellularly) or harvesting of culture supernatant in either batch or continuous formats. For production in prokaryotic host cells, batch culture and cell harvest are preferred.

The IFNL3 polypeptides of the present invention are normally purified after expression in recombinant systems. The IFNL3 polypeptide may be purified from host cells or culture medium by a variety of methods known to the art. IFNL3 polypeptides produced in bacterial host cells may be poorly soluble or insoluble (in the form of inclusion bodies). In one embodiment of the present invention, amino acid substitutions may readily be made in the IFNL3 polypeptide that are selected for the purpose of increasing the solubility of the recombinantly produced protein utilizing the methods disclosed herein as well as those known in the art. In the case of insoluble protein, the protein may be collected from host cell lysates by centrifugation and may further be followed by homogenization of the cells. In the case of poorly soluble protein, compounds including, but not limited to, polyethylene imine (PEI) may be added to induce the precipitation of partially soluble protein. The precipitated protein may then be conveniently collected by centrifugation. Recombinant host cells may be disrupted or homogenized to release the inclusion bodies from within the cells using a variety of methods known to those of ordinary skill in the art. Host cell disruption or homogenization may be performed using well known techniques including, but not limited to, enzymatic cell disruption, sonication, dounce homogenization, or high pressure release disruption. In one embodiment of the method of the present invention, the high pressure release technique is used to disrupt the E. coli host cells to release the inclusion bodies of the IFNL3 polypeptides. When handling inclusion bodies of IFNL3 polypeptide, it may be advantageous to minimize the homogenization time on repetitions in order to maximize the yield of inclusion bodies without loss due to factors such as solubilization, mechanical shearing or proteolysis.

Insoluble or precipitated IFNL3 polypeptide may then be solubilized using any of a number of suitable solubilization agents known to the art. The IFNL3 polypeptide may be solubilized with urea or guanidine hydrochloride. The volume of the solubilized IFNL3 polypeptide should be minimized so that large batches may be produced using conveniently manageable batch sizes. This factor may be significant in a large-scale commercial setting where the recombinant host may be grown in batches that are thousands of liters in volume. In addition, when manufacturing IFNL3 polypeptide in a large-scale commercial setting, in particular for human pharmaceutical uses, the avoidance of harsh chemicals that can damage the machinery and container, or the protein product itself, should be avoided, if possible. It has been shown in the method of the present invention that the milder denaturing agent urea can be used to solubilize the IFNL3 polypeptide inclusion bodies in place of the harsher denaturing agent guanidine hydrochloride. The use of urea significantly reduces the risk of damage to stainless steel equipment utilized in the manufacturing and purification process of IFNL3 polypeptide while efficiently solubilizing the IFNL3 polypeptide inclusion bodies.

In the case of soluble IFNL3 protein, the IFNL3 may be secreted into the periplasmic space or into the culture medium. In addition, soluble IFNL3 may be present in the cytoplasm of the host cells. It may be desired to concentrate soluble IFNL3 prior to performing purification steps. Standard techniques known to those of ordinary skill in the art may be used to concentrate soluble IFNL3 from, for example, cell lysates or culture medium. In addition, standard techniques known to those of ordinary skill in the art may be used to disrupt host cells and release soluble IFNL3 from the cytoplasm or periplasmic space of the host cells.

When IFNL3 polypeptide is produced as a fusion protein, the fusion sequence may be removed. Removal of a fusion sequence may be accomplished by enzymatic or chemical cleavage. Enzymatic removal of fusion sequences may be accomplished using methods known to those of ordinary skill in the art. The choice of enzyme for removal of the fusion sequence will be determined by the identity of the fusion, and the reaction conditions will be specified by the choice of enzyme as will be apparent to one of ordinary skill in the art. Chemical cleavage may be accomplished using reagents known to those of ordinary skill in the art, including but not limited to, cyanogen bromide, TEV protease, and other reagents. The cleaved IFNL3 polypeptide may be purified from the cleaved fusion sequence by methods known to those of ordinary skill in the art. Such methods will be determined by the identity and properties of the fusion sequence and the IFNL3 polypeptide, as will be apparent to one of ordinary skill in the art. Methods for purification may include, but are not limited to, size-exclusion chromatography, hydrophobic interaction chromatography, ion-exchange chromatography or dialysis or any combination thereof.

The IFNL3 polypeptide may also be purified to remove DNA from the protein solution. DNA may be removed by any suitable method known to the art, such as precipitation or ion exchange chromatography, but may be removed by precipitation with a nucleic acid precipitating agent, such as, but not limited to, protamine sulfate. The IFNL3 polypeptide may be separated from the precipitated DNA using standard well known methods including, but not limited to, centrifugation or filtration. Removal of host nucleic acid molecules is an important factor in a setting where the IFNL3 polypeptide is to be used to treat humans and the methods of the present invention reduce host cell DNA to pharmaceutically acceptable levels.

Methods for small-scale or large-scale fermentation can also be used in protein expression, including but not limited to, fermentors, shake flasks, fluidized bed bioreactors, hollow fiber bioreactors, roller bottle culture systems, and stirred tank bioreactor systems. Each of these methods can be performed in a batch, fed-batch, or continuous mode process.

IFNL3 polypeptides of the invention can generally be recovered using methods standard in the art. For example, culture medium or cell lysate can be centrifuged or filtered to remove cellular debris. The supernatant may be concentrated or diluted to a desired volume or diafiltered into a suitable buffer to condition the preparation for further purification. Further purification of the IFNL3 polypeptide of the present invention includes separating deamidated and clipped forms of the IFNL3 polypeptide variant from the intact form.

Any of the following exemplary procedures can be employed for purification of IFNL3 polypeptides of the invention: affinity chromatography; anion- or cation-exchange chromatography (using, including but not limited to, DEAE SEPHAROSE); chromatography on silica; high performance liquid chromatography (HPLC); reverse phase HPLC; gel filtration (using, including but not limited to, SEPHADEX G-75); hydrophobic interaction chromatography; size-exclusion chromatography; metal-chelate chromatography; ultrafiltration/diafiltration; ethanol precipitation; ammonium sulfate precipitation; chromatofocusing; displacement chromatography; electrophoretic procedures (including but not limited to preparative isoelectric focusing), differential solubility (including but not limited to ammonium sulfate precipitation), SDS-PAGE, or extraction.

IFNL3 proteins of the present invention, including but not limited to, IFNL3 proteins comprising unnatural amino acids, peptides comprising unnatural amino acids, antibodies to proteins comprising unnatural amino acids, binding partners for proteins comprising unnatural amino acids, etc., can be purified, either partially or substantially to homogeneity, according to standard procedures known to and used by those of skill in the art. Accordingly, polypeptides of the invention can be recovered and purified by any of a number of methods known to those of ordinary skill in the art, including but not limited to, ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against unnatural amino acids (or proteins or peptides comprising unnatural amino acids) are used as purification reagents, including but not limited to, for affinity-based purification of proteins or peptides comprising one or more unnatural amino acid(s). Once purified, partially or to homogeneity, as desired, the polypeptides are optionally used for a wide variety of utilities, including but not limited to, as assay components, therapeutics, prophylaxis, diagnostics, research reagents, and/or as immunogens for antibody production. Antibodies generated against polypeptides of the present invention may be obtained by administering the polypeptides or epitope-bearing fragments, or cells to an animal, preferably a non-human animal, using routine protocols. One of ordinary skill in the art could generate antibodies using a variety of known techniques. Also, transgenic mice, or other organisms, including other mammals, may be used to express humanized antibodies. The above-described antibodies may be employed to isolate or to identify clones expressing the polypeptide or to purify the polypeptides. Antibodies against polypeptides of the present invention may also be employed to treat diseases.

### Expression in Alternate Systems

Several strategies have been employed to introduce unnatural amino acids into proteins in non-recombinant host cells, mutagenized host cells, or in cell-free systems. These systems are also suitable for use in making the IFNL3 polypeptides of the present invention. Derivatization of amino acids with reactive side-chains such as Lys, Cys and Tyr resulted in the conversion of lysine to N²-acetyl-lysine. Chemical synthesis also provides a straightforward method to incorporate unnatural amino acids. With the recent development of enzymatic ligation and native chemical ligation of peptide fragments, it is possible to make larger proteins. See, e.g., P. E. Dawson and S. B. H. Kent, Annu. Rev. Biochem, 69:923 (2000). Chemical peptide ligation and native chemical ligation are described in U.S. Pat. No. 6,184,344, U.S. Patent Publication No. 2004/0138412, U.S. Patent Publication No. 2003/0208046, WO 02/098902, and WO 03/042235, which are incorporated by reference herein. A general in vitro biosynthetic method in which a suppressor tRNA chemically acylated with the desired unnatural amino acid is added to an in vitro extract capable of supporting protein biosynthesis, has been used to site-specifically incorporate over 100 unnatural amino acids into a variety of proteins of virtually any size. See, e.g., V. W. Cornish, D. Mendel and P. G. Schultz, Angew. Chem. Int. Ed. Engl., 1995, 34:621 (1995); C. J. Noren, S. J. Anthony-Cahill, M. C. Griffith, P. G. Schultz, A general method for site-specific incorporation of unnatural amino acids into proteins, Science 244:182-188 (1989); and, J. D. Bain, C. G. Glabe, T. A. Dix, A. R. Chamberlin, E. S. Diala, Biosynthetic site-specific incorporation of a non-natural amino acid into a polypeptide, J. Am. Chem. Soc. 111:8013-8014 (1989). A broad range of functional groups has been introduced into proteins for studies of protein stability, protein folding, enzyme mechanism, and signal transduction.

In addition to other references noted herein, a variety of purification/protein folding methods are known to those of ordinary skill in the art, including, but not limited to, those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana, (1997) Bioseparation of Proteins, Academic Press, Inc.; Bollag et al. (1996) Protein Methods, 2nd Edition Wiley-Liss, NY; Walker, (1996) The Protein Protocols Handbook Humana Press, N.J., Harris and Angal, (1990) Protein Purification Applications: A Practical Approach IRL Press at Oxford, Oxford, England; Harris and Angal, Protein Purification Methods: A Practical Approach IRL Press at Oxford, Oxford, England; Scopes, (1993) Protein Purification: Principles and Practice 3rd Edition Springer Verlag, N.Y.; Janson and Ryden, (1998) Protein Purification: Principles, High Resolution Methods and Applications, Second Edition Wiley-VCH, NY; and Walker (1998), Protein Protocols on CD-ROM Humana Press, N.J.; and the references cited therein.

One advantage of producing a protein or polypeptide of interest with an unnatural amino acid in a eukaryotic host cell or non-eukaryotic host cell is that typically the proteins or polypeptides will be folded in their native conformations. However, in certain embodiments of the invention, those of skill in the art will recognize that, after synthesis, expression and/or purification, proteins or peptides can possess a conformation different from the desired conformations of the relevant polypeptides. In one aspect of the invention, the expressed protein or polypeptide is optionally denatured and then renatured. This is accomplished utilizing methods known in the art, including but not limited to, by adding a chaperonin to the protein or polypeptide of interest, by solubilizing the proteins in a chaotropic agent such as guanidine HCl, utilizing protein disulfide isomerase, etc.

In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are known to those of ordinary skill in the art (see, the references above, and Debinski, et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski, et al., for example, describe the denaturation and reduction of inclusion body proteins in guanidine-DTE. The proteins can be refolded in a redox buffer containing, including but not limited to, oxidized glutathione and L-arginine. Refolding reagents can be flowed or otherwise moved into contact with the one or more polypeptide or other expression product, or vice-versa.

In the case of prokaryotic production of IFNL3 polypeptide, the IFNL3 polypeptide thus produced may be misfolded and thus lacks or has reduced biological activity. The bioactivity of the protein may be restored by “refolding”. In general, misfolded IFNL3 polypeptide is refolded by solubilizing (where the IFNL3 polypeptide is also insoluble), unfolding and reducing the polypeptide chain using, for example, one or more chaotropic agents (e.g. urea and/or guanidine) and a reducing agent capable of reducing disulfide bonds (e.g. dithiothreitol, DTT or 2-mercaptoethanol, 2-ME). At a moderate concentration of chaotrope, an oxidizing agent is then added (e.g., oxygen, cystine or cystamine), which allows the reformation of disulfide bonds. IFNL3 polypeptide may be refolded using standard methods known in the art, such as those described in U.S. Pat. Nos. 4,511,502, 4,511,503, and 4,512,922, which are incorporated by reference herein. The IFNL3 polypeptide may also be cofolded with other proteins to form heterodimers or heteromultimers.

After refolding, the IFNL3 may be further purified. Purification of IFNL3 may be accomplished using a variety of techniques known to those of ordinary skill in the art, including hydrophobic interaction chromatography, size exclusion chromatography, ion exchange chromatography, reverse-phase high performance liquid chromatography, affinity chromatography, and the like or any combination thereof. Additional purification may also include a step of drying or precipitation of the purified protein.

After purification, IFNL3 may be exchanged into different buffers and/or concentrated by any of a variety of methods known to the art, including, but not limited to, diafiltration and dialysis. IFNL3 that is provided as a single purified protein may be subject to aggregation and precipitation. The purified IFNL3 may be at least 90% pure (as measured by reverse phase high performance liquid chromatography, RP-HPLC, or sodium dodecyl sulfate-polyacrylamide gel electrophoresis, SDS-PAGE) or at least 95% pure, or at least 98% pure, or at least 99% or greater pure. Regardless of the exact numerical value of the purity of the IFNL3, the IFNL3 is sufficiently pure for use as a pharmaceutical product or for further processing, such as conjugation with a PKEM.

Certain IFNL3 molecules may be used as therapeutic agents in the absence of other active ingredients or proteins (other than excipients, carriers, and stabilizers, serum albumin and the like), or they may be complexed with another protein or a polymer.

General Purification Methods Any one of a variety of isolation steps may be performed on the cell lysate, extract, culture medium, inclusion bodies, periplasmic space of the host cells, cytoplasm of the host cells, or other material, comprising IFNL3 polypeptide or on any IFNL3 polypeptide mixtures resulting from any isolation steps including, but not limited to, affinity chromatography, ion exchange chromatography, hydrophobic interaction chromatography, gel filtration chromatography, high performance liquid chromatography (“HPLC”), reversed phase-HPLC (“RP-HPLC”), expanded bed adsorption, or any combination and/or repetition thereof and in any appropriate order.

Equipment and other necessary materials used in performing the techniques described herein are commercially available. Pumps, fraction collectors, monitors, recorders, and entire systems are available from, for example, Applied Biosystems (Foster City, Calif.), Bio-Rad Laboratories, Inc. (Hercules, Calif.), and Amersham Biosciences, Inc. (Piscataway, N.J.). Chromatographic materials including, but not limited to, exchange matrix materials, media, and buffers are also available from such companies. Equilibration, and other steps in the column chromatography processes described herein such as washing and elution, may be more rapidly accomplished using specialized equipment such as a pump. Commercially available pumps include, but are not limited to, HILOAD® Pump P-50, Peristaltic Pump P-1, Pump P-901, and Pump P-903 (Amersham Biosciences, Piscataway, N.J.).

Examples of fraction collectors include RediFrac Fraction Collector, FRAC-100 and FRAC-200 Fraction Collectors, and SUPERFRAC® Fraction Collector (Amersham Biosciences, Piscataway, N.J.). Mixers are also available to form pH and linear concentration gradients. Commercially available mixers include Gradient Mixer GM-1 and In-Line Mixers (Amersham Biosciences, Piscataway, N.J.). The chromatographic process may be monitored using any commercially available monitor. Such monitors may be used to gather information like UV, pH, and conductivity. Examples of detectors include Monitor UV-1, UVICORD® S II, Monitor UV-M II, Monitor UV-900, Monitor UPC-900, Monitor pH/C-900, and Conductivity Monitor (Amersham Biosciences, Piscataway, N.J.). Indeed, entire systems are commercially available including the various AKTA® systems from Amersham Biosciences (Piscataway, N.J.).

In one embodiment of the present invention, for example, the IFNL3 polypeptide may be reduced and denatured by first denaturing the resultant purified IFNL3 polypeptide in urea, followed by dilution into TRIS buffer containing a reducing agent (such as DTT) at a suitable pH. In another embodiment, the IFNL3 polypeptide is denatured in urea in a concentration range of between about 2 M to about 9 M, followed by dilution in TRIS buffer at a pH in the range of about 5.0 to about 8.0. The refolding mixture of this embodiment may then be incubated. In one embodiment, the refolding mixture is incubated at room temperature for four to twenty-four hours. The reduced and denatured IFNL3 polypeptide mixture may then be further isolated or purified.

As stated herein, the pH of the first IFNL3 polypeptide mixture may be adjusted prior to performing any subsequent isolation steps. In addition, the first IFNL3 polypeptide mixture or any subsequent mixture thereof may be concentrated using techniques known in the art. Moreover, the elution buffer comprising the first IFNL3 polypeptide mixture or any subsequent mixture thereof may be exchanged for a buffer suitable for the next isolation step using techniques known to those of ordinary skill in the art.

Ion Exchange Chromatography In one embodiment, and as an optional, additional step, ion exchange chromatography may be performed on the first IFNL3 polypeptide mixture. See generally ION EXCHANGE CHROMATOGRAPHY: PRINCIPLES AND METHODS (Cat. No. 18-1114-21, Amersham Biosciences (Piscataway, N.J.)). Commercially available ion exchange columns include HITRAP®, HIPREP®, and HILOAD® Columns (Amersham Biosciences, Piscataway, N.J.). Such columns utilize strong anion exchangers such as Q SEPHAROSE® Fast Flow, Q SEPHAROSE® High Performance, and Q SEPHAROSE® XL; strong cation exchangers such as SP SEPHAROSE® High Performance, SP SEPHAROSE® Fast Flow, and SP SEPHAROSE® XL; weak anion exchangers such as DEAE SEPHAROSE® Fast Flow; and weak cation exchangers such as CM SEPHAROSE® Fast Flow (Amersham Biosciences, Piscataway, N.J.). Anion or cation exchange column chromatography may be performed on the IFNL3 polypeptide at any stage of the purification process to isolate substantially purified IFNL3 polypeptide. The cation exchange chromatography step may be performed using any suitable cation exchange matrix. Useful cation exchange matrices include, but are not limited to, fibrous, porous, non-porous, microgranular, beaded, or cross-linked cation exchange matrix materials. Such cation exchange matrix materials include, but are not limited to, cellulose, agarose, dextran, polyacrylate, polyvinyl, polystyrene, silica, polyether, or composites of any of the foregoing.

The cation exchange matrix may be any suitable cation exchanger including strong and weak cation exchangers. Strong cation exchangers may remain ionized over a wide pH range and thus, may be capable of binding IFNL3 over a wide pH range. Weak cation exchangers, however, may lose ionization as a function of pH. For example, a weak cation exchanger may lose charge when the pH drops below about pH 4 or pH 5. Suitable strong cation exchangers include, but are not limited to, charged functional groups such as sulfopropyl (SP), methyl sulfonate (S), or sulfoethyl (SE). The cation exchange matrix may be a strong cation exchanger, preferably having an IFNL3 binding pH range of about 2.5 to about 6.0. Alternatively, the strong cation exchanger may have an IFNL3 binding pH range of about pH 2.5 to about pH 5.5. The cation exchange matrix may be a strong cation exchanger having an IFNL3 binding pH of about 3.0. Alternatively, the cation exchange matrix may be a strong cation exchanger, preferably having an IFNL3 binding pH range of about 6.0 to about 8.0. The cation exchange matrix may be a strong cation exchanger preferably having an IFNL3 binding pH range of about 8.0 to about 12.5. Alternatively, the strong cation exchanger may have an IFNL3 binding pH range of about pH 8.0 to about pH 12.0.

Prior to loading the IFNL3, the cation exchange matrix may be equilibrated, for example, using several column volumes of a dilute, weak acid, e.g., four column volumes of 20 mM acetic acid, pH 3. Following equilibration, the IFNL3 may be added and the column may be washed one to several times, prior to elution of substantially purified IFNL3, also using a weak acid solution such as a weak acetic acid or phosphoric acid solution. For example, approximately 2-4 column volumes of 20 mM acetic acid, pH 3, may be used to wash the column. Additional washes using, e.g., 2-4 column volumes of 0.05 M sodium acetate, pH 5.5, or 0.05 M sodium acetate mixed with 0.1 M sodium chloride, pH 5.5, may also be used. Alternatively, using methods known in the art, the cation exchange matrix may be equilibrated using several column volumes of a dilute, weak base.

Alternatively, substantially purified IFNL3 may be eluted by contacting the cation exchanger matrix with a buffer having a sufficiently low pH or ionic strength to displace the IFNL3 from the matrix. The pH of the elution buffer may range from about pH 2.5 to about pH 6.0. More specifically, the pH of the elution buffer may range from about pH 2.5 to about pH 5.5, about pH 2.5 to about pH 5.0. The elution buffer may have a pH of about 3.0. In addition, the quantity of elution buffer may vary widely and will generally be in the range of about 2 to about 10 column volumes.

Following adsorption of the IFNL3 polypeptide to the cation exchanger matrix, substantially purified IFNL3 polypeptide may be eluted by contacting the matrix with a buffer having a sufficiently high pH or ionic strength to displace the IFNL3 polypeptide from the matrix. Suitable buffers for use in high pH elution of substantially purified IFNL3 polypeptide may include, but not limited to, citrate, phosphate, formate, acetate, HEPES, and MES buffers ranging in concentration from at least about 5 mM to at least about 100 mM.

Reverse-Phase Chromatography RP-HPLC may be performed to purify proteins following suitable protocols that are known to those of ordinary skill in the art. See, e.g., Pearson et al., ANAL BIOCHEM. (1982) 124:217-230 (1982); Rivier et al., J. CHROM. (1983) 268:112-119; Kunitani et al., J. CHROM. (1986) 359:391-402. RP-HPLC may be performed on the IFNL3 polypeptide to isolate substantially purified IFNL3 polypeptide. In this regard, silica derivatized resins with alkyl functionalities with a wide variety of lengths, including, but not limited to, at least about C3 to at least about C30, at least about C3 to at least about C20, or at least about C3 to at least about C18, resins may be used. Alternatively, a polymeric resin may be used. For example, TosoHaas Amberchrome CG1000sd resin may be used, which is a styrene polymer resin. Cyano or polymeric resins with a wide variety of alkyl chain lengths may also be used. Furthermore, the RP-HPLC column may be washed with a solvent such as ethanol. The Source RP column is another example of a RP-HPLC column.

A suitable elution buffer containing an ion pairing agent and an organic modifier such as methanol, isopropanol, tetrahydrofuran, acetonitrile or ethanol, may be used to elute the IFNL3 polypeptide from the RP-HPLC column. The most commonly used ion pairing agents include, but are not limited to, acetic acid, formic acid, perchloric acid, phosphoric acid, trifluoroacetic acid, heptafluorobutyric acid, triethylamine, tetramethylammonium, tetrabutylammonium, and triethylammonium acetate. Elution may be performed using one or more gradients or isocratic conditions, with gradient conditions preferred to reduce the separation time and to decrease peak width. Another method involves the use of two gradients with different solvent concentration ranges. Examples of suitable elution buffers for use herein may include, but are not limited to, ammonium acetate and acetonitrile solutions.

Hydrophobic Interaction Chromatography Purification Techniques Hydrophobic interaction chromatography (HIC) may be performed on the IFNL3 polypeptide. See generally HYDROPHOBIC INTERACTION CHROMATOGRAPHY HANDBOOK: PRINCIPLES AND METHODS (Cat. No. 18-1020-90, Amersham Biosciences (Piscataway, N.J.) which is incorporated by reference herein. Suitable HIC matrices may include, but are not limited to, alkyl- or aryl-substituted matrices, such as butyl-, hexyl-, octyl- or phenyl-substituted matrices including agarose, cross-linked agarose, sepharose, cellulose, silica, dextran, polystyrene, poly(methacrylate) matrices, and mixed mode resins, including but not limited to, a polyethyleneamine resin or a butyl- or phenyl-substituted poly(methacrylate) matrix. Commercially available sources for hydrophobic interaction column chromatography include, but are not limited to, HITRAP®, HIPREP®, and HILOAD® columns (Amersham Biosciences, Piscataway, N.J.).

Briefly, prior to loading, the HIC column may be equilibrated using standard buffers known to those of ordinary skill in the art, such as an acetic acid/sodium chloride solution or HEPES containing ammonium sulfate. Ammonium sulfate may be used as the buffer for loading the HIC column. After loading the IFNL3 polypeptide, the column may then washed using standard buffers and conditions to remove unwanted materials but retaining the IFNL3 polypeptide on the HIC column. The IFNL3 polypeptide may be eluted with about 3 to about 10 column volumes of a standard buffer, such as a HEPES buffer containing EDTA and lower ammonium sulfate concentration than the equilibrating buffer, or an acetic acid/sodium chloride buffer, among others. A decreasing linear salt gradient using, for example, a gradient of potassium phosphate, may also be used to elute the IFNL3 molecules. The eluant may then be concentrated, for example, by filtration such as diafiltration or ultrafiltration. Diafiltration may be utilized to remove the salt used to elute the IFNL3 polypeptide.

Other Purification Techniques Yet another isolation step using, for example, gel filtration (GEL FILTRATION: PRINCIPLES AND METHODS (Cat. No. 18-1022-18, Amersham Biosciences, Piscataway, N.J.) which is incorporated by reference herein, hydroxyapatite chromatography (suitable matrices include, but are not limited to, HA-Ultrogel, High Resolution (Calbiochem), CHT Ceramic Hydroxyapatite (BioRad), Bio-Gel HTP Hydroxyapatite (BioRad)), HPLC, expanded bed adsorption, ultrafiltration, diafiltration, lyophilization, and the like, may be performed on the first IFNL3 polypeptide mixture or any subsequent mixture thereof, to remove any excess salts and to replace the buffer with a suitable buffer for the next isolation step or even formulation of the final drug product.

The yield of IFNL3 polypeptide, including substantially purified IFNL3 polypeptide, may be monitored at each step described herein using techniques known to those of ordinary skill in the art. Such techniques may also be used to assess the yield of substantially purified IFNL3 polypeptide following the last isolation step. For example, the yield of IFNL3 polypeptide may be monitored using any of several reverse phase high pressure liquid chromatography columns, having a variety of alkyl chain lengths such as cyano RP-HPLC, C18RP-HPLC; as well as cation exchange HPLC and gel filtration HPLC.

In specific embodiments of the present invention, the yield of IFNL3 after each purification step may be at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, or at least about 99.99%, of the IFNL3 in the starting material for each purification step.

Purity may be determined using standard techniques, such as SDS-PAGE, or by measuring IFNL3 polypeptide using Western blot and ELISA assays. For example, polyclonal antibodies may be generated against proteins isolated from negative control yeast fermentation and the cation exchange recovery. The antibodies may also be used to probe for the presence of contaminating host cell proteins.

RP-HPLC material Vydac C4 (Vydac) consists of silica gel particles, the surfaces of which carry C4-alkyl chains. The separation of IFNL3 polypeptide from the proteinaceous impurities is based on differences in the strength of hydrophobic interactions. Elution is performed with an acetonitrile gradient in diluted trifluoroacetic acid. Preparative HPLC is performed using a stainless steel column (filled with 2.8 to 3.2 liter of Vydac C4 silica gel). The Hydroxyapatite Ultrogel eluate is acidified by adding trifluoroacetic acid and loaded onto the Vydac C4 column. For washing and elution an acetonitrile gradient in diluted trifluoroacetic acid is used. Fractions are collected and immediately neutralized with phosphate buffer. The IFNL3 polypeptide fractions which are within the IPC limits are pooled.

DEAE Sepharose (Pharmacia) material consists of diethylaminoethyl (DEAE)-groups which are covalently bound to the surface of Sepharose beads. The binding of IFNL3 polypeptide to the DEAE groups is mediated by ionic interactions. Acetonitrile and trifluoroacetic acid pass through the column without being retained. After these substances have been washed off, trace impurities are removed by washing the column with acetate buffer at a low pH. Then the column is washed with neutral phosphate buffer and IFNL3 polypeptide is eluted with a buffer with increased ionic strength. The column is packed with DEAE Sepharose fast flow. The column volume is adjusted to assure an IFNL3 polypeptide load in the range of 3-10 mg IFNL3 polypeptide/ml gel. The column is washed with water and equilibration buffer (sodium/potassium phosphate). The pooled fractions of the HPLC eluate are loaded and the column is washed with equilibration buffer. Then the column is washed with washing buffer (sodium acetate buffer) followed by washing with equilibration buffer. Subsequently, IFNL3 polypeptide is eluted from the column with elution buffer (sodium chloride, sodium/potassium phosphate) and collected in a single fraction in accordance with the master elution profile. The eluate of the DEAE Sepharose column is adjusted to the specified conductivity. The resulting drug substance is sterile filtered into Teflon bottles and stored at −70° C.

Additional methods that may be employed include, but are not limited to, steps to remove endotoxins. Endotoxins are lipopoly-saccharides (LPSs) which are located on the outer membrane of Gram-negative host cells, such as, for example, Escherichia coli. Methods for reducing endotoxin levels are known to one of ordinary skill in the art and include, but are not limited to, purification techniques using silica supports, glass powder or hydroxyapatite, reverse-phase, affinity, size-exclusion, anion-exchange chromatography, hydrophobic interaction chromatography, a combination of these methods, and the like. Modifications or additional methods may be required to remove contaminants such as co-migrating proteins from the polypeptide of interest. Methods for measuring endotoxin levels are known to one of ordinary skill in the art and include, but are not limited to, Limulus Amebocyte Lysate (LAL) assays. The Endosafe™-PTS assay is a colorimetric, single tube system that utilizes cartridges preloaded with LAL reagent, chromogenic substrate, and control standard endotoxin along with a handheld spectrophotometer. Alternate methods include, but are not limited to, a Kinetic LAL method that is turbidimetric and uses a 96 well format.

A wide variety of methods and procedures can be used to assess the yield and purity of an IFNL3 protein comprising one or more non-naturally encoded amino acids, including but not limited to, the Bradford assay, SDS-PAGE, silver stained SDS-PAGE, coomassie stained SDS-PAGE, mass spectrometry (including but not limited to, MALDI-TOF) and other methods for characterizing proteins known to one of ordinary skill in the art.

Additional methods include, but are not limited to: SDS-PAGE coupled with protein staining methods, immunoblotting, matrix assisted laser desorption/ionization-mass spectrometry (MALDI-MS), liquid chromatography/mass spectrometry, isoelectric focusing, analytical anion exchange, chromatofocusing, and circular dichroism.

An in vivo method, termed selective pressure incorporation, was developed to exploit the promiscuity of wild-type synthetases. See, e.g., N. Budisa, C. Minks, S. Alefelder, W. Wenger, F. M. Dong, L. Moroder and R. Huber, FASEB J., 13:41 (1999). An auxotrophic strain, in which the relevant metabolic pathway supplying the cell with a particular natural amino acid is switched off, is grown in minimal media containing limited concentrations of the natural amino acid, while transcription of the target gene is repressed. At the onset of a stationary growth phase, the natural amino acid is depleted and replaced with the unnatural amino acid analog. Induction of expression of the recombinant protein results in the accumulation of a protein containing the unnatural analog. For example, using this strategy, o, m and p-fluorophenylalanines have been incorporated into proteins, and exhibit two characteristic shoulders in the UV spectrum which can be easily identified, see, e.g., C. Minks, R. Huber, L. Moroder and N. Budisa, Anal. Biochem., 284:29 (2000); trifluoromethionine has been used to replace methionine in bacteriophage T4 lysozyme to study its interaction with chitooligosaccharide ligands by ¹⁹F NMR, see, e.g., H. Duewel, E. Daub, V. Robinson and J. F. Honek, Biochemistry, 36:3404 (1997); and trifluoroleucine has been incorporated in place of leucine, resulting in increased thermal and chemical stability of a leucine-zipper protein. See, e.g., Y. Tang, G. Ghirlanda, W. A. Petka, T. Nakajima, W. F. DeGrado and D. A. Tirrell, Angew. Chem. Int. Ed. Engl., 40:1494 (2001). Moreover, selenomethionine and telluromethionine are incorporated into various recombinant proteins to facilitate the solution of phases in X-ray crystallography. See, e.g., W. A. Hendrickson, J. R. Horton and D. M. Lemaster, EMBO J., 9:1665 (1990); J. O. Boles, K. Lewinski, M. Kunkle, J. D. Odom, B. Dunlap, L. Lebioda and M. Hatada, Nat. Struct. Biol., 1:283 (1994); N. Budisa, B. Steipe, P. Demange, C. Eckerskorn, J. Kellermann and R. Huber, Eur. J. Biochem., 230:788 (1995); and, N. Budisa, W. Kambrock, S. Steinbacher, A. Humm, L. Prade, T. Neuefeind, L. Moroder and R. Huber, J. Mol. Biol., 270:616 (1997). Methionine analogs with alkene or alkyne functionalities have also been incorporated efficiently, allowing for additional modification of proteins by chemical means. See, e.g., J. C. van Hest and D. A. Tirrell, FEBS Lett., 428:68 (1998); J. C. van Hest, K. L. Kiick and D. A. Tirrell, J. Am. Chem. Soc., 122:1282 (2000); and, K. L. Kiick and D. A. Tirrell, Tetrahedron, 56:9487 (2000); U.S. Pat. No. 6,586,207; U.S. patent Pub 2002/0042097, which are incorporated by reference herein.

The success of this method depends on the recognition of the unnatural amino acid analogs by aminoacyl-tRNA synthetases, which, in general, require high selectivity to insure the fidelity of protein translation. One way to expand the scope of this method is to relax the substrate specificity of aminoacyl-tRNA synthetases, which has been achieved in a limited number of cases. For example, replacement of Ala²⁹⁴ by Gly in Escherichia coli phenylalanyl-tRNA synthetase (PheRS) increases the size of substrate binding pocket, and results in the acylation of tRNAPhe by p-Cl-phenylalanine (p-Cl-Phe). See, M. Ibba, P. Kast and H. Hennecke, Biochemistry, 33:7107 (1994). An Escherichia coli strain harboring this mutant PheRS allows the incorporation of p-Cl-phenylalanine or p-Br-phenylalanine in place of phenylalanine. See, e.g., M. Ibba and H. Hennecke, FEBS Lett., 364:272 (1995); and, N. Sharma, R. Furter, P. Kast and D. A. Tirrell, FEBS Lett., 467:37 (2000). Similarly, a point mutation Phe130Ser near the amino acid binding site of Escherichia coli tyrosyl-tRNA synthetase was shown to allow azatyrosine to be incorporated more efficiently than tyrosine. See, F. Hamano-Takaku, T. Iwama, S. Saito-Yano, K. Takaku, Y. Monden, M. Kitabatake, D. Soll and S. Nishimura, J. Biol. Chem., 275:40324 (2000).

Another strategy to incorporate unnatural amino acids into proteins in vivo is to modify synthetases that have proofreading mechanisms. These synthetases cannot discriminate and therefore activate amino acids that are structurally similar to the cognate natural amino acids. This error is corrected at a separate site, which deacylates the mischarged amino acid from the tRNA to maintain the fidelity of protein translation. If the proofreading activity of the synthetase is disabled, structural analogs that are misactivated may escape the editing function and be incorporated. This approach has been demonstrated recently with the valyl-tRNA synthetase (ValRS). See, V. Doring, H. D. Mootz, L. A. Nangle, T. L. Hendrickson, V. de Crecy-Lagard, P. Schimmel and P. Marliere, Science, 292:501 (2001). ValRS can misaminoacylate tRNAVal with Cys, Thr, or aminobutyrate (Abu); these noncognate amino acids are subsequently hydrolyzed by the editing domain. After random mutagenesis of the Escherichia coli chromosome, a mutant Escherichia coli strain was selected that has a mutation in the editing site of ValRS. This edit-defective ValRS incorrectly charges tRNAVal with Cys. Because Abu sterically resembles Cys (—SH group of Cys is replaced with —CH3 in Abu), the mutant ValRS also incorporates Abu into proteins when this mutant Escherichia coli strain is grown in the presence of Abu. Mass spectrometric analysis shows that about 24% of valines are replaced by Abu at each valine position in the native protein.

Solid-phase synthesis and semisynthetic methods have also allowed for the synthesis of a number of proteins containing novel amino acids. For example, see the following publications and references cited within, which are as follows: Crick, F. H. C., Barrett, L. Brenner, S. Watts-Tobin, R. General nature of the genetic code for proteins. Nature, 192:1227-1232 (1961); Hofmann, K., Bohn, H. Studies on polypeptides. XXXVI. The effect of pyrazole-imidazole replacements on the S-protein activating potency of an S-peptide fragment, J. Am Chem, 88(24):5914-5919 (1966); Kaiser, E. T. Synthetic approaches to biologically active peptides and proteins including enzymes, Acc Chem Res, 22:47-54 (1989); Nakatsuka, T., Sasaki, T., Kaiser, E. T. Peptide segment coupling catalyzed by the semisynthetic enzyme thiosubtilisin, J Am Chem Soc, 109:3808-3810 (1987); Schnolzer, M., Kent, S B H. Constructing proteins by dovetailing unprotected synthetic peptides: backbone-engineered HIV protease, Science, 256(5054):221-225 (1992); Chaiken, I. M. Semisynthetic peptides and proteins, CRC Crit Rev Biochem, 11(3):255-301 (1981); Offord, R. E. Protein engineering by chemical means?Protein Eng., 1(3):151-157 (1987); and, Jackson, D. Y., Burnier, J., Quan, C., Stanley, M., Tom, J., Wells, J. A. A Designed Peptide Ligase for Total Synthesis of Ribonuclease A with Unnatural Catalytic Residues, Science, 266(5183):243 (1994).

Chemical modification has been used to introduce a variety of unnatural side chains, including cofactors, spin labels and oligonucleotides into proteins in vitro. See, e.g., Corey, D. R., Schultz, P. G. Generation of a hybrid sequence-specific single-stranded deoxyribonuclease, Science, 238(4832):1401-1403 (1987); Kaiser, E. T., Lawrence D. S., Rokita, S. E. The chemical modification of enzymatic specificity, Annu Rev Biochem, 54:565-595 (1985); Kaiser, E. T., Lawrence, D. S. Chemical mutation of enzyme active sites, Science, 226(4674):505-511 (1984); Neet, K. E., Nanci A, Koshland, D. E. Properties of thiol-subtilisin, J Biol. Chem, 243(24):6392-6401 (1968); Polgar, L. et M. L. Bender. A new enzyme containing a synthetically formed active site. Thiol-subtilisin. J. Am Chem Soc, 88:3153-3154 (1966); and, Pollack, S. J., Nakayama, G. Schultz, P. G. Introduction of nucleophiles and spectroscopic probes into antibody combining sites, Science, 242(4881): 1038-1040 (1988).

Alternatively, biosynthetic methods that employ chemically modified aminoacyl-tRNAs have been used to incorporate several biophysical probes into proteins synthesized in vitro. See the following publications and references cited within: Brunner, J. New Photolabeling and crosslinking methods, Annu. Rev Biochem, 62:483-514 (1993); and, Krieg, U. C., Walter, P., Hohnson, A. E. Photocrosslinking of the signal sequence of nascent preprolactin of the 54-kilodalton polypeptide of the signal recognition particle, Proc. Natl. Acad. Sci, 83(22):8604-8608 (1986).

Previously, it has been shown that unnatural amino acids can be site-specifically incorporated into proteins in vitro by the addition of chemically aminoacylated suppressor tRNAs to protein synthesis reactions programmed with a gene containing a desired amber nonsense mutation. Using these approaches, one can substitute a number of the common twenty amino acids with close structural homologues, e.g., fluorophenylalanine for phenylalanine, using strains auxotrophic for a particular amino acid. See, e.g., Noren, C. J., Anthony-Cahill, Griffith, M. C., Schultz, P. G. A general method for site-specific incorporation of unnatural amino acids into proteins, Science, 244: 182-188 (1989); M. W. Nowak, et al., Science 268:439-42 (1995); Bain, J. D., Glabe, C. G., Dix, T. A., Chamberlin, A. R., Diala, E. S. Biosynthetic site-specific Incorporation of a non-natural amino acid into a polypeptide, J. Am Chem Soc, 111:8013-8014 (1989); N. Budisa et al., FASEB J. 13:41-51 (1999); Ellman, J. A., Mendel, D., Anthony-Cahill, S., Noren, C. J., Schultz, P. G. Biosynthetic method for introducing unnatural amino acids site-specifically into proteins, Methods in Enz., vol. 202, 301-336 (1992); and, Mendel, D., Cornish, V. W. & Schultz, P. G. Site-Directed Mutagenesis with an Expanded Genetic Code, Annu Rev Biophys. Biomol Struct. 24, 435-62 (1995).

For example, a suppressor tRNA was prepared that recognized the stop codon UAG and was chemically aminoacylated with an unnatural amino acid. Conventional site-directed mutagenesis was used to introduce the stop codon TAG, at the site of interest in the protein gene. See, e.g., Sayers, J. R., Schmidt, W. Eckstein, F. 5′-3′ Exonucleases in phosphorothioate-based olignoucleotide-directed mutagenesis, Nucleic Acids Res, 16(3):791-802 (1988). When the acylated suppressor tRNA and the mutant gene were combined in an in vitro transcription/translation system, the unnatural amino acid was incorporated in response to the UAG codon which gave a protein containing that amino acid at the specified position. Experiments using [³H]-Phe and experiments with α-hydroxy acids demonstrated that only the desired amino acid is incorporated at the position specified by the UAG codon and that this amino acid is not incorporated at any other site in the protein. See, e.g., Noren, et al, supra; Kobayashi et al., (2003) Nature Structural Biology 10(6):425-432; and, Ellman, J. A., Mendel, D., Schultz, P. G. Site-specific incorporation of novel backbone structures into proteins, Science, 255(5041):197-200 (1992).

A tRNA may be aminoacylated with a desired amino acid by any method or technique, including but not limited to, chemical or enzymatic aminoacylation. Aminoacylation may be accomplished by aminoacyl tRNA synthetases or by other enzymatic molecules, including but not limited to, ribozymes. The term “ribozyme” is interchangeable with “catalytic RNA.” Cech and coworkers (Cech, 1987, Science, 236:1532-1539; McCorkle et al., 1987, Concepts Biochem. 64:221-226) demonstrated the presence of naturally occurring RNAs that can act as catalysts (ribozymes). However, although these natural RNA catalysts have only been shown to act on ribonucleic acid substrates for cleavage and splicing, the recent development of artificial evolution of ribozymes has expanded the repertoire of catalysis to various chemical reactions. Studies have identified RNA molecules that can catalyze aminoacyl-RNA bonds on their own (2′)3′-termini (Illangakekare et al., 1995 Science 267:643-647), and an RNA molecule which can transfer an amino acid from one RNA molecule to another (Lohse et al., 1996, Nature 381:442-444).

U.S. Patent Application Publication 2003/0228593, which is incorporated by reference herein, describes methods to construct ribozymes and their use in aminoacylation of tRNAs with naturally encoded and non-naturally encoded amino acids. Substrate-immobilized forms of enzymatic molecules that can aminoacylate tRNAs, including but not limited to, ribozymes, may enable efficient affinity purification of the aminoacylated products. Examples of suitable substrates include agarose, sepharose, and magnetic beads. The production and use of a substrate-immobilized form of ribozyme for aminoacylation is described in Chemistry and Biology 2003, 10:1077-1084 and U.S. Patent Application Publication 2003/0228593, which are incorporated by reference herein.

Chemical aminoacylation methods include, but are not limited to, those introduced by Hecht and coworkers (Hecht, S. M. Acc. Chem. Res. 1992, 25, 545; Heckler, T. G.; Roesser, J. R.; Xu, C.; Chang, P.; Hecht, S. M. Biochemistry 1988, 27, 7254; Hecht, S. M.; Alford, B. L.; Kuroda, Y.; Kitano, S. J. Biol. Chem. 1978, 253, 4517) and by Schultz, Chamberlin, Dougherty and others (Cornish, V. W.; Mendel, D.; Schultz, P. G. Angew. Chem. Int. Ed. Engl. 1995, 34, 621; Robertson, S. A.; Ellman, J. A.; Schultz, P. G. J. Am. Chem. Soc. 1991, 113, 2722; Noren, C. J.; Anthony-Cahill, S. J.; Griffith, M. C.; Schultz, P. G. Science 1989, 244, 182; Bain, J. D.; Glabe, C. G.; Dix, T. A.; Chamberlin, A. R. J. Am. Chem. Soc. 1989, 111, 8013; Bain, J. D. et al. Nature 1992, 356, 537; Gallivan, J. P.; Lester, H. A.; Dougherty, D. A. Chem. Biol. 1997, 4, 740; Turcatti, et al. J. Biol. Chem. 1996, 271, 19991; Nowak, M. W. et al. Science, 1995, 268, 439; Saks, M. E. et al. J. Biol. Chem. 1996, 271, 23169; Hohsaka, T. et al. J. Am. Chem. Soc. 1999, 121, 34), which are incorporated by reference herein, to avoid the use of synthetases in aminoacylation. Such methods or other chemical aminoacylation methods may be used to aminoacylate tRNA molecules.

Methods for generating catalytic RNA may involve generating separate pools of randomized ribozyme sequences, performing directed evolution on the pools, screening the pools for desirable aminoacylation activity, and selecting sequences of those ribozymes exhibiting desired aminoacylation activity.

Ribozymes can comprise motifs and/or regions that facilitate acylation activity, such as a GGU motif and a U-rich region. For example, it has been reported that U-rich regions can facilitate recognition of an amino acid substrate, and a GGU-motif can form base pairs with the 3′ termini of a tRNA. In combination, the GGU and motif and U-rich region facilitate simultaneous recognition of both the amino acid and tRNA simultaneously, and thereby facilitate aminoacylation of the 3′ terminus of the tRNA. Ribozymes can be generated by in vitro selection using a partially randomized r24 mini conjugated with tRNA^{Asn}_{CCCG}, followed by systematic engineering of a consensus sequence found in the active clones. An exemplary ribozyme obtained by this method is termed “Fx3 ribozyme” and is described in U.S. Pub. App. No. 2003/0228593, the contents of which is incorporated by reference herein, acts as a versatile catalyst for the synthesis of various aminoacyl-tRNAs charged with cognate non-natural amino acids.

Immobilization on a substrate may be used to enable efficient affinity purification of the aminoacylated tRNAs. Examples of suitable substrates include, but are not limited to, agarose, sepharose, and magnetic beads. Ribozymes can be immobilized on resins by taking advantage of the chemical structure of RNA, such as the 3′-cis-diol on the ribose of RNA can be oxidized with periodate to yield the corresponding dialdehyde to facilitate immobilization of the RNA on the resin. Various types of resins can be used including inexpensive hydrazide resins wherein reductive amination makes the interaction between the resin and the ribozyme an irreversible linkage. Synthesis of aminoacyl-tRNAs can be significantly facilitated by this on-column aminoacylation technique. Kourouklis et al. Methods 2005; 36:239-4 describe a column-based aminoacylation system.

Isolation of the aminoacylated tRNAs can be accomplished in a variety of ways. One suitable method is to elute the aminoacylated tRNAs from a column with a buffer such as a sodium acetate solution with 10 mM EDTA, a buffer containing 50 mM N-(2-hydroxyethyl)piperazine-N′-(3-propanesulfonic acid), 12.5 mM KCl, pH 7.0, 10 mM EDTA, or simply an EDTA buffered water (pH 7.0). The aminoacylated tRNAs can be added to translation reactions in order to incorporate the amino acid with which the tRNA was aminoacylated in a position of choice in a polypeptide made by the translation reaction. Examples of translation systems in which the aminoacylated tRNAs of the present invention may be used include, but are not limited to cell lysates. Cell lysates provide reaction components necessary for in vitro translation of a polypeptide from an input mRNA. Examples of such reaction components include but are not limited to ribosomal proteins, rRNA, amino acids, tRNAs, GTP, ATP, translation initiation and elongation factors and additional factors associated with translation. Additionally, translation systems may be batch translations or compartmentalized translation. Batch translation systems combine reaction components in a single compartment while compartmentalized translation systems separate the translation reaction components from reaction products that can inhibit the translation efficiency. Such translation systems are available commercially.

Further, a coupled transcription/translation system may be used. Coupled transcription/translation systems allow for both transcription of an input DNA into a corresponding mRNA, which is in turn translated by the reaction components. An example of a commercially available coupled transcription/translation is the Rapid Translation System (RTS, Roche Inc.). The system includes a mixture containing E. coli lysate for providing translational components such as ribosomes and translation factors. Additionally, an RNA polymerase is included for the transcription of the input DNA into an mRNA template for use in translation. RTS can use compartmentalization of the reaction components by way of a membrane interposed between reaction compartments, including a supply/waste compartment and a transcription/translation compartment.

Aminoacylation of tRNA may be performed by other agents, including but not limited to, transferases, polymerases, catalytic antibodies, multi-functional proteins, and the like. Stephan in Scientist Oct. 10, 2005; pages 30-33 describes additional methods to incorporate non-naturally encoded amino acids into proteins. Lu et al. in Mol Cell. 2001 October; 8(4):759-69 describe a method in which a protein is chemically ligated to a synthetic peptide containing unnatural amino acids (expressed protein ligation).

Microinjection techniques have also been use incorporate unnatural amino acids into proteins. See, e.g., M. W. Nowak, P. C. Kearney, J. R. Sampson, M. E. Saks, C. G. Labarca, S. K. Silverman, W. G. Zhong, J. Thorson, J. N. Abelson, N. Davidson, P. G. Schultz, D. A. Dougherty and H. A. Lester, Science, 268:439 (1995); and, D. A. Dougherty, Curr. Opin. Chem. Biol., 4:645 (2000). A Xenopus oocyte was coinjected with two RNA species made in vitro: an mRNA encoding the target protein with a UAG stop codon at the amino acid position of interest and an amber suppressor tRNA aminoacylated with the desired unnatural amino acid. The translational machinery of the oocyte then inserts the unnatural amino acid at the position specified by UAG. This method has allowed in vivo structure-function studies of integral membrane proteins, which are generally not amenable to in vitro expression systems. Examples include the incorporation of a fluorescent amino acid into tachykinin neurokinin-2 receptor to measure distances by fluorescence resonance energy transfer, see, e.g., G. Turcatti, K. Nemeth, M. D. Edgerton, U. Meseth, F. Talabot, M. Peitsch, J. Knowles, H. Vogel and A. Chollet, J. Biol. Chem., 271:19991 (1996); the incorporation of biotinylated amino acids to identify surface-exposed residues in ion channels, see, e.g., J. P. Gallivan, H. A. Lester and D. A. Dougherty, Chem. Biol., 4:739 (1997); the use of caged tyrosine analogs to monitor conformational changes in an ion channel in real time, see, e.g., J. C. Miller, S. K. Silverman, P. M. England, D. A. Dougherty and H. A. Lester, Neuron, 20:619 (1998); and, the use of alpha hydroxy amino acids to change ion channel backbones for probing their gating mechanisms. See, e.g., P. M. England, Y. Zhang, D. A. Dougherty and H. A. Lester, Cell, 96:89 (1999); and, T. Lu, A. Y. Ting, J. Mainland, L. Y. Jan, P. G. Schultz and J. Yang, Nat. Neurosci., 4:239 (2001).

The ability to incorporate unnatural amino acids directly into proteins in vivo offers a wide variety of advantages including but not limited to, high yields of mutant proteins, technical ease, the potential to study the mutant proteins in cells or possibly in living organisms and the use of these mutant proteins in therapeutic treatments and diagnostic uses. The ability to include unnatural amino acids with various sizes, acidities, nucleophilicities, hydrophobicities, and other properties into proteins can greatly expand our ability to rationally and systematically manipulate the structures of proteins, both to probe protein function and create new proteins or organisms with novel properties. In one attempt to site-specifically incorporate para-F-Phe, a yeast amber suppressor tRNAPheCUA/phenylalanyl-tRNA synthetase pair was used in a p-F-Phe resistant, Phe auxotrophic Escherichia coli strain. See, e.g., R. Furter, Protein Sci., 7:419 (1998).

It may also be possible to obtain expression of an IFNL3 polynucleotide of the present invention using a cell-free (in-vitro) translational system. Translation systems may be cellular or cell-free, and may be prokaryotic or eukaryotic. Cellular translation systems include, but are not limited to, whole cell preparations such as permeabilized cells or cell cultures wherein a desired nucleic acid sequence can be transcribed to mRNA and the mRNA translated. Cell-free translation systems are commercially available and many different types and systems are well-known. Examples of cell-free systems include, but are not limited to, prokaryotic lysates such as Escherichia coli lysates, and eukaryotic lysates such as wheat germ extracts, insect cell lysates, rabbit reticulocyte lysates, rabbit oocyte lysates and human cell lysates. Eukaryotic extracts or lysates may be preferred when the resulting protein is glycosylated, phosphorylated or otherwise modified because many such modifications are only possible in eukaryotic systems. Some of these extracts and lysates are available commercially (Promega; Madison, Wis.; Stratagene; La Jolla, Calif.; Amersham; Arlington Heights, Ill.; GIBCO/BRL; Grand Island, N.Y.). Membranous extracts, such as the canine pancreatic extracts containing microsomal membranes, are also available which are useful for translating secretory proteins. In these systems, which can include either mRNA as a template (in-vitro translation) or DNA as a template (combined in-vitro transcription and translation), the in vitro synthesis is directed by the ribosomes. Considerable effort has been applied to the development of cell-free protein expression systems. See, e.g., Kim, D. M. and J. R. Swartz, Biotechnology and Bioengineering, 74:309-316 (2001); Kim, D. M. and J. R. Swartz, Biotechnology Letters, 22, 1537-1542, (2000); Kim, D. M., and J. R. Swartz, Biotechnology Progress, 16, 385-390, (2000); Kim, D. M., and J. R. Swartz, Biotechnology and Bioengineering, 66, 180-188, (1999); and Patnaik, R. and J. R. Swartz, Biotechniques 24, 862-868, (1998); U.S. Pat. No. 6,337,191; U.S. Patent Publication No. 2002/0081660; WO 00/55353; WO 90/05785, which are incorporated by reference herein. Another approach that may be applied to the expression of IFNL3 polypeptides comprising a non-naturally encoded amino acid includes the mRNA-peptide fusion technique. See, e.g., R. Roberts and J. Szostak, Proc. Natl Acad. Sci. (USA) 94:12297-12302 (1997); A. Frankel, et al., Chemistry & Biology 10:1043-1050 (2003). In this approach, an mRNA template linked to puromycin is translated into peptide on the ribosome. If one or more tRNA molecules has been modified, non-natural amino acids can be incorporated into the peptide as well. After the last mRNA codon has been read, puromycin captures the C-terminus of the peptide. If the resulting mRNA-peptide conjugate is found to have interesting properties in an in vitro assay, its identity can be easily revealed from the mRNA sequence. In this way, one may screen libraries of IFNL3 polypeptides comprising one or more non-naturally encoded amino acids to identify polypeptides having desired properties. More recently, in vitro ribosome translations with purified components have been reported that permit the synthesis of peptides substituted with non-naturally encoded amino acids. See, e.g., A. Forster et al., Proc. Natl Acad. Sci. (USA) 100:6353 (2003).

Reconstituted translation systems may also be used. Mixtures of purified translation factors have also been used successfully to translate mRNA into protein as well as combinations of lysates or lysates supplemented with purified translation factors such as initiation factor-1 (IF-1), IF-2, IF-3 (α or β), elongation factor T (EF-Tu), or termination factors. Cell-free systems may also be coupled transcription/translation systems wherein DNA is introduced to the system, transcribed into mRNA and the mRNA translated as described in Current Protocols in Molecular Biology (F. M. Ausubel et al. editors, Wiley Interscience, 1993), which is hereby specifically incorporated by reference. RNA transcribed in eukaryotic transcription system may be in the form of heteronuclear RNA (hnRNA) or 5′-end caps (7-methyl guanosine) and 3′-end poly A tailed mature mRNA, which can be an advantage in certain translation systems. For example, capped mRNAs are translated with high efficiency in the reticulocyte lysate system.

### Macromolecular Polymers Coupled to IFNL3 Polypeptides

Various modifications to the non-natural amino acid polypeptides described herein can be effected using the compositions, methods, techniques and strategies described herein. These modifications include the incorporation of further functionality onto the non-natural amino acid component of the polypeptide, including but not limited to, a PKEM, a label; a dye; a polymer; a water-soluble polymer; a derivative of polyethylene glycol; a photocrosslinker; a radionuclide; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance. As an illustrative, non-limiting example of the compositions, methods, techniques and strategies described herein, the following description will focus on adding macromolecular polymers to the non-natural amino acid polypeptide with the understanding that the compositions, methods, techniques and strategies described thereto are also applicable (with appropriate modifications, if necessary and for which one of skill in the art could make with the disclosures herein) to adding other functionalities, including but not limited to those listed above.

A wide variety of macromolecular polymers and other molecules can be linked to IFNL3 polypeptides of the present invention to modulate biological properties of the IFNL3 polypeptide, and/or provide new biological properties to the IFNL3 molecule. These macromolecular polymers can be linked to the IFNL3 polypeptide via a naturally encoded amino acid, via a non-naturally encoded amino acid, or any functional substituent of a natural or non-natural amino acid, or any substituent or functional group added to a natural or non-natural amino acid. The molecular weight of the polymer may be of a wide range, including but not limited to, between about 100 Da and about 100,000 Da or more. The molecular weight of the polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 5,000 Da and about 40,000 Da. In some embodiments, the molecular weight of the polymer is between about 10,000 Da and about 40,000 Da.

The present invention provides substantially homogenous preparations of polymer:protein conjugates. “Substantially homogenous” as used herein means that polymer:protein conjugate molecules are observed to be greater than half of the total protein. The polymer:protein conjugate has biological activity and the present “substantially homogenous” modified IFNL3 preparations provided herein are those which are homogenous enough to display the advantages of a homogenous preparation, e.g., ease in clinical application in predictability of lot to lot pharmacokinetics.

One may also choose to prepare a mixture of polymer:protein conjugate molecules, and the advantage provided herein is that one may select the proportion of mono-polymer:protein conjugate to include in the mixture. Thus, if desired, one may prepare a mixture of various proteins with various numbers of polymer moieties attached (i.e., di-, tri-, tetra-, etc.) and combine said conjugates with the mono-polymer:protein conjugate prepared using the methods of the present invention, and have a mixture with a predetermined proportion of mono-polymer:protein conjugates.

The polymer selected may be water soluble so that the protein to which it is attached does not precipitate in an aqueous environment, such as a physiological environment. The polymer may be branched or unbranched. For therapeutic use of the end-product preparation, the polymer will be pharmaceutically acceptable.

Examples of polymers include but are not limited to certain half-life extending moieties, polyalkyl ethers and alkoxy-capped analogs thereof (e.g., polyoxyethylene glycol, polyoxyethylene/propylene glycol, and methoxy or ethoxy-capped analogs thereof, especially polyoxyethylene glycol, the latter is also known as polyethyleneglycol or PEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof, hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof; terpolymers thereof, mixtures thereof; and derivatives of the foregoing.

Those of ordinary skill in the art will recognize that the foregoing list for substantially water soluble backbones is by no means exhaustive and is merely illustrative, and that all polymeric materials having the qualities described above are contemplated as being suitable for use in the present invention.

In some embodiments of the present invention the polymer derivatives are “multi-functional”, meaning that the polymer backbone has at least two termini, and possibly as many as about 300 termini, functionalized or activated with a functional group. Multifunctional polymer derivatives include, but are not limited to, linear polymers having two termini, each terminus being bonded to a functional group which may be the same or different.

In one embodiment, the polymer derivative has the structure:

X-A-POLY-B—N═N═N

wherein:
N═N═N is an azide moiety;
B is a linking moiety, which may be present or absent;
POLY is a water-soluble non-antigenic polymer;
A is a linking moiety, which may be present or absent and which may be the same as B or different; and
X is a second functional group.

Examples of a linking moiety for A and B include, but are not limited to, a multiply-functionalized alkyl group containing up to 18, and may contain between 1-10 carbon atoms. A heteroatom such as nitrogen, oxygen or sulfur may be included with the alkyl chain. The alkyl chain may also be branched at a heteroatom. Other examples of a linking moiety for A and B include, but are not limited to, a multiply functionalized aryl group, containing up to 10 and may contain 5-6 carbon atoms. The aryl group may be substituted with one more carbon atoms, nitrogen, oxygen or sulfur atoms. Other examples of suitable linking groups include those linking groups described in U.S. Pat. Nos. 5,932,462; 5,643,575; and U.S. Pat. Appl. Publication 2003/0143596, each of which is incorporated by reference herein. Those of ordinary skill in the art will recognize that the foregoing list for linking moieties is by no means exhaustive and is merely illustrative, and that all linking moieties having the qualities described above are contemplated to be suitable for use in the present invention.

Examples of suitable functional groups for use as X include, but are not limited to, hydroxyl, protected hydroxyl, alkoxyl, active ester, such as N-hydroxysuccinimidyl esters and 1-benzotriazolyl esters, active carbonate, such as N-hydroxysuccinimidyl carbonates and 1-benzotriazolyl carbonates, acetal, aldehyde, aldehyde hydrates, alkenyl, acrylate, methacrylate, acrylamide, active sulfone, amine, aminooxy, protected amine, hydrazide, protected hydrazide, protected thiol, carboxylic acid, protected carboxylic acid, isocyanate, isothiocyanate, maleimide, vinylsulfone, dithiopyridine, vinylpyridine, iodoacetamide, epoxide, glyoxals, diones, mesylates, tosylates, tresylate, alkene, ketone, and azide. As is understood by those of ordinary skill in the art, the selected X moiety should be compatible with the azide group so that reaction with the azide group does not occur. The azide-containing polymer derivatives may be homobifunctional, meaning that the second functional group (i.e., X) is also an azide moiety, or heterobifunctional, meaning that the second functional group is a different functional group.

The term “protected” refers to the presence of a protecting group or moiety that prevents reaction of the chemically reactive functional group under certain reaction conditions. The protecting group will vary depending on the type of chemically reactive group being protected. For example, if the chemically reactive group is an amine or a hydrazide, the protecting group can be selected from the group of tert-butyloxycarbonyl (t-Boc) and 9-fluorenylmethoxycarbonyl (Fmoc). If the chemically reactive group is a thiol, the protecting group can be orthopyridyldisulfide. If the chemically reactive group is a carboxylic acid, such as butanoic or propionic acid, or a hydroxyl group, the protecting group can be benzyl or an alkyl group such as methyl, ethyl, or tert-butyl. Other protecting groups known in the art may also be used in the present invention.

Specific examples of terminal functional groups in the literature include, but are not limited to, N-succinimidyl carbonate (see e.g., U.S. Pat. Nos. 5,281,698, 5,468,478), amine (see, e.g., Buckmann et al. Makromol. Chem. 182:1379 (1981), Zalipsky et al. Eur. Polym. J. 19:1177 (1983)), hydrazide (See, e.g., Andresz et al. Makromol. Chem. 179:301 (1978)), succinimidyl propionate and succinimidyl butanoate (see, e.g., Olson et al. in Poly(ethylene glycol) Chemistry & Biological applications, pp 170-181, Harris & Zalipsky Eds., ACS, Washington, D.C., 1997; see also U.S. Pat. No. 5,672,662), succinimidyl succinate (See, e.g., Abuchowski et al. Cancer Biochem. Biophys. 7:175 (1984) and Joppich et al. Makromol. Chem. 180:1381 (1979), succinimidyl ester (see, e.g., U.S. Pat. No. 4,670,417), benzotriazole carbonate (see, e.g., U.S. Pat. No. 5,650,234), glycidyl ether (see, e.g., Pitha et al. Eur. J Biochem. 94:11 (1979), Elling et al., Biotech. application. Biochem. 13:354 (1991), oxycarbonylimidazole (see, e.g., Beauchamp, et al., Anal. Biochem. 131:25 (1983), Tondelli et al. J. Controlled Release 1:251 (1985)), p-nitrophenyl carbonate (see, e.g., Veronese, et al., Appl. Biochem. Biotech., 11: 141 (1985); and Sartore et al., application. Biochem. Biotech., 27:45 (1991)), aldehyde (see, e.g., Harris et al. J. Polym. Sci. Chem. Ed. 22:341 (1984), U.S. Pat. Nos. 5,824,784, 5,252,714), maleimide (see, e.g., Goodson et al. Biotechnology (NY) 8:343 (1990), Romani et al. in Chemistry of Peptides and Proteins 2:29 (1984)), and Kogan, Synthetic Comm. 22:2417 (1992)), orthopyridyl-disulfide (see, e.g., Woghiren, et al. Bioconj. Chem. 4:314 (1993)), acrylol (see, e.g., Sawhney et al., Macromolecules, 26:581 (1993)), vinylsulfone (see, e.g., U.S. Pat. No. 5,900,461). All of the above references and patents are incorporated herein by reference.

In certain embodiments of the present invention, the polymer derivatives of the invention comprise a polymer backbone having the structure:

X—CH₂CH₂O—(CH₂CH₂O)ₙ—CH₂CH₂—N═N═N

wherein:
X is a functional group as described above; and
n is about 20 to about 4000.

In another embodiment, the polymer derivatives of the invention comprise a polymer backbone having the structure:

X—CH₂CH₂O—(CH₂CH₂O)ₙ—CH₂CH₂—O—(CH₂)ₘ—W—N═N═N

wherein:
W is an aliphatic or aromatic linker moiety comprising between 1-10 carbon atoms;
n is about 20 to about 4000; and
X is a functional group as described above. m is between 1 and 10.

The azide-containing PKEM derivatives of the invention can be prepared by a variety of methods known in the art and/or disclosed herein. In one method, shown below, a water soluble polymer backbone having an average molecular weight from about 800 Da to about 100,000 Da, the polymer backbone having a first terminus bonded to a first functional group and a second terminus bonded to a suitable leaving group, is reacted with an azide anion (which may be paired with any of a number of suitable counter-ions, including sodium, potassium, tert-butylammonium and so forth). The leaving group undergoes a nucleophilic displacement and is replaced by the azide moiety, affording the desired azide-containing PKEM polymer. X-PKEM-L+N₃⁻→X-PKEM-N₃

As shown, a suitable polymer backbone for use in the present invention has the formula X-PKEM-L, wherein PKEM is poly(ethylene glycol) and X is a functional group which does not react with azide groups and L is a suitable leaving group. Examples of suitable functional groups include, but are not limited to, hydroxyl, protected hydroxyl, acetal, alkenyl, amine, aminooxy, protected amine, protected hydrazide, protected thiol, carboxylic acid, protected carboxylic acid, maleimide, dithiopyridine, and vinylpyridine, and ketone. Examples of suitable leaving groups include, but are not limited to, chloride, bromide, iodide, mesylate, tresylate, and tosylate.

In another method for preparation of the azide-containing polymer derivatives of the present invention, a linking agent bearing an azide functionality is contacted with a water soluble polymer backbone having an average molecular weight from about 800 Da to about 100,000 Da, wherein the linking agent bears a chemical functionality that will react selectively with a chemical functionality on the PKEM polymer, to form an azide-containing polymer derivative product wherein the azide is separated from the polymer backbone by a linking group.

An exemplary reaction scheme is shown below:

X-PKEM-M+N-linker-N═N═N→PG-X-PKEM-linker-N═N═N

wherein:
PKEM is poly(ethylene glycol) and X is a capping group such as alkoxy or a functional group as described above; and
M is a functional group that is not reactive with the azide functionality but that will react efficiently and selectively with the N functional group.

Examples of suitable functional groups include, but are not limited to, M being a carboxylic acid, carbonate or active ester if N is an amine; M being a ketone if N is a hydrazide or aminooxy moiety; M being a leaving group if N is a nucleophile. Purification of the crude product may be accomplished by known methods including, but are not limited to, precipitation of the product followed by chromatography, if necessary.

A more specific example is shown below in the case of PKEM diamine, in which one of the amines is protected by a protecting group moiety such as tert-butyl-Boc and the resulting mono-protected PKEM diamine is reacted with a linking moiety that bears the azide functionality:

BocHN-PKEM-NH₂+HO₂C—(CH₂)₃—N═N═N

In this instance, the amine group can be coupled to the carboxylic acid group using a variety of activating agents such as thionyl chloride or carbodiimide reagents and N-hydroxysuccinimide or N-hydroxybenzotriazole to create an amide bond between the monoamine PKEM derivative and the azide-bearing linker moiety. After successful formation of the amide bond, the resulting N-tert-butyl-Boc-protected azide-containing derivative can be used directly to modify bioactive molecules or it can be further elaborated to install other useful functional groups. For instance, the N-t-Boc group can be hydrolyzed by treatment with strong acid to generate an omega-amino-PKEM-azide. The resulting amine can be used as a synthetic handle to install other useful functionality such as maleimide groups, activated disulfides, activated esters and so forth for the creation of valuable heterobifunctional reagents.

Heterobifunctional derivatives are particularly useful when it is desired to attach different molecules to each terminus of the polymer. For example, the omega-N-amino-N-azido PKEM would allow the attachment of a molecule having an activated electrophilic group, such as an aldehyde, ketone, activated ester, activated carbonate and so forth, to one terminus of the PKEM and a molecule having an acetylene group to the other terminus of the PKEM.

In another embodiment of the invention, the polymer derivative has the structure:

X-A-POLY-B—C═C—R

wherein:
R can be either H or an alkyl, alkene, alkyoxy, or aryl or substituted aryl group;
B is a linking moiety, which may be present or absent;
POLY is a water-soluble non-antigenic polymer;
A is a linking moiety, which may be present or absent and which may be the same as B or different; and
X is a second functional group.

Examples of a linking moiety for A and B include, but are not limited to, a multiply-functionalized alkyl group containing up to 18, and may contain between 1-10 carbon atoms. A heteroatom such as nitrogen, oxygen or sulfur may be included with the alkyl chain. The alkyl chain may also be branched at a heteroatom. Other examples of a linking moiety for A and B include, but are not limited to, a multiply functionalized aryl group, containing up to 10 and may contain 5-6 carbon atoms. The aryl group may be substituted with one more carbon atoms, nitrogen, oxygen, or sulfur atoms. Other examples of suitable linking groups include those linking groups described in U.S. Pat. Nos. 5,932,462 and 5,643,575 and U.S. Pat. Appl. Publication 2003/0143596, each of which is incorporated by reference herein. Those of ordinary skill in the art will recognize that the foregoing list for linking moieties is by no means exhaustive and is intended to be merely illustrative, and that a wide variety of linking moieties having the qualities described above are contemplated to be useful in the present invention.

Examples of suitable functional groups for use as X include hydroxyl, protected hydroxyl, alkoxyl, active ester, such as N-hydroxysuccinimidyl esters and 1-benzotriazolyl esters, active carbonate, such as N-hydroxysuccinimidyl carbonates and 1-benzotriazolyl carbonates, acetal, aldehyde, aldehyde hydrates, alkenyl, acrylate, methacrylate, acrylamide, active sulfone, amine, aminooxy, protected amine, hydrazide, protected hydrazide, protected thiol, carboxylic acid, protected carboxylic acid, isocyanate, isothiocyanate, maleimide, vinylsulfone, dithiopyridine, vinylpyridine, iodoacetamide, epoxide, glyoxals, diones, mesylates, tosylates, and tresylate, alkene, ketone, and acetylene. As would be understood, the selected X moiety should be compatible with the acetylene group so that reaction with the acetylene group does not occur. The acetylene-containing polymer derivatives may be homobifunctional, meaning that the second functional group (i.e., X) is also an acetylene moiety, or heterobifunctional, meaning that the second functional group is a different functional group.

In another embodiment of the present invention, the polymer derivatives comprise a polymer backbone having the structure:

X—CH₂CH₂O—(CH₂CH₂O)ₙ—CH₂CH₂—O—(CH₂)ₘ—C≡CH

wherein:
X is a functional group as described above;
n is about 20 to about 4000; and
m is between 1 and 10.

Specific examples of each of the heterobifunctional PKEM polymers are shown below.

The acetylene-containing PKEM derivatives of the invention can be prepared using methods known to those of ordinary skill in the art and/or disclosed herein. In one method, a water soluble polymer backbone having an average molecular weight from about 800 Da to about 100,000 Da, the polymer backbone having a first terminus bonded to a first functional group and a second terminus bonded to a suitable nucleophilic group, is reacted with a compound that bears both an acetylene functionality and a leaving group that is suitable for reaction with the nucleophilic group on the PKEM. When the PKEM polymer bearing the nucleophilic moiety and the molecule bearing the leaving group are combined, the leaving group undergoes a nucleophilic displacement and is replaced by the nucleophilic moiety, affording the desired acetylene-containing polymer.

X-PKEM-Nu+L-A-C→X-PKEM-Nu-A-C≡CR′

As shown, a preferred polymer backbone for use in the reaction has the formula X-PKEM-Nu, wherein PKEM is poly(ethylene glycol), Nu is a nucleophilic moiety and X is a functional group that does not react with Nu, L or the acetylene functionality.

Examples of Nu include, but are not limited to, amine, alkoxy, aryloxy, sulfhydryl, imino, carboxylate, hydrazide, aminoxy groups that would react primarily via a SN2-type mechanism. Additional examples of Nu groups include those functional groups that would react primarily via an nucleophilic addition reaction. Examples of L groups include chloride, bromide, iodide, mesylate, tresylate, and tosylate and other groups expected to undergo nucleophilic displacement as well as ketones, aldehydes, thioesters, olefins, alpha-beta unsaturated carbonyl groups, carbonates and other electrophilic groups expected to undergo addition by nucleophiles.

In another embodiment of the present invention, A is an aliphatic linker of between 1-10 carbon atoms or a substituted aryl ring of between 6-14 carbon atoms. X is a functional group which does not react with azide groups and L is a suitable leaving group

In another method for preparation of the acetylene-containing polymer derivatives of the invention, a PKEM polymer having an average molecular weight from about 800 Da to about 100,000 Da, bearing either a protected functional group or a capping agent at one terminus and a suitable leaving group at the other terminus is contacted by an acetylene anion.

An exemplary reaction scheme is shown below:

X-PKEM-L+-C≡CR′→X-PKEM-C≡CR′

wherein:
PKEM is poly(ethylene glycol) and X is a capping group such as alkoxy or a functional group as described above; and
R′ is either H, an alkyl, alkoxy, aryl or aryloxy group or a substituted alkyl, alkoxyl, aryl or aryloxy group.

In the example above, the leaving group L should be sufficiently reactive to undergo SN2-type displacement when contacted with a sufficient concentration of the acetylene anion. The reaction conditions required to accomplish SN2 displacement of leaving groups by acetylene anions are known to those of ordinary skill in the art.

Purification of the crude product can usually be accomplished by methods known in the art including, but are not limited to, precipitation of the product followed by chromatography, if necessary.

PKEM can be linked to the IFNL3 polypeptides of the invention. The PKEM may be linked via a naturally encoded amino acid, a derivatized naturally encoded amino acid, or a non-naturally encoded amino acid incorporated in the IFNL3 polypeptide or any functional group or substituent of a non-naturally encoded or naturally encoded amino acid, or any functional group or substituent added to a non-naturally encoded or naturally encoded amino acid. Alternatively, the PKEM are linked to an IFNL3 polypeptide incorporating a non-naturally encoded amino acid via a naturally-occurring amino acid (including but not limited to, cysteine, lysine or the amine group of the N-terminal residue). In some cases, the IFNL3 polypeptides of the invention comprise 1, 2, 3, 4, 5, 6, 7, 8, 9, 10 non-natural amino acids, wherein one or more non-naturally-encoded amino acid(s) are linked to a PKEM or moieties. In some cases, the IFNL3 polypeptides of the invention further comprise 1, 2, 3, 4, 5, 6, 7, 8, 9, 10, or more naturally-encoded amino acid(s) linked to a PKEM or moieties. In some cases, the IFNL3 polypeptides of the invention comprise one or more non-naturally encoded amino acid(s) linked to PKEM and one or more naturally-occurring amino acids linked to PKEM. In some embodiments, the PKEM used in the present invention enhance the serum half-life of the IFNL3 polypeptide relative to the unconjugated form.

The number of PKEM linked to an IFNL3 polypeptide of the present invention can be adjusted to provide an altered (including but not limited to, increased or decreased) pharmacologic, pharmacokinetic or pharmacodynamic characteristic such as in vivo half-life. In some embodiments, the half-life of IFNL3 is increased at least about 10, 20, 30, 40, 50, 60, 70, 80, 90 percent, 2-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 50-fold, or at least about 100-fold over an unmodified polypeptide.

PKEM Derivatives Containing a Strong Nucleophilic Group (i.e., Hydrazide, Hydrazine, Hydroxylamine or Semicarbazide)

In one embodiment of the present invention, an IFNL3 polypeptide comprising a carbonyl-containing non-naturally encoded amino acid is modified with a PKEM derivative that contains a terminal hydrazine, hydroxylamine, hydrazide or semicarbazide moiety that is linked directly to the PKEM backbone.

In some embodiments, the hydroxylamine-terminal PKEM derivative will have the structure:

RO—(CH2CH2O)n-O—(CH2)m-O—NH2

where R is a simple alkyl (methyl, ethyl, propyl, etc.), m is 2-10 and n is 100-1,000 (i.e., average molecular weight is between 5-40 kDa).

In some embodiments, the hydrazine- or hydrazide-containing PKEM derivative will have the structure:

RO—(CH2CH2O)n-O—(CH2)m-X—NH—NH2

where R is a simple alkyl (methyl, ethyl, propyl, etc.), m is 2-10 and n is 100-1,000 and X is optionally a carbonyl group (C═O) that can be present or absent.

In some embodiments, the semicarbazide-containing PKEM derivative will have the structure:

RO—(CH2CH2O)n-O—(CH2)m-NH—C(O)—NH—NH2

where R is a simple alkyl (methyl, ethyl, propyl, etc.), m is 2-10 and n is 100-1,000.

In another embodiment of the invention, an IFNL3 polypeptide comprising a carbonyl-containing amino acid is modified with a PKEM derivative that contains a terminal hydroxylamine, hydrazide, hydrazine, or semicarbazide moiety that is linked to the PKEM backbone by means of an amide linkage.

In some embodiments, the hydroxylamine-terminal PKEM derivatives have the structure:

RO—(CH2CH2O)n-O—(CH2)₂—NH—C(O)(CH2)m-O—NH2

where R is a simple alkyl (methyl, ethyl, propyl, etc.), m is 2-10 and n is 100-1,000 (i.e., average molecular weight is between 5-40 kDa).

In some embodiments, the hydrazine- or hydrazide-containing PKEM derivatives have the structure:

RO—(CH2CH2O)n-O—(CH2)₂—NH—C(O)(CH2)m-X—NH—NH2

where R is a simple alkyl (methyl, ethyl, propyl, etc.), m is 2-10, n is 100-1,000 and X is optionally a carbonyl group (C═O) that can be present or absent.

In some embodiments, the semicarbazide-containing PKEM derivatives have the structure:

RO—(CH2CH2O)n-O—(CH2)₂—NH—C(O)(CH2)m-NH—C(O)—NH—NH2

where R is a simple alkyl (methyl, ethyl, propyl, etc.), m is 2-10 and n is 100-1,000.

In another embodiment of the invention, an IFNL3 polypeptide comprising a carbonyl-containing amino acid is modified with a branched PKEM derivative that contains a terminal hydrazine, hydroxylamine, hydrazide or semicarbazide moiety, with each chain of the branched PKEM having a MW ranging from 10-40 kDa and, may be from 5-20 kDa.

In another embodiment of the invention, an IFNL3 polypeptide comprising a non-naturally encoded amino acid is modified with a PKEM derivative having a branched structure. For instance, in some embodiments, the hydrazine- or hydrazide-terminal PKEM derivative will have the following structure:

[RO—(CH2CH2O)n-O—(CH2)₂—NH—C(O)]2CH(CH2)m-X—NH—NH2

where R is a simple alkyl (methyl, ethyl, propyl, etc.), m is 2-10 and n is 100-1,000, and X is optionally a carbonyl group (C═O) that can be present or absent.

In some embodiments, the PKEM derivatives containing a semicarbazide group will have the structure:

[RO—(CH2CH2O)n-O—(CH2)₂—C(O)—NH—CH2-CH2]2CH—X—(CH2)m-NH—C(O)—NH—NH2

where R is a simple alkyl (methyl, ethyl, propyl, etc.), X is optionally NH, O, S, C(O) or not present, m is 2-10 and n is 100-1,000.

In some embodiments, the PKEM derivatives containing a hydroxylamine group will have the structure:

[RO—(CH2CH2O)n-O—(CH2)₂—C(O)—NH—CH2-CH2]2CH—X—(CH2)m-O—NH2

where R is a simple alkyl (methyl, ethyl, propyl, etc.), X is optionally NH, O, S, C(O) or not present, m is 2-10 and n is 100-1,000.

Methods and chemistry for activation of polymers as well as for conjugation of peptides are described in the literature and are known in the art. Commonly used methods for activation of polymers include, but are not limited to, activation of functional groups with cyanogen bromide, periodate, glutaraldehyde, biepoxides, epichlorohydrin, divinylsulfone, carbodiimide, sulfonyl halides, trichlorotriazine, etc. (see, R. F. Taylor, (1991), PROTEIN IMMOBILISATION. FUNDAMENTAL AND APPLICATIONS, Marcel Dekker, N.Y.; S. S. Wong, (1992), CHEMISTRY OF PROTEIN CONJUGATION AND CROSSLINKING, CRC Press, Boca Raton; G. T. Hermanson et al., (1993), IMMOBILIZED AFFINITY LIGAND TECHNIQUES, Academic Press, N.Y.; Dunn, R. L., et al., Eds. POLYMERIC DRUGS AND DRUG DELIVERY SYSTEMS, ACS Symposium Series Vol. 469, American Chemical Society, Washington, D.C. 1991).

Several reviews and monographs on the functionalization and conjugation of PKEM are available. See, for example, Harris, Macromol. Chem. Phys. C25: 325-373 (1985); Scouten, Methods in Enzymology 135: 30-65 (1987); Wong et al., Enzyme Microb. Technol. 14: 866-874 (1992); Delgado et al., Critical Reviews in Therapeutic Drug Carrier Systems 9: 249-304 (1992); Zalipsky, Bioconjugate Chem. 6: 150-165 (1995).

Methods for activation of polymers can also be found in WO 94/17039, U.S. Pat. No. 5,324,844, WO 94/18247, WO 94/04193, U.S. Pat. Nos. 5,219,564, 5,122,614, WO 90/13540, U.S. Pat. No. 5,281,698, and WO 93/15189, and for conjugation between activated polymers and enzymes including but not limited to Coagulation Factor VIII (WO 94/15625), hemoglobin (WO 94/09027), oxygen carrying molecule (U.S. Pat. No. 4,412,989), ribonuclease and superoxide dismutase (Veronese at al., application. Biochem. Biotech. 11: 141-52 (1985)). All references and patents cited are incorporated by reference herein.

PKEMylation (i.e., addition of any water soluble polymer) of IFNL3 polypeptides containing a non-naturally encoded amino acid, such as p-azido-L-phenylalanine, is carried out by any convenient method. For example, IFNL3 polypeptide is PKEMylated with an alkyne-terminated PKEM derivative. Briefly, an excess of solid PKEM(5000)-O—CH2-C□CH is added, with stirring, to an aqueous solution of p-azido-L-Phe-containing IFNL3 polypeptide at room temperature. Typically, the aqueous solution is buffered with a buffer having a pKa near the pH at which the reaction is to be carried out (generally about pH 4-10). Examples of suitable buffers for PKEMylation at pH 7.5, for instance, include, but are not limited to, HEPES, phosphate, borate, TRIS-HCl, EPPS, and TES. The pH is continuously monitored and adjusted if necessary. The reaction is typically allowed to continue for between about 1-48 hours.

The reaction products are subsequently subjected to hydrophobic interaction chromatography to separate the PKEMylated IFNL3 polypeptide variants from free PKEM(5000)-O—CH2-C≡CH and any high-molecular weight complexes of the pegylated IFNL3 polypeptide which may form when unblocked PKEM is activated at both ends of the molecule, thereby crosslinking IFNL3 polypeptide variant molecules. The conditions during hydrophobic interaction chromatography are such that free PKEM(5000)-O—CH2-C≡CH flows through the column, while any crosslinked PKEMylated IFNL3 polypeptide variant complexes elute after the desired forms, which contain one IFNL3 polypeptide variant molecule conjugated to one or more PKEM groups. Suitable conditions vary depending on the relative sizes of the cross-linked complexes versus the desired conjugates and are readily determined by those of ordinary skill in the art. The eluent containing the desired conjugates is concentrated by ultrafiltration and desalted by diafiltration.

If necessary, the PKEMylated IFNL3 polypeptide obtained from the hydrophobic chromatography can be purified further by one or more procedures known to those of ordinary skill in the art including, but are not limited to, affinity chromatography; anion- or cation-exchange chromatography (using, including but not limited to, DEAE SEPHAROSE); chromatography on silica; reverse phase HPLC; gel filtration (using, including but not limited to, SEPHADEX G-75); hydrophobic interaction chromatography; size-exclusion chromatography, metal-chelate chromatography; ultrafiltration/diafiltration; ethanol precipitation; ammonium sulfate precipitation; chromatofocusing; displacement chromatography; electrophoretic procedures (including but not limited to preparative isoelectric focusing), differential solubility (including but not limited to ammonium sulfate precipitation), or extraction. Apparent molecular weight may be estimated by GPC by comparison to globular protein standards (Preneta, A Z in PROTEIN PURIFICATION METHODS, A PRACTICAL APPROACH (Harris & Angal, Eds.) IRL Press 1989, 293-306). The purity of the IFNL3 can be assessed by proteolytic degradation (including but not limited to, trypsin cleavage) followed by mass spectrometry analysis. Pepinsky R B., et al., J. Pharmcol. & Exp. Ther. 297(3):1059-66 (2001). A PKEM linked to an amino acid of an IFNL3 polypeptide of the invention can be further derivatized or substituted without limitation.

### Azide-Containing PKEM Derivatives

In another embodiment of the invention, an IFNL3 polypeptide is modified with a PKEM derivative that contains an azide moiety that will react with an alkyne moiety present on the side chain of the non-naturally encoded amino acid. In general, the PKEM derivatives will have an average molecular weight ranging from 1-100 kDa and, in some embodiments, from 10-40 kDa.

In some embodiments, the azide-terminal PKEM derivative will have the structure:

RO—(CH2CH2O)n-O—(CH2)m-N3

where R is a simple alkyl (methyl, ethyl, propyl, etc.), m is 2-10 and n is 100-1,000 (i.e., average molecular weight is between 5-40 kDa).

In another embodiment, the azide-terminal PKEM derivative will have the structure:

RO—(CH2CH2O)n-O—(CH2)m-NH—C(O)—(CH2)p-N3

where R is a simple alkyl (methyl, ethyl, propyl, etc.), m is 2-10, p is 2-10 and n is 100-1,000 (i.e., average molecular weight is between 5-40 kDa).

In another embodiment of the invention, an IFNL3 polypeptide comprising a alkyne-containing amino acid is modified with a branched PKEM derivative that contains a terminal azide moiety, with each chain of the branched PKEM having a MW ranging from 10-40 kDa and may be from 5-20 kDa. For instance, in some embodiments, the azide-terminal PKEM derivative will have the following structure:

[RO—(CH2CH2O)n-O—(CH2)₂—NH—C(O)]2CH(CH2)m-X—(CH2)pN3

where R is a simple alkyl (methyl, ethyl, propyl, etc.), m is 2-10, p is 2-10, and n is 100-1,000, and X is optionally an O, N, S or carbonyl group (C═O), in each case that can be present or absent.

### Alkyne-Containing PKEM Derivatives

In another embodiment of the invention, an IFNL3 polypeptide is modified with a PKEM derivative that contains an alkyne moiety that will react with an azide moiety present on the side chain of the non-naturally encoded amino acid.

In some embodiments, the alkyne-terminal PKEM derivative will have the following structure:

RO—(CH2CH2O)n-O—(CH2)m-C□CH

where R is a simple alkyl (methyl, ethyl, propyl, etc.), m is 2-10 and n is 100-1,000 (i.e., average molecular weight is between 5-40 kDa).

In another embodiment of the invention, an IFNL3 polypeptide comprising an alkyne-containing non-naturally encoded amino acid is modified with a PKEM derivative that contains a terminal azide or terminal alkyne moiety that is linked to the PKEM backbone by means of an amide linkage.

In some embodiments, the alkyne-terminal PKEM derivative will have the following structure:

RO—(CH2CH2O)n-O—(CH2)m-NH—C(O)—(CH2)p-C□CH

where R is a simple alkyl (methyl, ethyl, propyl, etc.), m is 2-10, p is 2-10 and n is 100-1,000.

In another embodiment of the invention, an IFNL3 polypeptide comprising an azide-containing amino acid is modified with a branched PKEM derivative that contains a terminal alkyne moiety, with each chain of the branched PKEM having a MW ranging from 10-40 kDa and may be from 5-20 kDa. For instance, in some embodiments, the alkyne-terminal PKEM derivative will have the following structure:

[RO—(CH2CH2O)n-O—(CH2)₂—NH—C(O)]2CH(CH2)m-X—(CH2)pC□CH

where R is a simple alkyl (methyl, ethyl, propyl, etc.), m is 2-10, p is 2-10, and n is 100-1,000, and X is optionally an O, N, S or carbonyl group (C═O), or not present.

### Phosphine-Containing PKEM Derivatives

In another embodiment of the invention, an IFNL3 polypeptide is modified with a PKEM derivative that contains an activated functional group (including but not limited to, ester, carbonate) further comprising an aryl phosphine group that will react with an azide moiety present on the side chain of the non-naturally encoded amino acid. In general, the PKEM derivatives will have an average molecular weight ranging from 1-100 kDa and, in some embodiments, from 10-40 kDa.

In some embodiments, the PKEM derivative will have the structure:

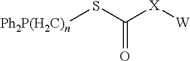

wherein n is 1-10; X can be O, N, S or not present, Ph is phenyl, and W is a PKEM.

In some embodiments, the PKEM derivative will have the structure:

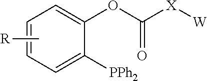

wherein X can be O, N, S or not present, Ph is phenyl, W is a PKEM and R can be H, alkyl, aryl, substituted alkyl and substituted aryl groups. Exemplary R groups include but are not limited to —CH₂, —C(CH₃)₃, —OR′, —NR′R″, —SR′, -halogen, —C(O)R′, —CONR′R″, —S(O)₂R′, —S(O)₂NR′R″, —CN and —NO₂. R′, R″, R′″ and R″″ each independently refer to hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (including but not limited to, —CF₃ and —CH₂CF₃) and acyl (including but not limited to, —C(O)CH3, —C(O)CF3, —C(O)CH2OCH3, and the like).

### Other PKEM Derivatives and General PKEMylation Techniques

Other exemplary PEG molecules that may be linked to IFNL3 polypeptides, as well as PEGylation methods include, but are not limited to, those described in, e.g., U.S. Patent Publication No. 2004/0001838; 2002/0052009; 2003/0162949; 2004/0013637; 2003/0228274; 2003/0220447; 2003/0158333; 2003/0143596; 2003/0114647; 2003/0105275; 2003/0105224; 2003/0023023; 2002/0156047; 2002/0099133; 2002/0086939; 2002/0082345; 2002/0072573; 2002/0052430; 2002/0040076; 2002/0037949; 2002/0002250; 2001/0056171; 2001/0044526; 2001/0021763; U.S. Pat. Nos. 6,646,110; 5,824,778; 5,476,653; 5,219,564; 5,629,384; 5,736,625; 4,902,502; 5,281,698; 5,122,614; 5,473,034; 5,516,673; 5,382,657; 6,552,167; 6,610,281; 6,515,100; 6,461,603; 6,436,386; 6,214,966; 5,990,237; 5,900,461; 5,739,208; 5,672,662; 5,446,090; 5,808,096; 5,612,460; 5,324,844; 5,252,714; 6,420,339; 6,201,072; 6,451,346; 6,306,821; 5,559,213; 5,747,646; 5,834,594; 5,849,860; 5,980,948; 6,004,573; 6,129,912; WO 97/32607, EP 229,108, EP 402,378, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, WO 98/05363, EP 809 996, WO 96/41813, WO 96/07670, EP 605 963, EP 510 356, EP 400 472, EP 183 503 and EP 154 316, which are incorporated by reference herein. Any of the PEG molecules described herein may be used in any form, including but not limited to, single chain, branched chain, multiarm chain, single functional, bi-functional, multi-functional, or any combination thereof.

Additional polymer and PEG derivatives including but not limited to, hydroxylamine (aminooxy) PEG derivatives, are described in the following patent applications which are all incorporated by reference in their entirety herein: U.S. Patent Publication No. 2006/0194256, U.S. Patent Publication No. 2006/0217532, U.S. Patent Publication No. 2006/0217289, U.S. Provisional Patent No. 60/755,338; U.S. Provisional Patent No. 60/755,711; U.S. Provisional Patent No. 60/755,018; International Patent Application No. PCT/US06/49397; WO 2006/069246; U.S. Provisional Patent No. 60/743,041; U.S. Provisional Patent No. 60/743,040; International Patent Application No. PCT/US06/47822; U.S. Provisional Patent No. 60/882,819; U.S. Provisional Patent No. 60/882,500; and U.S. Provisional Patent No. 60/870,594.

A naturally encoded or non-naturally encoded amino acid that is incorporated into a modified IFNL3 polypeptide may comprise a first functional group and the PKEM may comprise a second functional group, wherein the first functional group and second functional group are not identical and each comprise a carbonyl group, an aminooxy group, a hydrazide group, a hydrazine group, a semicarbazide group, an azide group, or an alkyne group.

Said PKEM may comprise at least one acyl group, lipid, alkyl group, serum albumin, XTEN molecule, Fc molecule, adnectin, or a combination thereof. Said PKEM may comprise at least one acyl group. Said acyl group may comprise a branched or unbranched C8-C30 acyl. Said acyl group may comprise a branched or unbranched C14 acyl, C16 acyl, C18 acyl, or C20 acyl. Said acyl group may be of the formula:

CH₃(CH₂)₁₂C(═O)— or CH₃(CH₂)₁₄C(═O)—.

Said acyl group may be of the formula: CH₃(CH₂)₁₆C(═O)— or CH₃(CH₂)₁₈C(═O)—.

Said PKEM may comprise at least one alkyl group. Said alkyl group may be branched or unbranched Said alkyl group may be a C8-C30 alkyl group. Said alkyl group may be a C14, C16, C18, or C20 alkyl group.

Said PKEM may comprise at least one serum albumin. Said serum albumin may comprise human serum albumin. For example, the IFNL3 or modified IFNL3 polypeptide may be linked to the Cys 34 residue of said human serum albumin.

Said PKEM may comprise at least one XTEN molecule. Said XTEN molecule may be linked to a single modified IFNL3 polypeptide molecule. The IFNL3 or modified IFNL3 polypeptide may be linked to a site at or near the N-terminus of said XTEN molecule. Said XTEN molecule may be linked to multiple modified IFNL3 polypeptide molecules. Each said XTEN molecule may be linked to one, two, three, four, or five modified IFNL3 polypeptide molecules. Each said XTEN molecule may be linked to three modified IFNL3 polypeptide molecules. Said three modified IFNL3 polypeptide molecules are linked to the XTEN molecule at or near the N-terminus, C-terminus, and middle of the XTEN molecule, respectively. Said XTEN molecule may comprise an unstructured recombinant polymer (URP) comprising at least 40 contiguous amino acids, wherein: (a) the URP comprises at least three different types of amino acids selected from the group consisting of glycine (G), aspartate (D), alanine (A), serine (S), threonine (T), glutamate (E) and proline (P) residues, wherein the sum of said group of amino acids contained in the URP constitutes more than about 80% of the total amino acids of the URP, and wherein said URP comprises more than one proline residue, and wherein said URP possesses reduced sensitivity to proteolytic degradation relative to a corresponding URP lacking said more than one proline residue; (b) at least 50% of the amino acids of said URP are devoid of secondary structure as determined by Chou-Fasman algorithm; and (c) the Tepitope score of said URP is less than −5. Said XTEN molecule may comprise an unstructured recombinant polymer (URP) comprising at least about 40 contiguous amino acids, and wherein (a) the sum of glycine (G), aspartate (D), alanine (A), serine (S), threonine (T), glutamate (E) and proline (P) residues contained in the URP, constitutes at least 80% of the total amino acids of the URP, and the remainder, when present, consists of arginine or lysine, and the remainder does not contain methionine, cysteine, asparagine, and glutamine, wherein said URP comprises at least three different types of amino acids selected from glycine (G), aspartate (D), alanine (A), serine (S), threonine (T), glutamate (E) and proline (P); (b) at least 50% of the at least 40 contiguous amino acids in said URP are devoid of secondary structure as determined by Chou-Fasman algorithm; and (c) wherein the URP has a Tepitope score less than −4. Each modified IFNL3 polypeptide may be linked to said XTEN molecule through a dibenzylcyclooctyne (DBCO). Said XTEN molecule may be further linked to a polyethylene glycol molecule.

Said PKEM may comprise at least one adnectin. Said adnectin may comprise one or more of a BC loop, a DE loop, and an FG loop.

Said PKEM may comprise one or more additional IFNL3 polypeptides or modified IFNL3 polypeptides in combination, linked to form a dimer, homodimer, heterodimer, multimer, homomultimer, heteromultimer, or other combinations of IFNL3 polypeptides and/or modified IFNL3 polypeptides. Each of these may also be linked to a PKEM, such as those disclosed herein, other than an IFNL3 polypeptide or modified IFNL3 Polypeptide. Said PKEM may comprise one or more additional interferon polypeptides, such as interferon alpha, interferon beta, interferon gamma, interferon lambda 1, 2, or 4 polypeptides or modified polypeptides in combination, linked to form a dimer, homodimer, heterodimer, multimer, homomultimer, heteromultimer, or other combinations of IFNL3 polypeptides and/or modified IFNL3 polypeptides.

Said PKEM may comprise at least one lipid. Said lipid may comprise a fat-soluble vitamin, fat, wax, sterol, monoglyceride, diglyceride, triglyceride, or phospholipid.

The IFNL3 or modified IFNL3 polypeptide may exhibit an in vivo half-life of at least 1, 2, 5, 10, 12, 15, 20, 25 hours, or multiple days or a week or more. Said in vivo half-life may be determined in human, mouse, rat, dog, cynomolgus monkey, rabbit, horse, cattle, cat, pig, sheep, chicken, hamster, or rhesus macaque. Said in vivo half-life may be determined following subcutaneous or intravenous administration of said IFNL3 or modified IFNL3 polypeptide.

The IFNL3 or modified IFNL3 polypeptide may be attached to another biologically active moiety, including but not limited to one or more IFNL3 or modified IFNL3 polypeptide, or another interferon or cytokine.

The IFNL3 or modified IFNL3 polypeptide may be further modified from its naturally occurring form to include at least one, at least two, or three disulfide bonds introduced into a modified IFNL3 by amino acid substitution.

Multiple IFNL3 polypeptides may be joined by a linker polypeptide, wherein said linker polypeptide optionally is 6-14, 7-13, 8-12, 7-11, 9-11, or 9 amino acids in length. Other linkers include but are not limited to small polymers such as PEG, which may be multi-armed allowing for multiple IFNL3 molecules to be linked together. Multiple IFNL3 polypeptides and modified IFNL3 polypeptides may be linked to each other via their N-termini in a head-to-head configuration through the use of such a linker or by direct chemical bonding between the respective N-terminus of each polypeptide. For example, two IFNL3 polypeptides may be linked to form a dimer by chemical bonding between their N-terminal amino groups or modified N-terminal amino groups, Also, a linking molecule that is designed to comprise multiple chemical functional groups for bonding with the N-terminus of each IFNL3 polypeptide may be used to join multiple IFNL3 polypeptides each at their respective N-terminus. In addition, multiple IFNL3 polypeptides may be linked through bonding between amino acids other than the N-terminal amino acid or C-terminal amino acid. An example of covalent bonds that may be utilized to form the dimmers and multimers of IFNL3 that are described herein include, but are not limited to disulphide or sulfhydryl or thiol bonds. In addition, certain enzymes, such as sortase, may be used to form covalent bonds between the IFNL3 polypeptides and the linker, including at the N-termini of the IFNL3 polypeptides.

In another aspect, the disclosure provides an IFNL3 or modified IFNL3 polypeptide or composition containing an IFNL3 or modified IFNL3 polypeptide as herein described, wherein said IFNL3 polypeptide may be conjugated to at least one substance including but not limited to a label, a dye, a polymer, a water-soluble polymer, a derivative of polyethylene glycol, a photocrosslinker, a radionuclide, a cytotoxic compound, a drug, an affinity label, a photoaffinity label, a reactive compound, a resin, another polypeptide or protein, a polypeptide analog, an antibody, an antibody fragment, a metal chelator, a cofactor, a fatty acid, a carbohydrate, a polynucleotide, a DNA, a RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, a neutron-capture agent, or any combination of the above.

### Heterologous Fc Fusion Proteins

The IFNL3 compounds described above may be fused directly or via a peptide linker to the Fc portion of an immunoglobulin. Immunoglobulins are molecules containing polypeptide chains held together by disulfide bonds, typically having two light chains and two heavy chains. In each chain, one domain (V) has a variable amino acid sequence depending on the antibody specificity of the molecule. The other domains (C) have a rather constant sequence common to molecules of the same class.

As used herein, the Fc portion of an immunoglobulin has the meaning commonly given to the term in the field of immunology. Specifically, this term refers to an antibody fragment which is obtained by removing the two antigen binding regions (the Fab fragments) from the antibody. One way to remove the Fab fragments is to digest the immunoglobulin with papain protease. Thus, the Fc portion is formed from approximately equal sized fragments of the constant region from both heavy chains, which associate through non-covalent interactions and disulfide bonds. The Fc portion can include the hinge regions and extend through the CH2 and CH3 domains to the C-terminus of the antibody. Representative hinge regions for human and mouse immunoglobulins can be found in Antibody Engineering, A Practical Guide, Borrebaeck, C. A. K., ed., W. H. Freeman and Co., 1992, the teachings of which are herein incorporated by reference. The Fc portion can further include one or more glycosylation sites. The amino acid sequences of numerous representative Fc proteins containing a hinge region, CH2 and CH3 domains, and one N-glycosylation site are well known in the art.

There are five types of human immunoglobulin Fc regions with different effector functions and pharmacokinetic properties: IgG, IgA, IgM, IgD, and IgE. IgG is the most abundant immunoglobulin in serum. IgG also has the longest half-life in serum of any immunoglobulin (23 days). Unlike other immunoglobulins, IgG is efficiently recirculated following binding to an Fc receptor. There are four IgG subclasses G1, G2, G3, and G4, each of which has different effector functions. G1, G2, and G3 can bind C1q and fix complement while G4 cannot. Even though G3 is able to bind C1q more efficiently than G1, G1 is more effective at mediating complement-directed cell lysis. G2 fixes complement very inefficiently. The C1q binding site in IgG is located at the carboxy terminal region of the CH2 domain.

All IgG subclasses are capable of binding to Fc receptors (CD16, CD32, CD64) with G1 and G3 being more effective than G2 and G4. The Fc receptor binding region of IgG is formed by residues located in both the hinge and the carboxy terminal regions of the CH2 domain.

IgA can exist both in a monomeric and dimeric form held together by a J-chain. IgA is the second most abundant Ig in serum, but it has a half-life of only 6 days. IgA has three effector functions. It binds to an IgA specific receptor on macrophages and eosinophils, which drives phagocytosis and degranulation, respectively. It can also fix complement via an unknown alternative pathway.

IgM is expressed as either a pentamer or a hexamer, both of which are held together by a J-chain. IgM has a serum half-life of 5 days. It binds weakly to C1q via a binding site located in its CH3 domain. IgD has a half-life of 3 days in serum. It is unclear what effector functions are attributable to this Ig. IgE is a monomeric Ig and has a serum half-life of 2.5 days. IgE binds to two Fc receptors which drives degranulation and results in the release of proinflammatory agents.

Depending on the desired in vivo effect, the heterologous fusion proteins of the present invention may contain any of the isotypes described above or may contain mutated Fc regions wherein the complement and/or Fc receptor binding functions have been altered. Thus, the heterologous fusion proteins of the present invention may contain the entire Fc portion of an immunoglobulin, fragments of the Fc portion of an immunoglobulin, or analogs thereof fused to an IFNL3 polypeptide. The equivalent forms of Fc regions from any species of non-human animal is suitable for use herein. In particular, the species of IFNL3 can be matched to the same species of origin for the Fc to produce a fusion protein that is species-specific for both the IFNL3 and Fc portions.

The fusion proteins of the present invention can consist of single chain proteins or as multi-chain polypeptides. Two or more Fc fusion proteins can be produced such that they interact through disulfide bonds that naturally form between Fc regions. These multimers can be homogeneous with respect to the IFNL3 polypeptide or they may contain different IFNL3 polypeptide fused at the N-terminus of the Fc portion of the fusion protein.

Regardless of the final structure of the fusion protein, the Fc or Fc-like region may serve to prolong the in vivo plasma half-life of the IFNL3 polypeptide fused at the N-terminus. Also, the interferon beta component of a fusion protein compound should retain at least one biological activity of interferon beta. An increase in therapeutic or circulating half-life can be demonstrated using the method described herein or known in the art, wherein the half-life of the fusion protein is compared to the half-life of the IFNL3 polypeptide alone. Biological activity can be determined by in vitro and in vivo methods known in the art.

Since the Fc region of IgG produced by proteolysis has the same in vivo half-life as the intact IgG molecule and Fab fragments are rapidly degraded, it is believed that the relevant sequence for prolonging half-life reside in the CH2 and/or CH3 domains. Further, it has been shown in the literature that the catabolic rates of IgG variants that do not bind the high-affinity Fc receptor or C1q are indistinguishable from the rate of clearance of the parent wild-type antibody, indicating that the catabolic site is distinct from the sites involved in Fc receptor or C1q binding. [Wawrzynczak et al., (1992) Molecular Immunology 29:221]. Site-directed mutagenesis studies using a murine IgG1 Fc region suggested that the site of the IgG1 Fc region that controls the catabolic rate is located at the CH2-CH3 domain interface. Fc regions can be modified at the catabolic site to optimize the half-life of the fusion proteins. The Fc region used for the fusion proteins of the present invention may be derived from an IgG1 or an IgG4 Fc region, and may contain both the CH2 and CH3 regions including the hinge region.

### Heterologous Albumin Fusion Proteins

IFNL3 described herein may be fused to albumin, such as directly or via a peptide linker, water soluble polymer, or prodrug linker to albumin or an analog, fragment, or derivative thereof. Generally, the albumin proteins that are part of the fusion proteins of the present invention may be derived from albumin cloned from any species, including human. Human serum albumin (HSA) consists of a single non-glycosylated polypeptide chain of 585 amino acids with a formula molecular weight of 66,500. The amino acid sequence of human HSA is known [See Meloun, et al. (1975) FEBS Letters 58:136; Behrens, et al. (1975) Fed. Proc. 34:591; Lawn, et al. (1981) Nucleic Acids Research 9:6102-6114; Minghetti, et al. (1986) J. Biol. Chem. 261:6747, each of which are incorporated by reference herein]. A variety of polymorphic variants as well as analogs and fragments of albumin have been described. [See Weitkamp, et al., (1973) Ann. Hum. Genet. 37:219]. For example, in EP 322,094, various shorter forms of HSA. Some of these fragments of HSA are disclosed, including HSA (1-373), HSA (1-388), HSA (1-389), HSA (1-369), and HSA (1-419) and fragments between 1-369 and 1-419. EP 399,666 discloses albumin fragments that include HSA (1-177) and HSA (1-200) and fragments between HSA (1-177) and HSA (1-200).

It is understood that the heterologous fusion proteins of the present invention include IFNL3 compounds that are coupled to any albumin protein including fragments, analogs, and derivatives wherein such fusion protein is biologically active and has a longer plasma half-life than the IFNL3 compound alone. Thus, the albumin portion of the fusion protein need not necessarily have a plasma half-life equal to that of native human albumin. Fragments, analogs, and derivatives are known or can be generated that have longer half-lives or have half-lives intermediate to that of native human albumin and the IFNL3 compound of interest.

The heterologous fusion proteins of the present invention encompass proteins having conservative amino acid substitutions in the IFNL3 compound and/or the Fc or albumin portion of the fusion protein. A “conservative substitution” is the replacement of an amino acid with another amino acid that has the same net electronic charge and approximately the same size and shape. Amino acids with aliphatic or substituted aliphatic amino acid side chains have approximately the same size when the total number carbon and heteroatoms in their side chains differs by no more than about four. They have approximately the same shape when the number of branches in their side chains differs by no more than one. Amino acids with phenyl or substituted phenyl groups in their side chains are considered to have about the same size and shape. Except as otherwise specifically provided herein, conservative substitutions are preferably made with naturally occurring amino acids.

Wild-type albumin and immunoglobulin proteins can be obtained from a variety of sources. For example, these proteins can be obtained from a cDNA library prepared from tissue or cells which express the mRNA of interest at a detectable level. Libraries can be screened with probes designed using the published DNA or protein sequence for the particular protein of interest. For example, immunoglobulin light or heavy chain constant regions are described in Adams, et al. (1980) Biochemistry 19:2711-2719; Goughet, et al. (1980) Biochemistry 19:2702-2710; Dolby, et al. (1980) Proc. Natl. Acad. Sci. USA 77:6027-6031; Rice et al. (1982) Proc. Natl. Acad. Sci. USA 79:7862-7862; Falkner, et al. (1982) Nature 298:286-288; and Morrison, et al. (1984) Ann. Rev. Immunol. 2:239-256. Some references disclosing albumin protein and DNA sequences include Meloun, et al. (1975) FEBS Letters 58:136; Behrens, et al. (1975) Fed. Proc. 34:591; Lawn, et al. (1981) Nucleic Acids Research 9:6102-6114; and Minghetti, et al. (1986) J. Biol. Chem. 261:6747.

### Characterization of the Heterologous Fusion Proteins

Numerous methods exist to characterize the fusion proteins of the present invention. Some of these methods include, but are not limited to: SDS-PAGE coupled with protein staining methods or immunoblotting using anti-IgG or anti-HSA antibodies. Other methods include matrix assisted laser desorption/ionization-mass spectrometry (MALDI-MS), liquid chromatography/mass spectrometry, isoelectric focusing, analytical anion exchange, chromatofocusing, and circular dichroism, for example.

### Enhancing Affinity for Serum Albumin

Various molecules can also be fused to the IFNL3 polypeptides of the invention to modulate the half-life of IFNL3 polypeptides in serum. In some embodiments, molecules are linked or fused to IFNL3 polypeptides of the invention to enhance affinity for endogenous serum albumin in an animal.

For example, in some cases, a recombinant fusion of an IFNL3 polypeptide and an albumin binding sequence is made. Exemplary albumin binding sequences include, but are not limited to, the albumin binding domain from streptococcal protein G (see. e.g., Makrides et al., J. Pharmacol. Exp. Ther. 277:534-542 (1996) and Sjolander et al., J, Immunol. Methods 201:115-123 (1997)), or albumin-binding peptides such as those described in, e.g., Dennis, et al., J. Biol. Chem. 277:35035-35043 (2002).

In other embodiments, the IFNL3 polypeptides of the present invention are acylated with fatty acids. In some cases, the fatty acids promote binding to serum albumin. See, e.g., Kurtzhals, et al., Biochem. J. 312:725-731 (1995).

In other embodiments, the IFNL3 polypeptides of the invention are fused directly with serum albumin (including but not limited to, human serum albumin). Those of skill in the art will recognize that a wide variety of other molecules can also be linked to IFNL3 in the present invention to modulate binding to serum albumin or other serum components.

### IFNL3 Dimers and Multimers

The present invention also provides for IFNL3 and IFNL3 analog combinations such as dimmers, homodimers, heterodimers, multimers, homomultimers, or heteromultimers (i.e., trimers, tetramers, etc.) where an IFNL3 or IFNL3 variant polypeptide is bound to another IFNL3 or IFNL3 variant thereof or any other polypeptide that is not IFNL3 or IFNL3 variant thereof, either directly to the polypeptide N-terminus, C-terminus, or peptide backbone or via a linker or directly through the functional groups or modified functional groups of an amino acid in the IFNL3 polypeptide. Due to its increased molecular weight compared to monomers, the IFNL3 dimer or multimer conjugates may exhibit new or desirable properties, including but not limited to different pharmacological, pharmacokinetic, pharmacodynamic, modulated therapeutic half-life, or modulated plasma half-life relative to the monomeric IFNL3. In some embodiments, IFNL3 dimmers or multimers of the invention will modulate enzymatic activity of the IFNL3.

In some embodiments, one or more of the IFNL3 molecules present in an IFNL3 containing dimer or multimer is linked to a PKEM. In some embodiments, the IFNL3 polypeptides are linked directly, including but not limited to, at their N-termini, via a Gly residue at the N-terminus through the enzyme sortase, via an Asn-Lys amide linkage or Cys-Cys disulfide linkage. In some embodiments, the IFNL3 polypeptides, and/or the linked non-IFNL3 molecule, will comprise different amino acids to facilitate dimerization, including but not limited to, an alkyne in one non-naturally encoded amino acid of a first IFNL3 polypeptide and an azide in a second amino acid of a second molecule will be conjugated via a Huisgen [3+2] cycloaddition. Alternatively, IFNL3, and/or the linked non-IFNL3 molecule comprising a ketone-containing amino acid can be conjugated to a second polypeptide comprising a hydroxylamine-containing amino acid and the polypeptides are reacted via formation of the corresponding oxime.

Alternatively, the two IFNL3 polypeptides, and/or the linked non-IFNL3 molecule, are linked via a linker. Any hetero- or homo-bifunctional linker can be used to link the two molecules, and/or the linked non-IFNL3 molecules, which can have the same or different primary sequence. In some cases, the linker used to tether the IFNL3, and/or the linked non-IFNL3 molecules together can be a bifunctional PKEM. The linker may have a wide range of molecular weight or molecular length. Larger or smaller molecular weight linkers may be used to provide a desired spatial relationship or conformation between IFNL3 and the linked entity or between the linked entity and its binding partner, if any. Linkers having longer or shorter molecular length may also be used to provide a desired space or flexibility between IFNL3 and the linked entity, or between the linked entity and its binding partner

In some embodiments, the invention provides water-soluble bifunctional linkers that have a dumbbell structure that includes: a) an azide, an alkyne, a hydrazine, a hydrazide, a hydroxylamine, or a carbonyl-containing moiety on at least a first end of a polymer backbone; and b) at least a second functional group on a second end of the polymer backbone. The second functional group can be the same or different as the first functional group. The second functional group, in some embodiments, is not reactive with the first functional group. The invention provides, in some embodiments, water-soluble compounds that comprise at least one arm of a branched molecular structure. For example, the branched molecular structure can be dendritic.

In some embodiments, the invention provides multimers comprising one or more IFNL3 polypeptide, formed by reactions with water soluble activated polymers that have the structure:

R—(CH2CH2O)n-O—(CH2)m-X

wherein n is from about 5 to 3,000, m is 2-10, X can be an azide, an alkyne, a hydrazine, a hydrazide, an aminooxy group, a hydroxylamine, an acetyl, or carbonyl-containing moiety, and R is a capping group, a functional group, or a leaving group that can be the same or different as X. R can be, for example, a functional group selected from the group consisting of hydroxyl, protected hydroxyl, alkoxyl, N-hydroxysuccinimidyl ester, 1-benzotriazolyl ester, N-hydroxysuccinimidyl carbonate, 1-benzotriazolyl carbonate, acetal, aldehyde, aldehyde hydrates, alkenyl, acrylate, methacrylate, acrylamide, active sulfone, amine, aminooxy, protected amine, hydrazide, protected hydrazide, protected thiol, carboxylic acid, protected carboxylic acid, isocyanate, isothiocyanate, maleimide, vinylsulfone, dithiopyridine, vinylpyridine, iodoacetamide, epoxide, glyoxals, diones, mesylates, tosylates, and tresylate, alkene, and ketone.

Purification of recombinant His-IFNL3: Cell pellets are lysed in 1.5 ml of cold lysis buffer (50 mM Tris HCl, pH 8.5, 100 mM KCl, 5 mM 2-ME, 1% NP40, 1 mM PMSF, 10 mM benzamidine, 1 μg/ml pepstatin), centrifuged at 10000×g for 15 min and clarified supernatant will be used for binding with 50 μl Ni-NTA agarose for 1 h at 4° C. The beads will be washed with 10 ml of ice-cold buffer A (20 mM Tris HCl, pH 8.5, 500 mM KCl, 5 mM 2-ME, 10% glycerol, 10 mM imidazole), 2 ml buffer B (20 mM Tris HCl, pH 8.5, 1000 mM KCl, 5 mM 2-ME, 10% glycerol) and 2 ml buffer A again. The bound proteins will be eluted buffer C (20 mM Tris HCl, pH 8.5, 100 mM KCl, 5 mM 2-ME, 10% glycerol, 300 mM imidazole). The purity of IFNL3 will be determined by SDS-PAGE electrophoresis and Western blotting.

Western blotting: ECL nitrocellulose membranes (Amersham) with proteins transferred from SDS-PAGE will be incubated with IFNL3 monoclonal antibodies and will be probed with anti-mouse IgG horseradish peroxidase (HRP)-linked antibody (Amersham, U.S.A.) followed by detection by ECL Western blotting reagents (Amersham).

IFNL3 anti-virus assay: IFNL3 activity is determined by standard in vitro procedures known in the art and commercially available, including but not limited to the MTT cell viability assay for IC50 determination, and a virus plaque assay. The in vitro testing is typically done at multiple IFNL3 dilutions in triplicate, for activity in an IC50 inhibition assays against a livestock virus such as Bovine Diarrhea Virus (BVD) on susceptible cells such as Madin Darby Bovine Kidney (MDBK) cells.

### MTT Cell Viability Assay for IC50 Determination.

Sterile, solubilized or pre-weighed sample compound(s) “Sample” sufficient to meet highest desired final concentration to be tested are used. Samples are solubilized in in a suitable buffer such as phosphate buffered saline. Samples will be tested at multiple dilutions, in triplicate, in suitable plates such as 96-well plates of the appropriate cells. Samples are tested at the highest concentration as directed, and 6 additional half-log dilutions. Monolayers of cells in the wells of a tissue culture dish will be exposed to the compound for 60 minutes to 24 hours, and then approximately 50 to 100 Infectious Units (PFU) of virus will be added to each well. Back titration of virus will be performed on the same day. After the appropriate number of days for the virus system, monolayers will be observed microscopically for cytopathic effect, fixed, stained and photographed by plate. Infected wells will be scored and raw data will be provided in table format.

Cytotoxicity as measured by colorimetric MTT conversion is utilized to determine the extent of virus cytopathic effect on the cells. Companion plates identical to the plates described above will be set up with Compound dilutions only, no virus, and will be incubated along with the Assay plate. After at least 3 days of incubation, the plates will be incubated with a solution of MTT for several hours until color change is obvious, then the cells will be solubilized and the OD600 determined. Drugs which may inhibitory for BVD will be included in each assay at two concentrations as positive controls. A negative control is also included.

A wide variety of viruses may be utilized in this assay with the appropriate cell lines to test for anti-virus activity of the IFNL3 proteins of the present invention. A non-limiting selection of livestock viruses is shown in Table 3.

The accurate isolation and quantification of viable viral samples has consistently been an ongoing research goal in virology. It was not until the advent of the plaque assay that a means to quantitatively and qualitatively calculate animal viral titers was first developed. This technique was first adapted and modified from phage assays, which had previously been used to calculate titers of stock bacteriophages in plant biology. While alternative means for viral quantification have since been developed and adapted, such as immunoassays, fluorescence and transmission electron microscopy, tunable resistive pulse sensing (TRPS), flow cytometry, recombinant reporter systems, and quantitative reverse transcription polymerase chain reaction (qRT-PCR), these methods fail to identify and quantitate replication competent virons.

During a plaque assay, a confluent monolayer of host cells is infected with a lytic virus of an unknown or known concentration that has been serially diluted to a countable range, typically between 5-100 virions. Infected monolayers are then covered with an immobilizing overlay medium to prevent viral infection from indiscriminately spreading through either the mechanical or convectional flow of the liquid medium during viral propagation. While solid or semisolid overlays such as agarose, methyl cellulose or carboxymethyl cellulose (CMC) have traditionally been used, liquid overlays are also available. After the initial infection and application of the immobilizing overlay, individual plaques, or zones of cell death, will begin to develop as viral infection and replication are constrained to the surrounding monolayer. Infected cells will continue the replication-lysis-infection cycle, further propagating the infection, resulting in increasingly distinct and discrete plaques. Depending on the viral growth kinetics and host cell used, a visible plaque will normally form within 2-14 days. Cellular monolayers may then be counted with a standard bright field microscope, or more typically fixed and counterstained by neutral red or crystal violent in order to readily identify plaques with the naked eye. There are a wide variety of plaque counter stains available, each offering their specific advantages and disadvantages. Crystal violet is typically added at the point of collection and after the fixation/removal of the overlay, providing a rapid and distinct counter stain which allows for the identification of very small plaques when mixed morphology is present. Neutral red has the advantage of early application and constant contact with the overlay, allowing for the live monitoring of developing plaque formation, which is particularly useful when working with an unknown virus or replication kinetics.

The high contrast between live and dead cells afforded by MTT also permits the detection of small plaques at an earlier time point post infection, although storage would still require removal of the overlay. As crystal violet can be simply made in a solution of water and alcohol, and provides a high degree of sensitivity for mixed plaque morphology, it is a preferred and simplified counter stain for the protocol. After fixing and staining the infected cellular monolayer, plaques are counted in order to titer viral stock samples in terms of plaque forming units (pfu) per milliliter. A log drop should be noted between serial dilutions and, depending on plate size, between 5-100 plaques counted, with a negative control used as a reference. Statistically samples will vary by 10% for every 100 plaques counted when comparing sample replicates. The advantage of using plaque assays to determine viral titers lies in their ability to quantitate the actual number of infectious viral particles within the sample. As multiple virions could potentially infect a single cell, the terminology of units versus virons is used during plaque titrations. Plaque morphology can vary dramatically under differing growth conditions and between viral species. Plaque size, clarity, border definition, and distribution should all be noted as they can provide valuable information on the growth and virulence factors of the virus in question. Basic plaque assay principles can also be adapted and modified in a number of different ways, such as in the use of focus forming assays (FFAs). FFAs do not rely on cell lysis and counterstaining to detect plaque formation, but rather employ immunostaining techniques to directly detect intracellular viral proteins through tagged antibodies. Increased sensitivity, decreased incubation times after infection, and most importantly the ability to quantitate non-lytic viruses are all distinct advantages when employing FFAs. A listing of suitable livestock viruses for testing in a plaque assay are shown in Table 3.

TABLE 3
Prevalent Livestock Viruses

| | | Bovine | Porcine | Ovine | Avian |
|---|---|---|---|---|---|
| Post | Enteric | Bovine Viral | Coronavirus, | Coronavirus, | Avian |
| Partum | Virus | Diaahreea | Rotatvirus, | Rotavirus | Influenza |
| | | (BVD), | Porcine Epic | | (AI) |
| | | Coronavirus, | Diahrea | | |
| | | Rotavirus | Virus | | |
| | | | (PEDV) | | |
| | Res- | BHV-1/IBR, | Porcine | Nil | Bron- |
| | piratory | PI-3 | Reproductive | | chitis |
| | Virus | | and | | |
| | | | Respiratory | | |
| | | | Syndrom | | |
| | | | (PRRS) | | |
| Acute | | Foot and | Food and | Blue | Nil |
| Outbreak | | Mouth | Mouth | Tongue | |
| | | Disease, | Disease, | Virus | |
| | | Bovine | Porcine | | |
| | | Viral | Reproductive | | |
| | | Diaherea | and | | |
| | | | Respiratory | | |
| | | | Syndrom | | |
| Prod- | Trans- | Multiple | Multiple | Nil | Nil |
| uction | port | | | | |
| Specific | Farm to | | | | |
| | Feed lot | | | | |

Anti-virus activity characterization: Using the above assays, the IFNL3 and modified IFNL3 antivirus replication activity are characterized. Protein stability is determined under different temperature and storage conditions.

First, PKEMylation of IFNL3 is performed to generate a longer half life IFNL3 adduct. PEGylation of proteins is widely used in order to prolong their in vivo half life. Several PEGylation sites are identified to be present in IFNL3. PEGylation conditions will be determined to generate an active protein with extended half-life, such as a circulating half life of 6, 12, 24, 36, 48 hours or longer. This will enable 1-2 injections for a desired treatment duration depending upon the condition of the subject.

Pharmacokinetic studies with IFNL3 and sustained delivery formulation: The clearance of IFNL3, modified IFNL3 or other form of IFNL3 will be determined by intravenous (i.v.) and intraperitoneal (i.p.) administration of different doses of the preparations to rats or to the corresponding species of the protein. Blood and tissue samples will be collected at different intervals, and plasma IFNL3 protein concentration and IFNL3 activity will be determined. These studies will allow us to quantitatively determine the dose of IFNL3 and route of administration required to achieve different magnitude of activity.

In vivo studies measuring anti-virus activity following systemic delivery of IFNL3: Different concentrations of IFNL3 formulation or the vehicle control (formulation without IFNL3) will be administered to subjects by intravenous or intraperitoneal routes. Plasma and tissue samples will be collected at different intervals. Plasma and tissue extracts will be assayed for virus levels. Based on experience, statistical differentiation between control and treated animals will be achieved by using 6 animals in each group, but can be adjusted up or down as the data shows.

Administered quantities of IFNL3, IFNL3 polypeptides, and/or IFNL3 analogues of the present invention may vary and in particular should be based upon the recommendations and prescription of a qualified veterinarian. The exact amount of IFNL3, IFNL3 polypeptides, and/or IFNL3 analogues of the present invention is a matter of preference subject to such factors as the exact type and/or severity of the condition being treated, the condition of the subject being treated, as well as the other ingredients in the composition. The invention also provides for administration of a therapeutically effective amount of another active agent. The amount to be given may be readily determined by one of ordinary skill in the art based upon therapy with IFNL3, available IFNL3 therapies, and/or other IFNL3 analogues.

Formulations

In the broad practice of the present invention, it also is contemplated that a formulation may contain a mixture of two or more of an IFNL3, an IFNL3 dimer, an IFNL3 multimer, an IFNL3 variant, an IFNL3 analog, an acylated IFNL3, a PKEMylated or acylated or PEGylated IFNL3 analog. In another embodiment of the present invention, the formulations containing a mixture of two or more of IFNL3, an IFNL3 analog, an acylated IFNL3, or acylated IFNL3 analog also includes at least one PKEM attached to at least one of the IFNL3 polypeptides.

The present invention also includes heterogeneous mixtures wherein IFNL3 polypeptides and IFNL3 analogs are prepared by the methods disclosed in this invention and are then mixed so that a formulation may be administered to a subject in need thereof which contains, for example, various percentages of different forms of IFNL3 polypeptides which have been coupled to a particular PKEM, and the remainder consisting of IFNL3 polypeptide having a different or no PKEM. All different mixtures of different percentage amounts of IFNL3 polypeptide variants wherein the IFNL3 polypeptides include a variety (1) with differently sized PKEM, or (2) PKEM are included at different positions in the sequence. This is intended as an example and should in no way be construed as limiting to the formulations made possible by the present invention and will be apparent to those of skill in the art. In an additional embodiment, the IFNL3 polypeptide variants to include in the formulation mixture will be chosen by their varying dissociation times so that the formulation may provide a sustained release of IFNL3 for a subject in need thereof, or the formulation may provide immediate or fast acting IFNL3 as well as longer acting IFNL3 molecules including one or more PKEM's.

Other embodiments of the present invention including formulation for inhalation. In an additional embodiment of the present invention, it is possible to use the technology disclosed herein for the production of IFNL3 analogs with increased pharmacokinetic and pharmacodynamic properties for subject use via administration to the lung, resulting in elevated blood levels of IFNL3 that are sustained for at least 6 hours, and more typically for at least 8, 10, 12, 14, 18, 24 hours or greater post-administration. Another embodiment of the present invention allows for advantageous mixtures of IFNL3 analogs suitable for therapeutic formulations designed to be administered to subjects as an inhalant.

This embodiment of the invention is particularly useful for introducing additional, customized sites within the IFNL3 molecule, for example, for forming an IFNL3 or modified IFNL3 having improved resistance to enzymatic degradation. Such an approach provides greater flexibility in the design of an optimized IFNL3 conjugate having the desired balance of activity, stability, solubility, and pharmacological properties. Mutations can be carried out, i.e., by site specific mutagenesis, at any number of positions within the IFNL3 molecule. Typically, a PKEM is activated with a suitable activating group appropriate for coupling a desired site or sites on the IFNL3 molecule. An activated PKEM may possess a reactive group at a terminus for reaction with IFNL3.

Branched PKEMs such as PEGs for use in embodiments of the invention include those described in International Patent Publication WO 96/21469, the contents of which is expressly incorporated herein by reference in its entirety. Generally, branched PKEMs can be represented by the formula R(PKEM-OH)ₙ, where R represents the central “core” molecule and n represents the number of arms. Branched PKEMs have a central core from which extend 2 or more “PKEM” arms. In a branched configuration, the branched polymer core possesses a single reactive site for attachment to IFNL3. Branched PKEMs for use in the present invention will typically comprise fewer than 4 PKEM arms, and more preferably, will comprise fewer than 3 PKEM arms. Branched PKEMs offer the advantage of having a single reactive site, coupled with a larger, denser polymer cloud than their linear PKEM counterparts. One particular type of branched PKEM can be represented as (MeO-PKEM-)ₚ R—X, where p equals 2 or 3, R is a central core structure such as lysine or glycerol having 2 or 3 PKEM arms attached thereto, and X represents any suitable functional group that is or that can be activated for coupling to IFNL3. One particularly preferred branched PEG is mPEG2-NHS (Shearwater Corporation, Ala.) having the structure mPEG2-lysine-succinimide.

In yet another branched architecture, “pendant PKEM” has reactive groups for protein coupling positioned along the PKEM backbone rather than at the end of PKEM chains. The reactive groups extending from the PKEM backbone for coupling to IFNL3 may be the same or different. Pendant PKEM structures may be useful but are generally less preferred, particularly for compositions for inhalation.

Alternatively, the PKEM may possess a forked structure having a branched moiety at one end of the polymer chain and two free reactive groups (or any multiple of 2) linked to the branched moiety for attachment to IFNL3. Exemplary forked PKEMs are described in International Patent Publication No. WO 99/45964, the content of which is expressly incorporated herein by reference. The forked polyethylene glycol may optionally include an alkyl or “R” group at the opposing end of the polymer chain. More specifically, a forked PKEM-IFNL3 conjugate in accordance with embodiments of the invention has the formula: R-PKEM-L(Y-IFNL3)n where R is alkyl, L is a hydrolytically stable branch point and Y is a linking group that provides chemical linkage of the forked polymer to IFNL3, and n is a multiple of 2. L may represent a single “core” group, such as “—CH—”, or may comprise a longer chain of atoms. Exemplary L groups include lysine, glycerol, pentaerythritol, or sorbitol. Typically, the particular branch atom within the branching moiety is carbon.

In one particular embodiment of the invention, the linkage of the forked PKEM to the IFNL3 molecule, (Y), is hydrolytically stable. In a preferred embodiment, n is 2. Suitable Y moieties, prior to conjugation with a reactive site on IFNL3, include but are not limited to active esters, active carbonates, aldehydes, isocyanates, isothiocyanates, epoxides, alcohols, maleimides, vinylsulfones, hydrazides, dithiopyridines, and iodacetamides. Selection of a suitable activating group will depend upon the intended site of attachment on the IFNL3 molecule and can be readily determined by one of skill in the art. The corresponding Y group in the resulting PKEM-IFNL3 conjugate is that which results from reaction of the activated forked polymer with a suitable reactive site on IFNL3. The specific identity of such the final linkage will be apparent to one skilled in the art. For example, if the reactive forked PKEM contains an activated ester, such as a succinimide or maleimide ester, conjugation via an amine site on IFNL3 will result in formation of the corresponding amide linkage. These particular forked polymers are particularly attractive since they provide conjugates having a molar ratio of IFNL3 to of 2:1 or greater. Such conjugates may be less likely to block the IFNL3 receptor binding or other binding site, while still providing the flexibility in design to protect the IFNL3 against enzymatic degradation, e.g., by IFNL3 degrading enzymes.

In a related embodiment, a forked PKEM-IFNL3 conjugate may be used in the present invention, represented by the formula: R-[PKEM-L(Y-IFNL3)2]n. In this instance R represents a natural or non-naturally encoded amino acid having attached thereto at least one PKEM-di-IFNL3 conjugate. Specifically, preferred forked polymers in accordance with this aspect of the invention are those were n is selected from the group consisting of 1, 2, 3, 4, 5, and 6. In an alternative embodiment, the chemical linkage between the non-natural amino acid within IFNL3, IFNL3 polypeptide, or IFNL3 analog and the polymer branch point may be degradable (i.e., hydrolytically unstable). Alternatively, one or more degradable linkages may be contained in the polymer backbone to allow generation in vivo of a PKEM-IFNL3 conjugate having a smaller PKEM chain than in the initially administered conjugate. For example, a large and relatively inert conjugate (i.e., having one or more high molecular weight PKEM chains attached thereto, e.g., one or more PKEM chains having a molecular weight greater than about 10,000, wherein the conjugate possesses essentially no bioactivity) may be administered, which then either in the lung or in the bloodstream, is hydrolyzed to generate a bioactive conjugate possessing a portion of the originally present PKEM chain. Upon in-vivo cleavage of the hydrolytically degradable linkage, either free IFNL3 (depending upon the position of the degradable linkage) or IFNL3 having a small polyethylene tag attached thereto, is then released and more readily absorbed through the lung and/or circulated in the blood.

In some embodiments, the poly(ethylene glycol) molecule has a molecular weight of between about 0.1 kDa and about 100 kDa. In some embodiments, the poly(ethylene glycol) molecule has a molecular weight of between 0.1 kDa and 30, 40, or 50 kDa. In some embodiments, the poly(ethylene glycol) molecule is a branched polymer. In some embodiments, each branch of the poly(ethylene glycol) branched polymer has a molecular weight of between 1 kDa and 100 kDa, or between 1 kDa and 10, 20, 30, 40, or 50 kDa.

In some cases, a PKEM used in the invention terminates on one end with hydroxy or methoxy, i.e., X is H or CH₃ (“methoxy PEG”). Alternatively, the PKEM can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester) and aldehydes) as well as functional groups that are inert to the 20 common amino acids but that react specifically with complementary functional groups (including but not limited to, azide groups, alkyne groups). It is noted that the other end of the PKEM, which is shown in the above formula by Y, will attach either directly or indirectly to an IFNL3 polypeptide via a naturally-occurring or non-naturally encoded amino acid. For instance, Y may be an amide, carbamate or urea linkage to an amine group (including but not limited to, the epsilon amine of lysine or the N-terminus) of the polypeptide. Alternatively, Y may be a maleimide linkage to a thiol group (including but not limited to, the thiol group of cysteine). Alternatively, Y may be a linkage to a residue not commonly accessible via the 20 common amino acids. For example, an azide group on the PKEM can be reacted with an alkyne group on the IFNL3 polypeptide to form a Huisgen [3+2] cycloaddition product. Alternatively, an alkyne group on the PKEM can be reacted with an azide group present in an IFNL3 polypeptide to form a similar product. In some embodiments, a strong nucleophile (including but not limited to, hydrazine, hydrazide, hydroxylamine, semicarbazide) can be reacted with an aldehyde or ketone group present in an IFNL3 polypeptide to form a hydrazone, oxime or semicarbazone, as applicable, which in some cases can be further reduced by treatment with an appropriate reducing agent. Alternatively, the strong nucleophile can be incorporated into the IFNL3 polypeptide via a non-naturally encoded amino acid and used to react preferentially with a ketone or aldehyde group present in the water soluble polymer.

Any molecular mass for a PKEM can be used as practically desired, including but not limited to, from about 100 Daltons (Da) to 100,000 Da or more as desired (including but not limited to, sometimes 0.1-50 kDa or 10-40 kDa). The molecular weight of PKEM may be of a wide range, including but not limited to, between about 100 Da and about 100,000 Da or more. PKEM may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, PKEM is between about 100 Da and about 50,000 Da. Branched chain PKEMs, including but not limited to, PKEM molecules with each chain having a MW ranging from 1-100 kDa (including but not limited to, 1-50 kDa or 5-20 kDa) can also be used. The molecular weight of each chain of the branched chain PKEM may be, including but not limited to, between about 1,000 Da and about 100,000 Da or more. The molecular weight of each chain of the branched chain PKEM may be between about 1,000 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, and 1,000 Da. In some embodiments, the molecular weight of each chain of the branched chain PKEM is between about 1,000 Da and about 50,000 Da. In some embodiments, the molecular weight of each chain of the branched chain PKEM is between about 1,000 Da and about 40,000 Da. In some embodiments, the molecular weight of each chain of the branched chain PKEM is between about 5,000 Da and about 40,000 Da. In some embodiments, the molecular weight of each chain of the branched chain PKEM is between about 5,000 Da and about 20,000 Da. A wide range of PKEM molecules are described in, including but not limited to, the Shearwater Polymers, Inc. catalog, Nektar Therapeutics catalog, incorporated herein by reference.

The invention provides in some embodiments azide- and acetylene-containing polymer derivatives comprising a water soluble polymer backbone having an average molecular weight from about 800 Da to about 100,000 Da. The polymer backbone of the water-soluble polymer can be poly(ethylene glycol). However, it should be understood that a wide variety of water soluble polymers including but not limited to poly(ethylene)glycol and other related polymers, including poly(dextran) and poly(propylene glycol), are also suitable for use in the practice of this invention and that the use of the term PEG or poly(ethylene glycol) is intended to encompass and include all such molecules. The term PEG includes, but is not limited to, poly(ethylene glycol) in any of its forms, including bifunctional PEG, multiarmed PEG, derivatized PEG, forked PEG, branched PEG, pendent PEG (i.e. PEG or related polymers having one or more functional groups pendent to the polymer backbone), or PEG with degradable linkages therein.

In addition to these forms of PKEM, the polymer can also be prepared with weak or degradable linkages in the backbone. For example, PKEM can be prepared with ester linkages in the polymer backbone that are subject to hydrolysis. As shown below, this hydrolysis results in cleavage of the polymer into fragments of lower molecular weight:

-PKEM-CO₂-PKEM-+H₂O-PKEM-CO₂H+HO-PKEM-

Many polymers are also suitable for use in the present invention. In some embodiments, polymer backbones that are water-soluble, with from 2 to about 300 termini, are particularly useful in the invention. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da. The molecular weight of each chain of the polymer backbone may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of each chain of the polymer backbone is between about 100 Da and about 50,000 Da. In some embodiments, the molecular weight of each chain of the polymer backbone is between about 100 Da and about 40,000 Da. In some embodiments, the molecular weight of each chain of the polymer backbone is between about 1,000 Da and about 40,000 Da. In some embodiments, the molecular weight of each chain of the polymer backbone is between about 5,000 Da and about 40,000 Da. In some embodiments, the molecular weight of each chain of the polymer backbone is between about 10,000 Da and about 40,000 Da.

In one feature of this embodiment of the invention, the intact polymer-conjugate, prior to hydrolysis, is minimally degraded upon administration, such that hydrolysis of the cleavable bond is effective to govern the slow rate of release of active IFNL3 into the bloodstream, as opposed to enzymatic degradation of IFNL3 prior to its release into the systemic circulation.

Appropriate physiologically cleavable linkages include but are not limited to ester, carbonate ester, carbamate, sulfate, phosphate, acyloxyalkyl ether, acetal, and ketal. Such conjugates should possess a physiologically cleavable bond that is stable upon storage and upon administration. For instance, an IFNL3 or modified IFNL3 linked to a PKEM should maintain its integrity upon manufacturing of the final pharmaceutical composition, upon dissolution in an appropriate delivery vehicle, if employed, and upon administration irrespective of route.

In some embodiments, the polypeptide of the invention comprises one or more naturally encoded or non-naturally encoded amino acid substitution, addition, or deletion in the signal sequence. In some embodiments, the polypeptide of the invention comprises one or more naturally encoded or non-naturally encoded amino acid substitution, addition, or deletion in the signal sequence for IFNL3 or any of the IFNL3 analogs or polypeptides disclosed within this specification. In some embodiments, the polypeptide of the invention comprises one or more naturally encoded amino acid substitution, addition, or deletion in the signal sequence as well as one or more non-naturally encoded amino acid substitutions, additions, or deletions in the signal sequence for IFNL3 or any of the IFNL3 analogs or polypeptides disclosed within this specification. In some embodiments, one or more non-natural amino acids are incorporated in the leader or signal sequence for IFNL3 or any of the IFNL3 analogs or polypeptides disclosed within this specification.

In some embodiments, the IFNL3 polypeptide comprises a substitution, addition or deletion that modulates affinity of the IFNL3 polypeptide for its receptor or other binding partner, including but not limited to, a protein, polypeptide, small molecule, or nucleic acid. In some embodiments, the IFNL3 polypeptide comprises a substitution, addition, or deletion that increases the stability of the IFNL3 polypeptide when compared with the stability of the corresponding IFNL3 without the substitution, addition, or deletion. Stability and/or solubility may be measured using a number of different assays known to those of ordinary skill in the art. In some embodiments, the IFNL3 polypeptide comprises a substitution, addition, or deletion that modulates the immunogenicity of the IFNL3 polypeptide when compared with the immunogenicity of the corresponding IFNL3 without the substitution, addition, or deletion. In some embodiments, the IFNL3 polypeptide comprises a substitution, addition, or deletion that modulates serum half-life or circulation time of the IFNL3 polypeptide when compared with the serum half-life or circulation time of the corresponding IFNL3 without the substitution, addition, or deletion. In some embodiments, the IFNL3 polypeptide comprises a substitution, addition, or deletion that modulates the enzymatic activity of the IFNL3 polypeptide when compared with the enzymatic activity of the corresponding IFNL3 without the substitution, addition, or deletion. In some embodiments, the IFNL3 polypeptide comprises a substitution, addition, or deletion that increases the biological activity of the IFNL3 polypeptide when compared with the biological activity of the corresponding IFNL3 without the substitution, addition, or deletion.

In some embodiments, the IFNL3 polypeptide comprises a substitution, addition, or deletion that increases the aqueous solubility of the IFNL3 polypeptide when compared to aqueous solubility of the corresponding IFNL3 without the substitution, addition, or deletion. In some embodiments, the IFNL3 polypeptide comprises a substitution, addition, or deletion that increases the solubility of the IFNL3 polypeptide produced in a host cell when compared to the solubility of the corresponding IFNL3 without the substitution, addition, or deletion. In some embodiments, the IFNL3 polypeptide comprises a substitution, addition, or deletion that increases the expression of the IFNL3 polypeptide in a host cell or increases synthesis in vitro when compared to the expression or synthesis of the corresponding IFNL3 without the substitution, addition, or deletion. The IFNL3 polypeptide comprising this substitution retains biologic activity and retains or improves expression levels in a host cell. In some embodiments, the IFNL3 polypeptide comprises a substitution, addition, or deletion that increases protease resistance of the IFNL3 polypeptide during manufacturing processes when compared to the protease resistance of the corresponding IFNL3 without the substitution, addition, or deletion. In some embodiments, the IFNL3 polypeptide comprises a substitution, addition, or deletion that modulates IFNL3 anti-virus replication activity when compared with the activity of the IFNL3 polypeptide without the substitution, addition, or deletion. In some embodiments, the IFNL3 polypeptide comprises a substitution, addition, or deletion that modulates its binding to another molecule such as a receptor or modulator or other IFNL3 polypeptide when compared to the binding of the corresponding IFNL3 polypeptide without the substitution, addition, or deletion. In some embodiments, the IFNL3 polypeptide comprises a substitution, addition, or deletion that modulates its enzymatic activity compared to the enzymatic activity of the corresponding IFNL3 polypeptide without the substitution, addition, or deletion.

In some embodiments, the IFNL3 polypeptide comprises a substitution, addition, or deletion that modulates the stability of the IFNL3 polypeptide when compared to stability of the corresponding IFNL3 without the substitution, addition, or deletion. In some embodiments, the IFNL3 polypeptide comprises a substitution, addition, or deletion that increases the stability of the IFNL3 polypeptide produced in a host cell when compared to the stability of the corresponding IFNL3 without the substitution, addition, or deletion. In some embodiments, the IFNL3 polypeptide comprises a substitution, addition, or deletion that increases the half-life of active circulating IFNL3 after administration to a subject when compared to the corresponding IFNL3 without the substitution, addition, or deletion. The IFNL3 polypeptide comprising this substitution retains receptor binding activity and yet is resistant to deactivation, destabilization, or destruction caused, for example, by proteases or other substances that affect the structural integrity or biologic activity of the IFNL3 polypeptides. In some embodiments, the IFNL3 polypeptide comprises a substitution, addition, or deletion that increases protease resistance of the IFNL3 polypeptide when compared to the protease resistance of the corresponding IFNL3 without the substitution, addition, or deletion. In some embodiments, the IFNL3 polypeptide comprises a substitution, addition, or deletion that decreases the half-life of enzymatically active circulating IFNL3 after administration to a subject when compared to the corresponding IFNL3 without the substitution, addition, or deletion. In some embodiments, the IFNL3 polypeptide comprises a substitution, addition, or deletion that decreases its binding to another molecule such as a receptor or modulator or other IFNL3 polypeptide when compared to the binding of the corresponding IFNL3 polypeptide without the substitution, addition, or deletion. In some embodiments, the IFNL3 polypeptide comprises a substitution, addition, or deletion that decreases its enzymatic activity compared to the enzymatic activity of the corresponding IFNL3 polypeptide without the substitution, addition, or deletion.

In some embodiments, the IFNL3 polypeptide comprises a substitution, addition, or deletion that increases compatibility of the IFNL3 polypeptide with pharmaceutical preservatives (e.g., m-cresol, phenol, benzyl alcohol) when compared to compatibility of the corresponding IFNL3 without the substitution, addition, or deletion. This increased compatibility would enable the preparation of a preserved pharmaceutical formulation that maintains the physiochemical properties and biological activity of the protein during storage.

In some embodiments, one or more engineered bonds are created with one or more non-natural amino acids. The intramolecular bond may be created in many ways, including but not limited to, a reaction between two amino acids in the protein under suitable conditions (one or both amino acids may be a non-natural amino acid); a reaction with two amino acids, each of which may be naturally encoded or non-naturally encoded, with a linker, polymer, or other molecule under suitable conditions; etc.

In some embodiments, one or more amino acid substitutions in the IFNL3 polypeptide may be with one or more naturally occurring or non-naturally encoded amino acids. In some embodiments the amino acid substitutions in the IFNL3 polypeptide may be with naturally occurring or non-naturally encoded amino acids, provided that at least one substitution is with a non-naturally encoded amino acid. In some embodiments, one or more amino acid substitutions in the IFNL3 polypeptide may be with one or more naturally occurring amino acids, and additionally at least one substitution is with a non-naturally encoded amino acid.

In some embodiments, the non-naturally encoded amino acid comprises a carbonyl group, an acetyl group, an aminooxy group, a hydrazine group, a hydrazide group, a semicarbazide group, an azide group, or an alkyne group.

In some embodiments, the non-naturally encoded amino acid comprises a carbonyl group. In some embodiments, the non-naturally encoded amino acid has the structure:

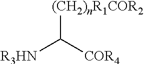

wherein n is 0-10; R1 is an alkyl, aryl, substituted alkyl, or substituted aryl; R2 is H, an alkyl, aryl, substituted alkyl, and substituted aryl; and R3 is H, an amino acid, a polypeptide, or an amino terminus modification group, and R4 is H, an amino acid, a polypeptide, or a carboxy terminus modification group.

The present invention also provides isolated nucleic acids comprising a polynucleotide that hybridizes under stringent conditions nucleic acids that encode IFNL3 polypeptides of SEQ ID NOs: 1, 2, 3, 4, 5, and 6. The present invention also provides isolated nucleic acids comprising a polynucleotide that hybridizes under stringent conditions to nucleic acids that encode IFNL3 polypeptides of SEQ ID NOs: 1, 2, 3, 4, 5, and 6. The present invention also provides isolated nucleic acids comprising a polynucleotide that encodes the polypeptides shown as SEQ ID NOs.: 1, 2, 3, 4, 5, and 6. It is readily apparent to those of ordinary skill in the art that a number of different polynucleotides can encode any polypeptide of the present invention.

Azide- and acetylene-containing amino acids may also be incorporated site-selectively into proteins such as IFNL3 using the methods described in L. Wang, et al., (2001), Science 292:498-500, J. W. Chin et al., Science 301:964-7 (2003)), J. W. Chin et al., (2002), Journal of the American Chemical Society 124:9026-9027; J. W. Chin, & P. G. Schultz, (2002), Chem Bio Chem 3(11):1135-1137; J. W. Chin, et al., (2002), PNAS United States of America 99:11020-11024: and, L. Wang, & P. G. Schultz, (2002), Chem. Comm., 1:1-11, each of which is hereby incorporated by reference in its entirety. Thereafter said azide- and acetylene-containing amino acids may be linked to a PKEM using methods known in the art.

In a further aspect, the invention provides recombinant nucleic acids encoding the IFNL3 proteins, expression vectors containing the variant nucleic acids, host cells comprising the variant nucleic acids and/or expression vectors, and methods for producing the variant proteins. In an additional aspect, the invention provides treating an IFNL3 responsive disorder by administering to a subject a variant protein, usually with a pharmaceutical carrier, in a therapeutically effective amount. In a further aspect, the invention provides methods for modulating immunogenicity (particularly reducing immunogenicity) of IFNL3 polypeptides by altering MHC Class II epitopes.

In therapeutic applications, compositions containing the modified non-natural amino acid polypeptide are administered to a subject already suffering from a disease, condition or disorder, in an amount sufficient to cure or at least partially arrest the symptoms of the disease, disorder or condition. Such an amount is defined to be a “therapeutically effective amount,” and will depend on the severity and course of the disease, disorder or condition, previous therapy, the subject's health status and response to the drugs, and the judgment of the treating physician. It is considered well within the skill of the art for one to determine such therapeutically effective amounts by routine experimentation (e.g., a dose escalation clinical trial).

In prophylactic applications, compositions containing the IFNL3 polypeptide are administered to a subject susceptible to or otherwise at risk of a particular disease, disorder or condition. Such an amount is defined to be a “prophylactically effective amount.” In this use, the precise amounts also depend on the subject's state of health, weight, and the like. It is considered well within the skill of the art for one to determine such prophylactically effective amounts by routine experimentation (e.g., a dose escalation clinical trial).

Pharmaceutical compositions of the invention may be manufactured in a conventional manner. The present disclosure also provides for pharmaceutical compositions comprising an IFNL3 or modified IFNL3 in a pharmacologically acceptable vehicle. The IFNL3 or modified IFNL3 may be administrated systemically or locally. Any appropriate mode of administration known in the art may be used including, but not limited to, intravenous, intraperitoneal, intraarterial, intranasal, by inhalation, oral, subcutaneous administration, transdermal, by local injection or in form of a surgical implant. For example, administration may be performed by any parenteral means such as subcutaneously, intramuscularly, intraperitoneally, and intravenously, or by enteral or enteric administration, such as orally, rectally, and by inhalation, or transdermally.

The present disclosure also provides pharmaceutical compositions which may comprise an IFNL3 or modified IFNL3, alone or in combination with at least one other agent, such as stabilizing compound, which may be administered in any sterile, biocompatible pharmaceutical carrier, including, but not limited to, saline, buffered saline, dextrose, and water. In one embodiment, the pharmaceutically acceptable carrier may be pharmaceutically inert. Any of these molecules can be administered to a subject alone, or in combination with other agents, drugs or hormones, in pharmaceutical compositions where it is mixed with excipient(s) or pharmaceutically acceptable carriers. In this context, the term combination encompasses any means of concurrent administration, whether or not the IFNL3 or modified IFNL3 and the other agent are contained in the same composition or administered separately, which administration may be through the same or different modes of administration. The present disclosure also provides methods comprising the administration of pharmaceutical compositions disclosed herein. Such administration is accomplished orally or parenterally. Methods of parenteral delivery include topical, transdermal, intra-arterial, intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical compositions suitable for use in the methods and compositions of the present disclosure include compositions wherein the active ingredients are contained in an effective amount to achieve the intended purpose, e.g. stimulation of the IIS. The determination of an effective dose is well within the capability of those skilled in the art in view of the present disclosure.

For any compound, the therapeutically effective dose can be estimated initially either in in vitro assays, e.g. those described in the Examples herein, or in animal models, such as mice, rabbits, dogs, horses, cows, chickens, or pigs. The animal model is also used to achieve a desirable concentration range and route of administration. Such information can then be used to determine useful doses and routes for administration to subjects.

A therapeutically effective dose refers to that amount of an IFNL3 or modified IFNL3 that stimulates the IIS in the subject. Therapeutic efficacy and toxicity of such compounds can be determined by standard pharmaceutical procedures in vitro or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Exemplary pharmaceutical compositions exhibit large therapeutic indices. The data obtained from in vitro assays and animal studies are used in formulating a range of dosage for human use. The dosage of such compounds lies for example within a range of circulating concentrations what include the ED50 with little or no toxicity. The dosage varies within this range depending upon the dosage form employed, sensitivity of the subject, and the route of administration.

Normal dosage amounts may vary from 0.01 to 1000 milligrams total dose, depending upon the route of administration. Guidance as to particular dosages and methods of delivery is provided in the literature. See U.S. Pat. Nos. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the subject.

Pharmaceutical formulations for parenteral administration include aqueous solutions of active compounds as well as lyophilized dry forms, powdered forms, and spray dried forms of the compound. For injection, the pharmaceutical compositions may be formulated in aqueous solutions, for example in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles may include fatty oils such as sesame oil, or synthetic fatty acid esters, such as ethyl oleate or triglycerides, or liposomes. Suitable liposomes include, but are not limited to, the phospholipid vesicles described in Geho, W., et. al., J Diabetes Sci Technol, Vol 3, Issue 6, November 2009, which is incorporated by reference herein. Optionally, the suspension may also contain suitable stabilizers or agents which increase the solubility of the compounds to allow for the preparation of highly concentrated solutions.

An IFNL3 or modified IFNL3 of this disclosure can be used alone or in combination with other active compounds. The present disclosure furthermore provides medicaments comprising at least one IFNL3 or modified IFNL3 and one or more further active ingredients, in particular for the treatment and/or prevention of the disorders mentioned above.

Suitable active ingredients for combination may include, by way of example: active ingredients which modulate the immune system such as vaccines or immune stimulants, antibiotics and anti viral drugs, and antiparasitic drugs.

The invention will now be described in more detail with respect to the following, specific, non-limiting examples.

### EXAMPLES

### Example 1

This example details general methods suitable for cloning and expression of an IFNL3 polypeptide, including those of the present invention, in E. coli. Methods for cloning DNA, including cDNA encoding IFNL3, and expression in host cells, such as bacteria or mammalian cell lines, are well known to those of ordinary skill in the art.

Amino acid sequences including a 6-His tag (SEQ ID NO: 20) at the N-terminus, and cDNA sequences encoding the various species of IFNL3 of the present invention are shown in Table 4 and Table 5, respectively. It is readily apparent to those of skill in the art that the 6-His tag (SEQ ID NO: 20) is optionally included in the amino acid sequence of the proteins, and may be eliminated if so desired. It is also readily apparent to those of skill in the art that all or some of the secretion signal sequence of the proteins may also be included if desired, for example if the proteins are to be secreted into the host cell culture medium, or into the periplasmic space of the desired recombinant host cell, selected for production of the proteins.

TABLE 4
IFNL3 Amino Acid Sequences

| SEQ ID | | |
|---|---|---|
| NO: | Sequence Name | Sequence |
| 1 | EXLT-01 | MHHHHHHRTGAVPVPSAPRALPPARGCHVAQFKSLSPQEL |
| | Bovine IFNL3 | QAFKTARDAFEDSFLPKDWDCSTHLFPRTRDLKHLQVWER |
| | amino acid | PVALEAELALTLTVLEAMANSSLGHSLEQPLLTLQNIHSK |
| | sequence | LQACVPAQPTASSRPRGRLHHWLHRLQEARKESQDCLEAS |
| | | VMFNLLRLLTRDLKCVASGDQCV |
| 2 | EXLT-02 | MHHHHHHRTGAVPVPSAPRALPPARGCHVAQFKSLSPQEL |
| | Bovine IFNL3 | QAFKTARDAFEDSFLPKDWDCSTHLFPRTRDLKHLQVWER |
| | amino acid | PVALEAELALTLTVLEAMANSSLGHSLEQPLLTLQNIHSK |
| | sequence | LQACVPAQPTASSRPRGRLHHWLHRLQEARKESQDCLEAS |
| | | VMFNLLRLLTRDLKCVASG |
| 3 | EXLT-03 | MHHHHHHRTGAVPVPSAPRALPPARGCHVAQFKSLSPQEL |
| | Bovine IFNL3 | QAFKTARDAFEDSFLPKDWDCSTHLFPRTRDLKHLQVWER |
| | amino acid | PVALEAELALTLTVLEAMANSSLGHSLEQPLLTLQNIHSK |
| | sequence | LQACVPAQPTASSRPRGRLHHWLHRLQEARKESQDCLEAS |
| | | VMFNLLRLLTRDLKCVA |
| 4 | EXLT-04 | MHHHHHHPVPEALRALPGARGCHLAQFKSLSPQALQAFKR |
| | Porcine IFNL3 | AKDAFEESLLEDWNCSSRIFPRSRDLKQLQVWERPVALEA |
| | amino acid | EVALTLSVLGSLANSSLHSSLDQPLHTLRHIHAQLQACVP |
| | sequence | AQPMAGPRPRGRLHHWLHRLQEAQKKEPQSCLEASVMFNL |
| | | FRLLTRDLKCVASGDLCV |
| 5 | EXLT-05 | MHHHHHHFPQVTPKKSCSLSKYQFPAPLELKAVWRMKEQF |
| | Avian IFNL3 | EDIMLLTNRKCNTRLFHRKWDIAELSVPDRITLVEAELDL |
| | amino acid | TITVLTNPTTQRLAETCQQPLAFLTQVQEDLRDCLALEAP |
| | sequence | SHQPSGKLRHWLQKLETAKKKETAGCLEASAILHIFQVLN |
| | | DLRCAAQREDCT |
| 6 | EXLT-06 | MHHHHHHGPVPRRHRALPGARGCHIAQYKSLSPGERLAFK |
| | Equine IFNL3 | NATDAFEESFWLKNHTCRSRPFPRTWDLRQLQVWERPVAL |
| | amino acid | EAELNLTLRVLANSSLGDVLDRPLGTLSHIHSELQACVAA |
| | sequence | QPTEGPRPRGRLHQWLRRLLEAQEEEPWGCLEASVMFNLF |
| | | RLLTKDLTCVASGDLCV |
| 7 | EXLT-07 | MHHHHHHGPVPTSKPTTTRRGCHMDRFQSLSPRELEAFKK |
| | Canine IFNL3 | AKDALEESLSWKNWSCSSRLFPRSRDLRLLQAWERPVALE |
| | amino acid | AELDLTLKVLENMTDSSLGVTLDQPLRTLHHIHSELQACV |
| | sequence | PAQPTADPRPHGRLHHWLHRLQKAPKESQGCLEASITFNL |
| | | FRLLTRDLKCVASRDLCV |
| 8 | EXLT-08 | MHHHHHHLQKESVAPQAWTCRPCLFPKTRELRQLQVRERS |
| | Feline IFNL3 | VALEAELALTLKVLGTVADASQGDILDQPLHTLCHIHSEL |
| | amino acid | WACVSTRPPAGPQTRGHLHHWLPWLHKAPKKSLSCLEASV |
| | sequence | MFNLFYLLIWDLKGVTSEDPCV |
| 9 | EXLT-09 | MHHHHHHPVPSAPRALPPARGCHMAQFKSLSPQELQAFKT |
| | Ovine IFNL3 | VRDAFEDSRLQKDWDCGGRRAALTCLLSPPQVWERPVALE |
| | amino acid | AELALTLTVLEAMANSSLGRSLEQPLLTLQHVHSKLQACV |
| | sequence | PAQPTAGPRPRGRLHHWLHRLQEAQKKESQDCLEASVMFN |
| | | LFRLLTRDLKCVASGDQCV |

TABLE 5
IFNL3 Nucleotide Sequences

| SEQ ID | | |
|---|---|---|
| NO: | Sequence Name | Sequence |
| 10 | Bovine IFNL3 | CATATGCACCACCACCATCACCACCGTACCGGTGCGGTT |
| | nucleotide | CCGGTTCCGAGCGCGCCGCGTGCGCTGCCGCCGGCGCGT |
| | sequence | GGTTGCCATGTTGCGCAGTTCAAGAGCCTGAGCCCGCAG |
| | mature | GAGCTGCAAGCGTTTAAAACCGCGCGTGACGCGTTCGAA |
| | | GATAGCTTTCTGCCGAAGGACTGGGATTGCAGCACCCAC |
| | | CTGTTCCCGCGTACCCGTGACCTGAAACACCTGCAAGTT |
| | | TGGGAGCGTCCGGTTGCGCTGGAGGCGGAACTGGCGCTG |
| | | ACCCTGACCGTGCTGGAGGCGATGGCGAACAGCAGCCTG |
| | | GGTCACAGCCTGGAACAGCCGCTGCTGACCCTGCAAAAC |
| | | ATCCACAGCAAGCTGCAGGCGTGCGTTCCGGCGCAACCG |
| | | ACCGCGAGCAGCCGTCCGCGTGGCCGTCTGCACCACTGG |
| | | CTGCACCGTCTGCAGGAAGCGCGTAAAGAAAGCCAAGAT |
| | | TGCCTGGAAGCGAGCGTGATGTTTAACCTGCTGCGTCTG |
| | | CTGACCCGTGACCTGAAATGCGTTGCGAGCGGTGACCAG |
| | | TGCGTGTAATGAAAGCTT |
| 11 | Bovine IFNL3 | CATATGCACCACCACCATCACCACCGTACCGGTGCGGTT |
| | nucleotide | CCGGTTCCGAGCGCGCCGCGTGCGCTGCCGCCGGCGCGT |
| | sequence | GGTTGCCATGTTGCGCAGTTCAAGAGCCTGAGCCCGCAG |
| | delta 4 | GAGCTGCAAGCGTTTAAAACCGCGCGTGACGCGTTCGAA |
| | | GATAGCTTTCTGCCGAAGGACTGGGATTGCAGCACCCAC |
| | | CTGTTCCCGCGTACCCGTGACCTGAAACACCTGCAAGTT |
| | | TGGGAGCGTCCGGTTGCGCTGGAGGCGGAACTGGCGCTG |
| | | ACCCTGACCGTGCTGGAGGCGATGGCGAACAGCAGCCTG |
| | | GGTCACAGCCTGGAACAGCCGCTGCTGACCCTGCAAAAC |
| | | ATCCACAGCAAGCTGCAGGCGTGCGTTCCGGCGCAACCG |
| | | ACCGCGAGCAGCCGTCCGCGTGGCCGTCTGCACCACTGG |
| | | CTGCACCGTCTGCAGGAAGCGCGTAAAGAAAGCCAAGAT |
| | | TGCCTGGAAGCGAGCGTTATGTTTAATCTGCTGCGTCTG |
| | | CTGACCCGTGACCTGAAATGCGTTGCGAGCGGCTAATGA |
| | | AAGCTT |
| 12 | Bovine IFNL3 | CATATGCACCACCACCATCACCACCGTACCGGTGCGGTT |
| | nucleotide | CCGGTTCCGAGCGCGCCGCGTGCGCTGCCGCCGGCGCGT |
| | sequence | GGTTGCCATGTGGCGCAGTTCAAGAGCCTGAGCCCGCAG |
| | delta 6 | GAGCTGCAAGCGTTTAAAACCGCGCGTGACGCGTTCGAA |
| | | GATAGCTTTCTGCCGAAGGACTGGGATTGCAGCACCCAC |
| | | CTGTTCCCGCGTACCCGTGACCTGAAACACCTGCAAGTT |
| | | TGGGAGCGTCCGGTTGCGCTGGAGGCGGAACTGGCGCTG |
| | | ACCCTGACCGTGCTGGAGGCGATGGCGAACAGCAGCCTG |
| | | GGTCACAGCCTGGAACAGCCGCTGCTGACCCTGCAAAAC |
| | | ATCCACAGCAAGCTGCAGGCGTGCGTTCCGGCGCAACCG |
| | | ACCGCGAGCAGCCGTCCGCGTGGCCGTCTGCACCACTGG |
| | | CTGCACCGTCTGCAGGAAGCGCGTAAAGAAAGCCAAGAT |
| | | TGCCTGGAAGCGAGCGTGATGTTTAATCTGCTGCGTCTG |
| | | CTGACCCGTGACCTGAAGTGCGTTGCGTAATGAAAGCTT |
| | | CATATGCACCACCACCACCACCACCCCGTACCAGAAGCT |
| 13 | Porcine IFNL3 | CTAAGAGCATTACCGGGTGCGCGTGGCTGCCACCTGGCG |
| | nucleotide | CAGTTCAAGAGCCTGAGCCCGCAGGCGCTGCAAGCGTTC |
| | sequence | AAGCGTGCGAAAGACGCGTTTGAGGAAAGCCTGCTGGAG |
| | | GATTGGAACTGCAGCAGCCGTATCTTTCCGCGTAGCCGT |
| | | GACCTGAAACAGCTGCAAGTGTGGGAACGTCCGGTTGCG |
| | | CTGGAGGCGGAAGTGGCGCTGACCCTGAGCGTTCTGGGT |
| | | AGCCTGGCGAACAGCAGCCTGCACAGCAGCCTGGATCAG |
| | | CCGCTGCACACCCTGCGTCACATTCATGCGCAGCTGCAA |
| | | GCGTGCGTGCCGGCGCAACCGATGGCGGGTCCGCGTCCG |
| | | CGTGGCCGTCTGCACCACTGGCTGCACCGTCTGCAGGAA |
| | | GCGCAAAAGAAAGAACCGCAAAGCTGCCTGGAAGCGAGC |
| | | GTTATGTTCAACCTGTTTCGTCTGCTGACCCGTGACCTG |
| | | AAGTGCGTGGCGAGCGGTGATCTGTGCGTTTAATGAAAG |
| | | CTT |
| 14 | Avian IFNL3 | CATATGCACCACCACCATCACCACTTTCCCCAAGTAACA |
| | nucleotide | CCCAAAAAATCATGCAGCCTGAGCAAATACCAATTTCCG |
| | sequence | GCGCCGCTGGAACTGAAAGCGGTTTGGCGTATGAAGGAG |
| | | CAGTTCGAAGACATCATGCTGCTGACCAACCGTAAATGC |
| | | AACACCCGTCTGTTTCACCGTAAGTGGGACATCGCGGAA |
| | | CTGAGCGTGCCGGATCGTATTACCCTGGTTGAGGCGGAA |
| | | CTGGATCTGACCATTACCGTGCTGACCAACCCGACCACC |
| | | CAACGTCTGGCGGAGACCTGCCAGCAACCGCTGGCGTTC |
| | | CTGACCCAGGTTCAAGAAGACCTGCGTGATTGCCTGGCG |
| | | CTGGAGGCGCCGAGCCACCAGCCGAGCGGTAAACTGCGT |
| | | CACTGGCTGCAAAAGCTGGAGACCGCGAAGAAAAAGGAA |
| | | ACCGCGGGTTGCCTGGAGGCGAGCGCGATCCTGCACATT |
| | | TTTCAGGTGCTGAACGACCTGCGTTGCGCGGCGCAACGT |
| | | GAGGATTGCACCTAATGAAAGCTT |
| 15 | Equine IFNL3 | CATATGCATCACCACCACCACCACGGACCCGTACCAAGG |
| | nucleotide | AGACACAGAGCTCTGCCGGGTGCGCGTGGCTGCCACATC |
| | sequence | GCGCAGTACAAGAGCCTGAGCCCGGGCGAACGTCTGGCG |
| | | TTCAAAAACGCGACCGACGCGTTCGAGGAGAGCTTCTGG |
| | | CTGAAGAACCACACCTGCCGTAGCCGTCCGTTTCCGCGT |
| | | ACCTGGGATCTGCGTCAGCTGCAAGTGTGGGAGCGTCCG |
| | | GTTGCGCTGGAAGCGGAGCTGAACCTGACCCTGCGTGTG |
| | | CTGGCGAACAGCAGCCTGGGTGACGTTCTGGATCGTCCG |
| | | CTGGGCACCCTGAGCCACATTCATAGCGAACTGCAGGCG |
| | | TGCGTGGCGGCGCAACCGACCGAGGGTCCGCGTCCGCGT |
| | | GGCCGTCTGCACCAGTGGCTGCGTCGTCTGCTGGAAGCG |
| | | CAAGAGGAAGAGCCGTGGGGTTGCCTGGAGGCGAGCGTT |
| | | ATGTTCAACCTGTTTCGTCTGCTGACCAAAGACCTGACC |
| | | TGCGTGGCGAGCGGCGATCTGTGCGTTTAATGAAAGCTT |
| 16 | Canine IFNL3 | CATATGCACCACCACCATCACCACGGACCCGTACCAACA |
| | nucleotide | TCAAAACCCACAACCACCCGTCGTGGCTGCCACATGGAC |
| | sequence | CGTTTCCAGAGCCTGAGCCCGCGTGAGCTGGAAGCGTTT |
| | | AAGAAAGCGAAGGATGCGCTGGAGGAAAGCCTGAGCTGG |
| | | AAAAACTGGAGCTGCAGCAGCCGTCTGTTCCCGCGTAGC |
| | | CGTGACCTGCGTCTGCTGCAGGCGTGGGAGCGTCCGGTG |
| | | GCGCTGGAGGCGGAACTGGATCTGACCCTGAAAGTGCTG |
| | | GAAAACATGACCGACAGCAGCCTGGGTGTTACCCTGGAT |
| | | CAACCGCTGCGTACCCTGCACCACATTCATAGCGAGCTG |
| | | CAGGCGTGCGTTCCGGCGCAACCGACCGCGGACCCGCGT |
| | | CCGCATGGTCGTCTGCACCACTGGCTGCACCGTCTGCAG |
| | | AAGGCGCCGAAAGAGAGCCAAGGCTGCCTGGAAGCGAGC |
| | | ATTACCTTCAACCTGTTTCGTCTGCTGACCCGTGACCTG |
| | | AAGTGCGTGGCGAGCCGTGATCTGTGCGTTTAATGAAAG |
| | | CTT |
| 17 | Feline IFNL3 | CATATGCACCACCACCACCACCACTTACAAAAAGAATCA |
| | nucleotide | GTAGCTCCCCAAGCGTGGACCTGCCGTCCGTGCCTGTTC |
| | sequence | CCGAAAACCCGTGAGCTGCGTCAGCTGCAAGTGCGTGAA |
| | | CGTAGCGTTGCGCTGGAGGCGGAACTGGCGCTGACCCTG |
| | | AAGGTTCTGGGCACCGTTGCGGATGCGAGCCAGGGTGAC |
| | | ATCCTGGATCAACCGCTGCACACCCTGTGCCACATTCAC |
| | | AGCGAACTGTGGGCGTGCGTGAGCACCCGTCCGCCGGCG |
| | | GGTCCGCAAACCCGTGGTCACCTGCACCATTGGCTGCCG |
| | | TGGCTGCACAAGGCGCCGAAGAAAAGCCTGAGCTGCCTG |
| | | GAGGCGAGCGTTATGTTCAACCTGTTTTACCTGCTGATC |
| | | TGGGACCTGAAAGGTGTGACCAGCGAAGATCCGTGCGTT |
| | | TAATGAAAGCTT |
| 18 | Ovine IFNL3 | CATATGCACCACCACCATCACCACCCCGTACCATCAGCT |
| | nucleotide | CCCAGAGCACTCCCGCCGGCGCGTGGTTGCCACATGGCG |
| | sequence | CAGTTCAAAAGCCTGAGCCCGCAGGAGCTGCAAGCGTTC |
| | | AAGACCGTGCGTGACGCGTTTGAAGATAGCCGTCTGCAG |
| | | AAAGATTGGGATTGCGGTGGCCGTCGTGCGGCGCTGACC |
| | | TGCCTGCTGAGCCCGCCGCAAGTTTGGGAGCGTCCGGTT |
| | | GCGCTGGAGGCGGAACTGGCGCTGACCCTGACCGTTCTG |
| | | GAGGCGATGGCGAACAGCAGCCTGGGTCGTAGCCTGGAA |
| | | CAGCCGCTGCTGACCCTGCAACACGTGCACAGCAAGCTG |
| | | CAGGCGTGCGTTCCGGCGCAACCGACCGCGGGTCCGCGT |
| | | CCGCGTGGCCGTCTGCACCACTGGCTGCACCGTCTGCAG |
| | | GAAGCGCAAAAGAAAGAAAGCCAGGACTGCCTGGAAGCG |
| | | AGCGTGATGTTCAACCTGTTCCGTCTGCTGACCCGTGAC |
| | | CTGAAATGCGTTGCGAGCGGCGACCAATGCGTTTAATGA |
| | | AAGCTT |

The transformation of E. coli with plasmids containing DNA encoding the IFNL3 or modified IFNL3 or IFNL3 analog protein allows for small and large scale biosynthesis of the IFNL3 polypeptide.

The DNA encoding bovine Interferon lambda 3 was found in GenBank accession number NM_001281901, avian Interferon lambda 3 was found in GenBank accession number NM_001128496, porcine Interferon lambda 3 was found in GenBank accession number NM_001166490, equine interferon lambda 3 was found in GenBank accession number XP_00191641, canine interferon lambda 3 was found at GenBank Accession number P_855366, ovine Interferon lambda 3 is found in GenBank accession number NC_019471.2, and feline was found in GenBank accession number P_855366.

Escherichia coli strain W3110 is used to produce a wild-type or modified IFNL3. A single research cell bank (RCB) vial is removed from −80° C. and thawed at room temperature, then 50 μL is used to inoculate 50 mL of Seed Media (a chemically defined medium) supplemented with 50 μg/mL kanamycin sulfate in a 250 mL baffled Erlenmeyer flask. The primary seed culture is grown for approximately 18 hours at 37° C. and 250 rpm (1-inch throw). The primary seed culture is sub-cultured into a secondary seed culture to an optical density measured at 600 nm wavelength (OD600) of 0.05 in a 500 mL baffled Erlenmeyer flask containing 100 mL of Seed Medium supplemented with 50 μg/mL kanamycin sulfate. The secondary seed culture is grown at 37° C. and 250 rpm (1-inch throw) for approximately 8 hours or when the OD600 reached between 2 and 4. IFNL3 polypeptide production can be scaled up using a five (5) liter fermentor. These methods and scale up may also be used for 10 L, 30 L, 150 L and 1000 L batches. In some embodiments of the present invention, at least 2 g of IFNL3 protein is produced for each liter of cell culture. In another embodiment of the present invention, at least 4 g of IFNL3 protein is produced for each liter of cell culture. In another embodiment of the present invention, at least 6 g of IFNL3 protein is produced for each liter of cell culture. In another embodiment of the present invention, at least 8 g of IFNL3 protein is produced for each liter of cell culture. In another embodiment of the present invention, at least 10 g of IFNL3 protein is produced for each liter of cell culture. In another embodiment of the present invention, at least 15 g of IFNL3 protein is produced for each liter of cell culture. In another embodiment of the present invention, at least 20 g of IFNL3 protein is produced for each liter of cell culture.

### Example 2

Cloning and expression of bovine IFNL3 proteins EXLT-01, EXLT-02, EXLT-03, EXLT-04, EXLT-05, EXLT-06, EXLT-07, EXLT-08, and EXLT-09

Cloning Strategy: EXLT-01

Full length protein: mature form of bovine IFNL3. Construct: NdeI-ATG-His tag-bIFNL3-Mature-Stop codon-HindIII. Protein Length=183 MW=20626.5; Predicted pI=8.42; vector: pET30a

Amino acid sequence:
(SEQ ID NO: 1)
MHHHHHHRTGAVPVPSAPRALPPARGCHVAQFKSLSPQELQAFKTARDAF
EDSFLPKDWDCSTHLFPRTRDLKHLQVWERPVALEAELALTLTVLEAMAN
SSLGHSLEQPLLTLQNIHSKLQACVPAQPTASSRPRGRLHHWLHRLQEAR
KESQDCLEASVMFNLLRLLTRDLKCVASGDQCV
DNA sequence:
(SEQ ID NO: 10)
CATATGCACCACCACCATCACCACCGTACCGGTGCGGTTCCGGTTCCGAG
CGCGCCGCGTGCGCTGCCGCCGGCGCGTGGTTGCCATGTTGCGCAGTTCA
AGAGCCTGAGCCCGCAGGAGCTGCAAGCGTTTAAAACCGCGCGTGACGCG
TTCGAAGATAGCTTTCTGCCGAAGGACTGGGATTGCAGCACCCACCTGTT
CCCGCGTACCCGTGACCTGAAACACCTGCAAGTTTGGGAGCGTCCGGTTG
CGCTGGAGGCGGAACTGGCGCTGACCCTGACCGTGCTGGAGGCGATGGCG
AACAGCAGCCTGGGTCACAGCCTGGAACAGCCGCTGCTGACCCTGCAAAA
CATCCACAGCAAGCTGCAGGCGTGCGTTCCGGCGCAACCGACCGCGAGCA
GCCGTCCGCGTGGCCGTCTGCACCACTGGCTGCACCGTCTGCAGGAAGCG
CGTAAAGAAAGCCAAGATTGCCTGGAAGCGAGCGTGATGTTTAACCTGCT
GCGTCTGCTGACCCGTGACCTGAAATGCGTTGCGAGCGGTGACCAGTGCG
TGTAATGAAAGCTT

Gene Synthesis and Subcloning: Target DNA sequence bIFNL3-Mature (EXLT-01) was optimized for E coli expression and synthesized. The synthesized sequence was cloned into vector pET30a with His tag for protein expression in E. coli. Expression Evaluation: E. coli strainBL21 Star (DE3) was transformed with recombinant plasmid. A single colony was inoculated into LB medium containing kanamycin; culture was incubated in 37° C. at 200 rpm and then induced with IPTG. SDS-PAGE and Western blot were used to monitor the expression.

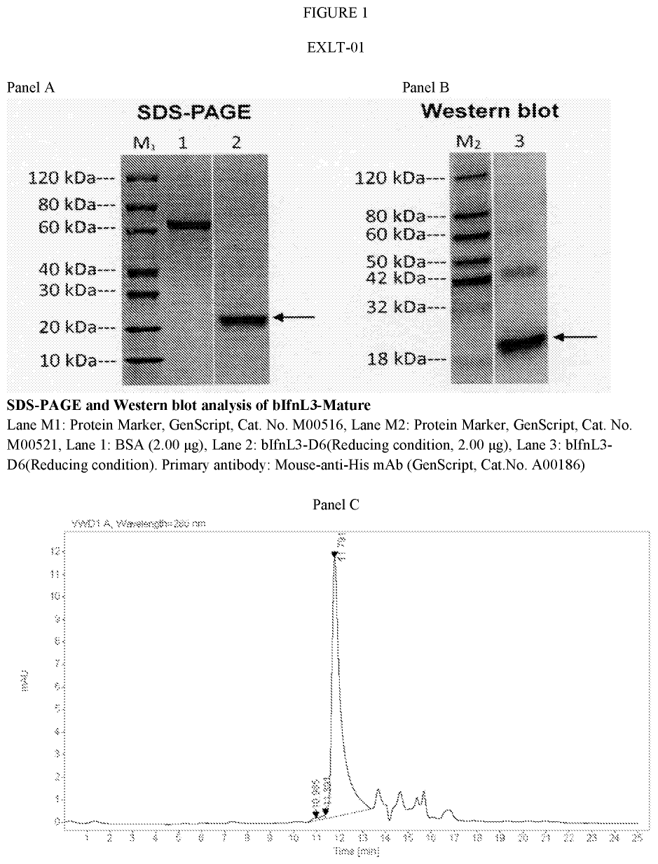

BL21Star (DE3) stored in glycerol was inoculated into 5052 auto-induced medium containing kanamycin and cultured at 37° C. When the OD600 reached about 1.2, the cell culture was cultured at 37° C. for 4 hours. Cells were harvested by centrifugation. Purification and Analysis: Cell pellets were resuspended with lysis buffer followed by sonication. The precipitate after centrifugation was dissolved using urea. Denatured supernatant after centrifugation was kept for future purification. Target protein were refolded and sterilized by 0.22 μm filter before stored in aliquots. IFNL3 EXLT-01 protein was purified by SEC-HPLC using an Agilent SEC_0.3 ml-min_25 min_20171122.M with an injection Volume of 5.0 ml. The concentration was determined by Bradford protein assay with BSA as standard. The protein purity and molecular weight were determined by standard SDS-PAGE along with western blot confirmation. Results are shown in FIG. 1 for EXLT-01.

Cloning Strategy: EXLT-02

Full length protein: Mature bovine IFNL3 having 4 amino acids deleted from the C-terminus.
Construct: NdeI-ATG-His tag-bIfnL3-D4-Stop codon-HindIII. Protein Length—179
MW=20181.0; predicted pI=8.82; vector: pET30a

Amino acid sequence:
(SEQ ID NO: 2)
MHHHHHHRTGAVPVPSAPRALPPARGCHVAQFKSLSPQELQAFKTARDAF
EDSFLPKDWDCSTHLFPRTRDLKHLQVWERPVALEAELALTLTVLEAMAN
SSLGHSLEQPLLTLQNIHSKLQACVPAQPTASSRPRGRLHHWLHRLQEAR
KESQDCLEASVMFNLLRLLTRDLKCVASG
DNA sequence: 552bp
(SEQ ID NO: 11)
CATATGCACCACCACCATCACCACCGTACCGGTGCGGTTCCGGTTCCGAG
CGCGCCGCGTGCGCTGCCGCCGGCGCGTGGTTGCCATGTTGCGCAGTTCA
AGAGCCTGAGCCCGCAGGAGCTGCAAGCGTTTAAAACCGCGCGTGACGCG
TTCGAAGATAGCTTTCTGCCGAAGGACTGGGATTGCAGCACCCACCTGTT
CCCGCGTACCCGTGACCTGAAACACCTGCAAGTTTGGGAGCGTCCGGTTG
CGCTGGAGGCGGAACTGGCGCTGACCCTGACCGTGCTGGAGGCGATGGCG
AACAGCAGCCTGGGTCACAGCCTGGAACAGCCGCTGCTGACCCTGCAAAA
CATCCACAGCAAGCTGCAGGCGTGCGTTCCGGCGCAACCGACCGCGAGCA
GCCGTCCGCGTGGCCGTCTGCACCACTGGCTGCACCGTCTGCAGGAAGCG
CGTAAAGAAAGCCAAGATTGCCTGGAAGCGAGCGTTATGTTTAATCTGCT
GCGTCTGCTGACCCGTGACCTGAAATGCGTTGCGAGCGGCTAATGAAAGC
TT

Gene Synthesis and Subcloning: Target DNA sequence bIfnL3-D4 (EXLT-02) was optimized for E coli expression and synthesized. The synthesized sequence was cloned into vector pET30a with His tag for protein expression in E. coli. E. coli strain BL21 Star (DE3) was transformed with recombinant plasmid. A single colony was inoculated into LB medium containing kanamycin; culture was incubated in 37° C. at 200 rpm and then induced with IPTG. SDS-PAGE and Western blot were used to monitor the expression. BL21Star (DE3) stored in glycerol was inoculated into TB medium containing kanamycin and cultured at 37° C. When the OD600 reached about 1.2, cell culture was induced with IPTG at 37° C. for 4 hours. Cells were harvested by centrifugation.

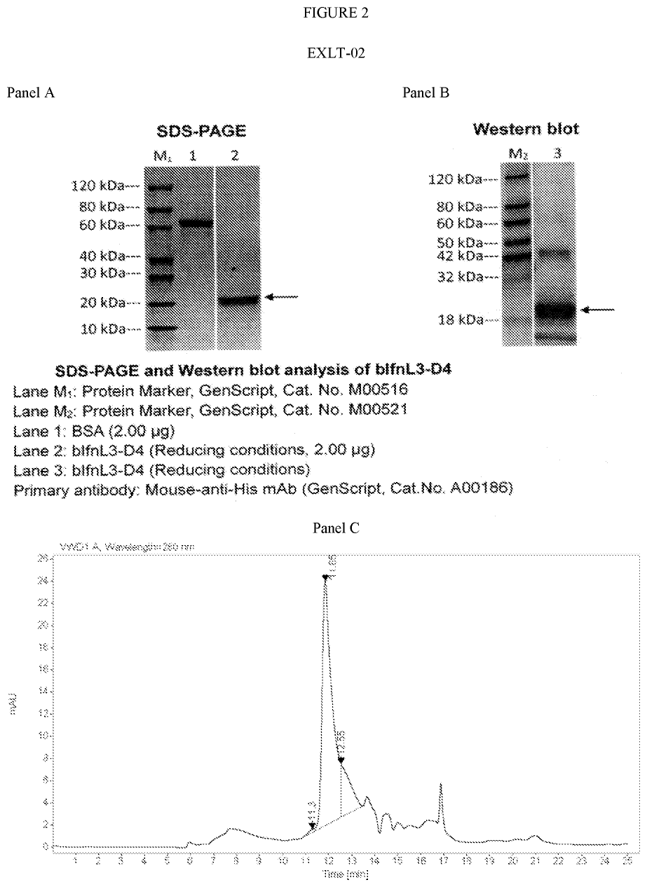

Cell pellets were resuspended with lysis buffer followed by sonication. The precipitate after centrifugation was dissolved using urea. Denatured supernatant after centrifugation was kept for future purification. Target protein were refolded and sterilized by 0.22 μm filter before stored in aliquots. IFNL3 EXLT-02 protein was purified by SEC-HPLC using an Agilent SEC_0.3 ml-min_25 min_20171122.M with an injection Volume of 30.0 ml. The concentration was determined by Bradford protein assay with BSA as standard. The protein purity and molecular weight were determined by standard SDS-PAGE along with western blot confirmation. Results are shown in FIG. 2 for EXLT-02. It was shown that deletion of amino acids from the C-terminus of the protein lead to decreased stability by HPLC (FIG. 2, panel C).

Cloning Strategy: EXLT-03

Full length protein: Mature bovine IFNL3 having 6 amino acids deleted from the C-terminus.

Construct: NdeI-ATG-His tag-bIfnL3-D6-Stop codon-HindIII. Protein Length—177 MW=20036.9; predicted pI=8.82; vector: pET30a

Amino acid sequence:
(SEQ ID NO: 3)
MHHHHHHRTGAVPVPSAPRALPPARGCHVAQFKSLSPQELQAFKTARDAF
EDSFLPKDWDCSTHLFPRTRDLKHLQVWERPVALEAELALTLTVLEAMAN
SSLGHSLEQPLLTLQNIHSKLQACVPAQPTASSRPRGRLHHWLHRLQEAR
KESQDCLEASVMFNLLRLLTRDLKCVA
DNA sequence: 546bp
(SEQ ID NO: 12)
CATATGCACCACCACCATCACCACCGTACCGGTGCGGTTCCGGTTCCGAG
CGCGCCGCGTGCGCTGCCGCCGGCGCGTGGTTGCCATGTGGCGCAGTTCA
AGAGCCTGAGCCCGCAGGAGCTGCAAGCGTTTAAAACCGCGCGTGACGCG
TTCGAAGATAGCTTTCTGCCGAAGGACTGGGATTGCAGCACCCACCTGTT
CCCGCGTACCCGTGACCTGAAACACCTGCAAGTTTGGGAGCGTCCGGTTG
CGCTGGAGGCGGAACTGGCGCTGACCCTGACCGTGCTGGAGGCGATGGCG
AACAGCAGCCTGGGTCACAGCCTGGAACAGCCGCTGCTGACCCTGCAAAA
CATCCACAGCAAGCTGCAGGCGTGCGTTCCGGCGCAACCGACCGCGAGCA
GCCGTCCGCGTGGCCGTCTGCACCACTGGCTGCACCGTCTGCAGGAAGCG
CGTAAAGAAAGCCAAGATTGCCTGGAAGCGAGCGTGATGTTTAATCTGCT
GCGTCTGCTGACCCGTGACCTGAAGTGCGTTGCGTAATGAAAGCTT

Gene Synthesis and Subcloning: Target DNA sequence bIfnL3-D6 (EXLT-03) was optimized for E coli expression and synthesized. The synthesized sequence was cloned into vector pET30a with His tag for protein expression in E. coli. E. coli strain BL21 Star (DE3) was transformed with the recombinant plasmid described above. A single colony was inoculated into LB medium containing kanamycin. The culture was incubated in 37° C. at 200 rpm and then induced with IPTG. SDS-PAGE and Western blot were used to monitor the expression. BL21Star (DE3) stored in glycerol was inoculated into TB medium containing kanamycin and cultured at 37° C. When the OD600 reached about 1.2, cell culture was induced with IPTG at 37° C. for 4 hours. Cells were harvested by centrifugation.

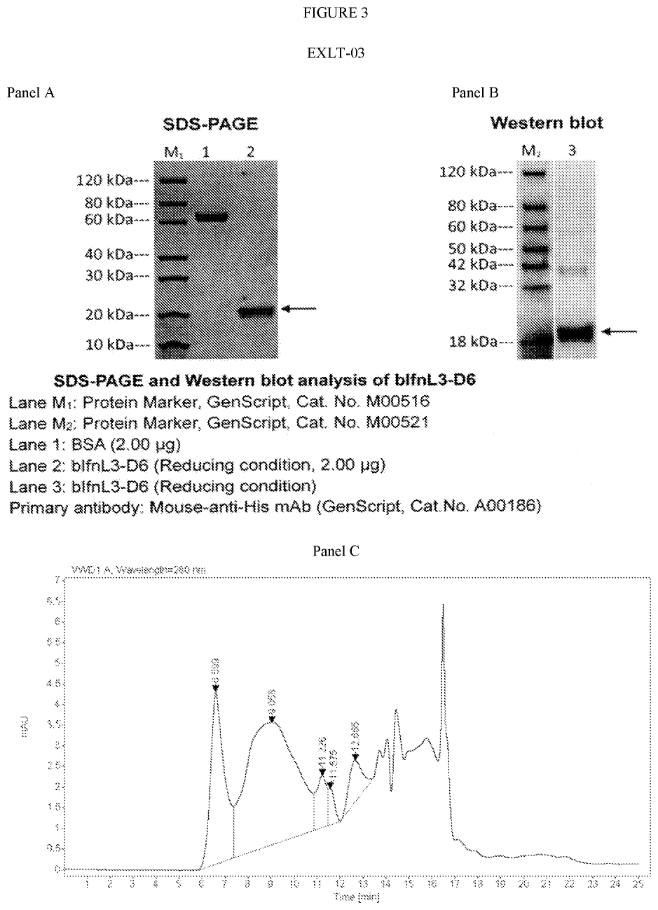

Cell pellets were resuspended with lysis buffer followed by sonication. The precipitate after centrifugation was dissolved using urea. Denatured supernatant after centrifugation was kept for future purification. Target protein were refolded and sterilized by 0.22 μm filter before stored in aliquots. IFNL3 EXLT-03 protein was purified by SEC-HPLC using an Agilent SEC_0.3 ml-min_25 min_20171122.M with an injection Volume of 25.0 ml. The concentration was determined by Bradford protein assay with BSA as standard. The protein purity and molecular weight were determined by standard SDS-PAGE along with western blot confirmation. Results are shown in FIG. 3 for EXLT-03. It was shown that deletion of six amino acids from the C-terminus of the protein lead to further decreased stability by HPLC (FIG. 3, panel C).

Cloning Strategy: Full length protein EXLT-04 Porcine IFNL3 mature amino acid sequence:

NdeI-ATG-His tag-EXLT-04 Porcine IFNL3 mature amino acid sequence-Stop codon-HindIII. Protein Length=178 amino acids; MW=20092.1; predicted pI=8.90; vector: pET30a

Amino acid sequence:
(SEQ ID NO: 4)
MHHHHHHPVPEALRALPGARGCHLAQFKSLSPQALQAFKRAKDAFEESLL
EDWNCSSRIFPRSRDLKQLQVWERPVALEAEVALTLSVLGSLANSSLHSS
LDQPLHTLRHIHAQLQACVPAQPMAGPRPRGRLHHWLHRLQEAQKKEPQS
CLEASVMFNLFRLLTRDLKCVASGDLCV
DNA sequence: 549bp
(SEQ ID NO: 13)
CATATGCACCACCACCACCACCACCCCGTACCAGAAGCTCTAAGAGCATT
ACCGGGTGCGCGTGGCTGCCACCTGGCGCAGTTCAAGAGCCTGAGCCCGC
AGGCGCTGCAAGCGTTCAAGCGTGCGAAAGACGCGTTTGAGGAAAGCCTG
CTGGAGGATTGGAACTGCAGCAGCCGTATCTTTCCGCGTAGCCGTGACCT
GAAACAGCTGCAAGTGTGGGAACGTCCGGTTGCGCTGGAGGCGGAAGTGG
CGCTGACCCTGAGCGTTCTGGGTAGCCTGGCGAACAGCAGCCTGCACAGC
AGCCTGGATCAGCCGCTGCACACCCTGCGTCACATTCATGCGCAGCTGCA
AGCGTGCGTGCCGGCGCAACCGATGGCGGGTCCGCGTCCGCGTGGCCGTC
TGCACCACTGGCTGCACCGTCTGCAGGAAGCGCAAAAGAAAGAACCGCAA
AGCTGCCTGGAAGCGAGCGTTATGTTCAACCTGTTTCGTCTGCTGACCCG
TGACCTGAAGTGCGTGGCGAGCGGTGATCTGTGCGTTTAATGAAAGCTT

Target DNA sequence of EXLT-04 Porcine IFNL3 mature amino acid sequence was optimized and synthesized. The synthesized sequence was cloned into vector pET-30a(+) with His tag for protein expression in E. coli. E. coli strain BL21 star (DE3) was transformed with recombinant plasmid. A single colony was inoculated into 5052 auto-induced medium containing related antibiotic; culture was incubated in 37° C. at 200 rpm and then induced with IPTG. SDS-PAGE was used to monitor the expression. Recombinant BL21 star (DE3) stored in glycerol was inoculated into 5052 auto-induced medium containing related antibiotic and cultured at 37° C. When the OD600 reached about 3, cell culture was cultured at 15° C./16 h. Cells were harvested by centrifugation. Cell pellets were resuspended with lysis buffer followed by sonication. The precipitate after centrifugation was dissolved using denaturing agent. Target protein was sterilized by 0.22 μm filter before stored in aliquots. The concentration was determined by BCA™ protein assay with BSA as standard. The protein purity and molecular weight were determined by standard SDS-PAGE along with western blot confirmation.

EXLT-05 Avian IFNL3 mature amino acid sequence

NdeI-ATG-His tag-EXLT-05 Avian IFNL3 mature amino acid sequence-Stop codon-HindIII.
Protein Length=172; MW=19899.9; predicted pI=7.85; vector: pET30a

(SEQ ID NO: 5)
MHHHHHHFPQVTPKKSCSLSKYQFPAPLELKAVWRMKEQFEDIMLLTNRK
CNTRLFHRKWDIAELSVPDRITLVEAELDLTITVLTNPTTQRLAETCQQP
LAFLTQVQEDLRDCLALEAPSHQPSGKLRHWLQKLETAKKKETAGCLEAS
AILHIFQVLNDLRCAAQREDCT
DNA sequence: 531bp
(SEQ ID NO: 14)
CATATGCACCACCACCATCACCACTTTCCCCAAGTAACACCCAAAAAATC
ATGCAGCCTGAGCAAATACCAATTTCCGGCGCCGCTGGAACTGAAAGCGG
TTTGGCGTATGAAGGAGCAGTTCGAAGACATCATGCTGCTGACCAACCGT
AAATGCAACACCCGTCTGTTTCACCGTAAGTGGGACATCGCGGAACTGAG
CGTGCCGGATCGTATTACCCTGGTTGAGGCGGAACTGGATCTGACCATTA
CCGTGCTGACCAACCCGACCACCCAACGTCTGGCGGAGACCTGCCAGCAA
CCGCTGGCGTTCCTGACCCAGGTTCAAGAAGACCTGCGTGATTGCCTGGC
GCTGGAGGCGCCGAGCCACCAGCCGAGCGGTAAACTGCGTCACTGGCTGC
AAAAGCTGGAGACCGCGAAGAAAAAGGAAACCGCGGGTTGCCTGGAGGCG
AGCGCGATCCTGCACATTTTTCAGGTGCTGAACGACCTGCGTTGCGCGGC
GCAACGTGAGGATTGCACCTAATGAAAGCTT

Target DNA sequence of EXLT-05 Avian IFNL3 mature amino acid sequence was optimized and synthesized. The synthesized sequence was cloned into vector pET-30a(+) with His tag for protein expression in E. coli. E. coli strain BL21 star (DE3) was transformed with recombinant plasmid. A single colony was inoculated into TB medium containing related antibiotic; culture was incubated in 37° C. at 200 rpm and then induced with IPTG. SDS-PAGE was used to monitor the expression. Recombinant BL21 star (DE3) stored in glycerol was inoculated into TB medium containing related antibiotic and cultured at 37° C. When the OD600 reached about 1.2, cell culture was induced with IPTG at 15° C./16 h. Cells were harvested by centrifugation.

Cell pellets were resuspended with lysis buffer followed by sonication. The precipitate after centrifugation was dissolved using denaturing agent. Target protein was sterilized by 0.22 μm filter before stored in aliquots. The concentration was determined by Bradford protein assay with BSA as standard. The protein purity and molecular weight were determined by standard SDS-PAGE along with Western blot confirmation. Freeze-thaw testing was conducted by freezing sample (approximately −80° C.) for more than 4 hours, followed by thawing it at room temperature. After two cycles of freeze-thaw testing, the sample was checked after centrifugation. No visible precipitation was observed.

EXLT-06 Equine IFNL3 mature amino acid sequence

NdeI-ATG-His tag-EXLT-06 Equine IFNL3 mature amino acid sequence-Stop codon-HindIII.
Protein Length=177; MW=20225.0; Predicted pI=8.42; vector: pET30a

Amino acid sequence:
(SEQ ID NO: 6)
MHHHHHHGPVPRRHRALPGARGCHIAQYKSLSPGERLAFKNATDAFEESF
WLKNHTCRSRPFPRTWDLRQLQVWERPVALEAELNLTLRVLANSSLGDVL
DRPLGTLSHIHSELQACVAAQPTEGPRPRGRLHQWLRRLLEAQEEEPWGC
LEASVMFNLFRLLTKDLTCVASGDLCV
DNA sequence: 546bp
(SEQ ID NO: 15)
CATATGCATCACCACCACCACCACGGACCCGTACCAAGGAGACACAGAGC
TCTGCCGGGTGCGCGTGGCTGCCACATCGCGCAGTACAAGAGCCTGAGCC
CGGGCGAACGTCTGGCGTTCAAAAACGCGACCGACGCGTTCGAGGAGAGC
TTCTGGCTGAAGAACCACACCTGCCGTAGCCGTCCGTTTCCGCGTACCTG
GGATCTGCGTCAGCTGCAAGTGTGGGAGCGTCCGGTTGCGCTGGAAGCGG
AGCTGAACCTGACCCTGCGTGTGCTGGCGAACAGCAGCCTGGGTGACGTT
CTGGATCGTCCGCTGGGCACCCTGAGCCACATTCATAGCGAACTGCAGGC
GTGCGTGGCGGCGCAACCGACCGAGGGTCCGCGTCCGCGTGGCCGTCTGC
ACCAGTGGCTGCGTCGTCTGCTGGAAGCGCAAGAGGAAGAGCCGTGGGGT
TGCCTGGAGGCGAGCGTTATGTTCAACCTGTTTCGTCTGCTGACCAAAGA
CCTGACCTGCGTGGCGAGCGGCGATCTGTGCGTTTAATGAAAGCTT

Gene Synthesis and Subcloning: Target DNA sequence of EXLT-06 Equine IFNL3 mature amino acid sequence was optimized and synthesized. The synthesized sequence was cloned into vector pET-30a(+) with His tag for protein expression in E. coli. E. coli strain BL21 star (DE3) was transformed with recombinant plasmid. A single colony was inoculated into 5052 auto-induced medium containing related antibiotic; culture was incubated in 37° C. at 200 rpm and then induced with IPTG. SDS-PAGE was used to monitor the expression. Recombinant BL21 star (DE3) stored in glycerol was inoculated into 5052 auto-induced medium containing related antibiotic and cultured at 37° C. When the OD600 reached about 3, cell culture was cultured at 15° C./16 h. Cells were harvested by centrifugation.

Cell pellets were resuspended with lysis buffer followed by sonication. The precipitate after centrifugation was dissolved using denaturing agent. Target protein was sterilized by 0.22 μm filter before stored in aliquots. The concentration was determined by BCA™ protein assay with BSA as standard. The protein purity and molecular weight were determined by standard SDS-PAGE along with western blot confirmation.

EXLT-07 Canine IFNL3 mature amino acid sequence

NdeI-ATG-His tag-EXLT-07 Canine IFNL3 mature amino acid sequence-Stop codon-HindIII.
Protein Length=178; MW=20476.4; Predicted pI=8.89; vector: pET30a

Amino acid sequence:
(SEQ ID NO: 7)
MHHHHHHGPVPTSKPTTTRRGCHMDRFQSLSPRELEAFKKAKDALEESLS
WKNWSCSSRLFPRSRDLRLLQAWERPVALEAELDLTLKVLENMTDSSLGV
TLDQPLRTLHHIHSELQACVPAQPTADPRPHGRLHHWLHRLQKAPKESQG
CLEASITFNLFRLLTRDLKCVASRDLCV
DNA sequence: 549bp
(SEQ ID NO: 16)
CATATGCACCACCACCATCACCACGGACCCGTACCAACATCAAAACCCAC
AACCACCCGTCGTGGCTGCCACATGGACCGTTTCCAGAGCCTGAGCCCGC
GTGAGCTGGAAGCGTTTAAGAAAGCGAAGGATGCGCTGGAGGAAAGCCTG
AGCTGGAAAAACTGGAGCTGCAGCAGCCGTCTGTTCCCGCGTAGCCGTGA
CCTGCGTCTGCTGCAGGCGTGGGAGCGTCCGGTGGCGCTGGAGGCGGAAC
TGGATCTGACCCTGAAAGTGCTGGAAAACATGACCGACAGCAGCCTGGGT
GTTACCCTGGATCAACCGCTGCGTACCCTGCACCACATTCATAGCGAGCT
GCAGGCGTGCGTTCCGGCGCAACCGACCGCGGACCCGCGTCCGCATGGTC
GTCTGCACCACTGGCTGCACCGTCTGCAGAAGGCGCCGAAAGAGAGCCAA
GGCTGCCTGGAAGCGAGCATTACCTTCAACCTGTTTCGTCTGCTGACCCG
TGACCTGAAGTGCGTGGCGAGCCGTGATCTGTGCGTTTAATGAAAGCTT

Gene Synthesis and Subcloning: Target DNA sequence of EXLT-07 Canine IFNL3 mature amino acid sequence was optimized and synthesized. The synthesized sequence was cloned into vector pET-30a(+) with His tag for protein expression in E. coli. E. coli strain BL21 star (DE3) was transformed with recombinant plasmid. A single colony was inoculated into TB medium containing related antibiotic; culture was incubated in 37° C. at 200 rpm and then induced with IPTG. SDS-PAGE was used to monitor the expression. Recombinant BL21 star (DE3) stored in glycerol was inoculated into TB medium containing related antibiotic and cultured at 37° C. When the OD600 reached about 1.2, cell culture was induced with IPTG at 15° C./16 h. Cells were harvested by centrifugation.

Purification and Analysis: Cell pellets were resuspended with lysis buffer followed by sonication. The precipitate after centrifugation was dissolved using denaturing agent. Target protein was sterilized by 0.22 m filter before stored in aliquots. The concentration was determined by Bradford protein assay with BSA as standard. The protein purity and molecular weight were determined by standard SDS-PAGE along with Western blot confirmation.

Freeze-Thaw Testing: Freeze-thaw testing was conducted by freezing sample (approximately −80° C.) for more than 4 hours, followed by thawing it at room temperature. After two cycles of freeze-thaw testing, the sample was checked after centrifugation. No visible precipitation was observed.

EXLT-08 Feline IFNL3 mature amino acid sequence NdeI-ATG-His tag-EXLT-08 Feline IFNL3 mature amino acid sequence-Stop codon-HindIII. Protein Length=142; MW=16179.7; Predicted pI=7.93 vector: pET30a

Amino acid sequence:
(SEQ ID NO: 8)
MHHHHHHLQKESVAPQAWTCRPCLFPKTRELRQLQVRERSVALEAELALT
LKVLGTVADASQGDILDQPLHTLCHIHSELWACVSTRPPAGPQTRGHLHH
WLPWLHKAPKKSLSCLEASVMFNLFYLLIWDLKGVTSEDPCV
DNA sequence: 441bp
(SEQ ID NO: 17)
CATATGCACCACCACCACCACCACTTACAAAAAGAATCAGTAGCTCCCCA
AGCGTGGACCTGCCGTCCGTGCCTGTTCCCGAAAACCCGTGAGCTGCGTC
AGCTGCAAGTGCGTGAACGTAGCGTTGCGCTGGAGGCGGAACTGGCGCTG
ACCCTGAAGGTTCTGGGCACCGTTGCGGATGCGAGCCAGGGTGACATCCT
GGATCAACCGCTGCACACCCTGTGCCACATTCACAGCGAACTGTGGGCGT
GCGTGAGCACCCGTCCGCCGGCGGGTCCGCAAACCCGTGGTCACCTGCAC
CATTGGCTGCCGTGGCTGCACAAGGCGCCGAAGAAAAGCCTGAGCTGCCT
GGAGGCGAGCGTTATGTTCAACCTGTTTTACCTGCTGATCTGGGACCTGA
AAGGTGTGACCAGCGAAGATCCGTGCGTTTAATGAAAGCTT

Gene Synthesis and Subcloning: Target DNA sequence of EXLT-08 Feline IFNL3 mature amino acid sequence was optimized and synthesized. The synthesized sequence was cloned into vector pET-30a(+) with His tag for protein expression in E. coli. E. coli strain BL21 star (DE3) was transformed with recombinant plasmid. A single colony was inoculated into 5052 auto-induced medium containing related antibiotic; culture was incubated in 37° C. at 200 rpm and then induced with IPTG. SDS-PAGE was used to monitor the expression. Recombinant BL21 star (DE3) stored in glycerol was inoculated into 5052 auto-induced medium containing related antibiotic and cultured at 37° C. When the OD600 reached about 3, cell culture was cultured at 15° C./16 h. Cells were harvested by centrifugation.

Purification and Analysis: Cell pellets were resuspended with lysis buffer followed by sonication. The precipitate after centrifugation was dissolved using denaturing agent. Target protein was sterilized by 0.22 m filter before stored in aliquots. The concentration was determined by BCA™ protein assay with BSA as standard. The protein purity and molecular weight were determined by standard SDS-PAGE along with western blot confirmation.

EXLT-09 Ovine IFNL3 mature amino acid sequence

NdeI-ATG-His tag-EXLT-09 Ovine IFNL3 mature amino acid sequence-Stop codon-HindIII.
Protein Length=179 MW=20075.0 Predicted pI=8.53 vector: pET30a

Amino acid sequence:
(SEQ ID NO: 9)
MHHHHHHPVPSAPRALPPARGCHMAQFKSLSPQELQAFKTVRDAFEDSRL
QKDWDCGGRRAALTCLLSPPQVWERPVALEAELALTLTVLEAMANSSLGR
SLEQPLLTLQHVHSKLQACVPAQPTAGPRPRGRLHHWLHRLQEAQKKESQ
DCLEASVMFNLFRLLTRDLKCVASGDQCV
DNA sequence: 552bp
(SEQ ID NO: 18)
CATATGCACCACCACCATCACCACCCCGTACCATCAGCTCCCAGAGCACT
CCCGCCGGCGCGTGGTTGCCACATGGCGCAGTTCAAAAGCCTGAGCCCGC
AGGAGCTGCAAGCGTTCAAGACCGTGCGTGACGCGTTTGAAGATAGCCGT
CTGCAGAAAGATTGGGATTGCGGTGGCCGTCGTGCGGCGCTGACCTGCCT
GCTGAGCCCGCCGCAAGTTTGGGAGCGTCCGGTTGCGCTGGAGGCGGAAC
TGGCGCTGACCCTGACCGTTCTGGAGGCGATGGCGAACAGCAGCCTGGGT
CGTAGCCTGGAACAGCCGCTGCTGACCCTGCAACACGTGCACAGCAAGCT
GCAGGCGTGCGTTCCGGCGCAACCGACCGCGGGTCCGCGTCCGCGTGGCC
GTCTGCACCACTGGCTGCACCGTCTGCAGGAAGCGCAAAAGAAAGAAAGC
CAGGACTGCCTGGAAGCGAGCGTGATGTTCAACCTGTTCCGTCTGCTGAC
CCGTGACCTGAAATGCGTTGCGAGCGGCGACCAATGCGTTTAATGAAAGC
TT

Gene Synthesis and Subcloning: Target DNA sequence of EXLT-09 Ovine IFNL3 mature amino acid sequence was optimized and synthesized. The synthesized sequence was cloned into vector pET-30a(+) with His tag for protein expression in E. coli. E. coli strain BL21 star (DE3) was transformed with recombinant plasmid. A single colony was inoculated into TB medium containing related antibiotic; culture was incubated in 37° C. at 200 rpm and then induced with IPTG. SDS-PAGE was used to monitor the expression. Recombinant BL21 star (DE3) stored in glycerol was inoculated into TB medium containing related antibiotic and cultured at 37° C. When the OD600 reached about 1.2, cell culture was induced with IPTG at 15° C./16 h. Cells were harvested by centrifugation.

Purification and Analysis: Cell pellets were resuspended with lysis buffer followed by sonication. The precipitate after centrifugation was dissolved using denaturing agent. Target protein was sterilized by 0.22 m filter before stored in aliquots. The concentration was determined by Bradford protein assay with BSA as standard. The protein purity and molecular weight were determined by standard SDS-PAGE along with Western blot confirmation.

Freeze-Thaw Testing: Freeze-thaw testing was conducted by freezing sample (approximately −80° C.) for more than 4 hours, followed by thawing it at room temperature. After two cycles of freeze-thaw testing, the sample was checked after centrifugation. No visible precipitation was observed.

### Example 3

PEGylation of IFNL3: EXLT-01 and EXLT-02 (as well as EXLT 3-EXLT 9) have a poly-His tag on the N-terminus. Accordingly, a selective procedure was used to accomplish N-terminus PEGylation, and this procedure is useful to produce all of the IFNL3 proteins disclosed herein including EXLT-01, EXLT-02, EXLT-03, EXLT-04, EXLT-05, EXLT-06, EXLT-07, EXLT-08, and EXLT-09. The synthesis method described in Bioconjugate Chem. 2012, 23 (2), 248-263, which is incorporated herein by reference, was used. The protein was treated with a PEG-functionalized bis-Michael acceptor at pH 5.3 to form bonds between the histidine residues and the PEG units (illustrated for EXLT-01 in Scheme 1).

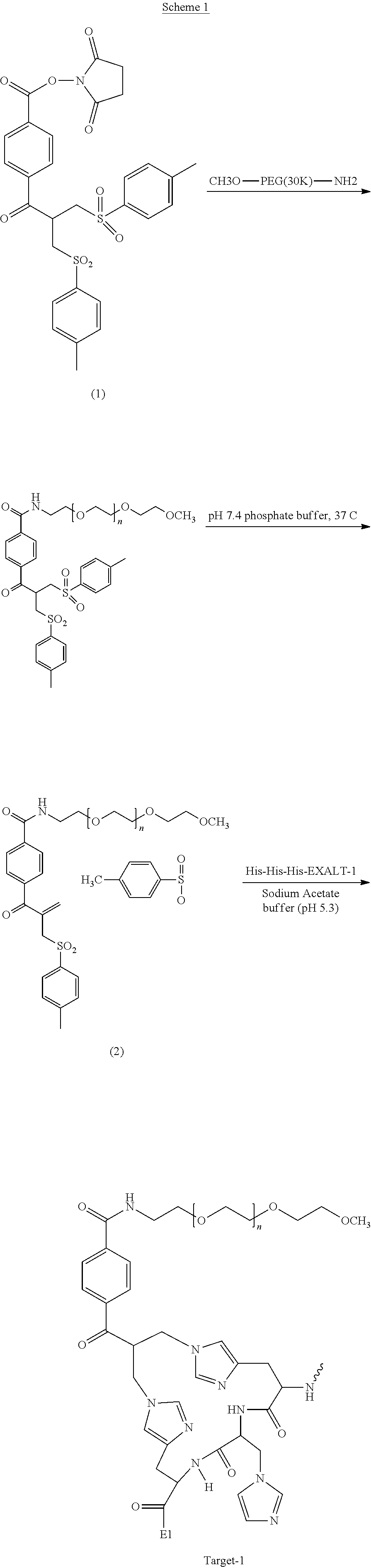

The precursor (1) was generated as described in Bioconjugate Chem. 2007, 18 (1), 61-76. and Nat. Chem. Bio. 2006, 312-313, both of which are incorporated herein by reference. Incubation of (1) at pH 7.4 phosphate buffer at 37° C. gave the monosulfonate (2) in situ and it was added to 0.1 equivalents of either EXLT-01 or EXLT-02 in sodium acetate buffer (pH 5.3) at 21° C. After 17 hour of incubation the PEGylated proteins were isolated, purified and characterized.

Precursor 1. To a solution of bis-sulfone NHS ester (BroadPharm, San Diego, Calif.) in dichloromethane at 0° C. added 0.2 molar equivalents of CH3O-PEG(30K)—NH2 (NANOCS, N.Y.). The resulting mixture was allowed to warm up to room temperature and stirred for additional 48 h. The solvent was evaporated under reduced pressure and dry acetone was added to afford Precursor-1 (CR-JGG-I-067). Precursor 1 was stored in the refrigerator at 4° C. and the precipitate that formed was filtered and rinsed with cold acetone. The solid was then vacuum dried to provided Precursor 1 as a solid that showed a single UV-active product by thin layer chromatography (Rf=0.71, 9:1 dichloromethane:methanol, silica gel).

PEGylation of EXLT-01: A solution of 2 molar equivalents of (1) as a 20 mg/mL solution in pH 7.4 phosphate buffer was incubated at 37° C. for 8 h. 1 was added to a solution of 7.48 mg of EXALT-1 in pH 5.3 buffer and incubated at 21° C. for 18 h. The reaction was cooled to 0° C. in an ice water bath. A single portion of 5 molar equivalents of sodium triacetoxyborohydride (NaBH(OAc)₃ was added. The mixture was stirred for an additional 1 h at 4° C. to afford CR-JGG-I-070 (pegylated EXLT-01).

PEGylation of EXLT-02: A solution of 2 molar equivalents of (1) as a 20 mg/mL solution in pH 7.4 phosphate buffer was incubated at 37° C. for 8 h. 1 was added to a solution of 4.9 mg of EXLT-01 in pH 5.3 buffer and incubated at 21° C. for 18 h. The reaction was cooled to 0° C. in an ice water bath a single portion of 5 molar equivalents of sodium triacetoxyborohydride (NaBH(OAc)₃ was added. The mixture was stirred for an additional 1 h at 4° C. to afford CR-JGG-I-071 (pegylated EXLT-02).

Purification of EXLT-01 and EXLT-02: In order to separate PEGylated protein from unlabeled protein and extra PEG, the mixture was purified using an Amicon Ultra Centrifugal Filter 50 kDa MWCO (EMD Millipore, Burlington, Mass.) followed by three washes using 50 mM sodium phosphate buffer (150 mM NaCl, pH 7.5 with 0.2 mg/mL polysorbate 80). The protein concentration of all the fractions were determined by BCA protein assay (Thermo Fisher, Waltham, Mass.) and the fractions were analyzed by SDS-PAGE followed by colloidal blue staining (Thermo Fisher, Waltham, Mass.) for proteins and barium iodide staining as described in Anal. Biochem. 1992, 200 (2), 244-248, which is incorporated herein by reference, for PEGylated product.

Barium iodide staining showed strong bands at −50 kDa (correct size for mono-30 kDa PEG-EXLT-01 or EXLT-02). Samples that were washed three times with sodium phosphate buffer removed most (>95%) of the unlabeled EXLT-01 or EXLT-02. Extra unreacted PEG (at 30 kDa) was not clearly detected by barium iodide staining. Based on barium iodide staining and a standard curve of unlabeled EXLT-01, the protein concentration was estimated and the protein samples were aliquoted and flash frozen in liquid nitrogen and stored at −80° C. Accordingly, this selective procedure which was used to accomplish N-terminus PEGylation, may be used to produce all of the IFNL3 proteins disclosed herein including EXLT-01, EXLT-02, EXLT-03, EXLT-04, EXLT-05, EXLT-06, EXLT-07, EXLT-08, and EXLT-09.

An alternative PEGylation process suitable for use with the IFNL3 polypeptides of the present invention is performed using a 30 kDa PEG reagent with an appropriate functionalization for coupling to its N-terminal extremity, for example using the standard PEGylation technology such as CyPEG™, HiPEG™ and TheraPEG™ (Abzena).

PEGylation of IFNL3 is performed in the following stages:

(i) Sufficient 30 kDa HiPEG™ (Abzena) reagent is synthesized with a suitable functionality for coupling to the His-tag of IFNL3;
(ii) Small-scale conjugation and purification screen of IFNL3 is performed with 30 kDa PEG reagent;
(iii) Analytical methods will be used for determining the purity of the PEGylated IFNL3 (e.g., SEC and/or SDS-PAGE/HIC);
(iv) Based on the results from the small-scale conjugation, larger-scale conjugation and purification of IFN1 is performed with the 30 kDa PEG reagent;
(v) PEGylated IFNL3 is characterized for purity (SEC, SDS-PAGE, HIC) and quantified;
(vi) Purified 30 kDa PEGylated IFNL3 is tested for antivirus activity.

Freeze-thaw evaluation is performed as follows to assess stability of the product. 30 kDa PEGylated IFNL3 is evaluated by SEC after 2 and 3 freeze-thaw cycles. The 30 kDa PEGylated IFNL3 will be stored at 22° C. and analyzed at 24 h and 48 h by SEC.

### Example 4

In vitro cytotoxicity and anti-virus testing for all species of IFNL3 was generally performed as follows.

The IFNL3 polypeptides in multiple dilutions were analyzed for any cytotoxic effect on epithelial cell lines (MDBK for bovine IFNL3). Each test article polypeptide was evaluated at 8 different 4-fold dilutions starting with a concentration of 1 μg/ml. Each test article concentration was assayed in triplicate. Cell lines were seeded in 24 (or 96) well plates and exposed to test article polypeptides dilutions in medium (400 μl/well for 24 well plates) for 24 hours after which the cells were evaluated visually using light microscopy. Cell density and percent viability was also determined using the MTT viability assay kit purchased from Biotium (catalog no. 30006) and following the manufacturer's instructions. All of the IFNL3 proteins for all species of IFNL3 were tested in the MTT assay, and all cell lines retained at least 95% viability following exposure to the test article polypeptides. Anti-virus testing was initiated following confirmation of non-toxicity of the IFNL3 proteins in the MTT assay.

Analysis for cytotoxic effects of EXLT-01 on MDBK cells: A cell viability assay using standard techniques was performed to test EXLT-01 or any cytotoxic effect on the bovine epithelial cell lines MDBK. Cells were seeded in 6 well plates and exposed to test article polypeptides dilutions in medium (400 ul/well) for 24 hours after which the cells were evaluated visually using light microscopy. Cell density and percent viability was a determined using the Biotium MTT Cell Viability Assay Kit according to kit insert instructions. The cells retained at least 95% viability following exposure to the test article polypeptides, thus anti-virus testing was initiated. Each of EXLT-01, EXLT-04, EXLT-05, EXLT-06, EXLT-07, EXLT-08, and EXLT-09 were tested for cytotoxicity in the assay described. The results show that all IFNL3 proteins had no cytotoxicity on the cells directly, with all cells tested having greater than 95% viability (see Table 6).

TABLE 6

| EXLT | | | Viability by |
|---|---|---|---|
| compound | Species | Cell Line | MTT |
| EXLT-01 | Bovine | MDBK | >95% |
| EXLT-02 | Bovine | MDBK | >95% |
| EXLT-03 | Bovine | MDBK | >95% |
| EXLT-04 | Porcine | PK-15 | >95% |
| EXLT-06 | Equine | ED | >95% |
| EXLT-07 | Canine | MDCK | >95% |
| EXLT-08 | Feline | CRFK | >95% |
| EXLT-09 | Ovine | OAEC | >95% |

### Example 5

To perform a virus plaque inhibition assay, 10-fold dilutions of a virus stock were prepared, and 0.1 ml aliquots were inoculated onto susceptible cell monolayers. Cells were then treated with the appropriate species of IFNL3 protein for 12 to 24 hours incubation. After the initial incubation period a known titer of the appropriate virus for each species of IFNL3 was added to the cells and incubated to allow virus to attach to cells. The monolayers were covered with a nutrient medium containing soft agar that causes the formation of a gel. When the plates were incubated, the originally infected cells released viral progeny. The spread of the new viruses was restricted to neighboring cells by the gel. Consequently, each infectious particle produced a circular zone of infected cells called a plaque. Eventually the plaque became large enough to be visible to the naked eye. Dyes that stain living cells can be used to enhance the contrast between the living cells and the plaques. Only viruses that cause visible damage of cells were assayed in this way.

The degree of inhibition of virus replication was calculated by comparing the number of plaques observed in untreated controls with IFNL3 treated cells. To minimize error, only plates containing between 10 and 100 plaques were counted, depending on the size of the cell culture plate that is used. Each dilution was plated in triplicate to enhance accuracy.

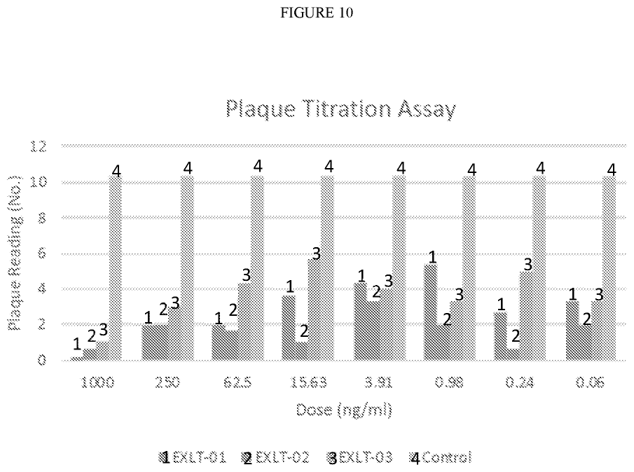

Determination of EXLT-01, EXLT-02, and EXLT-03 biological activity against BVD and BTV in vitro: The recombinant proteins were tested for their ability to inhibit virus replication in a plaque reduction assay using standard techniques. To perform the plaque assay, cells were treated with EXLT-01 at concentrations of 25 ng/ml, 50 ng/ml, 500 ng/ml, and 1000 ng/ml in cell growth media for a total of 1.5 ml EXLT-01 dilution per well and incubated 12 to 24 hours at 37° C. Each concentration was performed in a 6 well plate in triplicate. An untreated 6 well plate and a non-infected 6 well plate were set as positive and negative controls respectively. After incubation, the EXLT-01 solution was removed. Virus that was diluted to a concentration of 20-50 PFU in cell growth media, with enough virus for inoculation with 0.2 ml per well, was applied to the wells and incubated at 37° C. for 90 minutes. The cell monolayers were then covered with a 1.2% agarose in cell growth medium and incubated at 37° C. and 5% CO2 for 3-4 days and observed for cytopathic effects. Following incubation, a second overlay of 1.2% agarose in cell growth media and 2% neutral red was added to the wells and incubated at 37° C. and 5% CO2 for an additional 3-4 days. Plaques were counted day 6 through 9 of incubation post-infection. To minimize error, only plates containing between 10 and 100 plaques were counted. Statistical principles dictate that when 100 plaques are counted, the sample titer will vary by plus or minus 10%. As shown in FIG. 10 each of EXLT-01, EXLT-02, and EXLT-03 demonstrated potent antivirus replication activity in the virus plaque inhibition assay. It was also shown in FIG. 10 that the deletion of 6 amino acids from the C-terminus of the protein (EXLT-03) caused a reduction in the antivirus replication activity, but that deletion of 4 amino acids from the C-terminus (EXLT-02) did not reduce the antivirus activity compared to EXLT-01.

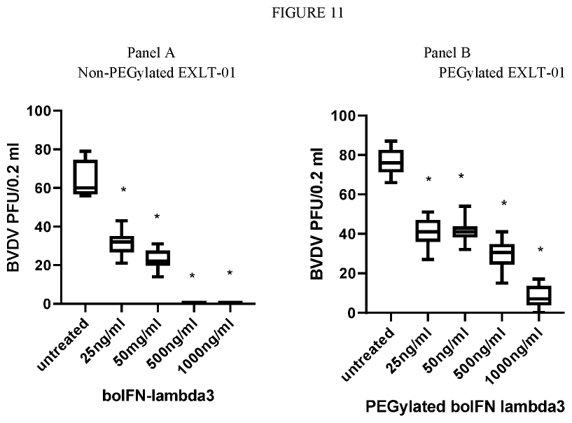

Determination of PEGylated EXLT-01 activity against BVD in vitro: EXLT-01 that has been PEGylated was tested in the virus plaque assay to determine retention of potency as described above. EXLT-01 Demonstrates antiviral activity against BVDV in vitro. We tested the bioactivity of EXLT-Olin MDBK cells using BVDV. We used this cell type as it is optimal in evaluating plaques and is an epithelial cell type that is targeted by EXLT-01. The BVDV strain that was used. Treatment of the cells with EXLT-01 reduced BVDV plaque forming units in a dose dependent manner from blank PFUs in the control to blank PFUs with statistically significant differences among the tested concentrations (p<0.05) (FIG. 11).

PEGylated EXLT-01 demonstrated antiviral activity against BVDV in vitro. As chemical modification can alter the biological effects of a therapeutic, we additionally assessed the antiviral effects of PEGylated EXLT-01. Similarly, the bioactivity of PEGylated EXLT-01 was assessed in MDBK cells applying BVDV as the challenge virus. Reduced BVDV plaque forming units were observed in a dose dependent manner from blank PFUs in the control to blank PFUs. Although the overall PFU formation was higher in the PEGylated EXLT-01, and the PEGylated EXLT-01 also demonstrated statistically significant differences among the tested concentrations (p<0.05) (FIG. 11). We concluded that both EXLT-01 and PEGylated EXLT-01 exhibit antiviral activity against BVDV in MDBK cell culture.

The same procedure was performed using EXLT-01 (bovine IFNL3) on MDBK cells with blue tongue virus (BTV). In this virus plaque inhibition assay, EXLT-01 showed a 25%-35% reduction of plaques, demonstrating the significant antivirus activity of EXLT-01 against BTV (see Table 7).

TABLE 7
BTV Plaque Reduction Assay Using EXLT-01

| EXLT-01 concentration | Plaque | Percent |
|---|---|---|
| (ng/ml) | Number | Inhibition |
| 0 | 26.4 | — |
| 31 | 19.7 | 25.4 |
| 62.5 | 18.5 | 30.8 |
| 125 | 14 | 47 |
| 250 | 10.3 | 61 |
| 500 | 7 | 73.5 |
| 750 | 3.7 | 86 |
| 1000 | 2.2 | 91.7 |
| 1250 | 1.5 | 94.4 |

The same procedure was performed using EXLT-06 (equine IFNL3) on ED cells with equine herpes virus (EHV). In this virus plaque inhibition assay, EXLT-06 showed about 25%-35% reduction of plaques, demonstrating the significant antivirus activity of EXLT-06 against EHV (see Table 8).

TABLE 8
EHV Plaque Reduction Assay Using EXLT-06

| EXLT-06 concentration | Plaque | Percent |
|---|---|---|
| (ng/ml) | Number | Inhibition |
| 0 | 41.7 | — |
| 62.5 | 33.8 | 19 |
| 250 | 26.5 | 36.5 |
| 1000 | 30.8 | 26.2 |

### Example 6

Pharmacokinetics of recombinant IFNL3, PKEMylated recombinant IFNL3, PEGylated recombinant IFNL3, or Acylated recombinant IFNL3.

In vivo studies are conducted in mice, rats, dogs, cows, horses, chickens, cats, and any other animal species to characterize the pharmacokinetics (PK) of the IFNL3 polypeptides of the present invention after intravenous (IV) and/or subcutaneous (s.c.) dosing. In some embodiments, recombinant IFNL3, PKEMylated recombinant IFNL3, PEGylated recombinant IFNL3, or Acylated recombinant IFNL3 polypeptides of the invention are observed to exhibit increased in vivo half-life relative to wild-type IFNL3.

Formulation: The IFNL3 test compound vehicle is PBS. Animal Dosing Design—In vivo PK, nonfasted animals Group 1: 3 animals per group+Control animals (for drug-free blood collection), n=2 rats. Plasma Sample Collection from rats (serial sampling): Blood aliquots (300 μL) are collected from jugular vein catheterized rats in tubes coated with lithium heparin, mixed gently, then kept on ice and centrifuged at 2,500×g for 15 minutes at 4° C., within 1 hour of collection. For control animals, blood is collected by cardiac puncture. The plasma is then harvested and kept frozen at −70° C. until further processing.

Quantitative Bioanalysis for Plasma: Rat drug levels in plasma are measured by a commercially available species-specific ELISA Kit (for example, bovine IFNL3 ELISA kit from MyBiospace, Cat.no. MBS100690; porcine IFNL3 ELISA kit from MyBiospace cat.no. MBS082640; sheep IFNL3 ELISA kit from MyBiospace cat.no. MBS088340; chicken ELISA kit from Creative Biomart cat.no. IFNL3-3848c, canine IFNL3 ELISA kit from MyBiospace cat.no. MBS606667) or similar kit from other manufacturers. The procedures are shown in kit instruction sheet. A plasma calibration curve is generated. Aliquots of drug-free plasma are spiked with the test compound at the specified concentration levels. The spiked plasma samples are processed together with the unknown plasma samples using the same procedure. The processed plasma samples are stored at −70° C. until the ELISA analysis, at which time the concentrations of the test compound in the unknown plasma samples are determined using the respective calibration curve. The reportable linear range of the assay is determined, along with the lower limit of quantitation.

Pharmacokinetics: Plots of plasma concentration of IFNL3 compounds versus time are constructed. The fundamental pharmacokinetic parameters of compound after intravenous (AUClast, AUCINF, T1/2, Cl, Vz, Vss, Tmax, and Cmax) are obtained from the non-compartmental analysis (NCA) of the plasma data using WinNonlin. Standard, commercially available or custom prepared ELISA assays are used to measure IFNL3 concentration in serum following the manufacturer's instructions.

Mice: In mice, the PK of the modified IFNL3 polypeptide and wild-type IFNL3 is determined following both i.v. (1 mg/kg) and s.c. (1 mg/kg) administration. Three mice are bled at each time point and serum samples are analyzed by the ELISA method or by IFNL3 activity method.

Rats: Sprague-Dawley rats are also dosed with wild-type and modified IFNL3 polypeptides (i.v., 1 mg/kg; s.c., 1 mg/kg) and the PK profile determined. Three rats are bled at each time point and serum samples are analyzed by the ELISA method or by IFNL3 activity method.

Beagle dogs: In beagle dogs, the PK of the IFNL3 or modified IFNL3 polypeptide and wild-type IFNL3 is determined following both i.v. (1 mg/kg) and s.c. (1 mg/kg) administration. Two dogs are bled at each time point after i.v. dosing and one dog per dose group is bled after s.c. dosing.

Other Corresponding Species: In the species of animal that corresponds to the species of IFNL3 (e.g. cow, pig, horse, sheep, dog, cat, chicken) the PK of the IFNL3 or modified IFNL3 polypeptide and wild-type IFNL3 is determined following both i.v. (1 mg/kg) and s.c. (0.2 mg/kg) administration. Two or more animals are bled at each time point and serum samples are analyzed by the ELISA method or IFNL3 activity method.

### Example 6

Generally applicable anti-virus assay: Plaque inhibition assay of the various species of IFNL3 proteins of the present invention are each tested for the ability to inhibit plaque formation in infected cells as generally described below.

1. Dilute products. Each of the IFNL3 protein products at the stock concentration of 500 μg/ml are first diluted in cell culture medium to generate the concentration of 1 μg/ml (1000 ng/ml) and then serially 4-fold diluted to generate the concentrations of 250, 62.5, 15.63, 3.91, 0.98, 0.24 and 0.06 ng/ml.
2. Cells are prepared in 12-well plates one day before treatment. On the day of testing, culture medium is removed from each well of cells. 800 μl of each IFNL3 protein product at the concentrations of 1000, 250, 62.5, 15.63, 3.91, 0.98, 0.24 and 0.06 ng/ml is added to each well of cells (duplicate or triplicate wells for each product at each dilution).
3. Incubate the plates at 37° C. in 5% CO2 incubator for 24 h prior to virus infection.

### Virus Plaque Assay Protocol

1. Prepare dilutions of virus in cell culture medium. Pipette at least 10 μl of virus or solution to reduce pipetting error.
2. Add virus dilution [100 μl-10 μl] in duplicate to each well, letting the virus flow gently into the media.
3. Incubate the infected monolayers at 37° C. for four to sixteen hours; mildly shake the plates gently several times during this adsorption period.

### Agar Overlay

1. Prepare a sterile solution of 4% agarose in dH2O by autoclaving at 121° C. for 20 minutes. Agarose may be stored on the shelf at room temp or used immediately after equilibrating in a 65° C. water bath.
2. Warm the plaquing media (see above, 2 ml per well to be overlaid) in a 37° C. water bath until equilibrated.
3. Gently draw media out of each infected monolayer well and discard.
4. Mix the volume of media (i.e. 5 plates, 30 wells, 60 ml) in a 37° C. pre-warmed container and add 0.11 volumes of liquid agarose to the bottle with swirling (1:10 dilution). Shake vigorously to mix.
5. With a new pipette, immediately but gently add 2 ml of the agarose/growth media mixture to each well, pipetting it down the side of the well.
6. Let the plate(s) sit for 15 minutes in the level hood at RT as the agar overlay turns solid.
7. Move the plate(s) to a humidified incubator at 37° C. and 7.5 to 10% CO2. Some viruses require different temperatures.

### Plaque Visualization

Plaques will be visible by day 5 to 7 after infection, and the monolayer of cells can be stained if desired to visualize and count the plaques on the final day of plaque development. Stain the plate(s) at 5-6 days post infection or when plaques have fully developed. Stain for 3 hr at 37° C. by adding 0.1 volume of MTT solution (5 mg/ml in PBS) (Sigma, M2128). The monolayer will appear blue/black and plaques will be clear areas.

Using the above assay, the IFNL3 and modified IFNL3 proteins of the present invention are characterized for concentration response, antivirus replication response, and potency. Protein stability will be determined under different temperature and storage conditions.

### Example 7

Pharmacokinetic Studies of C14-Acylated IFNL3s

The C14-acylated IFNL3s described herein and tested for activity and specificity in vitro as described herein are further tested to determine pharmacokinetic (PK) properties in the mouse and/or rat. As in the preceding examples, the modified IFNL3 polypeptides each consisted of the IFNL3 polypeptide of SEQ ID NO: 1, 2, 3, 4, 5, 6, 7, 8, or 9 linked to a linker comprising the PKEM, C12 or C14 acyl group, or a water soluble polymer such as PEG.

Methods: Coarse (three sampling time points) intravenous (i.v.) pharmacokinetics are investigated in male C57BL6 mice for the various IFNL3 polypeptides described above following a single i.v. bolus injection at a dose of 0.2 mg/kg (N=3 each time point, 5 mL/kg). Each IFNL3 protein is dosed as a solution in 0.2 M Tris, 1 M NaCl, 25% propylene glycol, pH 8.5 through injection into retro-orbital sinus. Serial blood samples are collected by retro-orbital bleeding or heart puncture at 1, 3 and 6 hours post dose.

Full i.v. pharmacokinetics is are also investigated in male Balb/c mice for PKEMylated IFNL3, C12 or C14-IFNL3 or PEG-IFNL3 following a single tail vein bolus injection at a dose of 0.5 mg/kg (N=3 each time point, 5 mL/kg). PKEMylated IFNL3, C12 or C14-IFNL3 or PEG-IFNL3 is dosed as a solution in 0.2 M Tris, 1 M NaCl, 25% propylene glycol, pH 8.5 and the blood samples are collected through retro-orbital bleeding at 0.05, 0.25, 0.5, 1, 3, 5, 7, 9, 24 hours post i.v. dose (0.05, 1, 7 h from one group of 3 mice, 0.25, 3, 9 h from the second group of 3 mice and 0.5, 5, 24 h from the third group of 3 mice).

Subcutaneous (s.c.) pharmacokinetics is investigated in male Balb/c mice for PKEMylated IFNL3, C12 or C14-IFNL3 or PEG-IFNL3 following a single s.c. injection at a dose of 1 mg/kg (N=3 each time point, 7 mL/kg). Each drug is dosed as a solution in 0.2 M Tris, 1 M NaCl, 25% propylene glycol, pH 8.5 and the blood samples are collected through retro-orbital bleeding at 0.25, 0.5, 1, 3, 7, and 24 hours post dose (0.25, 1, 7 h from one group of 3 mice, and 0.5, 3, 24 h from the other group of 3 mice). Additionally, intravenous pharmacokinetics is investigated in male Sprague-Dawley (SD) rats for PKEMylated IFNL3, C12 or C14-IFNL3 or PEG-IFNL3 following a 5 min i.v. infusion via the jugular vein at a dose of 0.5 mg/kg (N=3 rats, 3.57 mL/kg). PKEMylated IFNL3, C12 or C14-IFNL3 or PEG-IFNL3 is dosed as a solution in 0.2 M Tris, 1 M NaCl, 25% propylene glycol, pH 8.5 and serial blood samples are collected through direct tail vein bleeding at pre-dose, 0.167, 0.25, 0.5, 1, 3, 5, 7, 24, 48 hours post i.v. dose.

For the mouse samples, following collection, blood samples are centrifuged at 10,000 rpm for 10 min at 4° C. to obtain serum and serum samples are stored at −20° C. until analysis. The serum concentration of acylated IFNL3 is analyzed by enzyme-linked immunosorbent assay (ELISA) or IFNL3 activity method. Concentrations are calculated using a standard curve generated from the corresponding dosed compound. Pharmacokinetic parameters are estimated using non-compartmental analysis by Kinetica software (Thermo Fisher Scientific Corporation, version 5.0). The peak concentration (Cₘₐₓ) and time for Cₘₐₓ (Tₘₐₓ) are recorded directly from experimental observations. The area under the curve from time zero to the last sampling time [AUCₗₐₛₜ] and the area under the curve from time zero to infinity [AUCₜₒₜₐₗ] are calculated using a combination of linear and log trapezoidal summations. The total plasma clearance, steady-state volume of distribution (Vss), apparent elimination half-life (t_{half}), and mean residence time (MRT) are estimated after i.v. administration. Estimations of AUC and t_{half} are made using a minimum of 3 time points with quantifiable concentrations. The absolute s.c. bioavailability (F) is estimated as the ratio of dose-normalized AUC values following s.c. and i.v. doses. The PK parameters are calculated when applicable.

For the rat samples, following collection, blood samples are centrifuged at 10,000 rpm for 10 min at 4° C. to obtain serum and serum samples are stored at −20° C. until analysis. The serum concentration of acylated IFNL3 is analyzed by enzyme-linked immunosorbent assay (ELISA) or IFNL3 activity method. Concentrations are calculated using a standard curve generated from the corresponding dosed compound. Pharmacokinetic parameters are estimated using non-compartmental analysis by Kinetica software (Thermo Fisher Scientific Corporation, version 5.0). The peak concentration (Cₘₐₓ) and time for Cₘₐₓ (Tₘₐₓ) are recorded directly from experimental observations. The area under the curve from time zero to the last sampling time [AUCₗₐₛₜ] and the area under the curve from time zero to infinity [AUCₜₒₜₐₗ] are calculated using a combination of linear and log trapezoidal summations. The total plasma clearance, steady-state volume of distribution (Vss), apparent elimination half-life (t_{half}), and mean residence time (MRT) are estimated after i.v. administration. Estimations of AUC and t_{half} are made using a minimum of 3 time points with quantifiable concentrations. The absolute s.c. bioavailability (F) is estimated as the ratio of dose-normalized AUC values following s.c. and i.v. doses. The PK parameters are calculated when applicable.

While the invention has been described by way of examples and preferred embodiments, it is understood that the words which have been used herein are words of description, rather than words of limitation. It is understood that the examples and embodiments described herein are for illustrative purposes only and that various modifications or changes in light thereof will be suggested to those of ordinary skill in the art and are to be included within the spirit and purview of this application and scope of the appended claims. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims. All publications, patents, patent applications, and/or other documents cited in this application are incorporated by reference in their entirety for all purposes to the same extent as if each individual publication, patent, patent application, and/or other document are individually indicated to be incorporated by reference for all purposes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A non-human interferon lambda 3 (IFNL3) polypeptide or fragment thereof comprising at least one pharmacokinetic enhancing moiety (PKEM) and a polypeptide comprising a sequence having at least 80% identity to SEQ ID NO: 1; SEQ ID NO:2; SEQ ID NO:3; SEQ ID NO 4; SEQ ID NO 5; SEQ ID NO: 6; SEQ ID NO: 7; SEQ ID NO: 8; or SEQ ID NO: 9, or fragment thereof, wherein said IFNL3 polypeptide or fragment thereof is biologically active.

2. The IFNL3 polypeptide of claim 1, having at least one PKEM conjugated to a naturally occurring or non-naturally occurring amino acid of the IFNL3 polypeptide.

3. (canceled)

4. The IFNL3 polypeptide of claim 1, wherein said PKEM optionally comprises at least one acyl group, lipid, alkyl group, carbohydrate, polypeptide, polynucleotide, polysaccharide, antibody or antibody fragment, sialic acid(s), a prodrug, serum albumin, XTEN molecule, Fc molecule, adnectin, fibronectin, a biologically active molecule, water soluble polymer, one or more IFNL3 polypeptides, or a combination thereof

5. The IFNL3 polypeptide of claim 4, wherein the PKEM is a water soluble polymer selected from the group consisting of, polyethylene glycol, polyethylene glycol propionaldehyde, mono C1-C10 alkoxy or aryloxy derivatives thereof, monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, methylcellulose, carboxymethyl cellulose, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, or mixtures thereof, and has a molecular weight of between about 0.1 kDa and about 100 kDa.

6. The IFNL3 polypeptide of claim 5, wherein the water soluble polymer is polyethylene glycol and has a molecular weight selected from the group consisting of 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da.

7. The IFNL3 polypeptide of claim 2, wherein said PKEM comprises at least one acyl group and wherein said acyl group comprises a branched or unbranched C8-C30 acyl.

8. (canceled)

9. The IFNL3 polypeptide of claim 7, wherein said acyl group comprises a branched or unbranched C14 acyl, a branched or unbranched C16 acyl, a branched or unbranched C18 acyl, or a branched or unbranched C20 acyl.

10. (canceled)

11. (canceled)

12. The IFNL3 polypeptide of claim 2, wherein said PKEM comprises at least one alkyl group and wherein said alkyl group comprises a branched or unbranched C8-C30 alkyl.

13. (canceled)

14. (canceled)

15. The IFNL3 polypeptide of claim 12, wherein said alkyl group is a branched or unbranched C14, branched or unbranched C16, branched or unbranched C18, or branched or unbranched C20 alkyl group.

16. The IFNL3 polypeptide of claim 1, wherein said PKEM comprises at least one serum albumin molecule or Fc molecule.

17. (canceled)

18. The IFNL3 polypeptide of claim 16, wherein said PKEM is linked to the Cys 34 residue of said serum albumin.

19. The IFNL3 polypeptide of claim 1, wherein said PKEM comprises at least one XTEN molecule and wherein said XTEN molecule is fused with said IFNL3 polypeptide or is chemically conjugated to said IFNL3 polypeptide.

20-23. (canceled)

24. The IFNL3 polypeptide of claim 1, wherein said PKEM comprises at least one adnectin and wherein said adnectin comprises one or more of a BC loop, a DE loop, and an FG loop.

25. (canceled)

26. The IFNL3 polypeptide of claim 1 wherein said PKEM comprises at least one additional IFNL3 or modified IFNL3 polypeptide linked via their N-termini.

27. (canceled)

28. The IFNL3 polypeptide of claim 1, which exhibits an in vivo half-life of at least 1 hour.

29. (canceled)

30. The IFNL3 polypeptide of claim 1, which is attached to another biologically active moiety.

31. (canceled)

32. (canceled)

33. A pegylated non-human interferon lambda 3 (IFNL3) polypeptide or fragment thereof comprising a polypeptide having a sequence at least 80% identical to SEQ ID NO: 1; SEQ ID NO:2; SEQ ID NO:3; SEQ ID NO 4; SEQ ID NO 5; SEQ ID NO: 6; SEQ ID NO: 7; SEQ ID NO: 8; or SEQ ID NO: 9, wherein said polypeptide is covalently linked to at least one polyethylene glycol.

34. A composition comprising an IFNL3 polypeptide according to claim 1 and a pharmaceutically acceptable carrier.

35. (canceled)

36. A method of treating or preventing a disease in a non-human subject, comprising administering an IFNL3 polypeptide according to claim 1 to a subject in need thereof.

37. The method of claim 36, wherein the disease comprises a viral infection, a bacterial infection, inflammation, or parasitic infection.

38-48. (canceled)


SEQUENCE LISTING (20 sequences)

SEQ ID NO: 1 - protein, 183 aa, Artificial Sequence
  Description of Artificial Sequence Synthetic polypeptide
MHHHHHHRTGAVPVPSAPRALPPARGCHVAQFKSLSPQELQAFKTARDAFEDSFLPKDWDCSTHLFPRTRDLKHLQVWERPVALEAELALTLTVLEAMANSSLGHSLEQPLLTLQNIHSKLQACVPAQPTASSRPRGRLHHWLHRLQEARKESQDCLEASVMFNLLRLLTRDLKCVASGDQCV

SEQ ID NO: 2 - protein, 179 aa, Artificial Sequence
  Description of Artificial Sequence Synthetic polypeptide
MHHHHHHRTGAVPVPSAPRALPPARGCHVAQFKSLSPQELQAFKTARDAFEDSFLPKDWDCSTHLFPRTRDLKHLQVWERPVALEAELALTLTVLEAMANSSLGHSLEQPLLTLQNIHSKLQACVPAQPTASSRPRGRLHHWLHRLQEARKESQDCLEASVMFNLLRLLTRDLKCVASG

SEQ ID NO: 3 - protein, 177 aa, Artificial Sequence
  Description of Artificial Sequence Synthetic polypeptide
MHHHHHHRTGAVPVPSAPRALPPARGCHVAQFKSLSPQELQAFKTARDAFEDSFLPKDWDCSTHLFPRTRDLKHLQVWERPVALEAELALTLTVLEAMANSSLGHSLEQPLLTLQNIHSKLQACVPAQPTASSRPRGRLHHWLHRLQEARKESQDCLEASVMFNLLRLLTRDLKCVA

SEQ ID NO: 4 - protein, 178 aa, Artificial Sequence
  Description of Artificial Sequence Synthetic polypeptide
MHHHHHHPVPEALRALPGARGCHLAQFKSLSPQALQAFKRAKDAFEESLLEDWNCSSRIFPRSRDLKQLQVWERPVALEAEVALTLSVLGSLANSSLHSSLDQPLHTLRHIHAQLQACVPAQPMAGPRPRGRLHHWLHRLQEAQKKEPQSCLEASVMFNLFRLLTRDLKCVASGDLCV

SEQ ID NO: 5 - protein, 172 aa, Artificial Sequence
  Description of Artificial Sequence Synthetic polypeptide
MHHHHHHFPQVTPKKSCSLSKYQFPAPLELKAVWRMKEQFEDIMLLTNRKCNTRLFHRKWDIAELSVPDRITLVEAELDLTITVLTNPTTQRLAETCQQPLAFLTQVQEDLRDCLALEAPSHQPSGKLRHWLQKLETAKKKETAGCLEASAILHIFQVLNDLRCAAQREDCT

SEQ ID NO: 6 - protein, 177 aa, Artificial Sequence
  Description of Artificial Sequence Synthetic polypeptide
MHHHHHHGPVPRRHRALPGARGCHIAQYKSLSPGERLAFKNATDAFEESFWLKNHTCRSRPFPRTWDLRQLQVWERPVALEAELNLTLRVLANSSLGDVLDRPLGTLSHIHSELQACVAAQPTEGPRPRGRLHQWLRRLLEAQEEEPWGCLEASVMFNLFRLLTKDLTCVASGDLCV

SEQ ID NO: 7 - protein, 178 aa, Artificial Sequence
  Description of Artificial Sequence Synthetic polypeptide
MHHHHHHGPVPTSKPTTTRRGCHMDRFQSLSPRELEAFKKAKDALEESLSWKNWSCSSRLFPRSRDLRLLQAWERPVALEAELDLTLKVLENMTDSSLGVTLDQPLRTLHHIHSELQACVPAQPTADPRPHGRLHHWLHRLQKAPKESQGCLEASITFNLFRLLTRDLKCVASRDLCV

SEQ ID NO: 8 - protein, 142 aa, Artificial Sequence
  Description of Artificial Sequence Synthetic polypeptide
MHHHHHHLQKESVAPQAWTCRPCLFPKTRELRQLQVRERSVALEAELALTLKVLGTVADASQGDILDQPLHTLCHIHSELWACVSTRPPAGPQTRGHLHHWLPWLHKAPKKSLSCLEASVMFNLFYLLIWDLKGVTSEDPCV

SEQ ID NO: 9 - protein, 179 aa, Artificial Sequence
  Description of Artificial Sequence Synthetic polypeptide
MHHHHHHPVPSAPRALPPARGCHMAQFKSLSPQELQAFKTVRDAFEDSRLQKDWDCGGRRAALTCLLSPPQVWERPVALEAELALTLTVLEAMANSSLGRSLEQPLLTLQHVHSKLQACVPAQPTAGPRPRGRLHHWLHRLQEAQKKESQDCLEASVMFNLFRLLTRDLKCVASGDQCV

SEQ ID NO: 10 - DNA, 564 bp, Artificial Sequence
  Description of Artificial Sequence Synthetic polynucleotide
catatgcaccaccaccatcaccaccgtaccggtgcggttccggttccgagcgcgccgcgtgcgctgccgccggcgcgtggttgccatgttgcgcagttcaagagcctgagcccgcaggagctgcaagcgtttaaaaccgcgcgtgacgcgttcgaagatagctttctgccgaaggactgggattgcagcacccacctgttcccgcgtacccgtgacctgaaacacctgcaagtttgggagcgtccggttgcgctggaggcggaactggcgctgaccctgaccgtgctggaggcgatggcgaacagcagcctgggtcacagcctggaacagccgctgctgaccctgcaaaacatccacagcaagctgcaggcgtgcgttccggcgcaaccgaccgcgagcagccgtccgcgtggccgtctgcaccactggctgcaccgtctgcaggaagcgcgtaaagaaagccaagattgcctggaagcgagcgtgatgtttaacctgctgcgtctgctgacccgtgacctgaaatgcgttgcgagcggtgaccagtgcgtgtaatgaaagctt

SEQ ID NO: 11 - DNA, 552 bp, Artificial Sequence
  Description of Artificial Sequence Synthetic polynucleotide
catatgcaccaccaccatcaccaccgtaccggtgcggttccggttccgagcgcgccgcgtgcgctgccgccggcgcgtggttgccatgttgcgcagttcaagagcctgagcccgcaggagctgcaagcgtttaaaaccgcgcgtgacgcgttcgaagatagctttctgccgaaggactgggattgcagcacccacctgttcccgcgtacccgtgacctgaaacacctgcaagtttgggagcgtccggttgcgctggaggcggaactggcgctgaccctgaccgtgctggaggcgatggcgaacagcagcctgggtcacagcctggaacagccgctgctgaccctgcaaaacatccacagcaagctgcaggcgtgcgttccggcgcaaccgaccgcgagcagccgtccgcgtggccgtctgcaccactggctgcaccgtctgcaggaagcgcgtaaagaaagccaagattgcctggaagcgagcgttatgtttaatctgctgcgtctgctgacccgtgacctgaaatgcgttgcgagcggctaatgaaagctt

SEQ ID NO: 12 - DNA, 546 bp, Artificial Sequence
  Description of Artificial Sequence Synthetic polynucleotide
catatgcaccaccaccatcaccaccgtaccggtgcggttccggttccgagcgcgccgcgtgcgctgccgccggcgcgtggttgccatgtggcgcagttcaagagcctgagcccgcaggagctgcaagcgtttaaaaccgcgcgtgacgcgttcgaagatagctttctgccgaaggactgggattgcagcacccacctgttcccgcgtacccgtgacctgaaacacctgcaagtttgggagcgtccggttgcgctggaggcggaactggcgctgaccctgaccgtgctggaggcgatggcgaacagcagcctgggtcacagcctggaacagccgctgctgaccctgcaaaacatccacagcaagctgcaggcgtgcgttccggcgcaaccgaccgcgagcagccgtccgcgtggccgtctgcaccactggctgcaccgtctgcaggaagcgcgtaaagaaagccaagattgcctggaagcgagcgtgatgtttaatctgctgcgtctgctgacccgtgacctgaagtgcgttgcgtaatgaaagctt

SEQ ID NO: 13 - DNA, 549 bp, Artificial Sequence
  Description of Artificial Sequence Synthetic polynucleotide
catatgcaccaccaccaccaccaccccgtaccagaagctctaagagcattaccgggtgcgcgtggctgccacctggcgcagttcaagagcctgagcccgcaggcgctgcaagcgttcaagcgtgcgaaagacgcgtttgaggaaagcctgctggaggattggaactgcagcagccgtatctttccgcgtagccgtgacctgaaacagctgcaagtgtgggaacgtccggttgcgctggaggcggaagtggcgctgaccctgagcgttctgggtagcctggcgaacagcagcctgcacagcagcctggatcagccgctgcacaccctgcgtcacattcatgcgcagctgcaagcgtgcgtgccggcgcaaccgatggcgggtccgcgtccgcgtggccgtctgcaccactggctgcaccgtctgcaggaagcgcaaaagaaagaaccgcaaagctgcctggaagcgagcgttatgttcaacctgtttcgtctgctgacccgtgacctgaagtgcgtggcgagcggtgatctgtgcgtttaatgaaagctt

SEQ ID NO: 14 - DNA, 531 bp, Artificial Sequence
  Description of Artificial Sequence Synthetic polynucleotide
catatgcaccaccaccatcaccactttccccaagtaacacccaaaaaatcatgcagcctgagcaaataccaatttccggcgccgctggaactgaaagcggtttggcgtatgaaggagcagttcgaagacatcatgctgctgaccaaccgtaaatgcaacacccgtctgtttcaccgtaagtgggacatcgcggaactgagcgtgccggatcgtattaccctggttgaggcggaactggatctgaccattaccgtgctgaccaacccgaccacccaacgtctggcggagacctgccagcaaccgctggcgttcctgacccaggttcaagaagacctgcgtgattgcctggcgctggaggcgccgagccaccagccgagcggtaaactgcgtcactggctgcaaaagctggagaccgcgaagaaaaaggaaaccgcgggttgcctggaggcgagcgcgatcctgcacatttttcaggtgctgaacgacctgcgttgcgcggcgcaacgtgaggattgcacctaatgaaagctt

SEQ ID NO: 15 - DNA, 546 bp, Artificial Sequence
  Description of Artificial Sequence Synthetic polynucleotide
catatgcatcaccaccaccaccacggacccgtaccaaggagacacagagctctgccgggtgcgcgtggctgccacatcgcgcagtacaagagcctgagcccgggcgaacgtctggcgttcaaaaacgcgaccgacgcgttcgaggagagcttctggctgaagaaccacacctgccgtagccgtccgtttccgcgtacctgggatctgcgtcagctgcaagtgtgggagcgtccggttgcgctggaagcggagctgaacctgaccctgcgtgtgctggcgaacagcagcctgggtgacgttctggatcgtccgctgggcaccctgagccacattcatagcgaactgcaggcgtgcgtggcggcgcaaccgaccgagggtccgcgtccgcgtggccgtctgcaccagtggctgcgtcgtctgctggaagcgcaagaggaagagccgtggggttgcctggaggcgagcgttatgttcaacctgtttcgtctgctgaccaaagacctgacctgcgtggcgagcggcgatctgtgcgtttaatgaaagctt

SEQ ID NO: 16 - DNA, 549 bp, Artificial Sequence
  Description of Artificial Sequence Synthetic polynucleotide
catatgcaccaccaccatcaccacggacccgtaccaacatcaaaacccacaaccacccgtcgtggctgccacatggaccgtttccagagcctgagcccgcgtgagctggaagcgtttaagaaagcgaaggatgcgctggaggaaagcctgagctggaaaaactggagctgcagcagccgtctgttcccgcgtagccgtgacctgcgtctgctgcaggcgtgggagcgtccggtggcgctggaggcggaactggatctgaccctgaaagtgctggaaaacatgaccgacagcagcctgggtgttaccctggatcaaccgctgcgtaccctgcaccacattcatagcgagctgcaggcgtgcgttccggcgcaaccgaccgcggacccgcgtccgcatggtcgtctgcaccactggctgcaccgtctgcagaaggcgccgaaagagagccaaggctgcctggaagcgagcattaccttcaacctgtttcgtctgctgacccgtgacctgaagtgcgtggcgagccgtgatctgtgcgtttaatgaaagctt

SEQ ID NO: 17 - DNA, 441 bp, Artificial Sequence
  Description of Artificial Sequence Synthetic polynucleotide
catatgcaccaccaccaccaccacttacaaaaagaatcagtagctccccaagcgtggacctgccgtccgtgcctgttcccgaaaacccgtgagctgcgtcagctgcaagtgcgtgaacgtagcgttgcgctggaggcggaactggcgctgaccctgaaggttctgggcaccgttgcggatgcgagccagggtgacatcctggatcaaccgctgcacaccctgtgccacattcacagcgaactgtgggcgtgcgtgagcacccgtccgccggcgggtccgcaaacccgtggtcacctgcaccattggctgccgtggctgcacaaggcgccgaagaaaagcctgagctgcctggaggcgagcgttatgttcaacctgttttacctgctgatctgggacctgaaaggtgtgaccagcgaagatccgtgcgtttaatgaaagctt

SEQ ID NO: 18 - DNA, 552 bp, Artificial Sequence
  Description of Artificial Sequence Synthetic polynucleotide
catatgcaccaccaccatcaccaccccgtaccatcagctcccagagcactcccgccggcgcgtggttgccacatggcgcagttcaaaagcctgagcccgcaggagctgcaagcgttcaagaccgtgcgtgacgcgtttgaagatagccgtctgcagaaagattgggattgcggtggccgtcgtgcggcgctgacctgcctgctgagcccgccgcaagtttgggagcgtccggttgcgctggaggcggaactggcgctgaccctgaccgttctggaggcgatggcgaacagcagcctgggtcgtagcctggaacagccgctgctgaccctgcaacacgtgcacagcaagctgcaggcgtgcgttccggcgcaaccgaccgcgggtccgcgtccgcgtggccgtctgcaccactggctgcaccgtctgcaggaagcgcaaaagaaagaaagccaggactgcctggaagcgagcgtgatgttcaacctgttccgtctgctgacccgtgacctgaaatgcgttgcgagcggcgaccaatgcgtttaatgaaagctt

SEQ ID NO: 19 - protein, 24 aa, Homo sapiens
MPRLFFFHLLGVCLLLNQFSRAVA

SEQ ID NO: 20 - protein, 6 aa, Artificial Sequence
  Description of Artificial Sequence Synthetic 6xHis tag
HHHHHH